(19) United States
(12) Patent Application Publication
(10) Pub. No.: US 2023/0192699 A1
(43) Pub. Date: Jun. 22, 2023

(54) COMPOUNDS AS CASEIN KINASE INHIBITORS

(71) Applicant: GRITSCIENCE BIOPHARMACEUTICALS CO., LTD., Beijing (CN)

(72) Inventors: Enxing ZHOU, Beijing (CN); Yuan LIU, Beijing (CN); Hanping WANG, Beijing (CN); Jing WANG, Beijing (CN); Ning SHAO, Beijing (CN); Guanglong WU, Beijing (CN)

(73) Assignee: GRITSCIENCE BIOPHARMACEUTICALS CO., LTD., Beijing (CN)

(21) Appl. No.: 17/906,786

(22) PCT Filed: Mar. 26, 2021
(86) PCT No.: PCT/CN2021/083134

§ 371 Date: Sep. 20, 2022

(30) Foreign Application Priority Data
Mar. 27, 2020 (WO) PCT/CN2020/081804

(51) Int. Cl. C07D 487/04; C07D 519/00; C07D 498/04; A61P 35/00; C07D 403/04; C07D 401/04; C07D 491/048; C07D 471/04; C07D 405/14; C07D 413/14; C07D 413/04

(52) U.S. Cl. CPC C07D 487/04; C07D 519/00; C07D 498/04; A61P 35/00; C07D 403/04; C07D 401/04; C07D 491/048; C07D 471/04; C07D 405/14; C07D 413/14; C07D 413/04

(57) ABSTRACT

A compound of Formula I, Formula II, Formula III, Formula IV, Formula V, or Formula VI, or a pharmaceutically acceptable salt thereof. The structure of Formula I is:

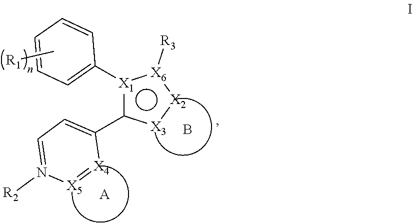

wherein R₁ is a halogen;
n is 0, 1, or 2;
X₁, X₂, X₃, X₄, X₅ and X₆ are each independently C or N;
R₂ is absent or O; and
R₃ is absent or —CN.

### BACKGROUND OF THE INVENTION

The circadian clock links our daily cycles of sleep and activity to the external environment. Deregulation of the clock is implicated in a number of human disorders, including depression, seasonal affective disorder, and metabolic disorders. For example, the circadian clock may regulate multiple downstream rhythms, such as those in sleep and awakening, body temperature, and hormone secretion (Ko and Takahashi, Hum Mol Gen 15: R271-R277.). Furthermore, diseases such as depression, seasonal affective disorder, and metabolic disorders, may have a circadian origin (Barnard and Nolan, PLoS Genet. 2008 May; 4(5): e1000040.).

Phosphorylation of circadian clock proteins is an essential element in controlling the cyclical rhythm of the clock. Casein kinase I delta (CK1δ) or CK1 epsilon (CK1ε) are closely related Ser-Thr protein kinases that serve as key clock regulators as demonstrated by mammalian mutations in each that dramatically alter the circadian period. Therefore, inhibitors of CK1δ/ε may be used for treating circadian disorders and other related disorders.

### SUMMARY OF THE INVENTION

The present application provides a series of novel compounds as potent inhibitors of CK1δ and/or CK1ε.

In one aspect, the present application provides a compound of Formula I, or a pharmaceutically acceptable salt thereof:

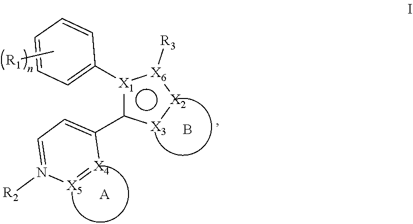

wherein R₁ is a halogen,
n is 0, 1, or 2,
X₁, X₂, X₃, X₄, X₅ and X₆ are each independently C or N,
R₂ is absent or O,
R₃ is absent or —CN,
A is absent,

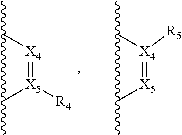

or ring A,
wherein R₄ is selected from the group consisting of: —NH₂, C₁-C₆ alkyl, alkyl-COO-alkyl, alkyl-NH-alkyl and alkyl-OH,
wherein R₅ is a halogen,
wherein ring A is a 4- to 7-membered cycloalkyl or heterocycloalkyl or a 5- to 6-membered heteroaryl, wherein up to 2 carbon atoms are replaced with a heteroatom selected from ═N— and —O—, and said ring A is optionally substituted with a R₈ substituent, R₈ is ═O,
B is a 4- to 7-membered cycloalkyl or heterocycloalkyl or a 5- to 6-membered heteroaryl, wherein up to 2 carbon atoms are replaced with a heteroatom selected from ═N— and —O—, and said B is optionally substituted with one or more R₆ substituents; said R₆ is further optionally substituted with a R₇ substituent,
wherein each R₆ is independently selected from the group consisting of: heterocycloalkyl, C₁-C₆ alkyl, CO-alkyl, CO-heterocycloalkyl, acyl-alkyl, benzyl, p-methoxybenzyl, O and CO-alkylcyano, and
wherein R₇ is C₁-C₆ alkyl.

In some embodiments, wherein R₁ is F.

In some embodiments, wherein n is 1.

In some embodiments, wherein n is 2.

In some embodiments, wherein R₄ is selected from the group consisting of: —NH₂, —CH₂—NH—CH₃, —CH₂OH, —CH₃ and —CH₂—O—CO—CH₃.

In some embodiments, wherein R₅ is F.

In some embodiments, wherein said A is selected from the group consisting of:

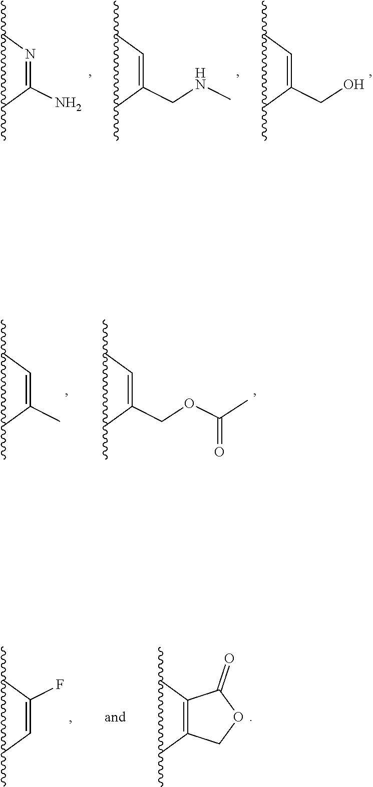

In some embodiments, wherein said A is

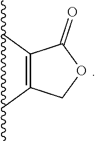

In some embodiments, wherein R₆ is selected from the group consisting of:

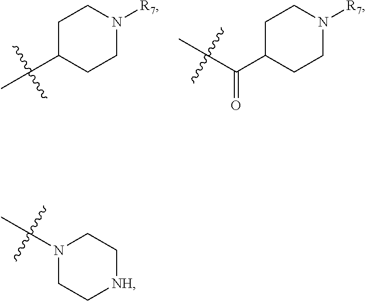

—CH₃, —CO—CH₃, —CO—NH-CH₃, —Bn, —PMB, —O, —CO—CH₂—CH₃, —CO—CH—(CH₃)₂ and —CO—CH₂—CN.

In some embodiments, wherein the number of R₆ is 0, 1 or 2.

In some embodiments, wherein R₇ is —CH₃.

In some embodiments, wherein B is selected from the group consisting of:

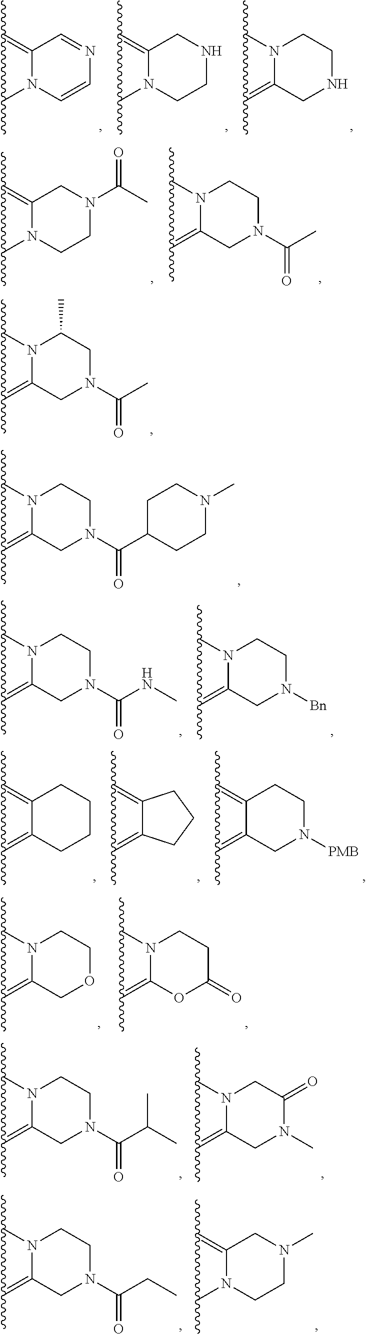

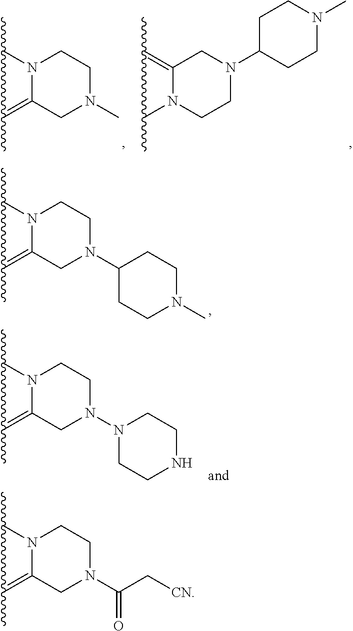

In some embodiments, wherein said A is

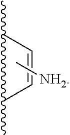

In some embodiments, wherein said A is

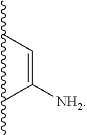

In some embodiments, wherein X₁ is C.

In some embodiments, wherein X₆ is N.

In one aspect, the present application provides a compound selected from the group consisting of:

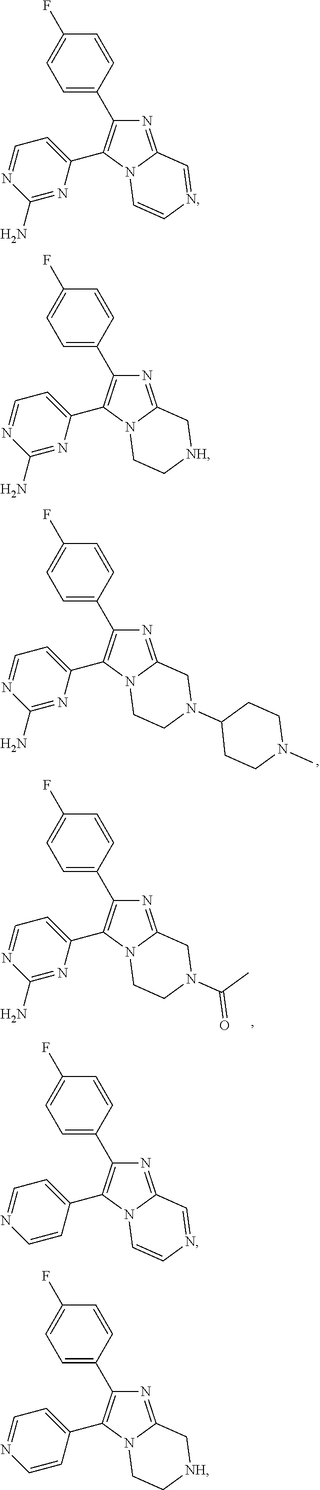

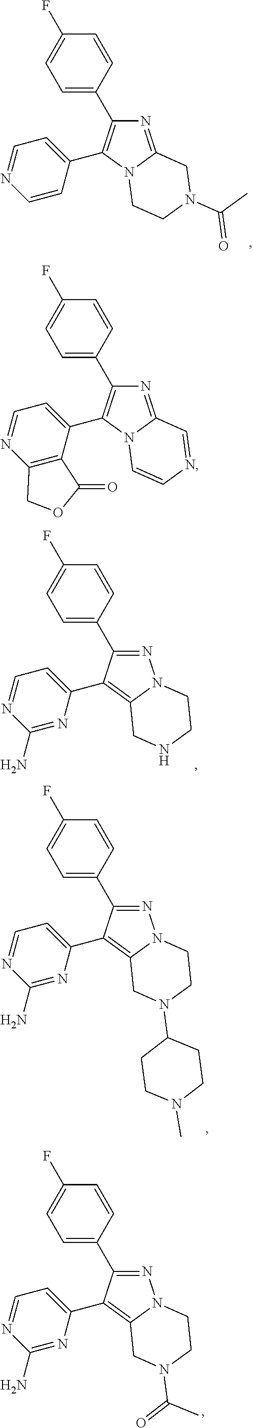

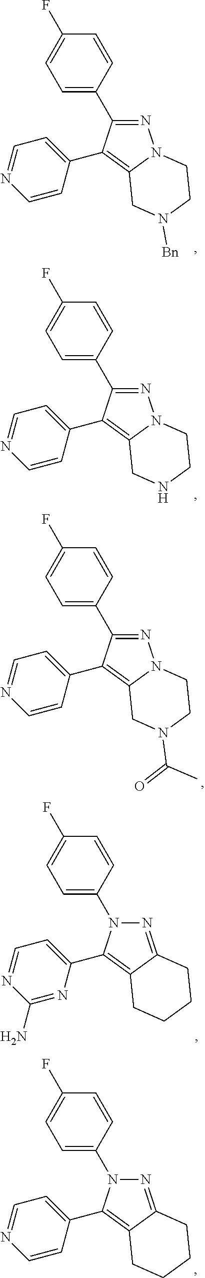

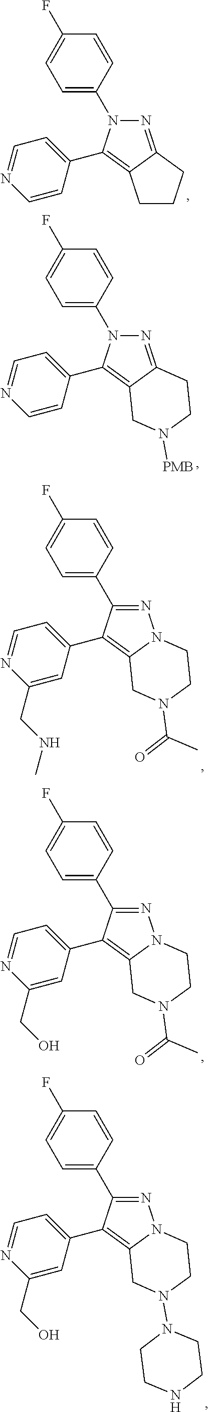

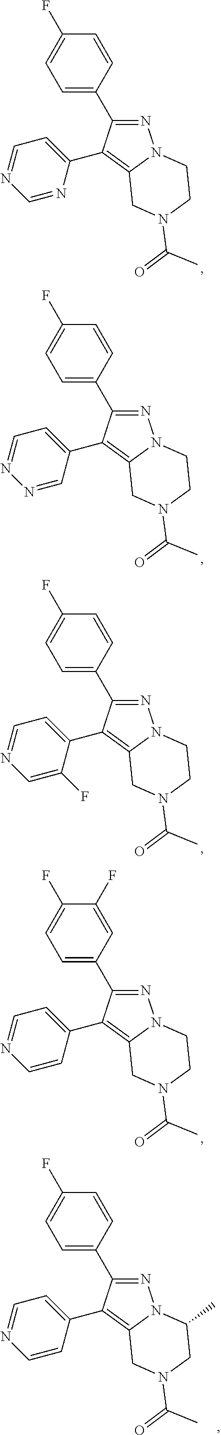

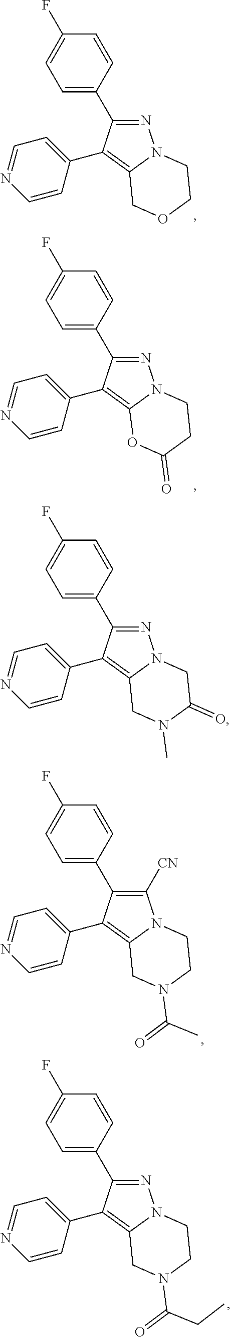

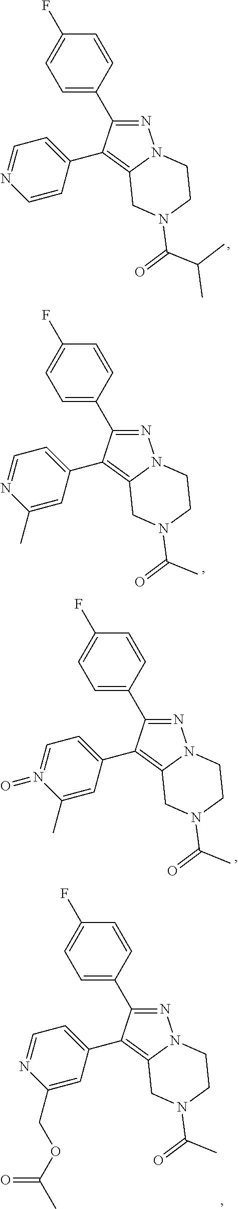 [SMILES: CC(C)C(=O)N1CCn2nc(-c3ccc(F)cc3)c(-c3ccncc3)c2C1 ; CC(=O)N1CCn2nc(-c3ccc(F)cc3)c(-c3ccnc(C)c3)c2C1 ; CC(=O)N1CCn2nc(-c3ccc(F)cc3)c(-c3cc[n+]([O-])c(C)c3)c2C1 ; CC(=O)OCc1cc(-c2c(-c3ccc(F)cc3)nn3c2CN(C(C)=O)CC3)ccn1]

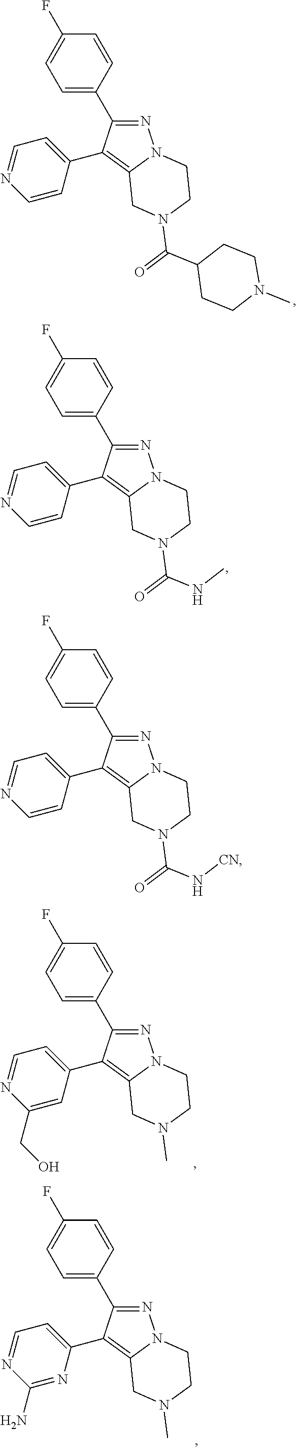

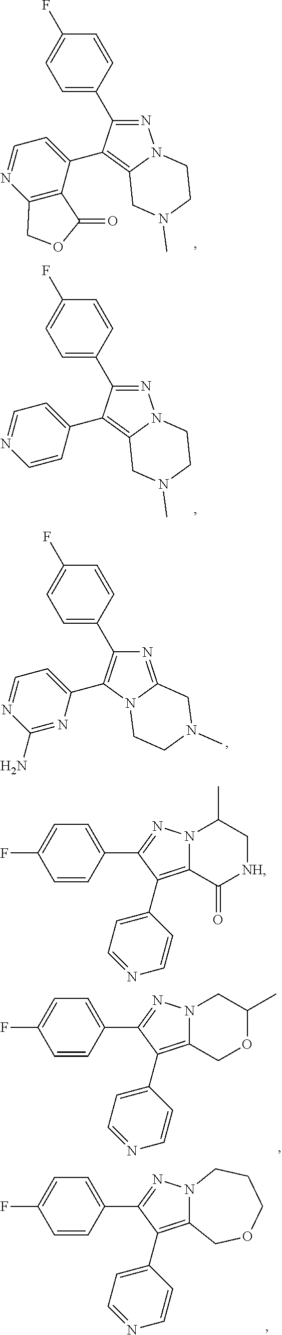

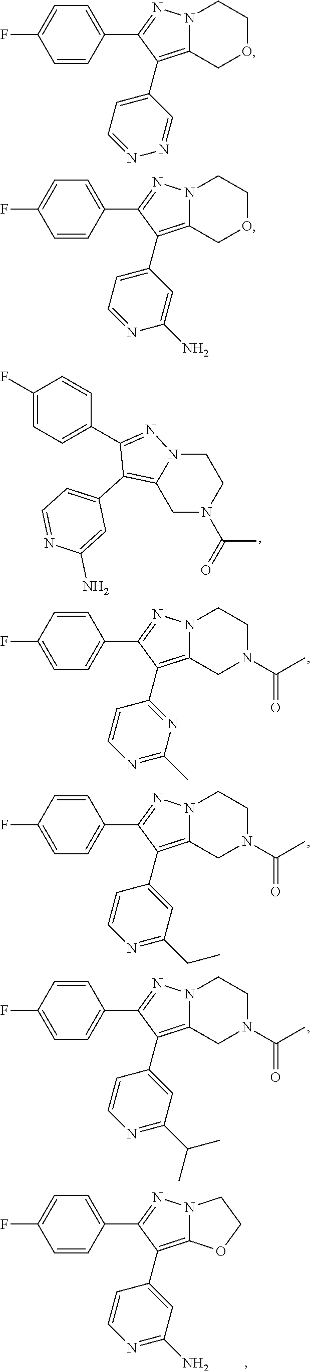

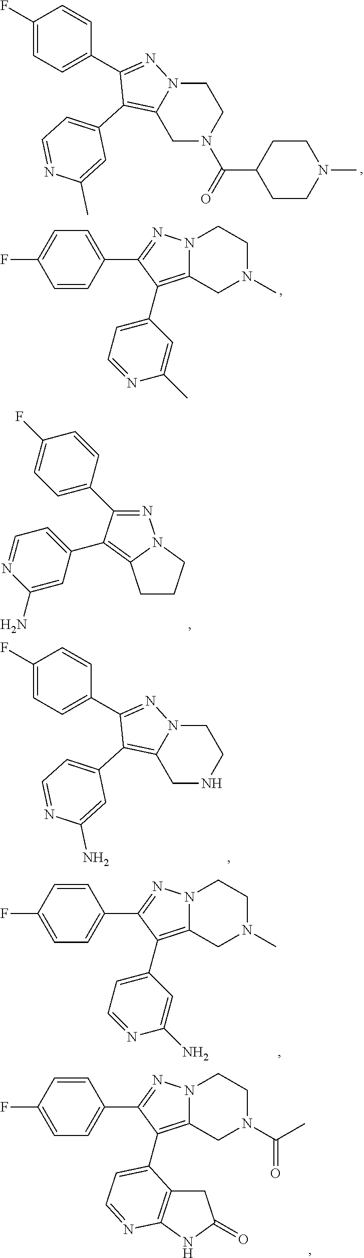

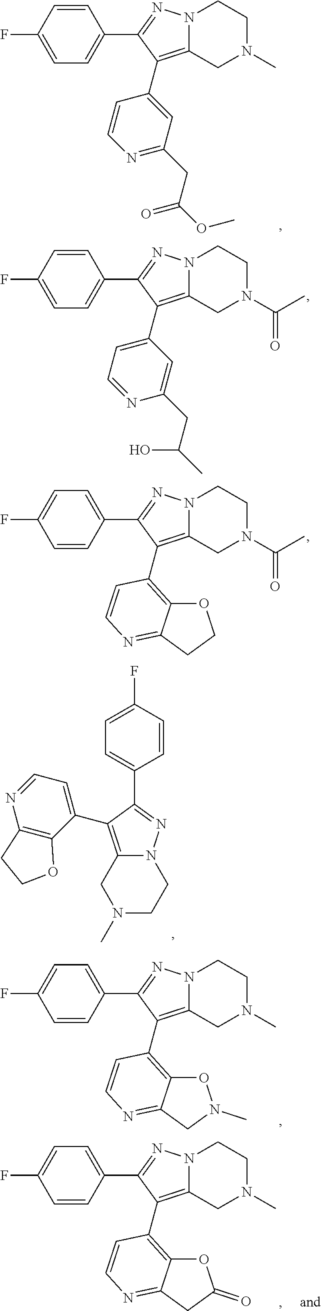

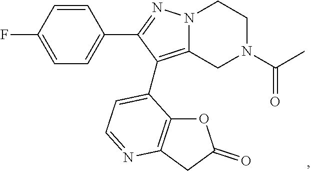 [SMILES: CC(=O)N1CCn2nc(-c3ccc(F)cc3)c(-c3ccnc4c3OC(=O)C4)c2C1]

or a pharmaceutically acceptable salt thereof.

In another aspect, the present application provides a compound of Formula II, or a pharmaceutically acceptable salt thereof:

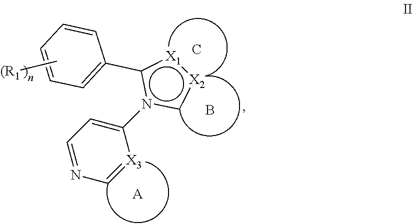

wherein R₁ is a halogen,

n is 0, 1, or 2,

X₁, X₂ and X₃ are each independently C or N,

A is absent,

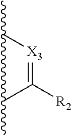

or ring A,

wherein ring A is a 4- to 7-membered cycloalkyl or heterocycloalkyl or a 5- to 6-membered heteroaryl, wherein up to 2 carbon atoms are replaced with a heteroatom selected from ═N— and —O—, and said ring A is optionally substituted with a R₃ substituent, R₃ is ═O;

wherein R₂ is —NH₂ or C₁-C₆ alkyl;

B is absent,

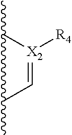

or ring B,

wherein R₄ is C₁-C₆ alkyl,

wherein ring B is a 4- to 7-membered cycloalkyl or heterocycloalkyl or a 5- to 6-membered heteroaryl, wherein up to 2 carbon atoms are replaced with a heteroatom selected from ═N— and —O—,

C is absent,

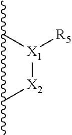

or ring C,

wherein R₅ is absent, a cyano or an amide group,

wherein ring C is a 4- to 7-membered cycloalkyl or heterocycloalkyl or a 5- to 6-membered heteroaryl, wherein up to 2 carbon atoms are replaced with a heteroatom selected from ═N— and —O—, and said ring C is optionally substituted with a R₆ substituent, and R₆ is ═O.

In some embodiments, wherein R₁ is F.

In some embodiments, wherein n is 1.

In some embodiments, wherein R₂ is —CH₃ or —NH₂.

In some embodiments, wherein A is selected from the group consisting of:

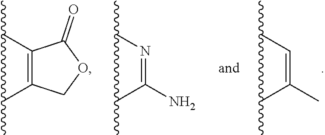

In some embodiments, wherein R₄ is —CH₃.

In some embodiments, wherein B is selected from the group consisting of:

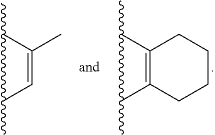

In some embodiments, wherein R₅ is —CN or —CO—NH₂.

In some embodiments, wherein C is selected from the group consisting of:

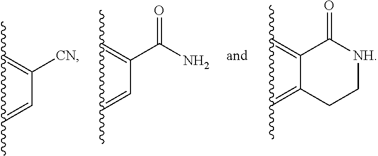

In another aspect, the present application provides a compound selected from the group consisting of:

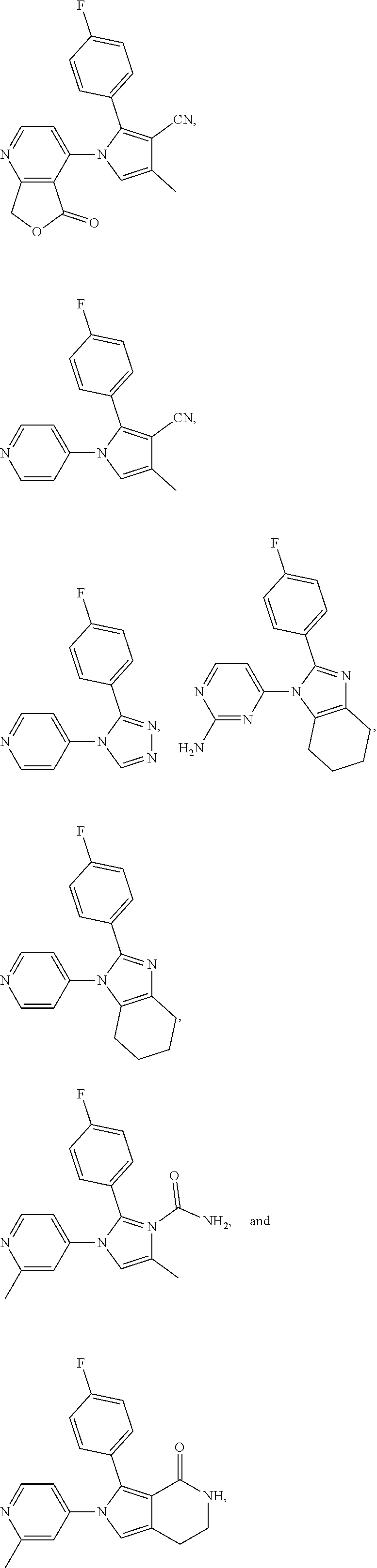

or a pharmaceutically acceptable salt thereof.

In another aspect, the present application provides a compound of Formula III, or a pharmaceutically acceptable salt thereof:

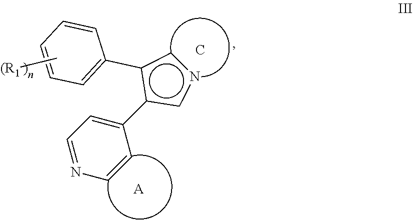

wherein R₁ is a halogen,

n is 0, 1, or 2,

A is absent or ring A,

wherein ring A is a 4- to 7-membered cycloalkyl or heterocycloalkyl or a 5- to 6-membered heteroaryl, wherein up to 2 carbon atoms are replaced with a heteroatom selected from ═N— and —O—, and said ring A is optionally substituted with a R₄ substituent, R₄ is ═O,

C is

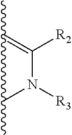

or ring C,

wherein R₂ is —CN, —CONH₂, or —COO-alkyl,

wherein R₃ is absent or C₁-C₆ alkyl,

wherein ring C is a 4- to 7-membered cycloalkyl or heterocycloalkyl or a 5- to 6-membered heteroaryl, wherein up to 2 carbon atoms are replaced with a heteroatom selected from ═N— and —O—, and said ring C is optionally substituted with a R₄ substituent, and R₄ is ═O.

The compound according to claim 28, wherein R₁ is F.

In some embodiments, wherein n is 1.

In some embodiments, wherein said ring A is a 5-membered heteroaryl.

In some embodiments, wherein 2 carbon atoms of said A are replaced with a heteroatom selected from ═N— and —O—.

In some embodiments, wherein said ring A is

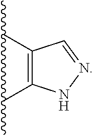

In some embodiments, wherein A is absent or

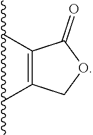

In some embodiments, wherein R₂ is —CN, —CONH₂, or —COO—CH₃.

In some embodiments, wherein R₃ is absent or —CH₃.

In some embodiments, wherein C is selected from the group consisting of:

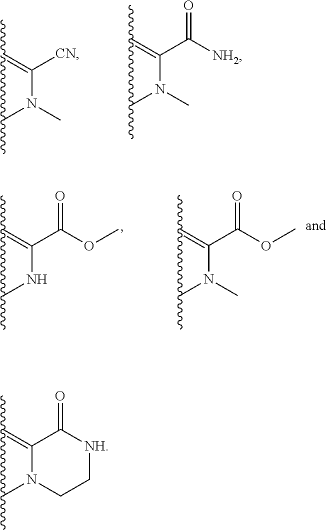

In another aspect, the present application provides a compound selected from the group consisting of:

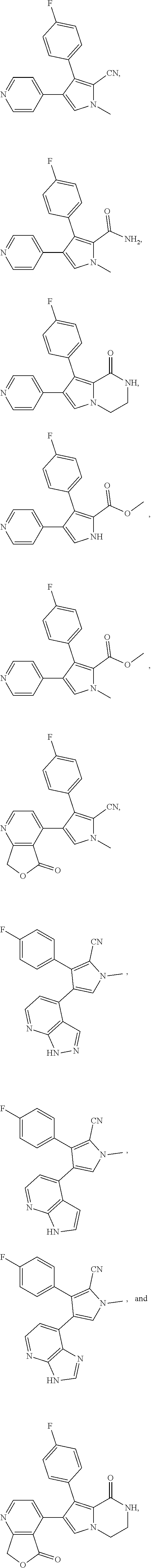

or a pharmaceutically acceptable salt thereof.

In another aspect, the present application provides a compound of Formula IV, or a pharmaceutically acceptable salt thereof:

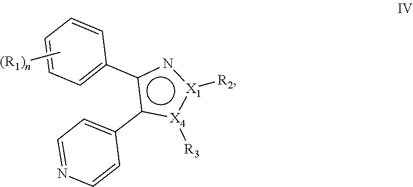

wherein R₁ is a halogen,

n is 0, 1, or 2,

X₁ is C, O, or N,

X₄ is C or N,

R₂ is absent or C₁-C₆ alkyl,

R₃ is absent, PMB, C₁-C₆ alkyl, or

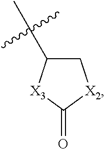

and

wherein X₂, X₃ are each independently C or O.

The compound according to claim 39, wherein R₁ is F.

In some embodiments, wherein n is 1.

In some embodiments, wherein the compound has the structure of

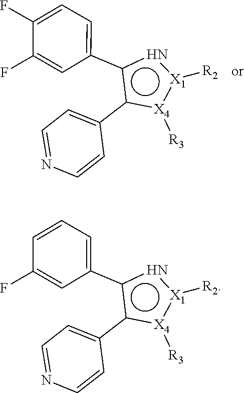

In some embodiments, wherein R₂ is absent or —CH₃.

In some embodiments, wherein R₃ is absent, PMB, —CH₃, or

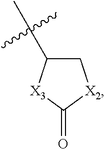

wherein X₂, X₃ are each independently C or O, wherein, PMB represents group

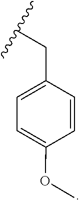 [SMILES: *Cc1ccc(OC)cc1]

In some embodiments, wherein X₂ is C and X₃ is O.

In some embodiments, wherein X₃ is C and X₂ is O.

In some embodiments, wherein X₄ is C.

In another aspect, the present application provides a compound selected from the group consisting of:

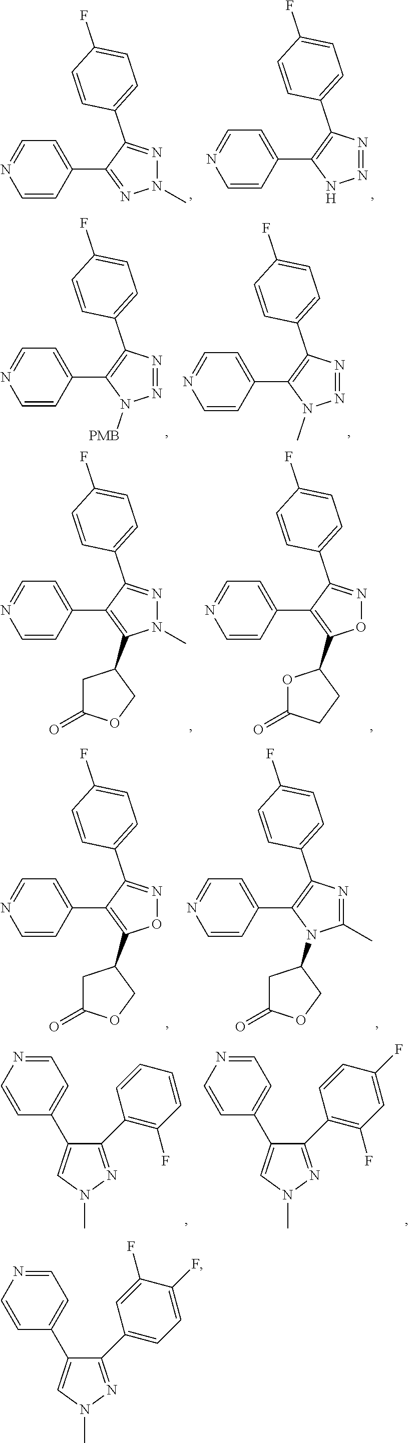

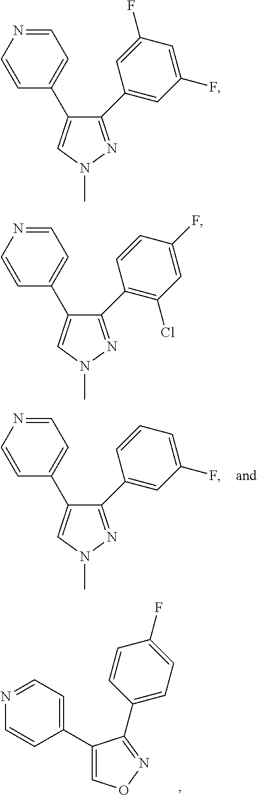

or a pharmaceutically acceptable salt thereof, wherein, PMB represents group

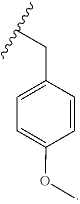 [SMILES: *Cc1ccc(OC)cc1]

In another aspect, the present application provides a compound of Formula V, or a pharmaceutically acceptable salt thereof:

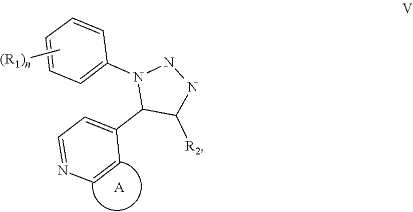

wherein R₁ is a halogen,

n is 0, 1, or 2,

R₂ is absent, —COO-alkyl, or —CO—R₃, wherein R₃ is a 4- to 7-membered cycloalkyl or heterocycloalkyl or a 5- to 6-membered heteroaryl, wherein up to 2 carbon atoms are replaced with a heteroatom selected from ═N— and —O—, and R₃ is optionally substituted with a R₄ substituent, R₄ is C₁-C₆ alkyl,

A is absent or ring A, and

wherein said ring A is a 4- to 7-membered cycloalkyl or heterocycloalkyl or a 5- to 6-membered heteroaryl, wherein up to 2 carbon atoms are replaced with a heteroatom selected from ═N— and —O—.

In some embodiments, wherein R₁ is F.

In some embodiments, wherein n is 1.

In some embodiments, wherein R₂ is —CO—R₃.

In some embodiments, wherein R₃ is

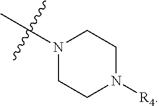

In some embodiments, wherein R₄ is —CH₃.

In some embodiments, wherein R₂ is selected from the group consisting of: —CO₂Et and

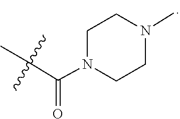 [SMILES: *C(=O)N1CCN(C)CC1]

In another aspect, the present application provides a compound selected from the group consisting of:

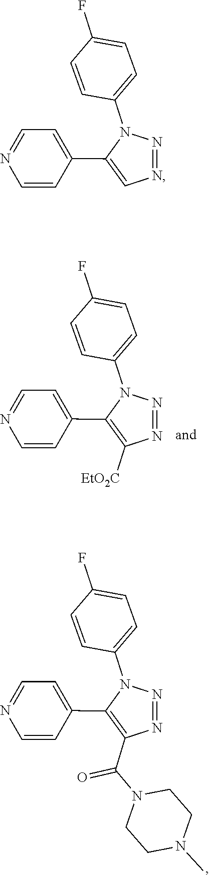 [SMILES: Fc1ccc(-n2nncc2-c2ccncc2)cc1 ; CCOC(=O)c1nnn(-c2ccc(F)cc2)c1-c1ccncc1 ; CN1CCN(C(=O)c2nnn(-c3ccc(F)cc3)c2-c2ccncc2)CC1]

or a pharmaceutically acceptable salt thereof.

In another aspect, the present application provides a compound of Formula VI, or a pharmaceutically acceptable salt thereof:

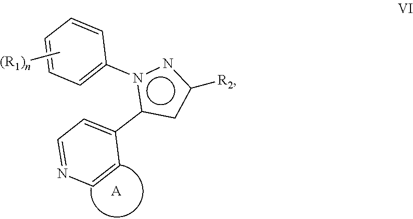

wherein R₁ is a halogen,

n is 0, 1, or 2,

R₂ is C₁-C₆ alkyl,

A is a 4- to 7-membered cycloalkyl or heterocycloalkyl or a 5- to 6-membered heteroaryl, wherein up to 2 carbon atoms are replaced with a heteroatom selected from ═N— and —O—, and said A is optionally substituted with a R₃ substituent, and R₃ is ═O.

The compound according to claim 57, wherein R₁ is F.

In some embodiments, wherein n is 1.

In some embodiments, wherein R₂ is —CH₃.

In some embodiments, wherein A is

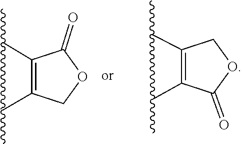

In another aspect, the present application provides a compound of

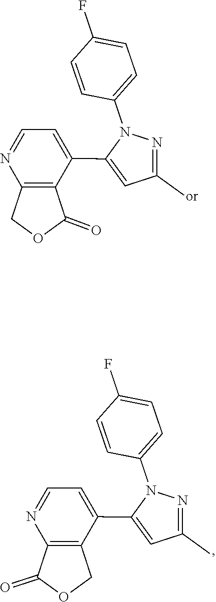 [SMILES: Cc1cc(-c2ccnc3c2C(=O)OC3)n(-c2ccc(F)cc2)n1 ; Cc1cc(-c2ccnc3c2COC3=O)n(-c2ccc(F)cc2)n1]

or a pharmaceutically acceptable salt thereof.

In another aspect, the present application provides a compound of

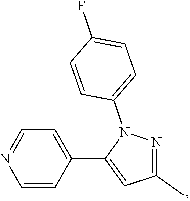 [SMILES: Cc1cc(-c2ccncc2)n(-c2ccc(F)cc2)n1]

or a pharmaceutically acceptable salt thereof.

In another aspect, the present application provides a pharmaceutical composition, comprising the compound of the present application or the pharmaceutically acceptable salt thereof, and optionally a pharmaceutically acceptable carrier.

In another aspect, the present application provides a method for inhibiting CK1 delta or CK1 epsilon activity, comprising administering an effective amount of the compound according to the present application, or a pharmaceutically acceptable salt thereof, or the pharmaceutical composition of the present application.

In some embodiments, wherein said method is an in vitro method, an ex vivo method, or an in vivo method.

In another aspect, the present application provides a method for treating a neurological and/or psychiatric disease or disorder in a mammal, which comprises administering to the mammal a therapeutically effective amount of a compound of the present application or a pharmaceutically acceptable salt thereof, or the pharmaceutical composition of the present application.

In some embodiments, wherein the disease or disorder is a mood disorder, a sleep disorder, or a circadian disorder.

In some embodiments, wherein the mood disorder is selected from the group consisting of: a depressive disorder and a bipolar disorder.

In another aspect, the present application provides a method for treating cancer in a mammal, which comprises administering to the mammal a therapeutically effective amount of a compound of the present application or a pharmaceutically acceptable salt thereof, or the pharmaceutical composition of the present application.

In some embodiments, wherein said cancer is a solid tumor, a blood cancer, or a lymphoma.

In some embodiments, wherein said cancer is selected from the group consisting of breast cancer, melanoma, leukemia, liver cancer, and brain cancer.

In some embodiments, the present application provides a compound selected from the group consisting of:
4-(2-(4-fluorophenyl)imidazo[1,2-a]pyrazin-3-yl)pyrimidin-2-amine,
4-(2-(4-fluorophenyl)-5,6,7,8-tetrahydroimidazo[1,2-a]pyrazin-3-yl)pyrimidin-2-amine,
4-(2-(4-fluorophenyl)-7-(1-methylpiperidin-4-yl)-5,6,7,8-tetrahydroimidazo[1,2-a]pyrazin-3-yl)pyrimidin-2-amine,
1-(3-(2-aminopyrimidin-4-yl)-2-(4-fluorophenyl)-5,6,7,8-tetrahydroimidazo[1,2-a]pyrazin-7-yl)ethan-1-one,
2-(4-fluorophenyl)-3-(pyridin-4-yl)imidazo[1,2-a]pyrazine,
2-(4-fluorophenyl)-3-(pyridin-4-yl)-5,6,7,8-tetrahydroimidazo[1,2-a]pyrazine,
1-(2-(4-fluorophenyl)-3-(pyridin-4-yl)-5,6-dihydroimidazo[1,2-a]pyrazin-7(8H)-yl)ethan-1-one,
4-(2-(4-fluorophenyl)imidazo[1,2-a]pyrazin-3-yl)furo[3,4-b]pyridin-5(7H)-one,
4-(2-(4-fluorophenyl)-4,5,6,7-tetrahydropyrazolo[1,5-a]pyrazin-3-yl)pyrimidin-2-amine,
4-(2-(4-fluorophenyl)-5-(1-methylpiperidin-4-yl)-4,5,6,7-tetrahydropyrazolo[1,5-a]pyrazin-3-yl)pyrimidin-2-amine,
1-(3-(2-aminopyrimidin-4-yl)-2-(4-fluorophenyl)-4, 5,6, 7-tetrahydropyrazolo [1,5-a]pyrazin-5(4H)-yl)ethan-1-one,
5-benzyl-2-(4-fluorophenyl)-3-(pyridin-4-yl)-4, 5,6,7-tetrahydropyrazolo[1,5-a]pyrazine,
2-(4-fluorophenyl)-3-(pyridin-4-yl)-4, 5, 6,7-tetrahydropyrazolo[1,5-a]pyrazine,
1-(2-(4-fluorophenyl)-3-(pyridin-4-yl)-6,7-dihydropyrazolo[1, 5-a]pyrazin-5(4H)-yl)ethan- 1-one,
4-(2-(4-fluorophenyl)-4,5,6,7-tetrahydro-2H-indazol-3-yl)pyrimidin-2-amine,
2-(4-fluorophenyl)-3-(pyridin-4-yl)-4, 5, 6,7-tetrahydro-2H-indazole,
2-(4-fluorophenyl)-3-(pyridin-4-yl)-2,4,5,6-tetrahydrocyclopenta[c]pyrazole,
2-(4-fluorophenyl)-5-(4-methoxybenzyl)-3-(pyridin-4-yl)-4, 5,6,7-tetrahydro-2H-pyrazolo[4,3-c]pyridine,
1-(2-(4-fluorophenyl)-3-(2-((methylamino)methyl)pyridin-4-yl)-4, 5,6, 7-tetrahydropyrazolo[1,5-a]pyrazin-5-yl)ethan-1-one,
1-(2-(4-fluorophenyl)-3-(2-(hydroxymethyl)pyridin-4-yl)-4, 5,6, 7-tetrahydropyrazolo[1,5-a]pyrazin-5-yl)ethan-1-one,
(4-(2-(4-fluorophenyl)-5-(piperazin-1-yl)-4,5,6,7-tetrahydropyrazolo[1, 5-a]pyrazin-3-yl)pyridin-2-yl)methanol,
1-(2-(4-fluorophenyl)-3-(pyrimidin-4-yl)-4,5,6,7-tetrahydropyrazolo[1, 5-a]pyrazin- 5-yl)ethan-1-one,
1-(2-(4-fluorophenyl)-3-(pyridazin-4-yl)-6,7-dihydropyrazolo[1, 5-a]pyrazin-5(4H)-yl)ethan-1-one,
1-(2-(4-fluorophenyl)-3-(3-fluoropyridin-4-yl)-6,7-dihydropyrazolo[1,5-a]pyrazin-5(4H)-yl)ethan one,
1-(2-(3,4-difluorophenyl)-3-(pyridin-4-yl)-6,7-dihydropyrazolo [1,5-a]pyrazin-5(4H)-yl)ethan-1-one,
(R)-1-(2-(4-fluorophenyl)-7-methyl-3-(pyridin-4-yl)-6, 7-dihydropyrazolo[1,5-a]pyrazin-5(4H)-yl)ethan-1-one,
2-(4-fluorophenyl)-3-(pyridin-4-yl)-6,7-dihydro-4H-pyrazolo[5, 1-c][1,4]oxazine,
2-(4-fluorophenyl)-3-(pyridin-4-yl)-6,7-dihydro-5H-pyrazolo[5, 1-b][1,3]oxazin-5-one,
2-(4-fluorophenyl)-5-methyl-3-(pyridin-4-yl)-4,5-dihydropyrazolo[1, 5-a]pyrazin-6(7H)-one,
2-acetyl-7-(4-fluorophenyl)-8-(pyridin-4-yl)-1,2,3,4-tetrahydropyrrolo[1,2-a]pyrazine-6-carbonitrile,
1-(2-(4-fluorophenyl)-3-(pyridin-4-yl)-6,7-dihydropyrazolo[1, 5-a]pyrazin-5 (4H)-yl)propan-1-one,
1-(2-(4-fluorophenyl)-3-(pyridin-4-yl)-6,7-dihydropyrazolo[1, 5-a]pyrazin-5(4H)-yl)-2-methylpropan-1-one,
1-(2-(4-fluorophenyl)-3-(2-methylpyridin-4-yl)-6,7-dihydropyrazolo[1, 5-a]pyrazin-5(4H)-yl)ethan-1-one,
4-(5-acetyl-2-(4-fluorophenyl)-4,5,6,7-tetrahydropyrazolo[1,5-a]pyrazin-3-yl)-2-methylpyridine-1-oxide,
(4-(5-acetyl-2-(4-fluorophenyl)-4,5,6,7-tetrahydropyrazolo[1,5-a]pyrazin-3-yl)pyridin-2-yl)methyl acetate,
(2-(4-fluorophenyl)-3-(pyridin-4-yl)-6,7-dihydropyrazolo[1,5-a]pyrazin-5(4H)-yl)(1-methylpiperidin-4-yl)methanone,
2-(4-fluorophenyl)-N-methyl-3-(pyridin-4-yl)-6,7-dihydropyrazolo[1,5-a]pyrazine-5(4H)-carboxamide,
3-(2-(4-fluorophenyl)-3-(pyridin-4-yl)-6,7-dihydropyrazolo[1,5-a]pyrazin-5(4H)-yl)-3-oxopropanenitrile,
(4-(2-(4-fluorophenyl)-5-methyl-4,5,6,7-tetrahydropyrazolo[1,5-a]pyrazin-3-yl)pyridin-2-yl)methanol,
4-(2-(4-fluorophenyl)-5-methyl-4,5,6,7-tetrahydropyrazolo[1,5-a]pyrazin-3-yl)pyrimidin-2-amine,
4-(2-(4-fluorophenyl)-5-methyl-4,5,6,7-tetrahydropyrazolo[1,5-a]pyrazin-3-yl)furo[3,4-b]pyridin-5(7H)-one,
2-(4-fluorophenyl)-5-methyl-3-(pyridin-4-yl)-4,5,6,7-tetrahydropyrazolo[1,5-a]pyrazine,
4-(2-(4-fluorophenyl)-7-methyl-5,6,7,8-tetrahydroimidazo[1,2-a]pyrazin-3-yl)pyrimidin-2-amine,
2-(4-fluorophenyl)-4-methyl-1-(5-oxo-5,7-dihydrofuro[3,4-b]pyridin-4-yl)-1H-pyrrole-3-carbonitrile,
2-(4-fluorophenyl)-4-methyl-1-(pyridin-4-yl)-1H-pyrrole-3-carbonitrile,
4-(3-(4-fluorophenyl)-4H-1,2,4-triazol-4-yl)pyridine,
4-(2-(4-fluorophenyl)-4,5,6,7-tetrahydro-1H-benzo[d]imidazol-1-yl)pyrimidin-2-amine,
2-(4-fluorophenyl)-1-(pyridin-4-yl)-4, 5, 6,7-tetrahydro-1H-benzo[d]imidazole,
2-(4-fluorophenyl)-4-methyl-1-(2-methylpyridin-4-yl)-1H-pyrrole-3-carboxamide,
3-(4-fluorophenyl)-2-(2-methylpyridin-4-yl)-2,5,6,7-tetrahydro-4H-pyrrolo[3,4-c]pyridin-4-one,
3-(4-fluorophenyl)-1-methyl-4-(pyridin-4-yl)-1H-pyrrole-2-carbonitrile,
3-(4-fluorophenyl)-1-methyl-4-(pyridin-4-yl)-1H-pyrrole-2-carboxamide,
8-(4-fluorophenyl)-7-(pyridin-4-yl)-3 ,4-dihydropyrrolo[1,2-a]pyrazin-1(2H)-one,
methyl 3-(4-fluorophenyl)-4-(pyridin-4-yl)-1H-pyrrole-2-carboxylate,
methyl 3-(4-fluorophenyl)-1-methyl-4-(pyridin-4-yl)-1H-pyrrole-2-carboxylate,
4-(5-(4-fluorophenyl)-2-methyl-2H-1,2,3-triazol-4-yl)pyridine,
4-(4-(4-fluorophenyl)-1H-1,2,3-triazol-5-yl)pyridine,
4-(4-(4-fluorophenyl)-1-(4-methoxybenzyl)-1H-1,2,3-triazol-5-yl)pyridine,
4-(4-(4-fluorophenyl)-1-methyl-1 H-1,2,3-triazol-5-yl)pyridine,
(S)-4-(3-(4-fluorophenyl)-1-methyl-4-(pyridin-4-yl)-1H-pyrazol-5-yl)dihydrofuran-2(3H)-one,
(R)-5-(3-(4-fluorophenyl)-4-(pyridin-4-ypisoxazol-5-yl)dihydrofuran-2(3H)-one,
(R)-4-(3-(4-fluorophenyl)-4-(pyridin-4-ypisoxazol-5-yl)dihydrofuran-2(3H)-one,
(R)-4-(4-(4-fluorophenyl)-2-methyl-5-(pyridin-4-yl)-1H-imidazol-1-yl)dihydrofuran-2(3H)-one,
4-(1-(4-fluorophenyl)-1H-1,2,3-triazol-5-yl)pyridine,
ethyl 1-(4-fluorophenyl)-5-(pyridin-4-yl)-1H-1,2,3-triazole-4-carboxylate,
(1-(4-fluorophenyl)-5-(pyridin-4-yl)-1H-1,2,3-triazol-4-yl)(4-methylpiperazin-1-yl)methanone,
3-(4-fluorophenyl)-1-methyl-4-(5-oxo-5 ,7-dihydrofuro[3,4-b]pyridin-4-yl)-1H-pyrrole-2-carbonitrile,
4-(1-(4-fluorophenyl)-3-methyl-1H-pyrazol-5-yl)furo[3 ,4-b]pyridin-5(7H)-one,
4-(1-(4-fluorophenyl)-3-methyl-1H-pyrazol-5-yl)furo[3,4-b]pyridin-7(5H)-one, and
4-(1-(4-fluorophenyl)-3-methyl-1H-pyrazol-5-yl)pyridine.

In a further aspect, the present application provides a method for inhibiting CK1 delta or CK1 epsilon activity, comprising administering an effective amount of the compound according to any one of Formulas I˜VI or the compound of

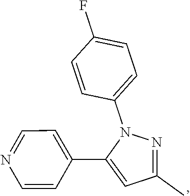 [SMILES: Cc1cc(-c2ccncc2)n(-c2ccc(F)cc2)n1]

or a pharmaceutically acceptable salt thereof.

In some embodiments, the method is an in vitro method, an ex vivo method, or an in vivo method.

In another aspect, the present application provides a method for treating a neurological and/or psychiatric disease or disorder in a mammal, which comprises administering to the mammal a therapeutically effective amount of a compound of any one of Formulas I—VI or the compound of

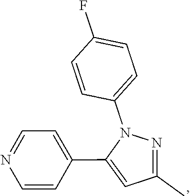 [SMILES: Cc1cc(-c2ccncc2)n(-c2ccc(F)cc2)n1]

or a pharmaceutically acceptable salt thereof.

In some embodiments, the disease or disorder is a mood disorder, a sleep disorder, or a circadian disorder.

In some embodiments, the mood disorder is selected from the group consisting of: a depressive disorder and a bipolar disorder.

In another aspect, the present application provides a method for treating cancer in a mammal, which comprises administering to the mammal a therapeutically effective amount of a compound of any one of Formulas I˜VI or the compound of

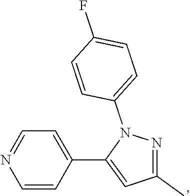 [SMILES: Cc1cc(-c2ccncc2)n(-c2ccc(F)cc2)n1]

or a pharmaceutically acceptable salt thereof.

In some embodiments, the cancer is selected from the group consisting of breast cancer, melanoma, leukemia, liver cancer, and brain cancer.

In a further aspect, the present application provides a pharmaceutical composition comprising a compound of any one of Formulas I˜VI or the compound of

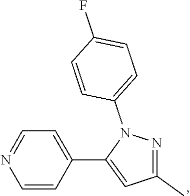 [SMILES: Cc1cc(-c2ccncc2)n(-c2ccc(F)cc2)n1]

or a pharmaceutically acceptable salt thereof, and optionally a pharmaceutically acceptable carrier.

The present application further provides kits that are suitable for use in performing the methods of treatment described above. In one embodiment, the kit contains a first dosage form comprising one or more of the compounds of the present application and a container for the dosage, in quantities sufficient to carry out the methods of the present application.

In another embodiment, the kit of the present application comprises one or more compounds of the invention.

In another aspect, the present application also provides novel intermediates useful for preparing the compounds of the present application.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates the synthetic scheme of compound 1-1.

FIG. 2 illustrates the synthetic scheme of compound 2-2.

FIG. 3 illustrates the synthetic scheme of compounds 1-5, 1-6, 1-7 and 1-8.

FIG. 4 illustrates the synthetic scheme of compound 6-1.

FIG. 5 illustrates the synthetic scheme of compounds 1-12, 1-13 and 1-14.

FIG. 6 illustrates the synthetic scheme of compound 1-16.

FIG. 7 illustrates the synthetic scheme of compound 2-5.

FIG. 8 illustrates the synthetic scheme of compounds 3-6, 3-7, 3-8 and 3-9.

FIG. 9 illustrates the synthetic scheme of compounds 3-1, 3-2, 3-4 and 3-5.

FIG. 10 illustrates the synthetic scheme of compound 4-1.

FIG. 11 illustrates the synthetic scheme of compound 5-1.

FIG. 12 illustrates the synthetic scheme of compound 1-31.

FIG. 13 illustrates the synthetic scheme of compound 1-32.

FIG. 14 illustrates the synthetic scheme of compounds 1-33 and 1-34.

FIG. 15 illustrates the synthetic scheme of compound 1-27.

FIG. 16 illustrates the synthetic scheme of compound 1-42.

FIG. 17 illustrates the synthetic scheme of compound 1-36.

FIG. 18 illustrates the synthetic scheme of compound 1-37.

FIG. 19 illustrates the synthetic scheme of compound 1-35.

FIG. 20 illustrates the synthetic scheme of compound 1-24.

FIG. 21 illustrates the synthetic scheme of compound 7-1.

FIGS. 22-24 illustrate the synthetic schemes of compounds 1-44 to 1-47.

FIG. 25 illustrates the synthetic scheme of compound 5-2.

FIG. 26 illustrates the synthetic scheme of compound 1-48.

FIG. 27 illustrates the synthetic scheme of compound 5-3.

FIGS. 28-29 illustrate the synthetic schemes of compounds 1-49 to 1-50.

FIG. 30 illustrates the synthetic scheme of compound 5-4.

FIGS. 31-36 illustrate the synthetic schemes of compounds 1-51 to 1-56.

FIG. 37 illustrates the synthetic scheme of compound 5-5.

FIGS. 38-49 illustrate the synthetic schemes of compounds 1-57 to 1-68.

FIGS. 50-56 illustrate the synthetic schemes of compounds 3-14 to 3-20.

FIGS. 57-60 demonstrate the relative proliferation (%) of cells after administering Compounds 1-1, 2-5, 1-14 and 3-8, respectively.

FIG. 61 demonstrates the relative migration (%) of cells after administering Compounds 2-5, 1-1, 2-2, 1-14 and 3-7.

FIGS. 62-67 demonstrate the migrated cells after administering Compound 2-5, DMSO, Compounds 1-1, 2-2, 1-14 and 3-7, respectively.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Definitions

As used herein, the term “alkyl” generally refers to a linear or branched-chain saturated hydrocarbyl substituent (i.e., a substituent obtained from a hydrocarbon by removal of a hydrogen) containing from one to twenty carbon atoms; for example, from one to twelve carbon atoms; in another example, from one to ten carbon atoms; in another embodiment, from one to six carbon atoms; and in another embodiment, from one to four carbon atoms (such as 1, 2, 3 or more carbon atoms). Examples of such substituents include e.g., methyl, ethyl, propyl (including n-propyl and isopropyl), butyl (including n-butyl, isobutyl, sec-butyl and terf-butyl), pentyl, isoamyl, hexyl and the like. In some instances, the number of carbon atoms in a hydrocarbyl substituent (i.e., alkyl, alkenyl, cycloalkyl, aryl, etc.) is indicated by the prefix “Cₐ-C_{b}” wherein a is the minimum and b is the maximum number of carbon atoms in the substituent. Thus, for example, “C₁-C₆ alkyl” refers to an alkyl substituent containing from 1 to 6 carbon atoms. For example, alkyl may be optionally further substituted.

As used herein, the term “cycloalkyl” generally refers to a carbocyclic substituent obtained by removing a hydrogen from a saturated carbocyclic molecule and having three to fourteen carbon atoms. In one embodiment, a cycloalkyl substituent has three to ten carbon atoms. Cycloalkyl may be a single ring, which typically contains from 4 to 7 ring atoms. Examples of cycloalkyl include cyclopropyl, cyclobutyl, cyclopentyl and cyclohexyl. Alternatively, cycloalkyl may be 2 or 3 rings fused together, such as bicyclo[4.2.0]octane and decalinyl and may also be referred to as “bicycloalkyl”. For example, cycloalkyl may be optionally further substituted.

As used herein, the term “cycloalkyl” also includes substituents that are fused to a C₆-C₁₀ aromatic ring or to a 5- to 10-membered heteroaromatic ring, wherein a group having such a fused cycloalkyl group as a substituent is bound to a carbon atom of the cycloalkyl group. When such a fused cycloalkyl group is substituted with one or more substituents, the one or more substituents, unless otherwise specified, are each bound to a carbon atom of the cycloalkyl group. The fused C₆-C₁₀ aromatic ring or 5-10-membered heteroaromatic ring may be optionally further substituted. For example, cycloalkyl may be optionally further substituted.

As used herein, the term “halogen” generally refers to fluorine (which may be depicted as —F), chlorine (which may be depicted as —Cl), bromine (which may be depicted as —Br), or iodine (which may be depicted as —I). In one embodiment, the halogen is chlorine. In another embodiment, the halogen is fluorine. In another embodiment, the halogen is bromine.

As used herein, the term “heterocycloalkyl” generally refers to a substituent obtained by removing a hydrogen from a saturated or partially saturated ring structure containing a total of 4 to 14 ring atoms, wherein at least one of the ring atoms is a heteroatom (e.g., an atom other than C), such as oxygen, nitrogen or sulfur. For example, as used herein, the term “4- to 7-membered heterocycloalkyl” means the substituent is a single ring with 4 to 7 total members. A heterocycloalkyl alternatively may comprise 2 or 3 rings fused together, wherein at least one such ring contains a heteroatom as a ring atom (i.e., nitrogen, oxygen or sulfur). In a group that has a heterocycloalkyl substituent, the ring atom of the heterocycloalkyl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heterocycloalkyl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. For example, heterocycloalkyl may be optionally further substituted.

As used herein, the term “heterocycloalkyl” also includes substituents that are fused to a C₆₋₁₀ aromatic ring or to a 5- to 10-membered heteroaromatic ring, wherein a group having such a fused heterocycloalkyl group as a substituent is bound to a heteroatom of the heterocycloalkyl group or to a carbon atom of the heterocycloalkyl group. When such a fused heterocycloalkyl group is substituted with one or more substituents, the one or more substituents, unless otherwise specified, are each bound to a heteroatom of the heterocycloalkyl group or to a carbon atom of the heterocycloalkyl group. The fused C₆-C₁₀ aromatic ring or 5- to 10-membered heteroaromatic ring may be optionally further substituted. For example, heterocycloalkyl may be optionally further substituted.

As used herein, the term “heteroaryl” generally refers to an aromatic ring structure containing from 5 to 14 ring atoms in which at least one of the ring atoms is a heteroatom (for example, oxygen, nitrogen, or sulfur), with the remaining ring atoms being independently selected from the group consisting of carbon, oxygen, nitrogen, and sulfur. A heteroaryl may be a single ring or 2 or 3 fused rings. Examples of heteroaryl substituents include but are not limited to: 6-membered ring substituents such as pyridyl, pyrazyl, pyrimidinyl, and pyridazinyl; 5-membered ring substituents such as triazolyl, imidazolyl, furanyl, thiophenyl, pyrazolyl, oxazolyl, isoxazolyl, thiazolyl, 1,2,3-, 1, 2, 4-, 1,2,5-, or 1,3,4-oxadiazolyl and isothiazolyl; 6/5-membered fused ring substituents such as benzothiofuranyl, isobenzothiofuranyl, benzisoxazolyl, benzoxazolyl, purinyl, and anthranilyl; and 6/6-membered fused ring substituents such as quinolinyl, isoquinolinyl, cinnolinyl, quinazolinyl, and 1,4-benzoxazinyl. In a group that has a heteroaryl substituent, the ring atom of the heteroaryl substituent that is bound to the group may be the at least one heteroatom, or it may be a ring carbon atom, where the ring carbon atom may be in the same ring as the at least one heteroatom or where the ring carbon atom may be in a different ring from the at least one heteroatom. Similarly, if the heteroaryl substituent is in turn substituted with a group or substituent, the group or substituent may be bound to the at least one heteroatom, or it may be bound to a ring carbon atom, where the ring carbon atom may be in the same ring as the at least one heteroatom or where the ring carbon atom may be in a different ring from the at least one heteroatom. For example, heteroaryl may be optionally further substituted.

As used herein, the term “heteroaryl” also includes substituents such as pyridyl and quinolinyl that are fused to a C₄₋₁₀ carbocyclic ring, such as a C₅ or a C₆ carbocyclic ring, or to a 4-10-mennbered heterocyclic ring, wherein a group having such a fused heteroaryl group as a substituent is bound to an aromatic carbon of the heteroaryl group or to a heteroatom of the heteroaryl group. When such a fused heteroaryl group is substituted with one or more substituents, the one or more substituents, unless otherwise specified, are each bound to an aromatic carbon of the heteroaryl group or to a heteroatom of the heteroaryl group. The fused C₄₋₁₀ carbocyclic or 4-10-membered heterocyclic ring may be optionally further substituted. For example, heteroaryl may be optionally further substituted.

As used herein, the term “aryl” generally refers to an aromatic substituent containing one ring or two or three fused rings. The aryl substituent may have six to eighteen carbon atoms. As an example, the aryl substituent may have six to fourteen carbon atoms. The term “aryl” may refer to substituents such as phenyl, naphthyl and anthracenyl. The term “aryl” may also include substituents such as phenyl, naphthyl and anthracenyl that are fused to a C₄₋₁₀ carbocyclic ring, such as a C₅ or a C₆ carbocyclic ring, or to a 4- to 10-membered heterocyclic ring, wherein a group having such a fused aryl group as a substituent is bound to an aromatic carbon of the aryl group. When such a fused aryl group is substituted with one more substituent, the one or more substituents, unless otherwise specified, are each bound to an aromatic carbon of the fused aryl group. The fused C₄₋₁₀ carbocyclic or 4- to 10-membered heterocyclic ring may optionally be further substituted. Examples of aryl groups include accordingly phenyl, naphthalenyl, tetrahydronaphthalenyl (also known as “tetralinyl”), indenyl, isoindenyl, indanyl, anthracenyl, phenanthrenyl, benzonaphthenyl (also known as “phenalenyl”), and fluorenyl. For example, aryl may be optionally further substituted.

In some instances, the number of atoms in a cyclic substituent containing one or more heteroatoms (i.e., heteroaryl or heterocycloalkyl) is indicated by the prefix “X-Y-membered”, wherein wherein x is the minimum and y is the maximum number of atoms forming the cyclic moiety of the substituent. Thus, for example, 5- to 8-membered heterocycloalkyl refers to a heterocycloalkyl containing from 5 to 8 atoms, including one or more heteroatoms, in the cyclic moiety of the heterocycloalkyl.

As used herein, the term “hydrogen” generally refers to a hydrogen substituent, and may be depicted as —H.

As used herein, the term “hydroxy” or “hydroxyl” generally refers to —OH. When used in combination with another term(s), the prefix “hydroxy” generally indicates that the substituent to which the prefix is attached is substituted with one or more hydroxy substituents. Compounds bearing a carbon to which one or more hydroxy substituents are attached include, for example, alcohols, enols and phenol. For example, hydroxy may be optionally further substituted.

As used herein, the term “cyano” (also referred to as “nitrile”) generally means —CN.

A substituent is “substitutable” or can be “substituted” if it comprises at least one carbon or nitrogen atom that is bonded to one or more hydrogen atoms. Thus, for example, hydrogen, halogen, and cyano do not fall within this definition.

If a substituent is described as being “substituted,” a non-hydrogen substituent is in the place of a hydrogen substituent on a carbon or nitrogen of the substituent. Thus, for example, a substituted alkyl substituent is an alkyl substituent wherein at least one non-hydrogen substituent is in the place of a hydrogen substituent on the alkyl substituent. To illustrate, monofluoroalkyl is alkyl substituted with a fluoro substituent, and difluoroalkyl is alkyl substituted with two fluoro substituents. It should be recognized that if there is more than one substitution on a substituent, each non-hydrogen substituent may be identical or different (unless otherwise stated).

As used herein, the terms “substituent,” “radical,” and “group” may be used interchangeably.

If a substituent is described as being “optionally substituted,” the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more of a list of substituents, one or more of the hydrogens on the nitrogen (to the extent there are any) may each be replaced with an independently selected optional substituent. One exemplary substituent may be depicted as —NR′R″, wherein R′ and R″ together with the nitrogen atom to which they are attached may form a heterocyclic ring comprising 1 or 2 heteroatoms independently selected from oxygen, nitrogen, and sulfur, wherein said heterocycloalkyl moiety may be optionally substituted. The heterocyclic ring formed from R′ and R″ together with the nitrogen atom to which they are attached may be partially or fully saturated, or aromatic. In one embodiment, the heterocyclic ring consists of 4 to 10 atoms.

If substituents are described as being “independently selected” from a group, each substituent is selected independent of the other(s). Each substituent therefore may be identical to or different from the other substituent(s).

As used herein, the term “Formula I” (or Formula II, Formula III, Formula IV, Formula V, or Formula VI) may be hereinafter referred to as a “compound(s) of the invention”. Such terms are also defined to include all forms of the compound of Formula I (or Formula II, Formula III, Formula IV, Formula V, or Formula VI), including hydrates, solvates, isomers, crystalline and non-crystalline forms, isomorphs, polymorphs, and metabolites thereof. For example, the compounds of Formula I, or pharmaceutically acceptable salts thereof, may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of “Formula I” (or Formula II, Formula III, Formula IV, Formula V, or Formula VI) may have asymmetric carbon atoms. The carbon-carbon bonds of the compounds of Formula I may be depicted herein using a solid line, a solid wedge, or a dotted wedge. The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the present application may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I (or Formula II, Formula III, Formula IV, Formula V, or Formula VI) can exist as enantiomers and diastereomers or as racemates and mixtures thereof. The use of a solid line to depict bonds to one or more asymmetric carbon atoms in a compound of Formula I (or Formula II, Formula III, Formula IV, Formula V, or Formula VI) and the use of a solid or dotted wedge to depict bonds to other asymmetric carbon atoms in the same compound is meant to indicate that a mixture of diastereomers is present.

The compounds of the present application (e.g., the compounds of Formula I, Formula II, Formula III, Formula IV, Formula V, or Formula VI) may exist as clathrates or other complexes. Included within the scope of the invention are complexes such as clathrates, drug- host inclusion complexes wherein, in contrast to the aforementioned solvates, the drug and host are present in stoichiometric or non-stoichiometric amounts. Also included are complexes of Formula I (or Formula II, Formula III, Formula IV, Formula V, Formula VI) containing two or more organic and/or inorganic components which may be in stoichiometric or non-stoichiometric amounts. The resulting complexes may be ionized, partially ionized, or non-ionized. For a review of such complexes, see J. Pharm. Sci., 64 (8), 1269-1288 by Haleblian (August 1975).

Stereoisomers of Formula I (or Formula II, Formula III, Formula IV, Formula V, Formula VI) include cis and trans isomers, optical isomers such as R and S enantiomers, diastereomers, geometric isomers, rotational isomers, conformational isomers, and tautomers of the compounds of Formula I (or Formula II, Formula III, Formula IV, Formula V, Formula VI), including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

The compounds of Formula I (or Formula II, Formula III, Formula IV, Formula V, Formula VI) may exhibit the phenomena of tautomerism and structural isomerism. For example, the compounds of Formula I may exist in several tautomeric forms, including the enol and imine forms, and the keto and enamine forms, and geometric isomers and mixtures thereof. All such tautomeric forms are included within the scope of compounds of Formula I (or Formula II, Formula III, Formula IV, Formula V, Formula VI). Tautomers exist as mixtures of a tautomeric set in solution. In solid form, usually one tautomer predominates. Even though one tautomer may be described, the present invention includes all tautomers of the compounds of Formula I (or Formula II, Formula III, Formula IV, Formula V, Formula VI).

The present invention also includes isotopically-labeled compounds, which are identical to those recited in Formula I (or Formula II, Formula III, Formula IV, Formula V, Formula VI) above, but for the fact that one or more atoms are replaced by an atom having an atomic mass or mass number different from the atomic mass or mass number usually found in nature. Examples of isotopes that may be incorporated into compounds of Formula I (or Formula II, Formula III, Formula IV, Formula V, Formula VI) include isotopes of hydrogen, carbon, nitrogen, oxygen, phosphorus, fluorine and chlorine, such as, but not limited to, ²H, ³H, ¹³C, ¹⁴C, ¹⁵N, ¹⁸O, ¹⁷O, ³¹P, ³²P, ³⁵S, ¹⁸F, and ³⁶Cl. Certain isotopically-labeled compounds of Formula I (or Formula II, Formula III, Formula IV, Formula V, Formula VI), for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are used for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be used in some circumstances. Isotopically-labeled compounds of Formula I (or Formula II, Formula III, Formula IV, Formula V, Formula VI) may generally be prepared by carrying out the procedures disclosed in the Schemes and/or in the Examples below, by substituting an isotopically-labeled reagent for a non-isotopically-labeled reagent.

The compounds of the present application may be used in the form of salts derived from inorganic or organic acids. Depending on the particular compound, a salt of the compound may be advantageous due to one or more of the salt's physical properties, such as enhanced pharmaceutical stability in differing temperatures and humidity, or a desirable solubility in water or oil. In some instances, a salt of a compound also may be used as an aid in the isolation, purification, and/or resolution of the compound.

As used herein, the term “treating”, unless otherwise indicated, generally means reversing, alleviating, inhibiting the progress of, or preventing the disorder or condition to which such term applies, or one or more symptoms of such disorder or condition. The term “treatment”, as used herein, unless otherwise indicated, generally refers to the act of treating as “treating” is defined immediately above. The term “treating” may also include adjuvant and neo-adjuvant treatment of a subject.

Compounds

In one aspect, the present application provides a compound of Formula I, or a pharmaceutically acceptable salt thereof:

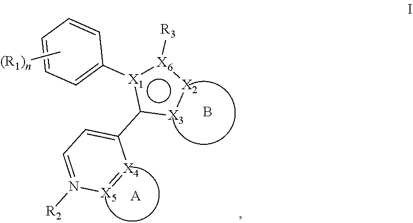

wherein R₁ may be a halogen,
n may be 0, 1, or 2,
X₁, X₂, X₃, X₄, X₅ and X₆ may each independently be C or N,
R₂ may be absent or O,
R₃ may be absent or —CN,
A may be absent,

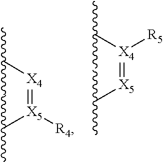

or ring A,
wherein R₄ may be selected from the group consisting of: —NH₂, C₁-C₆ alkyl, alkyl-COO-alkyl, alkyl-NH-alkyl and alkyl-OH,
wherein R₅ may be a halogen,
wherein ring A may be a 4- to 7-membered cycloalkyl or heterocycloalkyl or a 5- to 6-membered heteroaryl, wherein up to 2 carbon atoms may be replaced with a heteroatom selected from ═N— and —O—, and said ring A may optionally be substituted with a R₈ substituent, and R₈ may be ═O,
B may be a 4- to 7-membered cycloalkyl or heterocycloalkyl or a 5- to 6-membered heteroaryl, wherein up to 2 carbon atoms may be replaced with a heteroatom selected from ═N— and —O—, and said B may be optionally substituted with one or more (e.g., 1 or 2) R₆ substituents; R₆ may further be substituted with a R₇ substituent,
wherein each R₆ may be independently selected from the group consisting of: heterocycloalkyl, C₁-C₆ alkyl, CO-alkyl, CO-heterocycloalkyl, acyl-alkyl, benzyl, p-methoxybenzyl, O and CO-alkylcyano, and
wherein R₇ may be C₁-C₆ alkyl.

In the compound of Formula I, R₁ may be F.

In some cases, in the compound of Formula I, n may be 1 or 2.

In some embodiments, in the compound of Formula I, R₄ may be selected from the group consisting of: —NH₂, —CH₂—NH—CH₃, —CH₂OH, —CH₃ and —CH₂—O—CO—CH₃.

In some cases, in the compound of Formula I, R₅ may be F.

In some cases, in the compound of Formula I, ring A may be

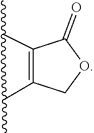

In some cases, in the compound of Formula I, A may be selected from the group consisting of:

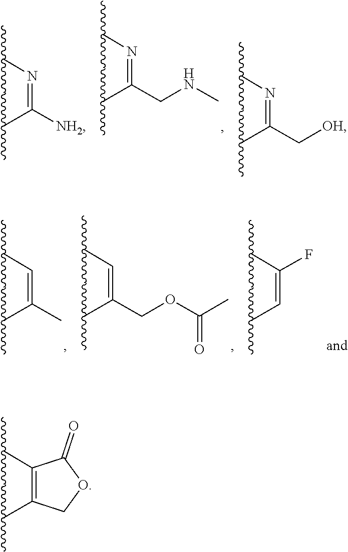

In some cases, in the compound of Formula I, each R₆ may independently be selected from the group consisting of:

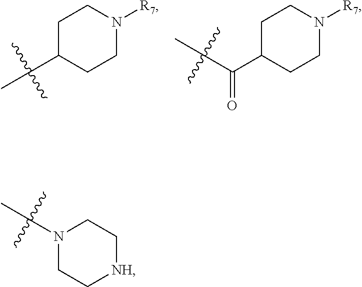

—CH₃, —CO—CH₃, —CO—NH—CH₃, —Bn, —PMB, —O, —CO—CH₂—CH₃, —CO—CH—(CH₃)₂ and —CO—CH₂—CN.

In some cases, in the compound of Formula I, R₇ may be —CH₃.

In some cases, in the compound of Formula I, the number of R₆ substituents may be 0, 1 or 2.

In some cases, in the compound of Formula I, B may be selected from the group consisting of:

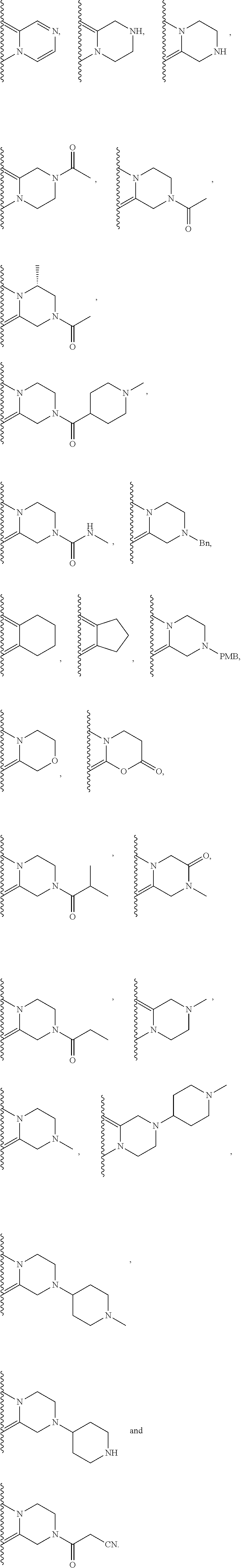

In some cases, wherein said A may be

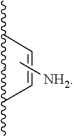

In some cases, wherein said A may be

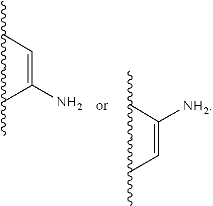

In some cases, wherein X₁ may be C.

In some cases, wherein X₆ may be N.

In some cases, wherein the compound may have a structure of

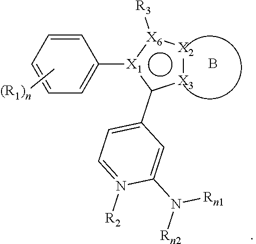

In some cases, wherein, R₁ may be F, n may be 1, X₁ and X₃ may be C, X₂ and X₆ may be N, R₂ and R₃ may be absent, Rₙ₁ and Rₙ₂ may independently be optionally substituted, B may be a 4- to 7-membered cycloalkyl or heterocycloalkyl or a 5- to 6-membered heteroaryl, wherein up to 2 carbon atoms may be replaced with a heteroatom selected from ═N— and —O—, and said B may be optionally substituted.

In some cases, wherein, R₁ may be F, n may be 1, X₁ and X₃ may be C, X₂ and X₆ may be N, R₂ and R₃ may be absent, B may be a 4- to 7-membered cycloalkyl or heterocycloalkyl, wherein up to 2 carbon atoms may be replaced with a heteroatom selected from ═N— and —O—, and said B may be optionally substituted.

In some cases, wherein, R₁ may be F, n may be 1, X₁ and X₃ may be C, X₂ and X₆ may be N, R₂ and R₃ may be absent, B may be selected from the group consisting of:

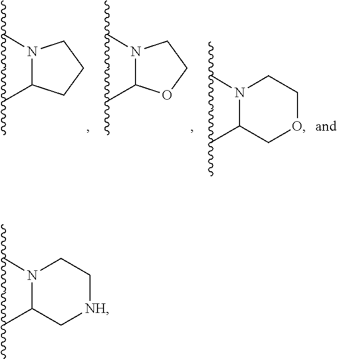

and said B may be optionally substituted.

In some cases, wherein, R₁ may be F, n may be 1, X₁ and X₃ may be C, X₂ and X₆ may be N, R₂ and R₃ may be absent, B may be

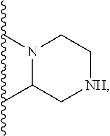

and said B may be optionally substituted with C₁-C₆ alkyl or CO—C₁-C₆ alkyl

In some cases, the compound of Formula I may be one of the compounds in tables 1-1 to 1-4.

TABLE 1-1
The compounds of Formula I

| Compound | Structure | IUPAC NAME |
|---|---|---|
| 1-1 | 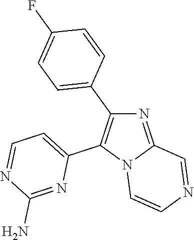 [SMILES: Nc1nccc(-c2c(-c3ccc(F)cc3)nc3cnccn23)n1] | 4-(2-(4-fluorophenyl)imidazo[1,2-a]pyrazin-3- yl)pyrimidin-2-amine |
| 1-2 | 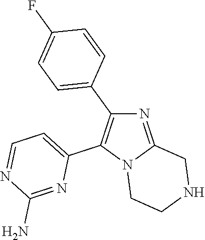 [SMILES: Nc1nccc(-c2c(-c3ccc(F)cc3)nc3n2CCNC3)n1] | 4-(2-(4-fluorophenyl)-5,6,7,8-tetrahydroimidazo[1,2- a]pyrazin-3-yl)pyrimidin-2-amine |
| 1-3 | 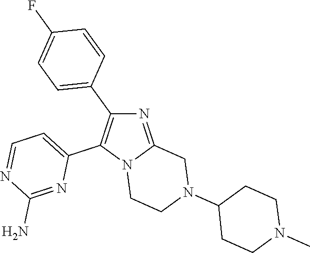 [SMILES: CN1CCC(N2CCn3c(nc(-c4ccc(F)cc4)c3-c3ccnc(N)n3)C2)CC1] | 4-(2-(4-fluorophenyl)-7-(1-methylpiperidin-4-yl)- 5,6,7,8-tetrahydroimidazo[1,2-a]pyrazin-3- yl)pyrimidin-2-amine |
| 1-4 | 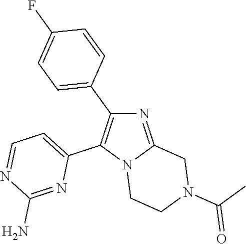 [SMILES: CC(=O)N1CCn2c(nc(-c3ccc(F)cc3)c2-c2ccnc(N)n2)C1] | 1-(3-(2-aminopyrimidin-4-yl)-2-(4-fluorophenyl)- 5,6,7,8-tetrahydroimidazo[1,2-a]pyrazin-7-yl)ethan-1- one |
| 1-5 | 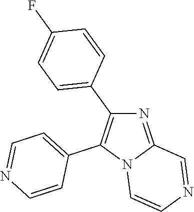 [SMILES: Fc1ccc(-c2nc3cnccn3c2-c2ccncc2)cc1] | 2-(4-fluorophenyl)-3-(pyridin-4-yl)imidazo[1,2- a]pyrazine |
| 1-6 | 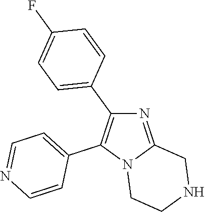 [SMILES: Fc1ccc(-c2nc3n(c2-c2ccncc2)CCNC3)cc1] | 2-(4-fluorophenyl)-3-(pyridin-4-yl)-5,6,7,8- tetrahydroimidazo[1,2-a]pyrazine |
| 1-7 | 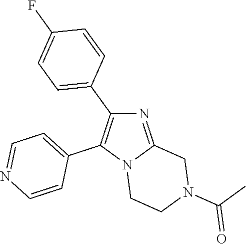 [SMILES: CC(=O)N1CCn2c(nc(-c3ccc(F)cc3)c2-c2ccncc2)C1] | 1-(2-(4-fluorophenyl)-3-(pyridin-4-yl)-5,6- dihydroimidazo[1,2-a]pyrazin-7(8H)-yl)ethan-1-one |
| 1-8 | 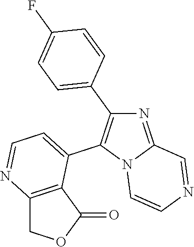 [SMILES: O=C1OCc2nccc(-c3c(-c4ccc(F)cc4)nc4cnccn34)c21] | 4-(2-(4-fluorophenyl)imidazo[1,2-a]pyrazin-3- yl)furo[3,4-b]pyridin-5(7H)-one |
| 1-9 | 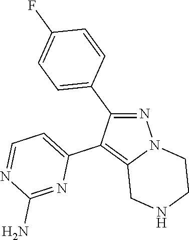 [SMILES: Nc1nccc(-c2c(-c3ccc(F)cc3)nn3c2CNCC3)n1] | 4-(2-(4-fluorophenyl)-4,5,6,7-tetrahydropyrazolo[1,5- a]pyrazin-3-yl)pyrimidin-2-amine |
| 1-10 | 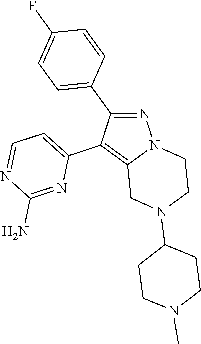 [SMILES: CN1CCC(N2CCn3nc(-c4ccc(F)cc4)c(-c4ccnc(N)n4)c3C2)CC1] | 4-(2-(4-fluorophenyl)-5-(1-methylpiperidin-4-yl)- 4,5,6,7-tetrahydropyrazolo[1,5-a]pyrazin-3- yl)pyrimidin-2-amine |

TABLE 1-2
The compounds of Formula I

| Compound | Structure | IUPAC NAME |
|---|---|---|
| 1-11 | 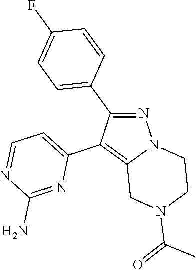 [SMILES: CC(=O)N1CCn2nc(-c3ccc(F)cc3)c(-c3ccnc(N)n3)c2C1] | 1-(3-(2-aminopyrimidin-4-yl)-2-(4-fluorophenyl)- 4,5,6,7-tetrahydropyrazolo[1,5-a]pyrazin-5(4H)- yl)ethan-1-one |
| 1-12 | 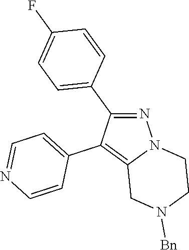 [SMILES: Fc1ccc(-c2nn3c(c2-c2ccncc2)CN(Cc2ccccc2)CC3)cc1] | 5-benzyl-2-(4-fluorophenyl)-3-(pyridin-4-yl)-4,5,6,7- tetrahydropyrazolo[1,5-a]pyrazine |
| 1-13 | 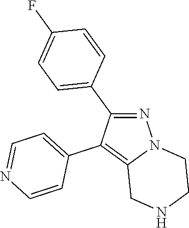 [SMILES: Fc1ccc(-c2nn3c(c2-c2ccncc2)CNCC3)cc1] | 2-(4-fluorophenyl)-3-(pyridin-4-yl)-4,5,6,7- tetrahydropyrazolo[1,5-a]pyrazine |
| 1-14 | 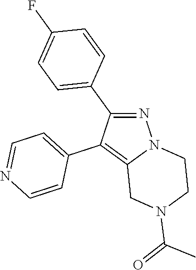 [SMILES: CC(=O)N1CCn2nc(-c3ccc(F)cc3)c(-c3ccncc3)c2C1] | 1-(2-(4-fluorophenyl)-3-(pyridin-4-yl)-6,7- dihydropyrazolo[1,5-a]pyrazin-5(4H)-yl)ethan-1-one |
| 1-15 | 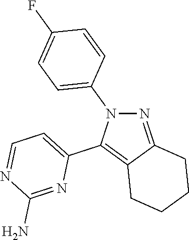 [SMILES: Nc1nccc(-c2c3c(nn2-c2ccc(F)cc2)CCCC3)n1] | 4-(2-(4-fluorophenyl)-4,5,6,7-tetrahydro-2H-indazol-3- yl)pyrimidin-2-amine |
| 1-16 | 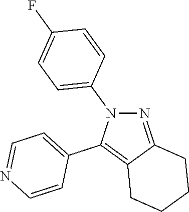 [SMILES: Fc1ccc(-n2nc3c(c2-c2ccncc2)CCCC3)cc1] | 2-(4-fluorophenyl)-3-(pyridin-4-yl)-4,5,6,7-tetrahydro- 2H-indazole |
| 1-17 | 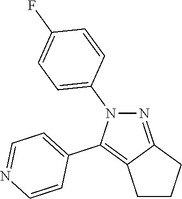 [SMILES: Fc1ccc(-n2nc3c(c2-c2ccncc2)CCC3)cc1] | 2-(4-fluorophenyl)-3-(pyridin-4-yl)-2,4,5,6- tetrahydrocyclopenta[c]pyrazole |
| 1-18 | 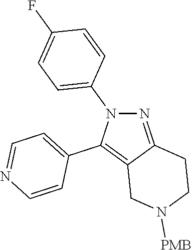 | 2-(4-fluorophenyl)-5-(4-methoxybenzyl)-3-(pyridin-4- yl)-4,5,6,7-tetrahydro-2H-pyrazolo[4,3-c]pyridine |
| 1-19 | 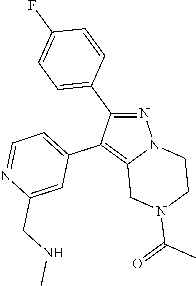 [SMILES: CNCc1cc(-c2c(-c3ccc(F)cc3)nn3c2CN(C(C)=O)CC3)ccn1] | 1-(2-(4-fluorophenyl)-3-(2- ((methylamino)methyl)pyridin-4-yl)-4,5,6,7- tetrahydropyrazolo[1,5-a]pyrazin-5-yl)ethan-1-one |
| 1-20 | 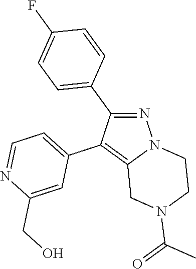 [SMILES: CC(=O)N1CCn2nc(-c3ccc(F)cc3)c(-c3ccnc(CO)c3)c2C1] | 1-(2-(4-fluorophenyl)-3-(2-(hydroxymethyl)pyridin-4- yl)-4,5,6,7-tetrahydropyrazolo[1,5-a]pyrazin-5- yl)ethan-1-one |

TABLE 1-3
The compounds of Formula I

| Compound | Structure | IUPAC NAME |
|---|---|---|
| 1-21 | 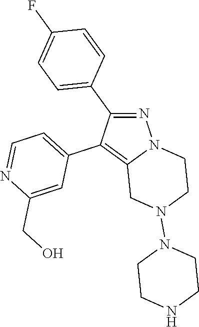 [SMILES: OCc1cc(-c2c(-c3ccc(F)cc3)nn3c2CN(N2CCNCC2)CC3)ccn1] | (4-(2-(4-fluorophenyl)-5-(piperazin-1-yl)-4,5,6,7- tetrahydropyrazolo[1,5-a]pyrazin-3-yl)pyridin-2-yl)methanol |
| 1-22 | 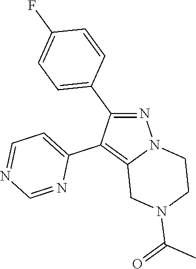 [SMILES: CC(=O)N1CCn2nc(-c3ccc(F)cc3)c(-c3ccncn3)c2C1] | 1-(2-(4-fluorophenyl)-3-(pyrimidin-4-yl)-4,5,6,7- tetrahydropyrazolo[1,5-a]pyrazin-5-yl)ethan-1-one |
| 1-23 | 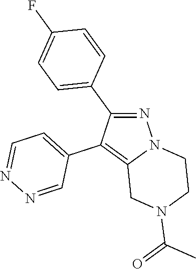 [SMILES: CC(=O)N1CCn2nc(-c3ccc(F)cc3)c(-c3ccnnc3)c2C1] | 1-(2-(4-fluorophenyl)-3-(pyridazin-4-yl)-6,7- dihydropyrazolo[1,5-a]pyrazin-5(4H)-yl)ethan-1-one |
| 1-24 | 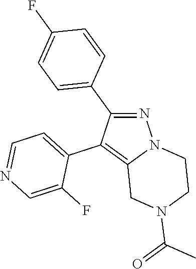 [SMILES: CC(=O)N1CCn2nc(-c3ccc(F)cc3)c(-c3ccncc3F)c2C1] | 1-(2-(4-fluorophenyl)-3-(3-fluoropyridin-4-yl)-6,7- dihydropyrazolo[1,5-a]pyrazin-5(4H)-yl)ethan-1-one |
| 1-25 | 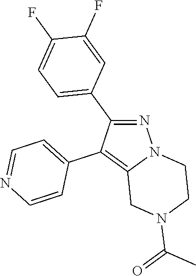 [SMILES: CC(=O)N1CCn2nc(-c3ccc(F)c(F)c3)c(-c3ccncc3)c2C1] | 1-(2-(3,4-difluorophenyl)-3-(pyridin-4-yl)-6,7- dihydropyrazolo[1,5-a]pyrazin-5(4H)-yl)ethan-1-one |
| 1-26 | 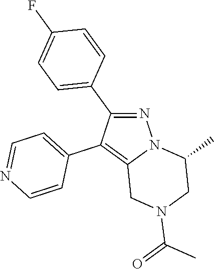 [SMILES: CC(=O)N1Cc2c(-c3ccncc3)c(-c3ccc(F)cc3)nn2[C@H](C)C1] | (R)-1-(2-(4-fluorophenyl)-7-methyl-3-(pyridin-4-yl)-6,7- dihydropyrazolo[1,5-a]pyrazin-5(4H)-yl)ethan-1-one |
| 1-27 | 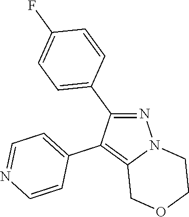 [SMILES: Fc1ccc(-c2nn3c(c2-c2ccncc2)COCC3)cc1] | 2-(4-fluorophenyl)-3-(pyridin-4-yl)-6,7-dihydro-4H- pyrazolo[5,1-c][1,4]oxazine |
| 1-28 | 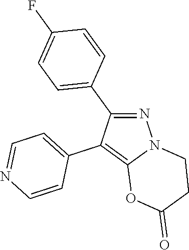 [SMILES: O=C1CCn2nc(-c3ccc(F)cc3)c(-c3ccncc3)c2O1] | 2-(4-fluorophenyl)-3-(pyridin-4-yl)-6,7-dihydro-5H- pyrazolo[5,1-b][1,3]oxazin-5-one |
| 1-29 | 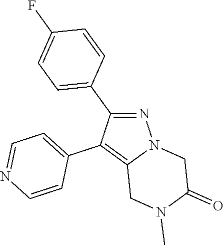 [SMILES: CN1Cc2c(-c3ccncc3)c(-c3ccc(F)cc3)nn2CC1=O] | 2-(4-fluorophenyl)-5-methyl-3-(pyridin-4-yl)-4,5- dihydropyrazolo[1,5-a]pyrazin-6(7H)-one |
| 1-30 | 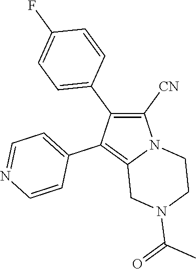 [SMILES: CC(=O)N1CCn2c(C#N)c(-c3ccc(F)cc3)c(-c3ccncc3)c2C1] | 2-acetyl-7-(4-fluorophenyl)-8-(pyridin-4-yl)-1,2,3,4- tetrahydropyrrolo[1,2-a]pyrazine-6-carbonitrile |

TABLE 1-4
The compounds of Formula 1

| Compound | Structure | IUPAC NAME |
|---|---|---|
| 1-31 | 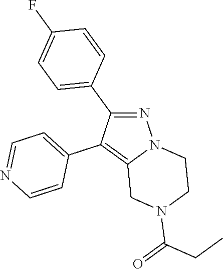 [SMILES: CCC(=O)N1CCn2nc(-c3ccc(F)cc3)c(-c3ccncc3)c2C1] | 1-(2-(4-fluorophenyl)-3-(pyridin-4-yl)-6,7- dihydropyrazolo[1,5-a]pyrazin-5(4H)-yl)propan- 1-one |
| 1-32 | 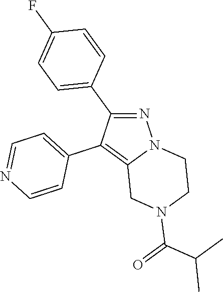 [SMILES: CC(C)C(=O)N1CCn2nc(-c3ccc(F)cc3)c(-c3ccncc3)c2C1] | 1-(2-(4-fluorophenyl)-3-(pyridin-4-yl)-6,7- dihydropyrazolo[1,5-a]pyrazin-5(4H)-yl)-2- methylpropan-1-one |
| 1-33 | 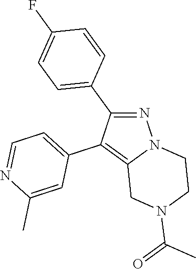 [SMILES: CC(=O)N1CCn2nc(-c3ccc(F)cc3)c(-c3ccnc(C)c3)c2C1] | 1-(2-(4-fluorophenyl)-3-(2-methylpyridin-4-yl)- 6,7-dihydropyrazolo[1,5-a]pyrazin-5(4H)- yl)ethan-1-one |
| 1-34 | 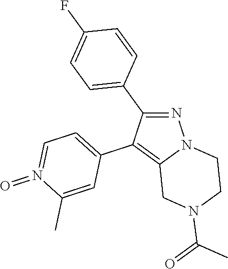 [SMILES: CC(=O)N1CCn2nc(-c3ccc(F)cc3)c(-c3cc[n+]([O-])c(C)c3)c2C1] | 4-(5-acetyl-2-(4-fluorophenyl)-4,5,6,7- tetrahydropyrazolo[1,5-a]pyrazin-3-yl)-2- methylpyridine-1-oxide |
| 1-35 | 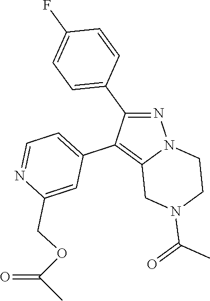 [SMILES: CC(=O)OCc1cc(-c2c(-c3ccc(F)cc3)nn3c2CN(C(C)=O)CC3)ccn1] | (4-(5-acetyl-2-(4-fluorophenyl)-4,5,6,7- tetrahydropyrazolo[1,5-a]pyrazin-3-yl)pyridin-2- yl)methyl acetate |
| 1-36 | 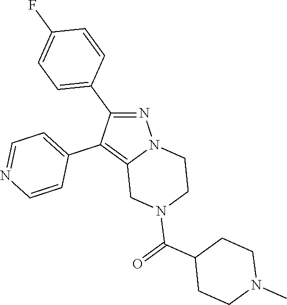 [SMILES: CN1CCC(C(=O)N2CCn3nc(-c4ccc(F)cc4)c(-c4ccncc4)c3C2)CC1] | (2-(4-fluorophenyl)-3-(pyridin-4-yl)-6,7- dihydropyrazolo[1,5-a]pyrazin-5(4H)-yl)(1- methylpiperidin-4-yl)methanone |
| 1-37 | 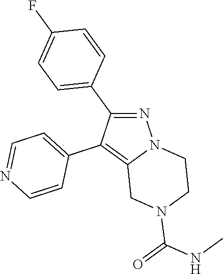 [SMILES: CNC(=O)N1CCn2nc(-c3ccc(F)cc3)c(-c3ccncc3)c2C1] | 2-(4-fluorophenyl)-N-methyl-3-(pyridin-4-yl)- 6,7-dihydropyrazolo[1,5-a]pyrazine-5(4H)- carboxamide |
| 1-38 | 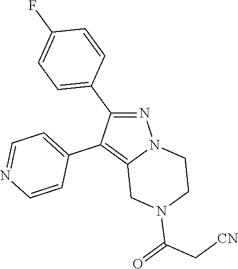 [SMILES: N#CCC(=O)N1CCn2nc(-c3ccc(F)cc3)c(-c3ccncc3)c2C1] | 3-(2-(4-fluorophenyl)-3-(pyridin-4-yl)-6,7- dihydropyrazolo[1,5-a]pyrazin-5(4H)-yl)-3- oxopropanenitrile |
| 1-39 | 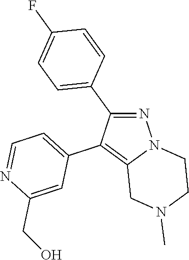 [SMILES: CN1CCn2nc(-c3ccc(F)cc3)c(-c3ccnc(CO)c3)c2C1] | (4-(2-(4-fluorophenyl)-5-methyl-4,5,6,7- tetrahydropyrazolo[1,5-a]pyrazin-3-yl)pyridin-2- yl)methanol |
| 1-40 | 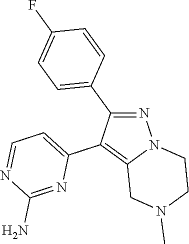 [SMILES: CN1CCn2nc(-c3ccc(F)cc3)c(-c3ccnc(N)n3)c2C1] | 4-(2-(4-fluorophenyl)-5-methyl-4,5,6,7- tetrahydropyrazolo[1,5-a]pyrazin-3-yl)pyrimidin- 2-amine |
| 1-41 | 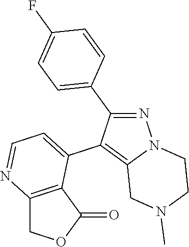 [SMILES: CN1CCn2nc(-c3ccc(F)cc3)c(-c3ccnc4c3C(=O)OC4)c2C1] | 4-(2-(4-fluorophenyl)-5-methyl-4,5,6,7- tetrahydropyrazolo[1,5-a]pyrazin-3-yl)furo[3,4- b]pyridin-5(7H)-one |
| 1-42 | 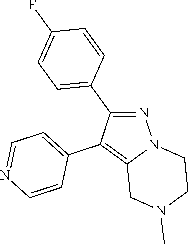 [SMILES: CN1CCn2nc(-c3ccc(F)cc3)c(-c3ccncc3)c2C1] | 2-(4-fluorophenyl)-5-methyl-3-(pyridin-4-yl)- 4,5,6,7-tetrahydropyrazolo[1,5-a]pyrazine |
| 1-43 | 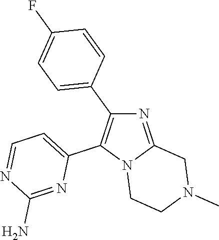 [SMILES: CN1CCn2c(nc(-c3ccc(F)cc3)c2-c2ccnc(N)n2)C1] | 4-(2-(4-fluorophenyl)-7-methyl-5,6,7,8- tetrahydroimidazo[1,2-a]pyrazin-3-yl)pyrimidin- 2-amine |
| 1-44 | 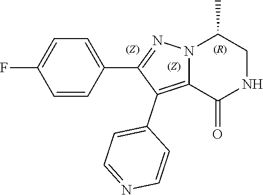 | (R)-2-(4-fluorophenyl)-7-methyl-3-(pyridin-4- yl)-6,7-dihydropyrazolo[1,5-a]pyrazin-4(5H)-one |
| 1-45 | 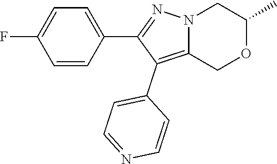 [SMILES: C[C@H]1Cn2nc(-c3ccc(F)cc3)c(-c3ccncc3)c2CO1] | (S)-2-(4-fluorophenyl)-6-methyl-3-(pyridin-4- yl)-6,7-dihydro-4H-pyrazolo[5,1-c][1,4]oxazine |
| 1-46 | 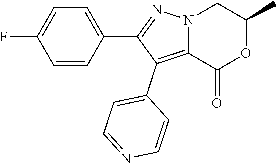 [SMILES: C[C@@H]1Cn2nc(-c3ccc(F)cc3)c(-c3ccncc3)c2C(=O)O1] | (R)-2-(4-fluorophenyl)-6-methyl-3-(pyridin-4- yl)-6,7-dihydro-4H-pyrazolo[5,1-c][1,4]oxazine |
| 1-47 | 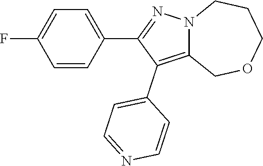 [SMILES: Fc1ccc(-c2nn3c(c2-c2ccncc2)COCCC3)cc1] | 2-(4-fluorophenyl)-3-(pyridin-4-yl)-7,8-dihydro- 4H,6H-pyrazolo[5,1-c][1,4]oxazepine |
| 1-48 | 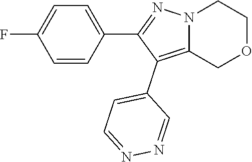 [SMILES: Fc1ccc(-c2nn3c(c2-c2ccnnc2)COCC3)cc1] | 2-(4-fluorophenyl)-3-(pyridazin-4-yl)-6,7- dihydro-4H-pyrazolo[5,1-c][1,4]oxazine |
| 1-49 | 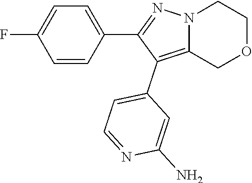 [SMILES: Nc1cc(-c2c(-c3ccc(F)cc3)nn3c2COCC3)ccn1] | 4-(2-(4-fluorophenyl)-6,7-dihydro-4H- pyrazolo[5,1-c][1,4]oxazin-3-yl)pyridin-2-amine |
| 1-50 | 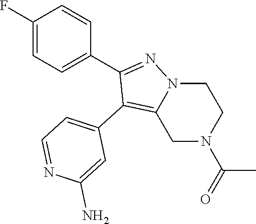 [SMILES: CC(=O)N1CCn2nc(-c3ccc(F)cc3)c(-c3ccnc(N)c3)c2C1] | 1-(3-(2-aminopyridin-4-yl)-2-(4-fluorophenyl)- 6,7-dihydropyrazolo[1,5-a]pyrazin-5(4H)- yl)ethan-1-one |
| 1-51 | 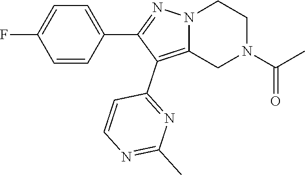 [SMILES: CC(=O)N1CCn2nc(-c3ccc(F)cc3)c(-c3ccnc(C)n3)c2C1] | 1-(2-(4-fluorophenyl)-3-(2-methylpyrimidin-4- yl)-6,7-dihydropyrazolo[1,5-a]pyrazin-5(4H)- yl)ethan-1-one |
| 1-52 | 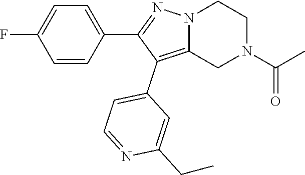 [SMILES: CCc1cc(-c2c(-c3ccc(F)cc3)nn3c2CN(C(C)=O)CC3)ccn1] | 1-(3-(2-ethylpyridin-4-yl)-2-(4-fluorophenyl)- 6,7-dihydropyrazolo[1,5-a]pyrazin-5(4H)- yl)ethan-1-one |
| 1-53 | 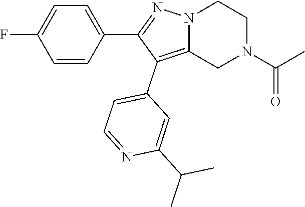 [SMILES: CC(=O)N1CCn2nc(-c3ccc(F)cc3)c(-c3ccnc(C(C)C)c3)c2C1] | 1-(2-(4-fluorophenyl)-3-(2-isopropylpyridin-4- yl)-6,7-dihydropyrazolo[1,5-a]pyrazin-5(4H)- yl)ethan-1-one |
| 1-54 | 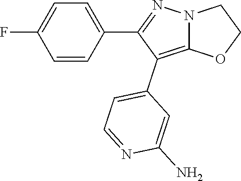 [SMILES: Nc1cc(-c2c(-c3ccc(F)cc3)nn3c2OCC3)ccn1] | 4-(6-(4-fluorophenyl)-2,3-dihydropyrazolo[5,1- b]oxazol-7-yl)pyridin-2-amine |
| 1-55 | 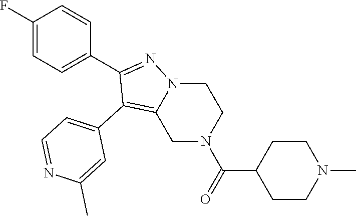 [SMILES: Cc1cc(-c2c(-c3ccc(F)cc3)nn3c2CN(C(=O)C2CCN(C)CC2)CC3)ccn1] | (2-(4-fluorophenyl)-3-(2-methylpyridin-4-yl)- 6,7-dihydropyrazolo[1,5-a]pyrazin-5(4H)-yl)(1- methylpiperidin-4-yl)methanone |
| 1-56 | 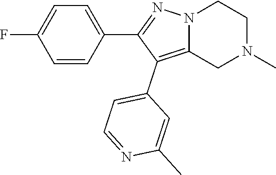 [SMILES: Cc1cc(-c2c(-c3ccc(F)cc3)nn3c2CN(C)CC3)ccn1] | 2-(4-fluorophenyl)-5-methyl-3-(2-methylpyridin- 4-yl)-4,5,6,7-tetrahydropyrazolo[1,5-a]pyrazine |
| 1-57 | 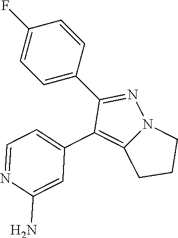 [SMILES: Nc1cc(-c2c(-c3ccc(F)cc3)nn3c2CCC3)ccn1] | 4-(2-(4-fluorophenyl)-5,6-dihydro-4H- pyrrolo[1,2-b]pyrazol-3-yl)pyridin-2-amine |
| 1-58 | 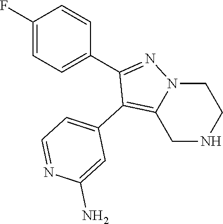 [SMILES: Nc1cc(-c2c(-c3ccc(F)cc3)nn3c2CNCC3)ccn1] | 4-(2-(4-fluorophenyl)-4,5,6,7- tetrahydropyrazolo[1,5-a]pyrazin-3-yl)pyridin-2- amine |
| 1-59 | 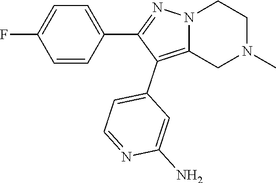 [SMILES: CN1CCn2nc(-c3ccc(F)cc3)c(-c3ccnc(N)c3)c2C1] | 4-(2-(4-fluorophenyl)-5-methyl-4,5,6,7- tetrahydropyrazolo[1,5-a]pyrazin-3-yl)pyridin-2- amine |
| 1-60 | 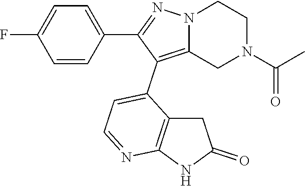 [SMILES: CC(=O)N1CCn2nc(-c3ccc(F)cc3)c(-c3ccnc4c3CC(=O)N4)c2C1] | 4-(5-acetyl-2-(4-fluorophenyl)-4,5,6,7- tetrahydropyrazolo[1,5-a]pyrazin-3-yl)-1,3- dihydro-2H-pyrrolo[2,3-b]pyridin-2-one |
| 1-61 | 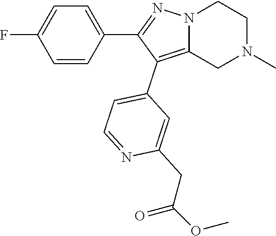 [SMILES: COC(=O)Cc1cc(-c2c(-c3ccc(F)cc3)nn3c2CN(C)CC3)ccn1] | methyl 2-(4-(2-(4-fluorophenyl)-5-methyl- 4,5,6,7-tetrahydropyrazolo[1,5-a]pyrazin-3- yl)pyridin-2-yl)acetate |
| 1-62 | 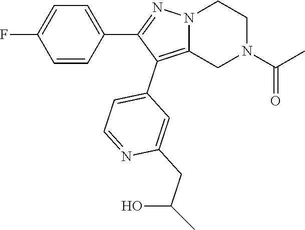 [SMILES: CC(=O)N1CCn2nc(-c3ccc(F)cc3)c(-c3ccnc(CC(C)O)c3)c2C1] | 1-(2-(4-fluorophenyl)-3-(2-(2- hydroxypropyl)pyridin-4-yl)-6,7- dihydropyrazolo[1,5-a]pyrazin-5(4H)-yl)ethan-1- one |
| 1-63 | 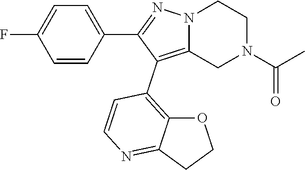 [SMILES: CC(=O)N1CCn2nc(-c3ccc(F)cc3)c(-c3ccnc4c3OCC4)c2C1] | 1-(3-(2,3-dihydrofuro[3,2-b]pyridin-7-yl)-2-(4- fluorophenyl)-6,7-dihydropyrazolo[1,5- a]pyrazin-5(4H)-yl)ethan-1-one |
| 1-64 | 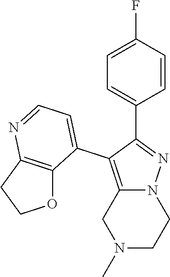 [SMILES: CN1CCn2nc(-c3ccc(F)cc3)c(-c3ccnc4c3OCC4)c2C1] | 7-(2-(4-fluorophenyl)-5-methyl-4,5,6,7- tetrahydropyrazolo[1,5-a]pyrazin-3-yl)-2,3- dihydrofuro[3,2-b]pyridine |
| 1-65 | 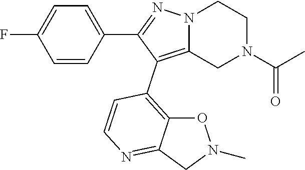 [SMILES: CC(=O)N1CCn2nc(-c3ccc(F)cc3)c(-c3ccnc4c3ON(C)C4)c2C1] | 1-(2-(4-fluorophenyl)-3-(2-methyl-2,3- dihydroisoxazolo[4,5-b]pyridin-7-yl)-6,7- dihydropyrazolo[1,5-a]pyrazin-5(4H)-yl)ethan-1- one |
| 1-66 | 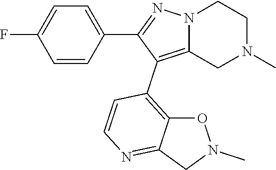 [SMILES: CN1CCn2nc(-c3ccc(F)cc3)c(-c3ccnc4c3ON(C)C4)c2C1] | 7-(2-(4-fluorophenyl)-5-methyl-4,5,6,7- tetrahydropyrazolo[1,5-a]pyrazin-3-yl)-2-methyl- 2,3-dihydroisoxazolo[4,5-b]pyridine |
| 1-67 | 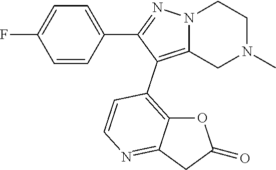 [SMILES: CN1CCn2nc(-c3ccc(F)cc3)c(-c3ccnc4c3OC(=O)C4)c2C1] | 7-(2-(4-fluorophenyl)-5-methyl-4,5,6,7- tetrahydropyrazolo[1,5-a]pyrazin-3-yl)furo[3,2- b]pyridin-2(3H)-one |
| 1-68 | 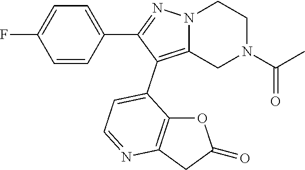 [SMILES: CC(=O)N1CCn2nc(-c3ccc(F)cc3)c(-c3ccnc4c3OC(=O)C4)c2C1] | 7-(5-acetyl-2-(4-fluorophenyl)-4,5,6,7- tetrahydropyrazolo[1,5-a]pyrazin-3-yl)furo[3,2- b]pyridin-2(3H)-one |

In another aspect, the present application also provides a compound of Formula II, or a pharmaceutically acceptable salt thereof:

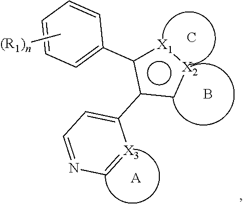

wherein R₁ may be a halogen,
n may be 0, 1, or 2,
X₁, X₂ and X₃ may each independently be C or N,
A may be absent,

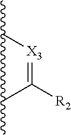

or ring A,
wherein ring A may be a 4- to 7-membered cycloalkyl or heterocycloalkyl or a 5- to 6-membered heteroaryl, wherein up to 2 carbon atoms may be replaced with a heteroatom selected from ═N— and —O—, and ring A may optionally be substituted with a R₃ substituent, R₃ may be ═O;
wherein R₂ may be —NH₂ or C₁-C₆ alkyl;
B may be absent,

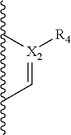

or ring B,
wherein R₄ may be C₁-C₆ alkyl,
wherein ring B may be a 4- to 7-membered cycloalkyl or heterocycloalkyl or a 5- to 6-membered heteroaryl, wherein up to 2 carbon atoms may be replaced with a heteroatom selected from ═N— and —O—;
C may be absent,

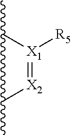

or ring C,
wherein R₅ may be absent, a cyano or an amide group, and
wherein ring C may be a 4- to 7-membered cycloalkyl or heterocycloalkyl or a 5- to 6-membered heteroaryl, wherein up to 2 carbon atoms are replaced with a heteroatom selected from ═N— and —O—, and said ring C may be optionally substituted with a R₆ substituent, R₆ may be ═O.

In some cases, in the compound of Formula II, R₁ may be F.

In some cases, in the compound of Formula II, n may be 1.

In some cases, in the compound of Formula II, R₂ may be —CH₃ or —NH₂.

In some cases, in the compound of Formula II, A may be selected from the group consisting of:

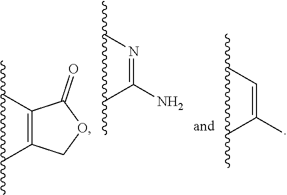

In some cases, in the compound of Formula II, R₄ may be —CH₃.

In some cases, in the compound of Formula II, B may be selected from the group consisting of:

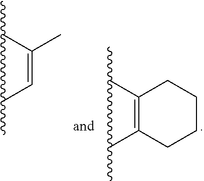

In some cases, in the compound of Formula II, R₅ may be —CN or —CO—NH₂.

In some cases, in the compound of Formula II, C may be selected from the group consisting of:

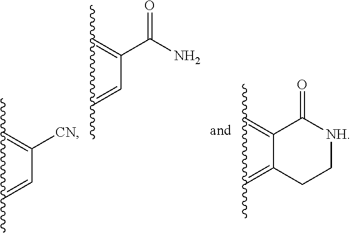

In some cases, the compound of Formula II may be one of the compounds in table 2.

TABLE 2
The compounds of Formula II

| Compound | Structure | IUPAC NAME |
|---|---|---|
| 2-1 | 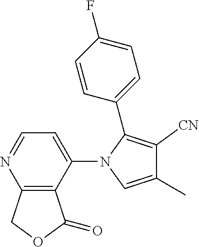 [SMILES: Cc1cn(-c2ccnc3c2C(=O)OC3)c(-c2ccc(F)cc2)c1C#N] | 2-(4-fluorophenyl)-4-methyl-1-(5-oxo-5,7- dihydrofuro[3,4-b]pyridin-4-yl)-1H-pyrrole-3- carbonitrile |
| 2-2 | 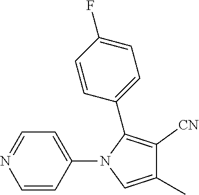 [SMILES: Cc1cn(-c2ccncc2)c(-c2ccc(F)cc2)c1C#N] | 2-(4-fluorophenyl)-4-methyl-1-(pyridin-4-yl)-1H- pyrrole-3-carbonitrile |
| 2-3 | 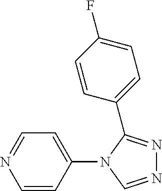 [SMILES: Fc1ccc(-c2nncn2-c2ccncc2)cc1] | 4-(3-(4-fluorophenyl)-4H-1,2,4-triazol-4-yl)pyridine |
| 2-4 | 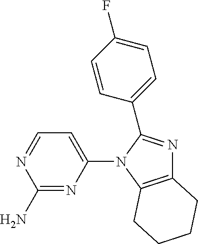 [SMILES: Nc1nccc(-n2c(-c3ccc(F)cc3)nc3c2CCCC3)n1] | 4-(2-(4-fluorophenyl)-4,5,6,7-tetrahydro-1H- benzo[d]imidazol-1-yl)pyrimidin-2-amine |
| 2-5 | 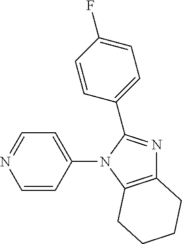 [SMILES: Fc1ccc(-c2nc3c(n2-c2ccncc2)CCCC3)cc1] | 2-(4-fluorophenyl)-1-(pyridin-4-yl)-4,5,6,7- tetrahydro-1H-benzo[d]imidazole |
| 2-6 | 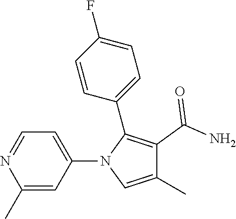 [SMILES: Cc1cc(-n2cc(C)c(C(N)=O)c2-c2ccc(F)cc2)ccn1] | 2-(4-fluorophenyl)-4-methyl-1-(2-methylpyridin-4- yl)-1H-pyrrole-3-carboxamide |
| 2-7 | 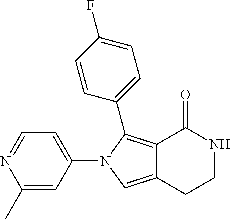 [SMILES: Cc1cc(-n2cc3c(c2-c2ccc(F)cc2)C(=O)NCC3)ccn1] | 3-(4-fluorophenyl)-2-(2-methylpyridin-4-yl)-2,5,6,7- tetrahydro-4H-pyrrolo[3,4-c]pyridin-4-one |

This present application also provides a compound of Formula III, or a pharmaceutically acceptable salt thereof:

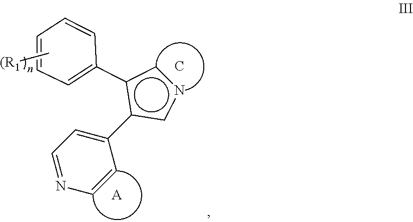

wherein R₁ may be a halogen,
n may be 0, 1, or 2,
A may be absent or ring A,
wherein ring A may be a 4- to 7-membered cycloalkyl or heterocycloalkyl or a 5- to 6-membered heteroaryl, wherein up to 2 carbon atoms may be replaced with a heteroatom selected from ═N— and —O—, and said ring A may optionally be substituted with a R₄ substituent, R₄ may be ═O,
C may be

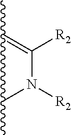

or ring C,
wherein R₂ may be —CN, —CONH₂, or —COO-alkyl,
wherein R₃ may be absent or C₁-C₆ alkyl, and
wherein ring C may be a 4- to 7-membered cycloalkyl or heterocycloalkyl or a 5- to 6-membered heteroaryl, wherein up to 2 carbon atoms may be replaced with a heteroatom selected from ═N— and —O—, and said ring C may optionally be substituted with a R₄ substituent, the R₄ may be ═O.

In some cases, in the compound of Formula III, R₁ may be F.

In some cases, in the compound of Formula III, n may be 1.

In some cases, in the compound of Formula III, wherein said ring A may be a 5-membered heteroaryl.

In some cases, in the compound of Formula III, wherein 2 carbon atoms of said A may be replaced with a heteroatom selected from ═N— and —O—.

In some cases, in the compound of Formula III, wherein said ring A may be

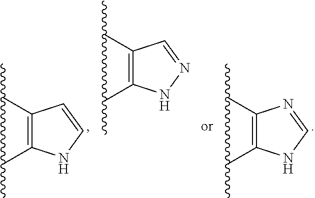

In some cases, wherein the compound may have a structure of

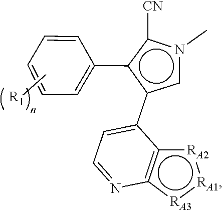

wherein, R₁ may be a halogen, n may be 0, 1, or 2, R_{A1}, R_{A2} and R_{A3} may independently be selected from C and N.

In some cases, wherein R_{A1} may be N.

In some cases, in the compound of Formula III, A may be absent or

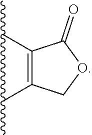

In some cases, in the compound of Formula III, R₂ may be —CN, —CONH₂, or —COO—CH₃.

In some cases, in the compound of Formula III, R₃ may be absent or —CH₃.

In some cases, in the compound of Formula III, C may be selected from the group consisting of:

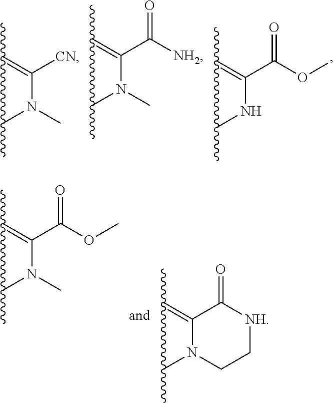

In some cases, the compound of Formula III may be one of the compounds in table 3.

TABLE 3
The compounds of Formula III

| Compound | Structure | IUPAC NAME |
|---|---|---|
| 3-1 | 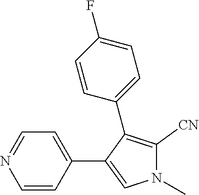 [SMILES: Cn1cc(-c2ccncc2)c(-c2ccc(F)cc2)c1C#N] | 3-(4-fluorophenyl)-1-methyl-4-(pyridin-4-yl)-1H- pyrrole-2-carbonitrile |
| 3-2 | 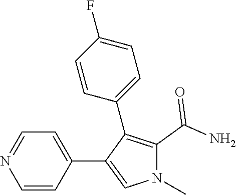 [SMILES: Cn1cc(-c2ccncc2)c(-c2ccc(F)cc2)c1C(N)=O] | 3-(4-fluorophenyl)-1-methyl-4-(pyridin-4-yl)-1H- pyrrole-2-carboxamide |
| 3-3 | 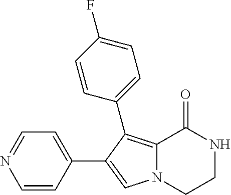 [SMILES: O=C1NCCn2cc(-c3ccncc3)c(-c3ccc(F)cc3)c21] | 8-(4-fluorophenyl)-7-(pyridin-4-yl)-3,4- dihydropyrrolo[1,2-a]pyrazin-1(2H)-one |
| 3-4 | 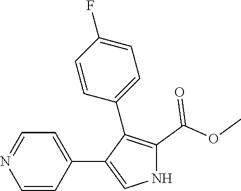 [SMILES: COC(=O)c1[nH]cc(-c2ccncc2)c1-c1ccc(F)cc1] | methyl 3-(4-fluorophenyl)-4-(pyridin-4-yl)-1H- pyrrole-2-carboxylate |
| 3-5 | 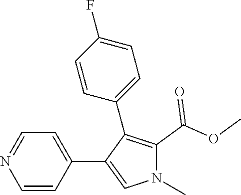 [SMILES: COC(=O)c1c(-c2ccc(F)cc2)c(-c2ccncc2)cn1C] | methyl 3-(4-fluorophenyl)-1-methyl-4-(pyridin-4- yl)-1H-pyrrole-2-carboxylate |
| 5-1 | 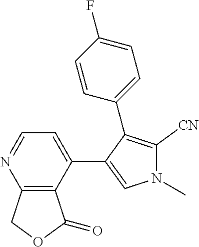 [SMILES: Cn1cc(-c2ccnc3c2C(=O)OC3)c(-c2ccc(F)cc2)c1C#N] | 3-(4-fluorophenyl)-1-methyl-4-(5-oxo-5,7- dihydrofuro[3,4-b]pyridin-4-yl)-1H-pyrrole-2- carbonitrile |
| 5-2 | 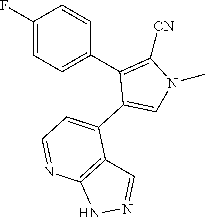 [SMILES: Cn1cc(-c2ccnc3[nH]ncc23)c(-c2ccc(F)cc2)c1C#N] | 3-(4-fluorophenyl)-1-methyl-4-(1H-pyrazolo[3,4- b]pyridin-4-yl)-1H-pyrrole-2-carbonitrile |
| 5-3 | 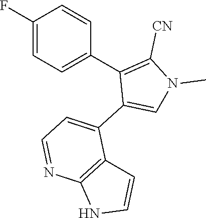 [SMILES: Cn1cc(-c2ccnc3[nH]ccc23)c(-c2ccc(F)cc2)c1C#N] | 3-(4-fluorophenyl)-1-methyl-4-(1H-pyrrolo[2,3- b]pyridin-4-yl)-1H-pyrrole-2-carbonitrile |
| 5-4 | 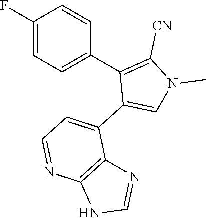 [SMILES: Cn1cc(-c2ccnc3[nH]cnc23)c(-c2ccc(F)cc2)c1C#N] | 3-(4-fluorophenyl)-4-(3H-imidazo[4,5-b]pyridin-7- yl)-1-methyl-1H-pyrrole-2-carbonitrile |
| 5-5 | 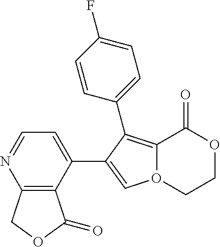 | 4-(8-(4-fluorophenyl)-1-oxo-1,2,3,4- tetrahydropyrrolo[1,2-a]pyrazin-7-yl)furo[3,4- b]pyridin-5(7H)-one |

The present application also provides a compound of Formula IV, or a pharmaceutically acceptable salt thereof:

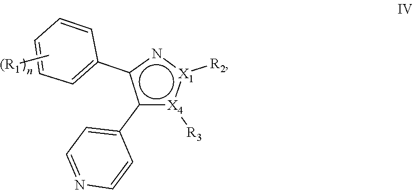

wherein R₁ may be a halogen,
n may be 0, 1, or 2,
X₁ may be C, O, or N,
X₄ may be C or N,
R₂ may be absent or C₁-C₆ alkyl,
R₃ may be absent, PMB, C₁-C₆ alkyl, or

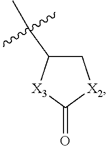

and
wherein X₂, X₃ may each independently be C or O.

In some cases, in the compound of Formula IV, R₁ may be F.

In some cases, in the compound of Formula IV, n may be 1.

In some cases, in the compound of Formula IV, wherein the compound may have the structure of

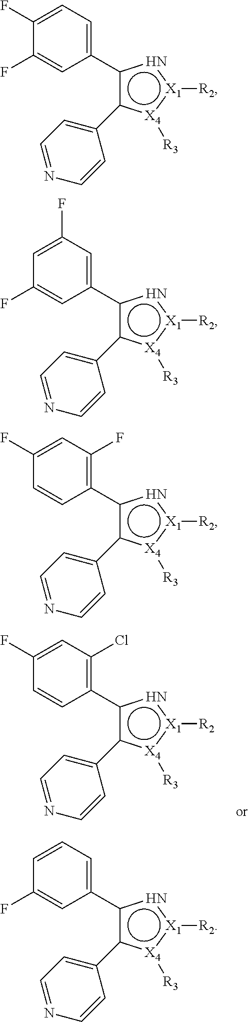

In some cases, in the compound of Formula IV, wherein R₂ may be absent or —CH₃.

In some cases, in the compound of Formula IV, wherein R₃ may be absent, PMB, —CH₃, or

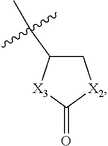

wherein X₂, X₃ may be each independently C or O, wherein, PMB represents group

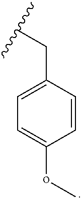

In some cases, in the compound of Formula IV, wherein X₂ may be C and X₃ may be O.

In some cases, in the compound of Formula IV, wherein X₃ may be C and X₂ may be O.

In some cases, in the compound of Formula IV, wherein X₄ may be C.

In some cases, the compound of Formula IV may be one of the compounds in table 4.

TABLE 4
The compounds of Formula IV

| Compound | Structure | IUPAC NAME |
|---|---|---|
| 3-6 | 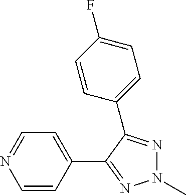 [SMILES: Cn1nc(-c2ccncc2)c(-c2ccc(F)cc2)n1] | 4-(5-(4-fluorophenyl)-2-methyl-2H-1,2,3-triazol-4- yl)pyridine |
| 3-7 | 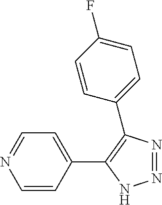 [SMILES: Fc1ccc(-c2nn[nH]c2-c2ccncc2)cc1] | 4-(4-(4-fluorophenyl)-1H-1,2,3-triazol-5- yl)pyridine |
| 3-8 | 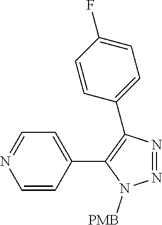 | 4-(4-(4-fluorophenyl)-1-(4-methoxybenzyl)-1H- 1,2,3-triazol-5-yl)pyridine |
| 3-9 | 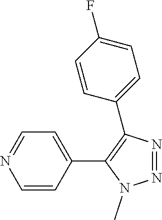 [SMILES: Cn1nnc(-c2ccc(F)cc2)c1-c1ccncc1] | 4-(4-(4-fluorophenyl)-1-methyl-1H-1,2,3-triazol-5- yl)pyridine |
| 3-10 | 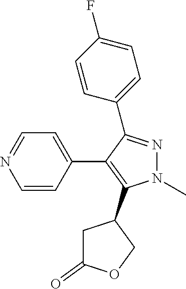 [SMILES: Cn1nc(-c2ccc(F)cc2)c(-c2ccncc2)c1[C@H]1COC(=O)C1] | (S)-4-(3-(4-fluorophenyl)-1-methyl-4-(pyridin-4- yl)-1H-pyrazol-5-yl)dihydrofuran-2(3H)-one |
| 3-11 | 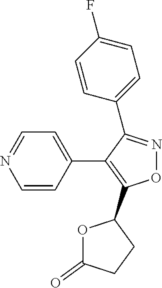 [SMILES: O=C1CC[C@H](c2onc(-c3ccc(F)cc3)c2-c2ccncc2)O1] | (R)-5-(3-(4-fluorophenyl)-4-(pyridin-4-yl)isoxazol- 5-yl)dihydrofuran-2(3H)-one |
| 3-12 | 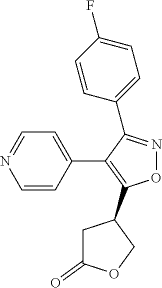 [SMILES: O=C1C[C@@H](c2onc(-c3ccc(F)cc3)c2-c2ccncc2)CO1] | (R)-4-(3-(4-fluorophenyl)-4-(pyridin-4-yl)isoxazol- 5-yl)dihydrofuran-2(3H)-one |
| 3-13 | 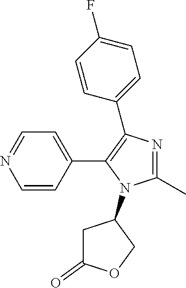 [SMILES: Cc1nc(-c2ccc(F)cc2)c(-c2ccncc2)n1[C@H]1COC(=O)C1] | (R)-4-(4-(4-fluorophenyl)-2-methyl-5-(pyridin-4- yl)-1H-imidazol-1-yl)dihydrofuran-2(3H)-one |
| 3-14 | 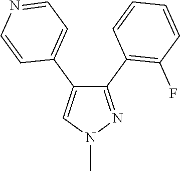 [SMILES: Cn1cc(-c2ccncc2)c(-c2ccccc2F)n1] | 4-(3-(2-fluorophenyl)-1-methyl-1H-pyrazol-4- yl)pyridine |
| 3-15 | 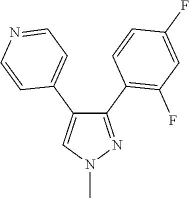 [SMILES: Cn1cc(-c2ccncc2)c(-c2ccc(F)cc2F)n1] | 4-(3-(2,4-difluorophenyl)-1-methyl-1H-pyrazol-4- yl)pyridine |
| 3-16 | 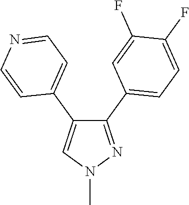 [SMILES: Cn1cc(-c2ccncc2)c(-c2ccc(F)c(F)c2)n1] | 4-(3-(3,4-difluorophenyl)-1-methyl-1H-pyrazol-4- yl)pyridine |
| 3-17 | 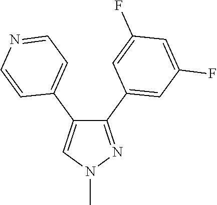 [SMILES: Cn1cc(-c2ccncc2)c(-c2cc(F)cc(F)c2)n1] | 4-(3-(3,5-difluorophenyl)-1-methyl-1H-pyrazol-4- yl)pyridine |
| 3-18 | 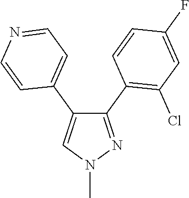 [SMILES: Cn1cc(-c2ccncc2)c(-c2ccc(F)cc2Cl)n1] | 4-(3-(2-chloro-4-fluorophenyl)-1-methyl-1H- pyrazol-4-yl)pyridine |
| 3-19 | 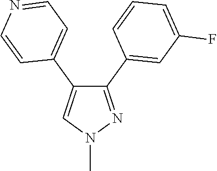 [SMILES: Cn1cc(-c2ccncc2)c(-c2cccc(F)c2)n1] | 4-(3-(3-fluorophenyl)-1-methyl-1H-pyrazol-4- yl)pyridine |
| 3-20 | 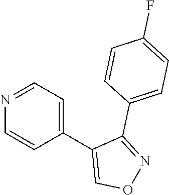 [SMILES: Fc1ccc(-c2nocc2-c2ccncc2)cc1] | 3-(4-fluorophenyl)-4-(pyridin-4-yl)isoxazole |

The present application also provides a compound of Formula V, or a pharmaceutically acceptable salt thereof:

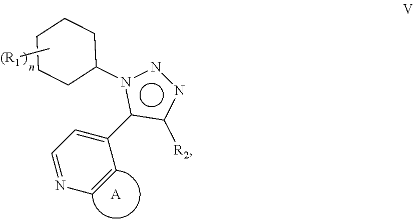

wherein R₁ may be a halogen,
n may be 0, 1, or 2,
R₂ may be absent, —COO-alkyl, or —CO—R₃, wherein R₃ may be a 4- to 7-membered cycloalkyl or heterocycloalkyl or a 5- to 6-membered heteroaryl, wherein up to 2 carbon atoms may be replaced with a heteroatom selected from ═N— and —O—, and said R₃ may optionally be substituted with a R₄ substituent, R₄ may be C₁-C₆ alkyl,
A may be absent or ring A, and
wherein said ring A may be a 4- to 7-membered cycloalkyl or heterocycloalkyl or a 5- to 6-membered heteroaryl, wherein up to 2 carbon atoms may be replaced with a heteroatom selected from ═N— and —O—.

In some cases, in the compound of Formula V, R₁ may be F.

In some cases, in the compound of Formula V, n may be 1.

In some cases, in the compound of Formula V, R₂ may be —CO—R₃.

In some cases, in the compound of Formula V, R₃ may be

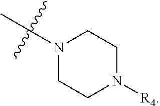

In some cases, in the compound of Formula V, R₄ may be —CH₃.

In some cases, in the compound of Formula V, R₂ may be selected from the group consisting of: —CO₂Et and

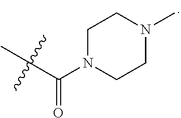 [SMILES: *C(=O)N1CCN(C)CC1]

In some cases, the compound of Formula V may be one of the compounds in table 5.

TABLE 5
The compounds of Formula V

| Compound | Structure | IUPAC NAME |
|---|---|---|
| 4-1 | 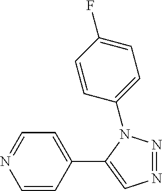 [SMILES: Fc1ccc(-n2nncc2-c2ccncc2)cc1] | 4-(1-(4-fluorophenyl)-1H-1,2,3-triazol-5-yl)pyridine |
| 4-2 | 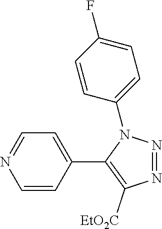 [SMILES: CCOC(=O)c1nnn(-c2ccc(F)cc2)c1-c1ccncc1] | ethyl 1-(4-fluorophenyl)-5-(pyridin-4-yl)-1H-1,2,3- triazole-4-carboxylate |
| 4-3 | 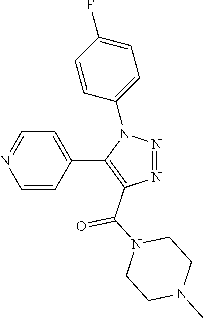 [SMILES: CN1CCN(C(=O)c2nnn(-c3ccc(F)cc3)c2-c2ccncc2)CC1] | (1-(4-fluorophenyl)-5-(pyridin-4-yl)-1H-1,2,3-triazol- 4-yl)(4-methylpiperazin-1-yl)methanone |

The present application also provides a compound of Formula VI, or a pharmaceutically acceptable salt thereof:

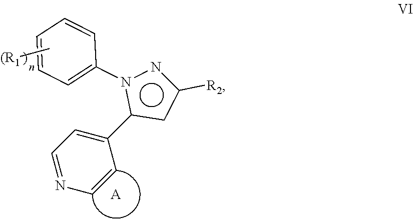

wherein R₁ may be a halogen,
n may be 0, 1, or 2,
R₂ may be C₁-C₆ alkyl, and
A may be a 4- to 7-membered cycloalkyl or heterocycloalkyl or a 5- to 6-membered heteroaryl, wherein up to 2 carbon atoms may be replaced with a heteroatom selected from ═N— and —O—, and said A may optionally be substituted with a R₃ substituent, R₃ may be ═O.

In some cases, in the compound of Formula VI, R₁ may be F.

In some cases, in the compound of Formula VI, n may be 1.

In some cases, in the compound of Formula VI, R₂ may be —CH₃.

In some cases, in the compound of Formula VI, A may be

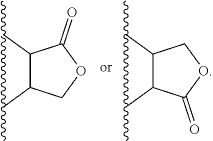

In some cases, the compound of Formula VI may be one of the compounds in table 6.

TABLE 6
The compounds of Formula VI

| Compound | Structure | IUPAC NAME |
|---|---|---|
| 6-1 | 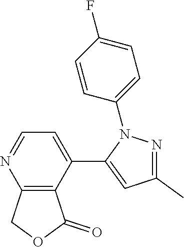 [SMILES: Cc1cc(-c2ccnc3c2C(=O)OC3)n(-c2ccc(F)cc2)n1] | 4-(1-(4-fluorophenyl)-3-methyl-1H-pyrazol-5- yl)furo[3,4-b]pyridin-5(7H)-one |
| 6-2 | 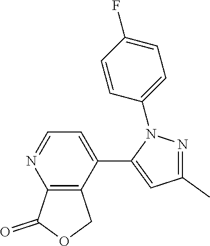 [SMILES: Cc1cc(-c2ccnc3c2COC3=O)n(-c2ccc(F)cc2)n1] | 4-(1-(4-fluorophenyl)-3-methyl-1H-pyrazol-5- yl)furo[3,4-b]pyridin-7(5H)-one |

The present application also provides a compound of

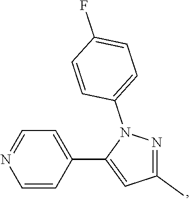 [SMILES: Cc1cc(-c2ccncc2)n(-c2ccc(F)cc2)n1]

or a pharmaceutically acceptable salt thereof.

In some cases, the compound may be described as in table 7.

TABLE 7
The compound 7-1

| Compound | Structure | IUPAC NAME |
|---|---|---|
| 7-1 | 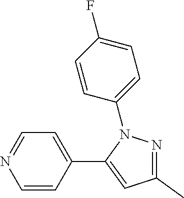 [SMILES: Cc1cc(-c2ccncc2)n(-c2ccc(F)cc2)n1] | 4-(1-(4-fluorophenyl)-3-methyl-1H-pyrazol-5- yl)pyridine |

Medical Use

The present application also provides a method for inhibiting CK1 delta or CK1 epsilon activity, comprising administering an effective amount of a compound of the present application (e.g., one or more of the compounds as described above, such as one or more compounds of Formula I, II, III, IV, V, VI or compound 7-1), or a pharmaceutically acceptable salt thereof.

The method may be an in vitro method, an ex vivo method, or an in vivo method. For example, the compounds of the present application may be administered in vitro to one or more cells. As another example, the compounds of the present application may be administered to a subject in need thereof.

The present application also provides a method for treating a neurological and/or psychiatric disease or disorder in a mammal, which comprises administering to the mammal a therapeutically effective amount of a compound of the present application (e.g., one or more of the compounds as described above, such as one or more compounds of Formula I, II, III, IV, V, VI or compound 7-1) or a pharmaceutically acceptable salt thereof.

The disease or disorder may be a mood disorder, a sleep disorder, or a circadian disorder.

In some cases, the mood disorder may be selected from the group consisting of: a depressive disorder and a bipolar disorder.

The present application also provides a method for treating cancer in a mammal, which comprises administering to the mammal a therapeutically effective amount of a compound of the present application (e.g., one or more of the compounds as described above, such as one or more compounds of Formula I, II, III, IV, V, VI or compound 7-1) or a pharmaceutically acceptable salt thereof.

In some cases, the cancer may be a solid tumor, a blood cancer or a lymphoma. For example, the cancer may be selected from the group consisting of breast cancer, melanoma, leukemia, liver cancer, and brain cancer.

The present application also provides a pharmaceutical composition comprising a compound of the present application (e.g., one or more of the compounds as described above, such as one or more compounds of Formula I, II, III, IV, V, VI or compound 7-1) or a pharmaceutically acceptable salt thereof, and optionally a pharmaceutically acceptable carrier.

Typically, a compound of the present application is administered in an amount effective to treat a condition as described herein. The compounds of the invention may be administered by any suitable route in the form of a pharmaceutical composition adapted to such a route, and in a dose effective for the treatment intended. Therapeutically effective doses of the compounds required to treat the progress of the medical condition are readily ascertained by one of ordinary skill in the art using preclinical and clinical approaches familiar to the medicinal arts. The term “therapeutically effective amount” as used herein generally refers to that amount of the compound being administered which will relieve to some extent one or more of the symptoms of the disorder being treated.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, generally refers to the act of treating as “treating” is defined immediately above. The term “treating” also includes adjuvant and neo-adjuvant treatment of a subject.

The compounds of the application may be administered orally. Oral administration may involve swallowing, so that the compound enters the gastrointestinal tract, or buccal or sublingual administration may be employed by which the compound enters the blood stream directly from the mouth.

In some cases, the compounds of the present application may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

The compounds of the present application may also be administered topically to the skin or mucosa, that is, dermally or transdermally. In some cases, the compounds of the present application can also be administered intranasally or by inhalation. In some cases, the compounds of the present application may be administered rectally or vaginally. In another embodiment, the compounds of the present application may also be administered directly to the eye or ear.

The dosage regimen for the compounds and/or compositions containing the compounds is based on a variety of factors, including the type, age, weight, sex and medical condition of the patient; the severity of the condition; the route of administration; and the activity of the particular compound employed. Thus, the dosage regimen may vary widely. Dosage levels of the order from about 0.01 mg to about 100 mg per kilogram of body weight per day are useful in the treatment of the above-indicated conditions. In one embodiment, the total daily dose of a compound of the invention (administered in single or divided doses) is typically from about 0.01 to about 100 mg/kg, such as from about 0.1 to about 50 mg/kg, from about 0.5 to about 30 mg/kg (i.e., mg compound of the present application per kg body weight). In one embodiment, dosing is from 0.01 to 10 mg/kg/day. In another embodiment, dosing is from 0.1 to 1.0 mg/kg/day. Dosage unit compositions may contain such amounts or submultiples thereof to make up the daily dose. In many instances, the administration of the compound may be repeated a plurality of times in a day (typically no greater than 4 times). Multiple doses per day typically may be used to increase the total daily dose, if desired.

For oral administration, the compositions may be provided in the form of tablets containing at least 0.01, 0.05, 0.1, 0.5, 1.0, 2.5, 5.0, 10.0, 15.0, 25.0, 50.0, 75.0, 100, 125, 150, 175, 200, 250 or 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.1 to about 10 mg/kg/minute during a constant rate infusion.

Suitable subjects according to the present invention include mammalian subjects. Mammals according to the present invention include, but are not limited to, canine, feline, bovine, caprine, equine, ovine, porcine, rodents, lagomorphs, primates, and the like, and encompass mammals in utero. In one embodiment, humans are suitable subjects. Human subjects may be of either gender and at any stage of development.

In another embodiment, the present application provides use of one or more compounds of the present application for the preparation of a medicament for the treatment of the conditions recited herein.

For the treatment of the conditions referred to above, the compounds of the present application can be administered as compound per se. Alternatively, pharmaceutically acceptable salts are suitable for medical applications because of their greater aqueous solubility relative to the parent compound.

In another embodiment, the present application provides pharmaceutical compositions. Such pharmaceutical compositions may comprise a compound of the present application presented with a pharmaceutically acceptable carrier. The carrier can be a solid product, a liquid, or both, and may be formulated with the compound as a unit-dose composition, for example, a tablet, which can contain from 0.05% to 95% by weight of the active compounds. A compound of the present application may be coupled with suitable polymers as targetable drug carriers. Other pharmacologically active substances can also be present.

The compounds of the present invention may be administered by any suitable route, may be in the form of a pharmaceutical composition adapted to such a route, and in a dose effective for the treatment intended. The active compounds and compositions, for example, may be administered orally, rectally, parenterally, or topically.

The compounds of the present application can be used, alone or in combination with other therapeutic agents, in the treatment of various conditions or disease states. The compound(s) of the present application and other therapeutic agent(s) may be administered simultaneously (either in the same dosage form or in separate dosage forms) or sequentially.

The administration of two or more compounds “in combination” means that the two compounds are administered closely enough in time that the presence of one alters the biological effects of the other. The two or more compounds may be administered simultaneously, concurrently or sequentially. Additionally, simultaneous administration may be carried out by mixing the compounds prior to administration or by administering the compounds at the same point in time but at different anatomic sites or using different routes of administration.

The phrases “concurrent administration,” “co-administration,” “simultaneous administration,” and “administered simultaneously” mean that the compounds are administered in combination.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1. Preparation of Compound 1-1

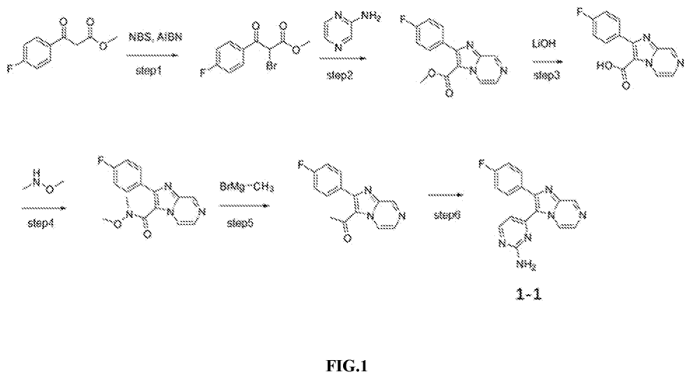

FIG. 1 illustrates the synthetic scheme of compound 1-1. As shown in FIG. 1, the specific synthesis steps are as follows:

Step 1: methyl 2-bromo-3-(4-fluorophenyl)-3-oxopropanoate

NBS (9.98 g, 56.1 mmol) and AIBN (0.73 g, 5.1 mmol) were added to a solution of methyl 3-(4-fluorophenyl)-3-oxopropanoate (10 g, 51 mmol) in 130 mL of CHCl₃. The mixture was stirred at 62° C. for 15 hours (h). The reaction mixture was concentrated under pressure. The crude material was then added to a silica gel column and was eluted with PE/EA (20:1). Chemical Formula: calculated for (M+H⁺) C₁₀H₈BrFO₃: 275.07, Found: 274.7.

Step 2: methyl 2-(4-fluorophenyl) imidazo[1,2-a] pyrazine-3-carboxylate

Methyl 2-bromo-3-(4-fluorophenyl)-3-oxopropanoate (14 g, 50.9 mmol) and pyrazin-2-amine (9.68 g, 101.8 mmol) were dissolved in 100 mL of EtOH. The mixture was heated to reflux for 18 h. The solvent was removed in vacuo and the residue was purified by column chromatography on silica gel and was eluted with DCM/MeOH (50:1). Chemical Formula: calculated for (M+H⁺) C₁₄H₁₀FN₃O₂: 271.25, Found: 271.8.

Step 3: 2-(4-fluorophenyl) imidazo[1,2-a]pyrazine-3-carboxylic acid

Methyl 2-(4-fluorophenyl) imidazo[1,2-a]pyrazine-3-carboxylate (0.7 g, 2.6 mmol) was dissolved in MeOH (40 mL). LiOH (0.31 g, 13 mmol), dissolved in 10 mL water, was added. The reaction mixture was stirred at room temperature for 15 h. The reaction mixture was adjusted to pH=5-6 with HCl (1 mol/L) and then concentrated under reduced pressure, and the crude product was dissolved in ethyl acetate and washed with water. The solvent was concentrated, and the crude material was added to a silica gel column and was eluted with DCM/MeOH (20:1). Chemical Formula: calculated for (M+H⁺) C13H8FN3O2: 257.22, Found: 257.8.

Step 4: 2-(4-fluorophenyl)-N-methoxy-N-methylimidazo[1,2-a]pyrazine-3-carboxamide

2-(4-fluorophenyl) imidazo[1,2-a]pyrazine-3-carboxylic acid (0.7g, 2.7 mmol) and SOCl₂ (0.026 mL, 3.5 mmol) were heated to reflux in anhydrous DCE (30 mL) for 1 h, after which the mixture was evaporated and distilled. The resulting acid chloride was dissolved in toluene and added dropwise over a period of 20 min to a stirred solution of N,O-dimethylhydroxylamine (0.17 g, 2.7 mmol) and K₂CO₃ (0.82 g, 5.8 mmol) in a mixture of H₂O-toluene(1:1, 100 mL) at 0° C. After stirring for 2 h, the organic phase was washed with dilute aqueous NaOH and H₂O. The solvents were evaporated in vacuo. The crude material was added to a silica gel column and was eluted with PE/EA (10:1). Chemical Formula: calculated for (M+H^{+) C}₁₅H₁₃FN₄O₂: 300.29, Found: 300.8.

Step 5: 1-(2-(4-fluorophenyl)imidazo[1,2-a]pyrazin-3-yl)ethan-1-one

A 3 M solution of CH₃MgBr in tetrahydrofuran (THF) (0.26 g, 2.2 mmol) was added to a stirred solution of 2-(4-fluorophenyl)-N-methoxy-N-methylimidazo[1,2-a] pyrazine-3-carboxamide (0.5 g, 1.7 mmol) in THF (20 mL) at −78° C. The mixture was allowed to rest at room temperature and then stirred for 16 h. A saturated solution of ammonium chloride was added, and the mixture was extracted with EtOAc. The solvents were evaporated in vacuo and crude material was added to a silica gel column and was eluted with PE/EA (1:2). Chemical Formula: calculated for (M+H⁺) C₁₄H₁₀FN₃O: 255.25, Found: 255.8.

Step 6: 4-(2-(4-fluorophenyl) imidazo[1,2-a] pyrazin-3-yl) pyrimidin-2-amine

1,1-dimethoxy-N,N-dimethylmethanamine (0.31 g, 2.5 mmol) were added successively to a solution of 1-(2-(4-fluorophenyl)imidazo[1,2-a]pyrazin-3-yl)ethan-1-one (100 mg, 0.4 mmol) in n-propanol (20 mL). The reaction mixture was heated at 92° C. for 3 h. And then, guanidine (0.12 g, 1.9 mmol) and K₂CO₃ (0.25 g, 1.8 mmol) were added. The reaction mixture was stirred at 92° C. for 16 h. Hydroxide sodium solution (5 N, 50 mg) was added, the reaction mixture was stirred at 92° C. for 8 h. Then, the solvent was removed in vacuo and the residue was dissolved in EtOAc, washed with water. The residue was purified by PTLC to obtain the product. Chemical Formula: calculated for (M+H⁺) C₁₆H₁₁FN₆: 306.3, Found: 306.9. ¹H NMR (400 MHz, CDCl₃) δ 9.35 (dd, J=4.7 Hz, 1.4 Hz, 1H), 9.22 (s, 1H), 8.22 (d, J=5.2 Hz, 1H), 8.05 (d, J=4.7 Hz, 1H), 7.71-7.67 (m, 2H), 7.18 (t, J=8.7 Hz, 2H), 6.60 (d, J=5.3 Hz, 1H).

### Example 2. Preparation of Compound 2-2

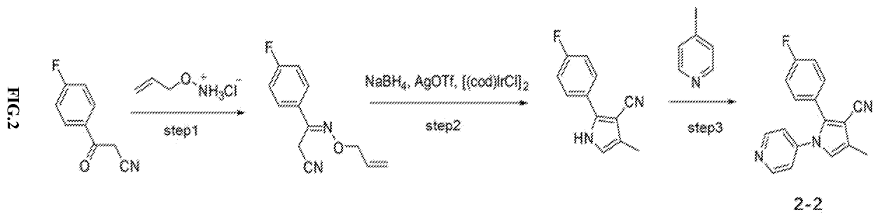

FIG. 2 illustrates the synthetic scheme of compound 2-2. As shown in FIG. 2, the specific synthesis steps are as follows:

Step 1: (Z)-3-((allyloxy)imino)-3-(4-fluorophenyl)propanenitrile

O-allylhydroxylammonium chloride (1.35 g, 12.3 mmol) and KOAc (1.2 g, 12.3 mmol) were decentralized in MeOH (5 mL). The mixture was allowed to stir at 25° C. for 30 min. Meanwhile, 3-(4-fluorophenyl)-3-oxopropanenitrile (2 g, 12.3 mmol) dissolved in MeOH (15 mL) was added dropwise over a 5 min time period. The reaction mixtures were then allowed to stir at 60° C. for 16 h. Then, the solvent was concentrated in vacuo. The crude material was added to a silica gel column and was eluted with PE/EA (20:2). Chemical Formula: calculated for (M+H^{'}) C₁₂H₁₁FN₂O: 218.23, Found: 218.9.

Step 2: 2-(4-fluorophenyl)-4-methyl-1H-pyrrole-3-carbonitrile

[(cod)IrCl]₂ (0.31 g, 0.4 mmol), AgOTf (0.24 g, 0.9 mmol) and NaBH₄ (35 mg, 0.9 mmol) were decentralized in THF (10 mL). This mixture was then allowed to stir at 25° C. for 20 min under nitrogen atmosphere. (Z)-3-((allyloxy)imino)-3-(4-fluorophenyl)propanenitrile (2 g, 9.2 mmol), dissolved in THF (20 mL), was added. The reaction mixture was then allowed to stir at 25° C. for 18 h, and then heated to 50° C. for 24 h. Then, the solvent was concentrated in vacuo. The crude material was added to a silica gel column and was eluted with PE/EA (10:2). Chemical Formula: calculated for (M+H⁺) C₁₂H₉FN₂: 200.22, Found: 200.9.

Step 3: 2-(4-fluorophenyl)-4-methyl-1-(pyridin-4-yl)-1H-pyrrole-3-carbonitrile

A mixture of 2-(4-fluorophenyl)-4-methyl-1H-pyrrole-3-carbonitrile (100 mg, 0.49 mmol), 4-iodopyridine (204 mg, 1.00 mmol), K₂CO₃ (207 mg, 1.50 mmol), and CuI (9.5 mg, 0.05 mmol) were mixed with NMP (4 mL). The system was evacuated and replaced with an argon atmosphere. Then, the mixture was stirred at 200° C. for 2.5 h on microwave. The mixture was extracted with EtOAc, the solvents were evaporated in vacuo and the crude material was purified by PTLC to obtain the product. Chemical Formula: calculated for (M+H⁺) C₁₇H₁₂FN₃: 277.30, Found: 277.8. ¹H NMR (400 MHz, CDCl₃) δ 8.60 (s, 2H), 7.28-7.20 (m, 2H), 7.12-7.05 (m, 2H), 7.02 (d, J=5.5 Hz, 2H), 6.79 (d, J=1.0 Hz, 1H), 2.30 (d, J=0.9 Hz, 3H).

### Example 3. Preparation of Compounds 1-5, 1-6, 1-7 and 1-8

FIG. 3 illustrates the synthetic scheme of compounds 1-5, 1-6, 1-7 and 1-8. As shown in FIG. 3, the specific synthesis steps are as follows:

Step 1: 2-(4-fluorophenyl) imidazo[1,2-a]pyrazine

2-bromo-1-(4-fluorophenyl) ethanone (10 g, 46.1 mmol) and NaHCO₃ (11.6 g, 138.3 mmol) were added to a stirred solution of pyrazin-2-amine (4.38 g, 46.1 mmol) in 300 mL of EtOH. The mixture was heated to reflux for 4 h. Then, the solvent was concentrated in vacuo. The crude material was added to a silica gel column and was eluted with DCM/MeOH (10:1). Chemical Formula: calculated for (M+H⁺) C₁₂H₈FN₃: 213.22, Found: 213.9.

Step 2: 3-bromo-2-(4-fluorophenyl)imidazo[1,2-a]pyrazine

NBS (1 g, 4.7 mmol) was dissolved in CH3CN (30 mL), the solution was stirred at 0° C. Then, 2-(4-fluorophenyl)imidazo[1,2-a]pyrazine (1 g,5.6 mmol) was added. The mixture was stirred at 0° C. for 2 h. The solvent was concentrated and dissolved in EA, washed with water and the solvent was concentrated in vacuo. The crude material was added to a silica gel column and was eluted with PE/EA (1:2). Chemical Formula: calculated for (M+H⁺) C₁₂H₇BrFN₃: 292.11, Found: 291.7.

Step 3: 2-(4-fluorophenyl)-3-(pyridin-4-yl)imidazo[1,2-a]pyrazine

Pyridin-4-ylboronic acid (63 mg, 0.51 mmol) and Pd(PPh₃)₄ (79 mg, 0.068 mmol) were added to a solution of 3-bromo-2-(4-fluorophenyl)imidazo[1,2-a]pyrazine (100 mg, 0.34 mmol) in EtOH (3 mL). Toluene (1 mL) and Sodium carbonate solution (2 N, 0.5 mL) were added. Then, the mixture was swept degas by nitrogen. The mixture was stirred at 100° C. for 3 h on microwave. The solvents were evaporated in vacuo and the crude material was purified by PTLC, to obtain the product. Chemical Formula: calculated for (M+H⁺) C₁₇H₁₁FN₄: 290.30, Found: 290.8. ¹H NMR (400 MHz, CDCl₃) δ 9.22 (d, J=1.2 Hz, 1H), 8.85 (d, J=5.9 Hz, 2H), 8.05 (dd, J=4.7, 1.4 Hz, 1H), 7.97 (d, J=4.7 Hz, 1H), 7.70-7.58 (m, 2H), 7.44 (dd, J=4.5, 1.5 Hz, 2H), 7.09 (t, J=8.7 Hz, 2H).

Step 4: 2-(4-fluorophenyl)-3-(pyridin-4-yl)-5,6,7,8-tetrahydroimidazo[1,2-a]pyrazine

NaBH₄ (26 mg, 0.69 mmol) was added to a solution of 4-[2-(4-fluorophenyl)imidazo[1,2-a]pyrazin-3-yl]pyridine (100 mg, 0.35 mmol) in EtOH (5 mL). The mixture was stirred at 80° C. for 16 h. Then, the solvent was concentrated, and the crude material was purified by PTLC, to obtain the product. Chemical Formula: calculated for (M+H⁺) C₁₇H₁₅FN₄: 294.33, Found: 294.9. ¹H NMR (400 MHz, CDCl₃) δ 8.68 (dd, J=4.5, 1.5 Hz, 2H), 7.50-7.34 (m, 2H), 7.26 (dd, J=4.5, 1.6 Hz, 2H), 6.97 (dd, J=9.7, 7.8 Hz, 2H), 4.28 (s, 2H), 3.86 (t, J=5.4 Hz, 2H), 3.29 (t, J=5.4 Hz, 2H).

Step 5: 1-(2-(4-fluorophenyl)-3-(pyridin-4-yl)-5,6-dihydroimidazo [1,2-a]pyrazin-7(8H)-yl)ethan-1-one

To a solution of 4-[2-(4-fluorophenyl)-5H,6H,7H,8H-imidazo[1,2-a]pyrazin-3-yl]pyridine (20 mg) in THF (5 mL), acetic anhydride (14 mg) was added. The reaction mixture was stirred at 25° C. for 6 h. Then, the solvent was concentrated and the crude material was purified by PTLC, to obtain the product. Chemical Formula: calculated for (M+H⁺) C₁₉H₁₇FN₄O: 336.37, Found: 336.9. ¹H NMR (400 MHz, CDCl₃) δ 8.70 (d, J=5.7 Hz, 2H), 7.49-7.33 (m, 2H), 7.28-7.18 (m, 2H), 6.98 (dd, J=12.0, 5.5 Hz, 2H), 4.91 (s, 2H), 4.06 (t, J=5.3 Hz, 2H), 3.90 (t, J=5.3 Hz, 2H), 2.26 (s, 3H).

Step 6: 2-(4-fluorophenyl)-3-(tributylstannyl)imidazo[1,2-a]pyrazine

A solution of 3-bromo-2-(4-fluorophenyl)imidazo[1,2-a]pyrazine (100 mg, 0.34 mmol) and n-BuLi (39 mg, 0.34 mmol) in THF (5 mL) was stirred at −78° C. under nitrogen atmosphere, then, 2.4 N TMEDA (0.43 mL) was added, the reaction mixture was stirred at −78° C. for 1 h. Then, tributylchlorostannane (166 mg, 0.51 mmol) was added. The reaction mixture was stirred at room temperature for 1 h. The reaction mixture was quenched with saturated solution of ammonium chloride and washed for several times with diethyl ether, the crude material was then purified by PTLC, to obtain the product. Chemical Formula: calculated for (M+H⁺) C₂₄H₃₄FN₃Sn: 502.27, Found: 503.7.

Step 7: 4-(2-(4-fluorophenyl)imidazo[1,2-a]pyrazin-3-yl)furo[3,4-b]pyridin-5(7H)-one

4-bromofuro[3,4-b]pyridin-5(7H)-one (8.5 mg, 0.04 mmol) and Pd(PPh3)4 (9 mg, 0.008 mmol) were added to a solution of 2-(4-fluorophenyl)-3-(tributylstannyl)imidazo[1,2-a]pyrazine (20 mg, 0.04 mmol) in 1,4-dioxane (5 mL). The reaction mixture was stirred at 80° C. for 16 h under nitrogen atmosphere. The mixture was extracted with EtOAc and the solvents were evaporated in vacuo and the crude material was purified by PTLC, to obtain the product. Chemical Formula: calculated for (M+H⁺) C₁₉H₁₁FN₄O₂: 346.32, Found: 346.8. ¹H NMR (400 MHz, CDCl₃) δ 9.15 (d, J=5.0 Hz, 1H), 7.93 (dd, J=8.8, 5.4 Hz, 2H), 7.81-7.69 (m, 2H), 7.66 (s, 1H), 7.55 (dd, J=5.7, 3.3 Hz, 1H), 7.15 (t, J=8.7 Hz, 2H), 5.52 (s, 2H).

### Example 4. Preparation of Compound 6-1

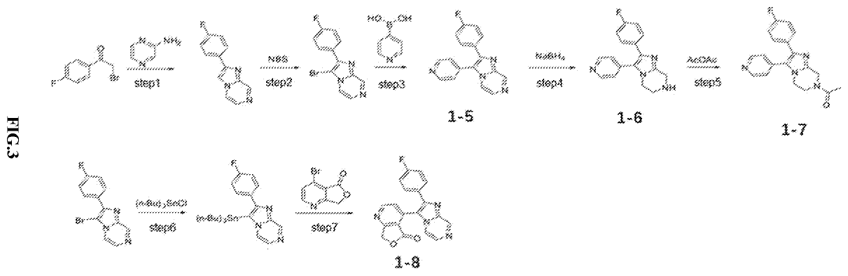

FIG. 4 illustrates the synthetic scheme of compound 6-1. As shown in FIG. 4, the specific synthesis steps are as follows:

Step 1: 4-(1-(4-fluorophenyl)-3-methyl-1H-pyrazol-5-yl)furo[3,4-]pyridin-5(7H)-one

4-bromo-7H-furo[3,4-b]pyridin-5-one (169.5007 mg, 0.792 mmol), Na₂CO₃ (139.9200 mg, 1.32 mmol), and Pd(dppf)C12 (48.2460 mg, 0.066 mmol) were added to a solution of 1-(4-fluorophenyl)-3-methyl-5-(4,4,5,5-tetramethyl-1,3,2-dioxaborolan-2-yl)pyrazole (200 mg, 0.66 mmol) in MePh/H₂O (11 mL). The reaction mixture was stirred at 100° C. for 16 h. The reaction mixture was filtered and concentrated to provide the crude product. The residue was purified by combi-flash with PE/EA (1:1) to provide 4-(1-(4-fluorophenyl)-3-methyl-1H-pyrazol-5-yl)furo[3,4-b]pyridin-5(7H)-one (170 mg, 79.1% yield) as a white solid product, which was determined by LCMS, MS (ESI): mass calculated for C₁₇H₁₂FN₃O₂ 309.1, m/z found 310.0[M+H]⁺. ¹H NMR (400 MHz, DMSO) δ 8.87 (d, J=5.1 Hz, 1H), 7.35 (d, J=5.1 Hz, 1H), 7.25-7.14 (m, 4H), 6.68 (s, 1H), 5.38 (s, 2H), 2.33 (s, 3H).

### Example 5. Preparation of Compounds 1-12, 1-13 and 1-14

FIG. 5 illustrates the synthetic scheme of compounds 1-12, 1-13 and 1-14. As shown in FIG. 5, the specific synthesis steps are as follows:

Step 1: ethyl 3-(4-fluorophenyl)-1H-pyrazole-5-carboxylate

2-ethoxy-2-oxoethanediazonium(19.2 g, 0.167 mmol) was added to a solution of Intermediate 1 (20.0 g, 0.167 mol) in toluene(200 mL) at room temperature and the resulting mixture was stirred at 130° C. for 3 h, The reaction mixture was cooled to room temperature and concentrated under pressure. The crude material was added to a silica gel column and was eluted with PE/EtOAc(3:1) to provide the 15.6 g(40%) of white solid product. Chemical Formula: calculated for (M+H⁺) C₁₂H₁₁FN₂O₂: 235.08, Found: 234.9.

Step 2: ethyl 1-(2-bromoethyl)-3-(4-fluorophenyl)-1H-pyrazole-5-carboxylate

1,2-dibromoethaneand (15 g) was added to a solution of ethyl 5-(4-fluorophenyl)-2H-pyrazole-3-carboxylate(15.6 g) and potassium carbonate(18.4 g) in acetonitrile(200 mL) at room temperature, the resulting mixture was stirred at 90° C. for 4h. The reaction mixture was cooled to room temperature and concentrated under pressure, diluted with water(200 mL). The reaction mixture was extracted with DCM (50 mL*3), filtered and concentrated under pressure. The crude material was added to a silica gel column and was eluted with PE/EtOAc(10:1) to provide 13.5 g(59.5%) of white solid product. Chemical Formula: calculated for (M+H⁺) C₁₄H₁₄BrFN₂O₂: 341.02, Found: 340.7.

Step 3: 5-benzyl-2-(4-fluorophenyl)-6,7-dihydropyrazolo[1,5-a]pyrazin-4(5H)-one

Phenylmethanamine (4.7 g) and NaHCO₃(3.7 g) were added to a solution of ethyl 2-(2-bromoethyl)-5-(4-fluorophenyl)pyrazole-3-carboxylate(13.5 g) and potassium iodide(13 g) in acetonitrile (200 mL) at room temperature. The resulting mixture was stirred at 90° C. for 16 h. The reaction mixture was cooled to room temperature and concentrated under pressure, diluted with water (100 mL). The reaction mixture was extracted with DCM(30 mL*3), filtered and concentrated under pressure. The crude material was added to a silica gel column and was eluted with PE/EtOAc(3:1) to provide 8.9 g(70.0%) of white solid product. Chemical Formula: calculated for (M+H⁺) C₁₉H₁₆FN₃O: 322.13, Found: 321.8.

Step 4: 5-benzyl-2-(4-fluorophenyl)-4,5,6,7-tetrahydropyrazolo[1,5-a]pyrazine

LAH (2.1 g) was added to a solution of 2-(4-fluorophenyl)-5-(1-methylphenyl)-6H,7H-pyrazolo[1,5-a]pyrazin-4-one(8.9 g) in THF (100 mL) at 0° C. The resulting mixture was stirred at 25° C. overnight. The reaction mixture was quenched with ice-cold water, diluted with water (100 mL). The reaction mixture was extracted with DCM (30 mL*3), filtered and concentrated under pressure. The crude material was added to a silica gel column and was eluted with PE/EtOAc (4:1) to provide 4.3 g (50.3%) of white solid product. Chemical Formula: calculated for (M+H⁺) C₁₉H₁₈FN₃: 308.15, Found: 307.8.

Step 5: 5-benzyl-3-bromo-2-(4-fluorophenyl)-4,5,6,7-tetrahydropyrazolo[1,5-a]pyrazine

NBS (1270 mg ,7.14 mmol) was added to a stirred solution of 2-(4-fluorophenyl)-5-(1-methylphenyl)-4H,6H,7H-pyrazolo[1,5-a]pyrazine(2000 mg,6.49 mmol) in acetonitrile (50 mL) with ice-cooling. The mixture was stirred for 1 h, diluted with water (100 mL). The reaction mixture was extracted with EA (50 mL*3), filtered and concentrated under pressure. The crude material was added to a silica gel column and was eluted with PE/EtOAc (4:1) to provide 1.3 g (51.7%) of white solid product. Chemical Formula: calculated for (M+H⁺) C₁₉H₁₇BrFN₃: 386.06, Found: 385.7.

Step 6: 5-benzyl-2-(4-fluorophenyl)-3-(pyridin-4-yl)-4,5,6,7-tetrahydropyrazolo[1,5-a]pyrazine

Pd(dppf)Cl₂(377 mg), pyridin-4-ylboranediol(476 mg), K₃PO₄(1.6g) was added to a solution of 3-bromo-2-(4-fluorophenyl)-5-(1-methylphenyl)-4H,6H,7H-pyrazolo[1,5-a]pyrazine(1 g) in DMF(30 mL), the mixture was allowed to react at 80° C. under N₂ for 16 h. The reaction mixture was cooled to room temperature and concentrated under pressure. The crude material was added to a silica gel column and was eluted with MeOH/DCM(20:1) to provide 0.55 g(55.3%) of white solid product. Chemical Formula: calculated for (M+H⁺) Chemical Formula: C₂₄H₂₁FN₄: 385.18, Found:384.8.

Step 7: 2-(4-fluorophenyl)-3-(pyridin-4-yl)-4,5,6,7-tetrahydropyrazolo[1,5-a]pyrazine

Ammonium formate (454 mg) ,Pd/C (140 mg) was added to a solution of 4-[2-(4-fluorophenyl)-5-(1-methylphenyl)-4H,6H,7H-pyrazolo[1,5-a]pyrazin-3-yl]pyridine(140 mg) in MeOH(20 mL), the mixture was heated to 50° C. for 3 h. The reaction mixture was cooled to room temperature and concentrated under pressure. The crude material was added to a silica gel column and was eluted with MeOH/DCM(20:1) to provide 60 mg(56.6%) of white solid product. Chemical Formula: calculated for (M+H⁺) Chemical Formula: C₁₇H₁₅FN₄: 295.13, Found:294.9.

Step 8: 1-(2-(4-fluorophenyl)-3-(pyridin-4-yl)-6,7-dihydropyrazolo[1,5-a]pyrazin-5(4H)-yl)ethan-1-one

Acetyl acetate was added to a solution of 4-[2-(4-fluorophenyl)-4H,5H,6H,7H-pyrazolo[1,5-a]pyrazin-3-yl]pyridine in THF, the mixture was stirred at room temperature (rt) for 1 h. The reaction mixture was concentrated under pressure. The crude material was added to a silica gel column and was eluted with MeOH/DCM (20:1) to provide 30 mg(33%) of white solid product. Chemical Formula: calculated for (M+H⁺) Chemical Formula: C₁₉H₁₇FN₄O: 337.14, Found:336.8. ¹H NMR (400 MHz, CDCl₃) δ 8.60 (dd, J=15.4, 5.2 Hz, 2H), 7.42-7.36 (m, 2H), 7.14 (dd, J=12.9, 5.4 Hz, 2H), 7.04 (t, J=8.7 Hz, 1H), 4.91 (s, 1H), 4.77 (s, 1H), 4.35 (dt, J=28.1, 5.5 Hz, 2H), 4.19 (t, J=5.5 Hz, 1H), 4.04 (t, J=5.5 Hz, 1H), 2.28 (s, 3H).

### Example 6. Preparation of Compound 1-16

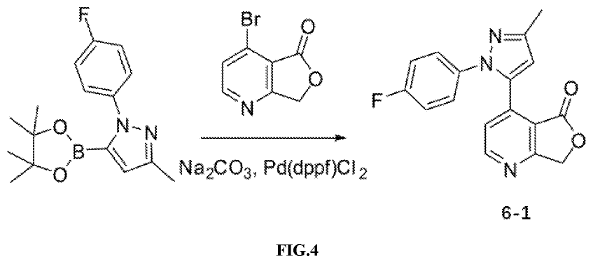

FIG. 6 illustrates the synthetic scheme of compound 1-16. As shown in FIG. 6, the specific synthesis steps are as follows:

Step 1: 2-(4-fluorophenyl)-2,3a,4,5,6,7-hexahydro-3H-indazol-3-one

Ethyl 2-oxocyclohexane-1-carboxylate (10.3911 g, 0.0610 mol) and DIEA (14.319 g, 0.111 mol) were added to a solution of (4-fluorophenyl) hydrazine (7 g, 0.0555 mol) in EtOH (100 mL). The reaction mixture was stirred at 100° C. for 16 h. The reaction was completed and examined with LCMS. The mixture was concentrated to provide the crude product. The mixture was quenched with H₂O (50 mL), filtered and concentrated to provide 2-(4-fluorophenyl)-2,3a,4,5,6,7-hexahydro-3H-indazol-3-one (9.5 g, 62.7% yield) as a yellow solid product, which was determined by LCMS, MS (ESI): mass calculated for C₁₃H₁₃FN₂O 232.1, m/z found 233.1 [M+H]⁺.

Step 2: 3-bromo-2-(4-fluorophenyl)-4,5,6,7-tetrahydro-2H-indazole

POBr3 (11.0682 g, 0.0387 mol) was added to a solution of 2-(4-fluorophenyl)-4,5,6,7-tetrahydro-3aH-indazol-3-one (3 g, 0.0129 mol) in ACN (30 mL). The reaction mixture was stirred at 90° C. for 16 h. The reaction was completed and examined with LCMS. Na₂CO₃ was used to adjust the pH of the mixture (60%SMA and 30% product) to be 7-8. The mixture was extracted with EA (30 mL*3). The combined organic layers were washed with brine (30 mL), dried over sodium sulfate, filtered and concentrated to provide the crude product. The residue was purified by combi-flash with PE/EA (5:1) to provide 3-bromo-2-(4-fluorophenyl)-4,5,6,7-tetrahydro-2H-indazole (0.6 g, 13.2% yield) as a yellow oil product, which was determined by LCMS, MS (ESI): mass calculated for C₁₃H₁₂BrFN₂ 294.0, m/z found 294.9 [M+H]⁺.

Step 3: 2-(4-fluorophenyl)-3-(pyridin-4-yl)-4,5,6,7-tetrahydro-2H-indazole

Pyridin-4-ylboranediol (62.6882 mg, 0.51 mmol), K₃PO₄ (216.2400 mg, 1.02 mmol), and Pd(dppf)Cl₂ (24.8540 mg, 0.034 mmol) were added to a solution of 3-bromo-2-(4-fluorophenyl)-4,5,6,7-tetrahydroindazole (100 mg, 0.34 mmol) in DMF (11 mL). The reaction mixture was stirred at 100° C. for 5 h. The reaction was completed and examined with LCMS. The reaction was quenched with H₂O (30 mL). The mixture was concentrated. The mixture was then extracted with EA (50 mL*3).

The combined organic layers were washed with brine (50 mL), dried over sodium sulfate, filtered and concentrated to provide the crude product. The residue was purified by combi-flash with PE/EA (1:1) to provide 2-(4-fluorophenyl)-3-(pyridin-4-yl)-4,5,6,7-tetrahydro-2H-indazole (24 mg, 24.1% yield) as a white solid product, which was determined by LCMS, MS (ESI): mass calculated for C₁₈H₁₆FN₃ 293.1, m/z found 293.9 [M+H]⁺. ¹H NMR (400 MHz, DMSO) δ 8.56 (dd, J=4.4, 1.6 Hz, 2H), 7.29-7.21 (m, 2H), 7.15 (dd, J=4.4, 1.6 Hz, 1H), 2.68 (t, J=6.0 Hz, 1H), 2.59 (t, J=6.0 Hz, 1H), 1.82 (d, J=5.6 Hz, 1H), 1.74 (d, J=5.6 Hz, 1H).

### Example 7. Preparation of Compound 2-5

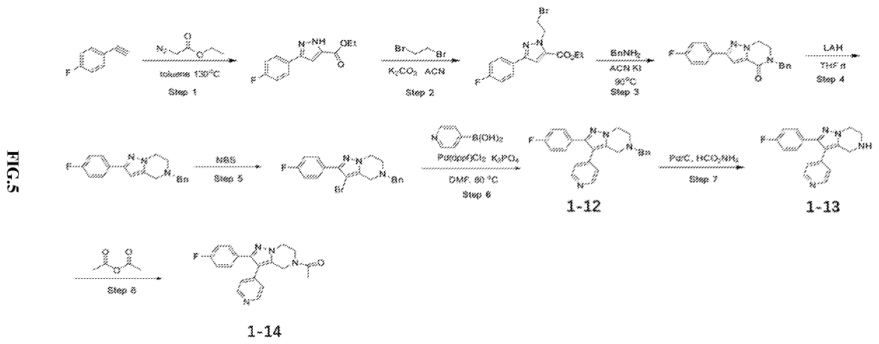

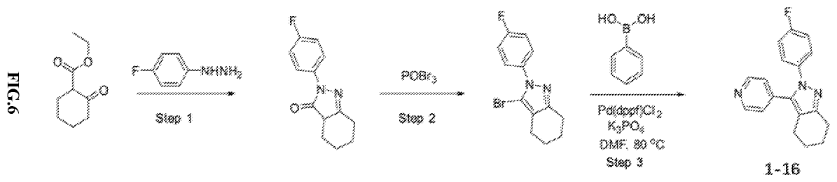

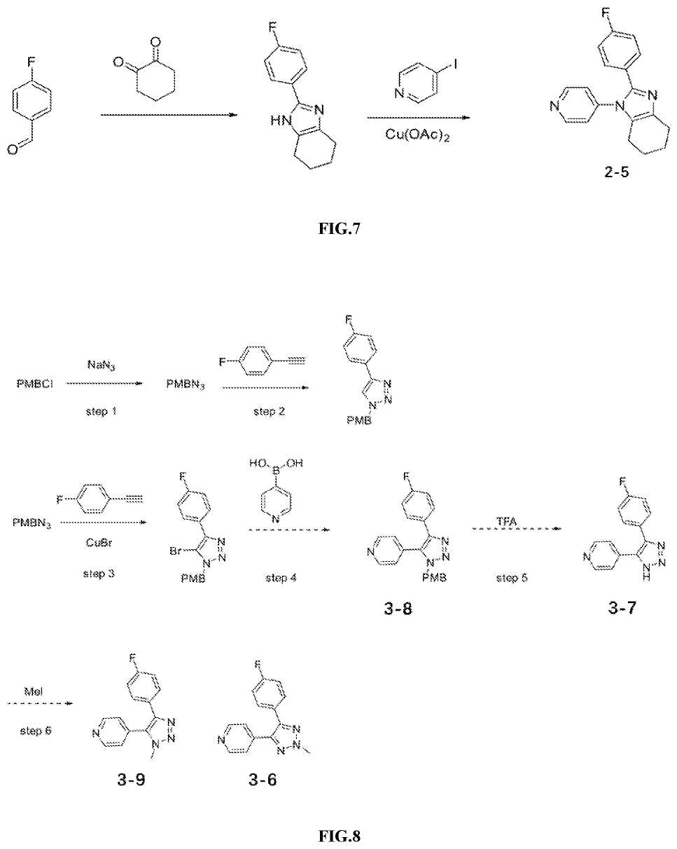

FIG. 7 illustrates the synthetic scheme of compound 2-5. As shown in FIG. 7, the specific synthesis steps are as follows:

Step 1: 2-(4-fluorophenyl)-4,5,6,7-tetrahydro-1H-benzo[d]imidazole

Cyclohexane-1,2-dione (2.7079 g, 0.02415 mol) and NH₄OAc (6.1985 g, 0.0805 mol) were added to a solution of 4-fluorobenzaldehyde (2 g, 0.0161 mol) in EtOH (30 mL). The reaction mixture was stirred at 80° C. for 2 h. The reaction was completed and examined with LCMS. The mixture was concentrated to provide the crude product, The residue was purified by combi-flash with PE/EA (0:1) to provide 2-(4-fluorophenyl)-4,5,6,7-tetrahydro-1H-benzo[d]imidazole (3.4 g, 92.6% yield) as a brown solid product, which was determined by LCMS, MS (ESI): mass calculated for C₁₃H₁₃FN₂ 216.1, m/z found 217.1 [M+H]⁺.

Step 2: 2-(4-fluorophenyl)-1-(pyridin-4-yl)-4,5,6,7-tetrahydro-1H-benzo[d]imidazole

4-iodopyridine (568.783 mg, 2.7746 mmol), K₂CO₃ (574.342 mg, 4.1619 mmol), and CuI (26.3587 mg, 0.13873 mmol) were added to a solution of 2-(4-fluorophenyl)-4,5,6,7-tetrahydro-1H-1,3-benzodiazole (300 mg, 1.3873 mmol) in NMP (4 mL) under microwave. The reaction mixture was stirred at 220° C. for 2.5 hours. The reaction was completed and examined with LCMS. The reaction was quenched with water (20 mL) slowly, the mixture was extracted with EA (30 mL*3). The combined organic layers were washed with brine (30 mL), dried over sodium sulfate, filtered and concentrated, The residue was purified by combi-flash with PE/EA (1:1) to provide 2-(4-fluorophenyl)-1-(pyridin-4-yl)-4,5,6,7-tetrahydro-1H-benzo[d]imidazole (5 mg, 1.20% yield) as a white solid product, which was determined by LCMS, MS (ESI): mass calculated for C₁₈H₁₆FN₃ 293.1, m/z found 293.9 [M+H]⁺. ¹H NMR (400 MHz, CDCl₃) δ 8.71 (dd, J=4.5, 1.6 Hz, 2H), 7.37-7.32 (m, 2H), 7.11 (dd, J=4.5, 1.6 Hz, 2H), 7.01-6.95 (m, 2H), 2.76 (t, J=5.8 Hz, 2H), 2.48 (t, J=5.8 Hz, 2H), 1.93-1.85 (m, 4H).

### Example 8. Preparation of Compounds 3-6, 3-7, 3-8 and 3-9

FIG. 8 illustrates the synthetic scheme of compounds 3-6, 3-7, 3-8 and 3-9. As shown in FIG. 8, the specific synthesis steps are as follows:

Step 1: 1-(azidomethyl)-4-methoxybenzene

Azidosodium (4.14 g, 63.8 mmol) was added to a solution of 1-(chloromethyl)-4-methoxybenzene (5 g, 31.9 mmol) in DMF (30 mL). The reaction mixture was stirred at 80° C. for 3 h. Water(50 mL) was added. The residue was extracted with EA(100 mL*2). The organic phase was dried over sodium sulphate and dried under a stream of nitrogen in the Radleys blowdown apparatus to provide the crude product. The mixture was concentrated under pressure.

Step 2: 4-(4-fluorophenyl)-1-(4-methoxybenzyl)-1H-1,2,3-triazole

1-ethynyl-4-fluorobenzene (809 mg, 6.74 mmol), Na ascorbate(133 mg, 0.674 mmol) and Copper sulfate pentahydrate (168 mg, 0.674 mmol) were added to a solution of 1-(azidomethyl)-4-methoxybenzene (1100 mg,6.74 mmol) in water (15 mL) and t-BuOH(15 mL). The reaction mixture was stirred at 25° C. for 1 h. The solution was filtered, and the filter cake was collected. The mixture was diluted with DCM. The mixture was dried over sodium sulphate and dried under a stream of nitrogen in the Radleys blowdown apparatus to provide the crude product. The reaction mixture was concentrated under pressure. The crude material was added to a silica gel column and was eluted with PE/EtOAc(1:1). ¹H NMR (400 MHz, CDCl₃) δ 7.83-7.73 (m, 2H), 7.60 (s, 1H), 7.32-7.25 (m, 2H), 7.10 (t, J=8.7 Hz, 2H), 6.97-6.91 (m, 2H), 5.52 (s, 2H), 3.83 (s, 3H).

Step 3: 5-bromo-4-(4-fluorophenyl)-1-(4-methoxybenzyl)-1H-1,2,3-triazole

1-ethynyl-4-fluorobenzene (1.47 g, 12.3 mmol), bromocopper (1.76 g, 12.3 mmol), tert-butyl(chloro)dimethylsilane (0.93 g, 6.1 mmol) and DIPEA(1.58 g, 12.3 mmol) were added to a solution of 1-(azidomethyl)-4-methoxybenzene (2 g, 12.3 mmol) in ACN(30 mL). The reaction mixture was stirred at 25° C. for 15 h. The mixture was filtered, and the filtrate was collected. The reaction mixture was concentrated under pressure. The crude material was added to a silica gel column and was eluted with PE/EtOAc(4:1). Chemical Formula: calculated for (M+H⁺) C₁₆H₁₄BrFN₃O: 362.03, Found: 361.6.

Step 4: 4-(4-(4-fluorophenyl)-1-(4-methoxybenzyl)-1H-1,2,3-triazol-5-yl)pyridine

Pyridin-4-ylboranediol (153 mg, 1.245 mmol), Pd(dppf)Cl₂(121.5 mg, 0.166 mmol) and K₃PO₄(352 mg, 1.66 mmol) were added to a solution of 5-bromo-4-(4-fluorophenyl)-1-(4-methoxybenzyl)-1H-1,2,3-triazole (300 mg,0.83 mmol) in DMF(30 mL) and water (5 mL). The reaction mixture was stirred at 80° C. for 5 h. The reaction mixture was concentrated under pressure. The crude material was added to a silica gel column and was eluted with PE/EtOAc(1:1). ¹H NMR (400 MHz, CDCl₃) δ 8.73 (s, 2H), 7.54-7.41 (m, 2H), 7.10 (s, 2H), 6.99 (dd, J=17.6, 8.7 Hz, 4H), 6.81 (d, J=8.7 Hz, 2H), 5.38 (s, 2H), 3.81 (s, 3H).

Step 5: 4-(4-(4-fluorophenyl)-1H-1,2,3-triazol-5-yl)pyridine

A solution of 4-(4-(4-fluorophenyl)-1-(4-methoxybenzyl)-1H-1,2,3-triazol-5-yl)pyridine (90 mg, 0.25 mmol) in TFA (5 mL) was stirred at 80° C. for 2 h. The reaction mixture was concentrated under pressure. The mixture was adjusted to PH=7 with NaHCO₃. The reaction mixture was concentrated under pressure. The crude material was added to a silica gel column and was eluted with CH₂Cl₂/MeOH(5:1). ¹H NMR (400 MHz, MeOD) δ 8.54 (d, J=5.8 Hz, 2H), 7.67-7.44 (m, 4H), 7.23 (t, J=8.7 Hz, 2H).

Step 6: 4-(4-(4-fluorophenyl)-1-methyl-1H-1,2,3-triazol-5-yl)pyridine and 4-(5-(4-fluorophenyl)-2-methyl-2H-1,2,3-triazol-4-yl)pyridine

NaH(7.6 mg, 0.315 mmol) and MeI(30 mg, 0.21 mmol) were added to a solution of 4-[5-(4-fluorophenyl)-3H-1,2,3-triazol-4-yl]pyridine (50 mg, 0.21 mmol) in ACN(5 mL). The reaction mixture was stirred at 25° C. for 3 h. The solution was filtered, and the filtrate was collected. The reaction mixture was concentrated under pressure. The residue was purified via Genal-Prep-HPLC, to obtain the product.

4-[5-(4-fluorophenyl)-3-methyl-1,2,3-triazol-4-yl]pyridine

¹H NMR (400 MHz, MeOD) δ 8.46 (dd, J=4.7, 1.6 Hz, 2H), 7.57-7.44 (m, 4H), 7.36 (t, J=8.8 Hz, 2H), 3.96 (s, 3H).

4-[5-(4-fluorophenyl)-2-methyl-1,2,3-triazol-4-yl]pyridine

¹H NMR (400 MHz, MeOD) δ 8.52 (d, J=5.6 Hz, 2H), 7.64-7.44 (m, 4H), 7.19 (t, J=8.8 Hz, 2H), 4.28 (s, 3H).

### Example 9. Preparation of Compounds 3-1, 3-2, 3-4 and 3-5

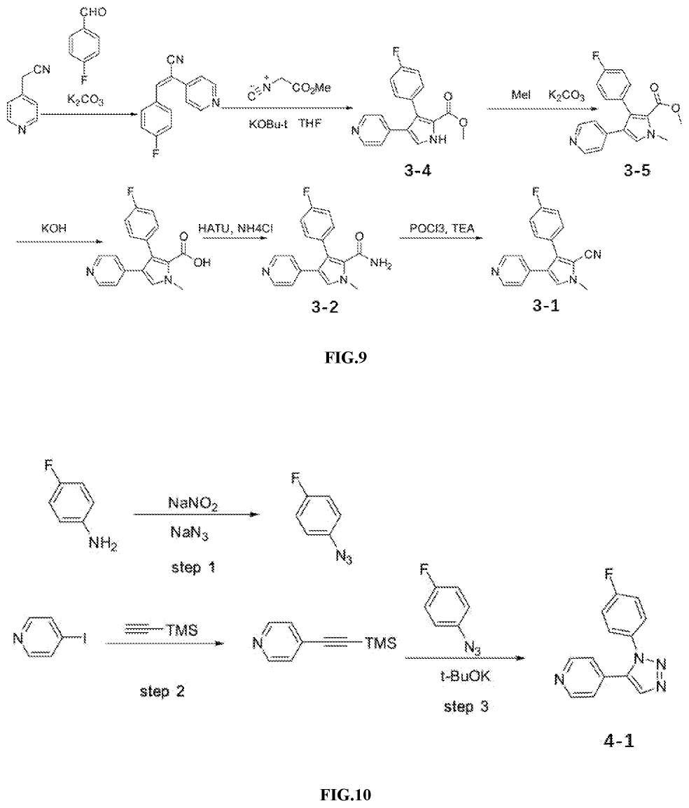

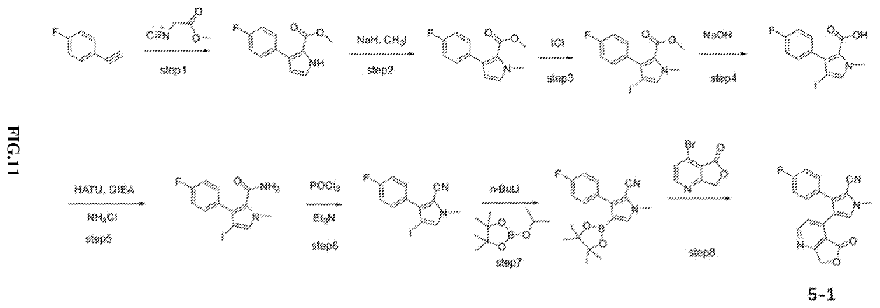

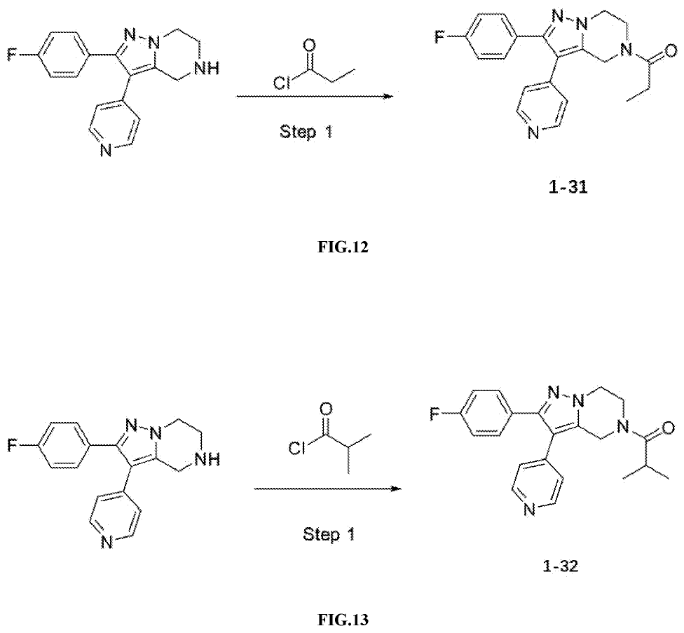

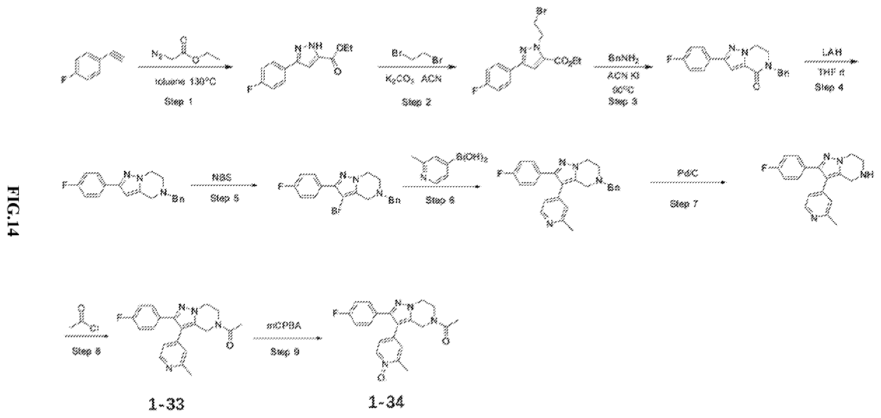

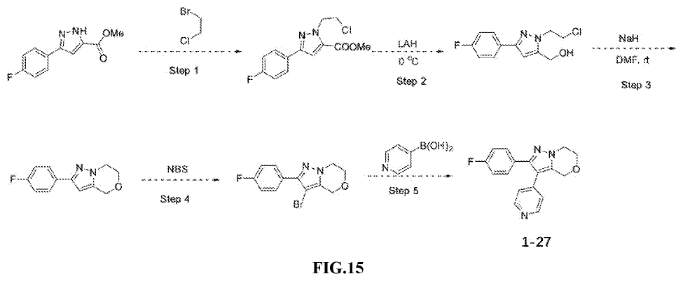

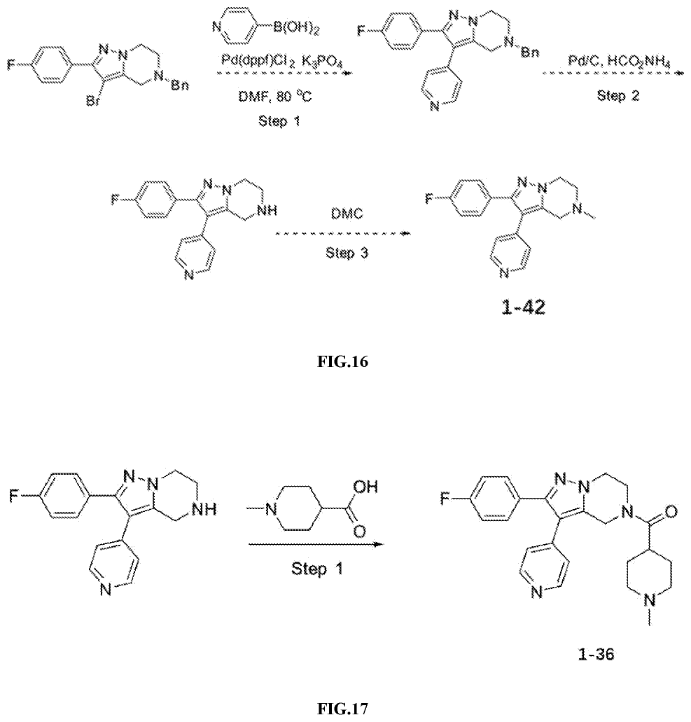

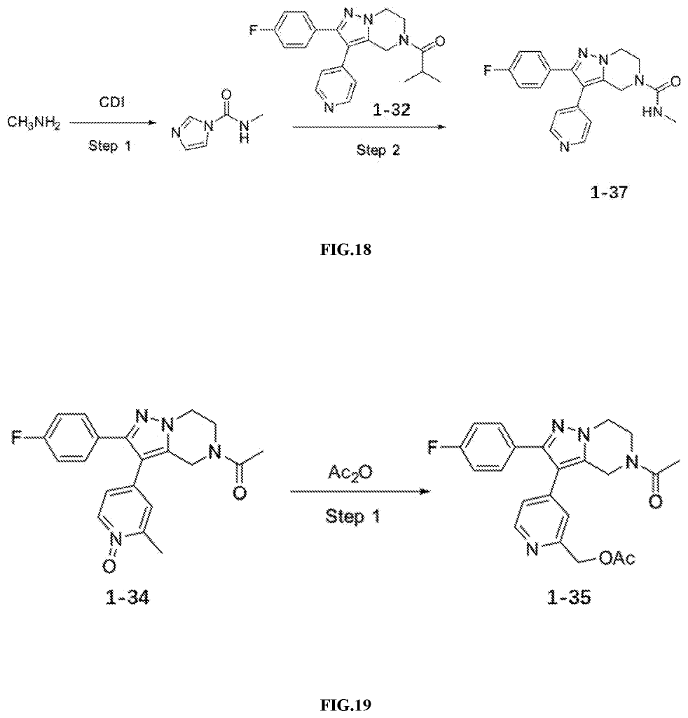

FIG. 9 illustrates the synthetic scheme of compounds 3-1, 3-2, 3-4 and 3-5. As shown in FIG. 9, the specific synthesis steps are as follows:

Step 1: (E)-3-(4-fluorophenyl)-2-(pyridin-4-yl) acrylonitrile

4-fluorobenzaldehyde (3.1462 g, 0.0253 mol) and K₂CO₃ (1.3993 g, 0.0101 mol) were added to a solution of 2-(pyridin-4-yl) acetonitrile (2 g, 0.0169 mol) in MeOH (30 mL). The reaction mixture was stirred at 80° C. for 4 h. The reaction was completed and examined with LCMS. The mixture was concentrated to provide the crude product. Then water was added, the reaction mixture was filtered and the filter cake was concentrated to provide (E)-3-(4-fluorophenyl)-2-(pyridin-4-yl) acrylonitrile (1.8 g, 40.2% yield) as a brown solid product, which was determined by LCMS, MS (ESI): mass calculated for C₁₄H₉FN₂ 224.1, m/z found 225.0 [M+H]⁺.

Step 2: methyl 3-(4-fluorophenyl)-4-(pyridin-4-yl)-1H-pyrrole-2-carboxylate

DBU (1322.4000 mg, 8.7 mmol) was added to a mixture of (2E)-3-(4-fluorophenyl)-2-(pyridin-4-yl)prop-2-enenitrile (1300 mg, 5.80 mmol) and methyl 2-isocyanoacetate (574.7140 mg, 5.8 mmol) in THF (210 mL) at room temperature. The mixture was stirred at 75° C. for 16 h. The reaction mixture was completed and examined by LCMS, and then the mixture was concentrated. The reaction was quenched with water (20 mL). The mixture was extracted with EA (50 mL*3). The combined organic layers were washed with brine (30 mL), dried over sodium sulfate, filtered and concentrated. The residue was purified by combi-flash with PE/EA (0:1) to provide methyl 3-(4-fluorophenyl)-4-(pyridin-4-yl)-1H-pyrrole-2-carboxylate (0.35 g, 0.02% yield) as a brown solid product, which was determined by LCMS, MS (ESI): mass calculated for C₁₇H₁₃FN₂O₂ 296.1, m/z found 296.9 [M+H]⁺.

Step 3: methyl 3-(4-fluorophenyl)-1-methyl-4-(pyridin-4-yl)-1H-pyrrole-2-carboxylate

DABCO (11.3120 mg, 0.101 mmol) and DMF (0.5 mL) were added to a solution of methyl 3-(4-fluorophenyl)-4-(pyridin-4-yl)-1H-pyrrole-2-carboxylate (300 mg, 1.01 mol) in DMC (10 mL). The resulting mixture was heated to 90° C. and stirred at that temperature for 16 h. LCMS (ENB190609-146-R1) showed that the reaction was completed. The reaction was quenched with water (20 mL) slowly, the mixture was extracted with EA (50 mL*3). The combined organic layers were washed with brine (30 mL), dried over sodium sulfate, filtered and concentrated to provide methyl 3-(4-fluorophenyl)-1-methyl-4-(pyridin-4-yl)-1H-pyrrole-2-carboxylate (120 mg, 36.8% yield) as a brown solid product, which was determined by LCMS, MS (ESI): mass calculated for C₁₈H₁₅FN₂O₂ 310.1, m/z found 311.0[M+H]⁺.

Step 4: 3-(4-fluorophenyl)-1-methyl-4-(pyridin-4-yl)-1H-pyrrole-2-carboxylic acid

KOH (53.7600 mg, 0.96 mmol) was added to a solution of methyl 3-(4-fluorophenyl)-1-methyl-4-(pyridin-4-yl) pyrrole-2-carboxylate (100 mg, 0.32 mmol) in MeOH/H2O=1:1 (6 mL) at 25° C. The mixture was stirred at 80° C. for 15 hrs. LCMS showed that the reaction was completed. 4 N HCl was used to adjust the pH of the mixture to 7-8. The mixture was extracted with EA (50 mL*3). The combined organic layers were washed with brine (30 mL), dried over sodium sulfate, filtered and concentrated to provide 3-(4-fluorophenyl)-1-methyl-4-(pyridin-4-yl)-1H-pyrrole-2-carboxylic acid (60 mg, 60.1% yield) as a brown solid product, which was determined by LCMS, MS (ESI): mass calculated for C₁₇H₁₃FN₂O₂ 296.1, m/z found 297.0 [M+H]⁺.

Step 5: 3-(4-fluorophenyl)-1-methyl-4-(pyridin-4-yl)-1H-pyrrole-2-carboxamide

NH₄Cl (31.8 mg, 0.6 mmol), DIEA (77.4 mg, 0.6 mmol), and HATU (91.2 mg, 0.24 mmol) were added to a solution of 3-(4-fluorophenyl)-1-methyl-4-(pyridin-4-yl)pyrrole-2-carboxylic acid (60 mg, 0.2 mmol) in DMF (10 mL) at 25° C. The mixture was stirred at 25° C. for 2 hrs. LCMS showed the reaction was completed. The reaction was quenched with water (20 mL), the mixture was extracted with EA (50 mL*3). The combined organic layers were washed with brine (30 mL), dried over sodium sulfate, filtered and concentrated to provide 3-(4-fluorophenyl)-1-methyl-4-(pyridin-4-yl)-1H-pyrrole-2-carboxamide (70 mg, 118.5% yield) as a brown solid product, which was determined by LCMS, MS (ESI): mass calculated for C₁₇H₁₄FN₃O 295.1, m/z found 296.0 [M+H]⁺. ¹H NMR (400 MHz, CDCl₃) δ 8.44 (s, 2H), 7.32 (dd, J=8.7, 5.4 Hz, 2H), 7.17 (t, J=8.6 Hz, 2H), 7.12 (s, 1H), 7.00 (d, J=11.8 Hz, 2H), 5.17 (s, 2H), 4.06 (s, 3H).

Step 6: 3-(4-fluorophenyl)-1-methyl-4-(pyridin-4-yl)-1H-pyrrole-2-carbonitrile

POCl₃ (93.2688 mg, 0.6096 mmol) and TEA (61.5696 mg, 0.6096 mmol) were added to a solution of 3-(4-fluorophenyl)-1-methyl-4-(pyridin-4-yl)pyrrole-2-carboxamide (60 mg, 0.02 mmol) in THF (10 mL) at 25° C. The mixture was stirred at 25° C. for 2 hrs. LCMS showed the reaction was completed. The reaction was quenched with water (20 mL), the mixture was extracted with EA (50 mL*3). The combined organic layers were washed with brine (30 mL), dried over sodium sulfate, filtered and concentrated. The residue was purified by combi-flash with PE/EA (0:1) to provide 3-(4-fluorophenyl)-1-methyl-4-(pyridin-4-yl)-1H-pyrrole-2-carbonitrile (7 mg, 11.9%yield) as a brown solid product, which was determined by LCMS, MS (ESI): mass calculated for C₁₇H₁₂FN₃ 277.1, m/z found 278.0[M+H]⁺. ¹H NMR (400 MHz, CDCl₃) δ 8.49 (d, J=5.3 Hz, 2H), 7.32-7.29 (m, 2H), 7.13-7.08 (m, 3H), 7.07-7.05 (m, 2H), 3.91 (s, 3H).

### Example 10. Preparation of Compound 4-1

FIG. 10 illustrates the synthetic scheme of compound 4-1. As shown in FIG. 10, the specific synthesis steps are as follows:

Step 1: 1-azido-4-Fluorobenzene

4-fluoroaniline (5 g, 45 mmol) was dissolved in a 3 M HCl aqueous solution (50 mL) at 0° C. NaNO₂ (3.72 g, 54 mmol) was slowly added and the resulting solution was stirred at the same temperature for 0.5 h. Then NaN₃ (4.38 g, 67.5 mmol) dissolved in water (20 mL) was added portion-wise and the mixture was stirred at room temperature for 0.5 h. The reaction mixture was extracted with MTBE (50 mL*2) and the combined organic layers were dried over Na₂SO₄, filtered off and the solvent was evaporated in vacuo to provide the crude product.

Step 2: 4-((trimethylsilyl)ethynyl)pyridine

TEA(9.9 mg, 98 mmol), PPh₃(77 mg, 0.2 mmol) and PdCl₂(PPh₃)₂(344 mg, 0.4 mmol) were added to a solution of 4-iodopyridine (2 g, 9.8 mmol) in THF (30 mL). This mixture was evacuated and backfilled with N₂ for several times to remove oxygen from the solution. The reaction mixture was stirred at 25° C. for 1 h. CuI (56 mg, 0.2 mmol) and ethynyltrimethylsilane (1.44 g, 14.7 mmol) were added. The reaction mixture was stirred at 25° C. for 15 h. Water (50 mL) was added. The residue was extracted with EA(30 mL*3). The reaction mixture was concentrated to provide the crude product. Chemical Formula: calculated for (M+H⁺) C₁₀H₁₄NSi, Molecular Weight: 176.31, Found: 176.0. ¹H NMR (400 MHz, CDCl₃) δ 8.57 (d, J=5.6 Hz, 2H), 7.31 (dd, J=4.5, 1.6 Hz, 2H), 0.27 (s, 9H).

Step 3: 4-(1-(4-fluorophenyl)-1H-1,2,3-triazol-5-yl)pyridine

4-((trimethylsilyl)ethynyl)pyridine (639 mg, 3.65 mmol) and t-BuOK(409 mg, 3.65 mmol) were added to a solution of 1-azido-4-fluorobenzene (500 mg, 3.65 mmol) in DMF(20 mL). The reaction mixture was stirred at 25° C. for 15 h. Water (20 mL) was added. The residue was extracted with EA(2*20 mL). The crude was dried over sodium sulphate and dried under a stream of nitrogen in the Radleys blowdown apparatus to provide the crude product. The reaction mixture was concentrated under pressure. The crude product was added to a silica gel column and was eluted with CH₂Cl₂/MeOH(15:1).The residue was purified via Genal-Prep-HPLC, to obtain the product. Chemical Formula: calculated for (M+H⁺) C₁₃H₁₀FN₄, Molecular Weight: 240.24, Found: 240.9. ¹H NMR (400 MHz, MeOD) δ 8.58 (dd, J=4.7, 1.4 Hz, 2H), 8.23 (s, 1H), 7.57-7.45 (m, 2H), 7.42-7.26 (m, 4H).

### Example 11. Preparation of Compound 5-1

FIG. 11 illustrates the synthetic scheme of compound 5-1. As shown in FIG. 11, the specific synthesis steps are as follows:

Step 1: methyl 3-(4-fluorophenyl)-1H-pyrrole-2-carboxylate

Methyl 2-isocyanoacetate (6.18 g, 62.4 mmol) and Ag₂CO₃ (1.15 g, 4.2 mmol) were added to a solution of 1-ethynyl-4-fluorobenzene (5 g, 42 mmol) in NMP (20 mL). The mixture was stirred at 80° C. for 1 h under nitrogen atmosphere. The mixture was extracted with EtOAc. The solvents were evaporated in vacuo and crude material was added to a silica gel column, was eluted with PE/EA(20:1). Chemical Formula: calculated for (M+H⁺) C₁₂H₁₀FNO₂: 219.22, Found: 219.9.

Step 2: methyl 3-(4-fluorophenyl)-1-methyl-1H-pyrrole-2-carboxylate

The solution of NaH (0.66 g, 27.4 mmol) in DMF (5 mL) was cooled to 0° C. in a dry ice-water bath. Methyl 3-(4-fluorophenyl)-1H-pyrrole-2-carboxylate (3 g, 13.7 mmol) in DMF (10 mL) were added. The mixture was stirred at 0° C. for 1 h, then, ICH₃ (2.9 g, 20.5 mmol) was added. The reaction mixture was stirred at 25° C. for 1 h. 5 mL water was added, and the mixture was extracted with EtOAc. The solvents were evaporated in vacuo and the crude material was added to a silica gel column and was eluted with PE/EA (20:1). Chemical Formula: calculated for (M+H⁺) C₁₃H₁₂FNO₂: 233.24, Found: 233.9.

Step 3: methyl 3-(4-fluorophenyl)-4-iodo-1-methyl-1H-pyrrole-2-carboxylate

ICl (8.4 g, 5.1 mmol) in CC14 was added to a solution of methyl 3-(4-fluorophenyl)-1-methyl-1H-pyrrole-2-carboxylate (1 g, 4.3 mmol) in CCl₄ (5 mL). The mixture was stirred at 0° C. for 15 min. The solvents were evaporated in vacuo and the crude material was added to a silica gel column and was eluted with PE/EA (20:1). Chemical Formula: calculated for (M+H⁺) C₁₃H₁₁FINO₂: 359.14, Found: 359.6.

Step 4: 3-(4-fluorophenyl)-4-iodo-1-methyl-1H-pyrrole-2-carboxylic acid

NaOH (0.22 g, 5.5 mmol) was added to a solution of methyl 3-(4-fluorophenyl)-4-iodo-1-methyl-1H-pyrrole-2-carboxylate (0.4 g, 1.1 mmol) in MeOH/H₂O (6 mL). The mixture was stirred at 60° C. for 5 h. The reaction was adjusted to pH 5-6 with HCl (6 mol/L) and was extracted with EtOAc. The solvents were evaporated in vacuo to provide the crude product. Chemical Formula: calculated for (M+H⁺) C₁₂H₉FINO₂: 345.11, Found: 345.7.

Step 5: 3-(4-fluorophenyl)-4-iodo-1-methyl-1H-pyrrole-2-carboxamide

HATU (502 mg, 1.32 mmol), DIEA (506 mg, 3.3 mmol) and NH4Cl (177 mg, 3.3 mmol) were added to a solution of 3-(4-fluorophenyl)-4-iodo-1-methyl-1H-pyrrole-2-carboxylic acid (380 mg, 1.1 mmol) in DMF (10 mL). The mixture was stirred at room temperature for 1 h. 5 mL water was added, and the mixture was extracted with EtOAc. The solvents were evaporated in vacuo to provide the crude product. Chemical Formula: calculated for (M+H+) C₁₂H₁₀FIN₂O: 344.13, Found: 344.7.

Step 6: 3-(4-fluorophenyl)-4-iodo-1-methyl-1H-pyrrole-2-carbonitrile

POCl₃ (468 mg, 3.05 mmol) and Et3N (309 mg, 3.05 mmol) were added to a solution of 3-(4-fluorophenyl)-4-iodo-1-methyl-1H-pyrrole-2-carboxamide (350 mg, 1.02 mmol) in THF (5 mL). The mixture was stirred at room temperature for 1 h. The solvents evaporated in vacuo and the crude material was added to a silica gel column and was eluted with PE/EA(20:1). Chemical Formula: calculated for (M+H+) C₁₂H₈FIN₂: 326.11, Found: 326.6.

Step 7: 3-(4-fluorophenyl)-1-methyl-4-(4,4,5,5-tetramethyl-1,3,2-dioxaborolan-2-yl)-1H-pyrrole-2-carbonitrile

A solution of 3-(4-fluorophenyl)-4-iodo-1-methyl-1H-pyrrole-2-carbonitrile (50 mg, 0.15 mmol) in THF (3 mL) was stirred at −78° C. under nitrogen atmosphere, then, 2.4 M n-BuLi (0.19 mL) was added, the reaction mixture was stirred at −78° C. for 1 h. Then, 2-isopropoxy-4,4,5,5-tetramethyl-1,3,2-dioxaborolane (86 mg,0.46 mmol) was added. The reaction mixture was stirred at room temperature for 1 h. The reaction was quenched with saturated solution of ammonium chloride and washed for several times with diethyl ether. The solvents were evaporated in vacuo and the crude material was purified by PTLC, to obtain the product. Chemical Formula: calculated for (M+H⁺) C₁₈H₂₀BFN₂O₂: 326.18, Found: 326.8.

Step 8: 3-(4-fluorophenyl)-1-methyl-4-(5-oxo-5,7-dihydrofuro[3,4-b]pyridin-4-yl)-1H-pyrrole-2-carbonitrile

4-bromofuro[3,4-b]pyridin-5(7H)-one (38 mg, 0.18 mmol), Na₂CO₃ (38 mg, 0.36 mmol) and Pd(dppf)Cl₂ (13 mg, 0.018 mmol) were added to a solution of 3-(4-fluorophenyl)-1-methyl-4-(4,4,5,5-tetramethyl-1,3,2-dioxaborolan-2-yl)-1H-pyrrole-2-carbonitrile (60 mg, 0.018 mmol) in toluene/H₂O (5.5 mL). The mixture was stirred at 100° C. for 16 h under nitrogen atmosphere. The solvents were evaporated in vacuo and the crude material was purified by PTLC, to obtain the product. Chemical Formula: calculated for (M+H⁺) C₁₉H₁₂FN₃O₂: 333.32, Found: 333.7. ¹H NMR (400 MHz, CDCl₃) δ 8.50 (d, J=5.3 Hz, 1H), 7.69 (s, 1H), 7.35-7.22 (m, 2H), 7.10 (t, J=8.6 Hz, 2H), 6.88 (d, J=5.3 Hz, 1H), 5.33 (s, 2H), 3.93 (s, 3H).

### Example 12. Preparation of Compound 1-31

FIG. 12 illustrates the synthetic scheme of compound 1-31. As shown in FIG. 12, the specific synthesis steps are as follows:

Step 1: 1-(2-(4-fluorophenyl)-3-(pyridin-4-yl)-6,7-dihydropyrazolo[1,5-a]pyrazin-5(4H)-yl)propan-1-one

Et₃N was added to a solution of 4-[2-(4-fluorophenyl)-4H,5H,6H,7H-pyrazolo[1,5-a]pyrazin-3-yl]-2-methylpyridine in DCM. Then propanoyl chloride was added, the mixture was stirred at room temperature for 2 h. The solvents were evaporated in vacuo and crude material was purified by PTLC, to obtain the product. Chemical Formula: calculated for (M+H⁺) C₂₀H₁₉FN₄O: 350.40, Found: 350.9. ¹H NMR (400 MHz, CDCl₃) δ 8.59 (d, J=10.7 Hz, 2H), 7.46-7.35 (m, 2H), 7.11 (s, 2H), 7.07-6.96 (m, 2H), 4.90 (s, 1H), 4.76 (s, 1H), 4.37 (s, 1H), 4.26 (d, J=43.4 Hz, 2H), 4.04 (s, 1H), 2.65-2.20 (m, 2H), 1.36-1.06 (m, 3H).

### Example 13. Preparation of Compound 1-32

FIG. 13 illustrates the synthetic scheme of compound 1-32. As shown in FIG. 13, the specific synthesis steps are as follows:

Step 1: 1-(2-(4-fluorophenyl)-3-(pyridin-4-yl)-6,7-dihydropyrazolo[1,5-a]pyrazin-5(4H)-yl)-2-methylpropan-1-one

Et₃N was added to a solution of 4-[2-(4-fluorophenyl)-4H,5H,6H,7H-pyrazolo[1,5-a]pyrazin-3-yl]-2-methylpyridine in DCM. Then 2-methylpropanoyl chloride was added, the mixture was stirred at room temperature for 2 h. The solvents were evaporated in vacuo and crude material was purified by PTLC, to obtain the product. Chemical Formula: calculated for (M+H⁺) C₂₁H₂₂FN₄O: 364.42, Found: 364.8. ¹H NMR (400 MHz, CDCl₃) δ 8.58 (s, 2H), 7.42-7.38 (m, 2H), 7.12 (d, J=3.7 Hz, 2H), 7.08-7.00 (m, 2H), 4.85 (d, J=37.0 Hz, 2H), 4.24 (dd, J=80.9, 32.3 Hz, 2H), 1.96 (s, 1H), 1.39-1.04 (m, 2H).

### Example 14. Preparation of Compounds 1-33 and 1-34

FIG. 14 illustrates the synthetic scheme of compounds 1-33 and 1-34. As shown in FIG. 14, the specific synthesis steps are as follows:

Step 1: ethyl 3-(4-fluorophenyl)-1H-pyrazole-5-carboxylate

2-ethoxy-2-oxoethanediazonium was added to a solution of 1-ethynyl-4-fluorobenzene in toluene(300 mL) at room temperature and the resulting mixture was stirred at 130° C. for 3 h. The mixture was cooled to room temperature. The reaction mixture was concentrated under pressure. The crude material was added to a flash chromatography and was eluted with PE/EA (3:1). Chemical Formula: calculated for (M+H⁺) C₁₂H₁₁FN₂O₂: 234.23, Found: 234.9.

Step 2: ethyl 1-(2-bromoethyl)-3-(4-fluorophenyl)-1H-pyrazole-5-carboxylate

1,2-dibromoethaneand was added to a solution of ethyl 3-(4-fluorophenyl)-1H-pyrazole-5-carboxylate and potassium carbonate in acetonitrile (200 mL) at room temperature, the resulting mixture was stirred at 90° C. for 4 h. The mixture was cooled to room temperature. The reaction mixture was concentrated under pressure. The crude material was added to a flash chromatography and was eluted with PE/EA(10:1). Chemical Formula: calculated for (M+H⁺) C₁₄H₁₄BrFN₂O₂: 341.18, Found: 342.02.

Step 3: 5-benzyl-2-(4-fluorophenyl)-6,7-dihydropyrazolo[1,5-a]pyrazin-4(5H)-one

Phenylmethanamine and NaHCO₃ were added to a solution of ethyl 1-(2-bromoethyl)-3-(4-fluorophenyl)-1H-pyrazole-5-carboxylate and potassium iodide in acetonitrile(200 mL) at room temperature, the resulting mixture was stirred at 90° C. for 16 h. The mixture was cooled to room temperature. The reaction mixture was concentrated under pressure. The crude material was added to a flash chromatography and was eluted with PE/EA (3:1). Chemical Formula: calculated for (M+H⁺) C₁₉H₁₆FN₃O: 324.36, Found: 321.9.

Step 4: 5-benzyl-2-(4-fluorophenyl)-4,5,6,7-tetrahydropyrazolo[1,5-a]pyrazine

LAH was added to a solution of 5-benzyl-2-(4-fluorophenyl)-6,7-dihydropyrazolo[1,5-a]pyrazin-4(5H)-one in THF (100 mL) at 0° C., and the resulting mixture was stirred at 25° C. overnight. The reaction mixture was quenched with ice-cold water. The mixture was filtered, and the filtrate was extracted with DCM. The solvents were evaporated in vacuo and crude material was added to a flash chromatography and was eluted with PE/EA (4:1). Chemical Formula: calculated for (M+H⁺) C₁₉H₁₈FN₃: 307.37, Found: 307.9.

Step 5: 5-benzyl-3-bromo-2-(4-fluorophenyl)-4,5,6,7-tetrahydropyrazolo[1,5-a]pyrazine

NBS was added to a stirred solution of 5-benzyl-2-(4-fluorophenyl)-4,5,6,7-tetrahydropyrazolo[1,5-a]pyrazine in acetonitrile with ice-cooling. The mixture was stirred for 2 h. The solvents were evaporated in vacuo and crude material was added to a flash chromatography and was eluted with PE/EA (4:1). Chemical Formula: calculated for (M+H⁺) C₁₉H₁₇BrFN₃: 386.27, Found: 387.7.

Step 6: 5-benzyl-2-(4-fluorophenyl)-3-(2-methylpyridin-4-yl)-4,5,6,7-tetrahydropyrazolo[1,5-a]pyrazine

Pd(dppf)Cl₂,pyridin-4-ylboranediol,K₃PO₄ was added to a solution of 5-benzyl-3-bromo-2-(4-fluorophenyl)-4,5,6,7-tetrahydropyrazolo[1,5-a]pyrazine in DMF, the mixture was allowed to react under N₂ at 80° C. for 3 h. The mixture was extracted with DCM. The solvents were evaporated in vacuo and crude material was added to a flash chromatography and was eluted with PE/EA (1:1). Chemical Formula: calculated for (M+H⁺) C₂₅H₂₃FN₄: 398.49, Found: 398.9.

Step 7: 2-(4-fluorophenyl)-3-(2-methylpyridin-4-yl)-4,5,6,7-tetrahydropyrazolo[1,5-a]pyrazine

Ammonium formate (454 mg) ,Pd/c (140 mg) was added to a solution of 5-benzyl-2-(4-fluorophenyl)-3-(2-methylpyridin-4-yl)-4,5,6,7-tetrahydropyrazolo[1,5-a]pyrazine(140 mg) in MeOH (20 mL), the mixture was heated to 50° C. for 3 h. The solvents were evaporated in vacuo and crude material was added to a flash chromatography and was eluted with DCM/MeOH(20:1). Chemical Formula: calculated for (M+H⁺) C₁₈H₁₇FN₄: 308.36, Found: 308.9.

Step 8: 1-(2-(4-fluorophenyl)-3-(2-methylpyridin-4-yl)-6,7-dihydropyrazolo[1,5-a]pyrazin-5(4H)-yl)ethan-1-one

Et₃N was added to a solution of 2-(4-fluorophenyl)-3-(2-methylpyridin-4-yl)-4,5,6,7-tetrahydropyrazolo[1,5-a]pyrazine in DCM. Then acetyl chloride was added, the mixture was stirred at room temperature for 1 h. The solvents were evaporated in vacuo and crude material was added to a flash chromatography and was eluted with DCM/MeOH(20:1). Chemical Formula: calculated for (M+H⁺) C₂₀H₁₉FN₄O: 350.40, Found: 350.9. ¹H NMR (400 MHz, CDCl₃) δ 8.47 (dd, J=20.0, 5.2 Hz, 1H), 7.47-7.36 (m, 2H), 7.02 (t, J=8.7 Hz, 2H), 6.93 (dd, J=26.4, 6.0 Hz, 2H), 4.87 (s, 1H), 4.74 (s, 1H), 4.37 (t, J=5.4 Hz, 1H), 4.30 (t, J=5.4 Hz, 1H), 4.18 (t, J=5.3 Hz, 1H), 4.02 (t, J=5.4 Hz, 1H), 2.55 (s, 3H), 2.22 (d, J=37.2 Hz, 3H).

Step 9: 4-(5-acetyl-2-(4-fluorophenyl)-4,5,6,7-tetrahydropyrazolo[1,5-a] pyrazine-3-yl)-2-methylpyridine-1-oxide

1-[2-(4-fluorophenyl)-3-(2-methylpyridin-4-yl)-4H,6H,7H-pyrazolo was dissolved in DCM and cooled to 0° C., m-Chloroperoxybenzoic acid was added, and the reaction mixture was stirred for 1 h at room temperature. The mixture was washed with saturated aqueous Na₂CO₃, dried over Na₂SO₄, filtered, and concentrated to provide the desired product. Chemical Formula: calculated for (M+H⁺) C₂₀H₁₉FN₄O₂: 366.40, Found: 366.8. ¹H NMR (400 MHz, CDCl₃) δ 8.28 (dd, J=13.4, 6.6 Hz, 1H), 7.47-7.33 (m, 2H), 7.08-6.94 (m, 4H), 4.88 (s, 1H), 4.74 (s, 1H), 4.37 (t, J=5.4 Hz, 1H), 4.30 (d, J=5.8 Hz, 1H), 4.19 (d, J=5.4 Hz, 1H), 4.03 (t, J=5.4 Hz, 1H), 2.54 (s, 3H), 2.24 (d, J=32.6 Hz, 3H).

### Example 15. Preparation of Compound 1-27

FIG. 15 illustrates the synthetic scheme of compound 1-27. As shown in FIG. 15, the specific synthesis steps are as follows:

Step 1: methyl 1-(2-chloroethyl)-3-(4-fluorophenyl)-1H-pyrazole-5-carboxylate

1-bromo-2-chloroethane (5.5213 g, 0.0385 mol) and K₂CO₃ (3.1878 g, 0.0231 mol) were added to a solution of ethyl 5-(4-fluorophenyl)-2H-pyrazole-3-carboxylate (1.8 g, 0.0077 mol) in acetone (20 mL). The reaction mixture was stirred at 70° C. for 4h. The reaction was completed and examined with LCMS. The mixture was concentrated, then H₂O was added, filtered and the filter cake was concentrated. Chemical Formula: calculated for (M+H⁺) C₁₃H₁₃ClFN₂O₂, Molecular Weight: 283.70, Found: 283.0.

Step 2: (1-(2-chloroethyl)-3-(4-fluorophenyl)-1H-pyrazol-5-yl)methanol

LAH (0.2160 g, 0.0054 mol) was added to a solution of methyl 1-(2-chloroethyl)-3-(4-fluorophenyl)-1H-pyrazole-5-carboxylate (1.6 g, 0.0054 mol) in tetrahydrofuran (30 mL) at 0° C., the reaction mixture was stirred at 0-25° C. for 2 h. The reaction was completed and examined with LCMS.

The reaction was quenched with H₂O (50 mL). The mixture was concentrated. The mixture was extracted with EA (50 mL*3). The combined organic layers were washed with brine (50 mL), dried over sodium sulfate, filtered and concentrated to provide the crude product. Chemical Formula: calculated for (M+H⁺) C₁₂H₁₃ClFN₂O, Molecular Weight: 255.69, Found: 255.0.

Step 3: 2-(4-fluorophenyl)-6,7-dihydro-4H-pyrazolo [5,1-c][1,4]oxazine

NaH (84.72 mg, 3.53 mmol) was added to a solution of (1-(2-chloroethyl)-3-(4-fluorophenyl)-1H-pyrazol-5-yl)methanol (900 mg, 3.53 mmol) in DMF (10 mL) at 0° C. and the reaction mixture was stirred at 0-25° C. for 2 h. The reaction was completed and examined with LCMS. The reaction was quenched with H₂O (50 mL), the mixture was concentrated. The mixture was extracted with EA (50 mL*3). The combined organic layers were washed with brine (50 mL), dried over sodium sulfate, filtered and concentrated to provide the crude product. Chemical Formula: calculated for (M+H⁺) C₁₂H₁₁FN₂O, Molecular Weight: 219.23, Found: 219.0.

Step 4: 3-bromo-2-(4-fluorophenyl)-6,7-dihydro-4H-pyrazolo [5,1-c][1,4]oxazine

NBS (284.8 mg, 1.6 mmol) was added to a solution of 2-(4-fluorophenyl)-6,7-dihydro-4H-pyrazolo[5,1-c][1,4]oxazine (350 mg, 1.6 mmol) in DCM (10 mL), the reaction mixture was stirred at 25° C. for 16 h. The reaction was completed and examined with LCMS. The reaction was quenched with H₂O (50 mL), The mixture was concentrated. The mixture was extracted with EA (50 mL*3). The combined organic layers were washed with brine (50 mL), dried over sodium sulfate, filtered and concentrated to provide the crude product. Chemical Formula: calculated for (M+H⁺) C₁₂H₁₁BrFN₂O, Molecular Weight: 298.13, Found: 298.9.

Step 5: 2-(4-fluorophenyl)-3-(pyridin-4-yl)-6,7-dihydro-4H-pyrazolo[5,1-c][1,4]oxazine

Pyridin-4-ylboranediol (184.3770 mg, 1.5 mmol), K₃PO₄ (106 mg, 0.5 mmol), X-phos (47.7000 mg, 0.1 mmol), and Pd₂(dba)₃ (45.8 mg, 0.05 mmol) were added to a solution of 3-bromo-2-(4-fluorophenyl)-6,7-dihydro-4H-pyrazolo[5,1-c][1,4]oxazine (150 mg, 0.50 mmol) in 1,4-Dioxane (10 mL). The reaction mixture was stirred at 90° C. for 16 h. The reaction was completed and examined with LCMS, then filtered and concentrated. ¹H NMR (400 MHz, DMSO) δ 8.50 (dd, J=4.5, 1.6 Hz, 2H), 7.43-7.36 (m, 2H), 7.25-7.17 (m, 2H), 7.10 (dd, J=4.5, 1.6 Hz, 2H), 4.92 (s, 2H), 4.22 (t, J=5.0 Hz, 2H), 4.14 (t, J=5.1 Hz, 2H) Chemical Formula: calculated for (M+H⁺) C₁₇H₁₅FN₃O, Molecular Weight: 296.32, Found: 296.0.

### Example 16. Preparation of Compound 1-42

FIG. 16 illustrates the synthetic scheme of compound 1-42. As shown in FIG. 16, the specific synthesis steps are as follows:

Step 1: 5-benzyl-2-(4-fluorophenyl)-3-(pyridin-4-yl)-4,5,6,7-tetrahydropyrazolo[1,5-a]pyrazine

Pd(dppf)Cl₂, pyridin-4-ylboranediol, and K₃PO₄ were added to a solution of 5-benzyl-3-bromo-2-(4-fluorophenyl)-4,5,6,7-tetrahydropyrazolo[1,5-a]pyrazine in DMF, the mixture was allowed to react under N2 at 80° C. for 3 h. The solvents were evaporated in vacuo and crude material was added to a flash chromatography and was eluted with PE/EA (1:1). Chemical Formula: calculated for (M+H⁺) C₂₄H₂₁FN₄: 384.46, Found: 384.8.

Step 2: 2-(4-fluorophenyl)-3-(pyridin-4-yl)-4,5,6,7-tetrahydropyrazolo[1,5-a]pyrazine

Ammonium formate (454 mg) and Pd/c (140 mg) were added to a solution of 5-benzyl-2-(4-fluorophenyl)-3-(pyridin-4-yl)-4,5,6,7-tetrahydropyrazolo[1,5-a]pyrazine (140 mg) in MeOH (20 mL), the mixture was heated to 50° C. for 3 h. The mixture was filtered, and the solvents were evaporated in vacuo and crude material was added to a flash chromatography and was eluted with DCM/MeOH (20:1). Chemical Formula: calculated for (M+H⁺) C₁₇H₁₅FN₄: 294.33, Found: 294.9.

Step 3: 2-(4-fluorophenyl)-5-methyl-3-(pyridin-4-yl)-4,5,6,7-tetrahydropyrazolo[1,5-a]pyrazine

Dimethyl carbonate (5 mL) and DABCO (0.8 mg, 0.007 mmol) were added to a solution of 2-(4-fluorophenyl)-3-(pyridin-4-yl)-4,5,6,7-tetrahydropyrazolo[1,5-a]pyrazine (20 mg, 0.07 mmol) in DMF (0.2 mL). The mixture was stirred at 90° C. for 16 h. The mixture was extracted with DCM. The solvents were evaporated in vacuo and crude material was purified by PTLC, to obtain the product. Chemical Formula: calculated for (M+H⁺) C₁₈H₁₇FN₄: 308.36, Found: 308.9. ¹H NMR (400 MHz, CDCl₃) δ 8.55 (d, J=6.0 Hz, 2H), 7.47-7.37 (m, 2H), 7.15 (d, J=6.1 Hz, 2H), 7.05 (dd, J=12.0, 5.4 Hz, 2H), 4.34 (t, J=5.6 Hz, 2H), 3.74 (s, 2H), 3.10-2.88 (m, 2H), 2.58 (d, J=3.8 Hz, 3H).

### Example 17. Preparation of Compound 1-36

FIG. 17 illustrates the synthetic scheme of compound 1-36. As shown in FIG. 17, the specific synthesis steps are as follows:

Step 1: (2-(4-fluorophenyl)-3-(pyridin-4-yl)-6,7-dihydropyrazolo[1,5-a]pyrazin-5(4H)-yl)(1-methylpiperidin-4-yl)methanone

1-methylpiperidine-4-carboxylic acid in oxalic dichloride was heated to 70° C. for 1 h, then the solvent was removed in vacuum, Et₃N,4-[2-(4-fluorophenyl)-4H,5H,6H,7H-pyrazolo[1,5-a]pyrazin-3-yl]pyridine was added, the mixture was stirred at room temperature for 1 h. The solvents were evaporated in vacuo and crude material was purified by PTLC, to obtain the product. Chemical Formula: calculated for (M+H⁺) C₂₄H₂₆FN₅O: 419.21, Found: 419.8. ¹H NMR (400 MHz, CDCl₃) δ 8.57 (s, 2H), 7.47-7.36 (m, 2H), 7.18-6.94 (m, 4H), 4.83 (d, J=43.8 Hz, 2H), 4.37 (s, 2H), 4.13 (d, J=45.8 Hz, 2H), 3.10-2.91 (m, 2H), 2.37 (s, 2H), 1.96 (s, 2H), 1.66 (s, 4H).

### Example 18. Preparation of Compound 1-37

FIG. 18 illustrates the synthetic scheme of compound 1-37. As shown in FIG. 18, the specific synthesis steps are as follows:

Step 1: N-methyl-1H-imidazole-1-carboxamide

CDI(500 mg, 3.08 mmol) and methenamine (95.6 mg, 3.08 mmol) were dissolved in DMF(1 mL) and acetonitrile(3 mL). The solution was stirred at room temperature for 2 h before being concentrated under an air stream to a thick oil. Flash chromatography (4% MeOH/CH₂Cl₂) provide the product.

Step 2: 2-(4-fluorophenyl)-N-methyl-3-(pyridin-4-yl)-6,7-dihydropyrazolo[1,5-a]pyrazine-5(4H)-carboxamide

N-methyl-1H-imidazole-1-carboxamide (8.7 mg, 0.07 mol) and TEA (7.78 mg, 0.077 mol) were added to a solution of 2-(4-fluorophenyl)-3-(pyridin-4-yl)-4,5,6,7-tetrahydropyrazolo[1,5-a]pyrazine (20 mg, 0.07 mol) in DCM (2 mL). The reaction mixture was stirred at 25° C. for 2 h. The mixture was concentrated, then was purified by prep-TLC and prep-HPLC to provide the product. Chemical Formula: calculated for (M+H⁺) C₁₉H₁₉FN₅O, Molecular Weight: 351.39, Found: 351.8. ¹H NMR (400 MHz, CDCl₃) δ 8.57 (s, 2H), 7.40 (dd, J=8.6, 5.4 Hz, 2H), 7.22 (s, 2H), 7.05 (t, J=8.7 Hz, 2H), 4.85 (s, 1H), 4.71 (s, 2H), 4.34 (t, J=5.3 Hz, 2H), 4.01 (t, J=5.3 Hz, 2H), 2.88 (s, 3H).

### Example 19. Preparation of Compound 1-35

FIG. 19 illustrates the synthetic scheme of compound 1-35. As shown in FIG. 19, the specific synthesis steps are as follows:

Step 1: (4-(5-acetyl-2-(4-fluorophenyl)-4,5,6,7-tetrahydropyrazolo[1,5-a] pyrazin-3-yl)pyridin-2-yl)methyl acetate

A solution of 4-(5-acetyl-2-(4-fluorophenyl)-4,5,6,7-tetrahydropyrazolo[1,5-a] pyrazin-3-yl)-2-methylpyridine-1-oxide (30 mg) in Ac₂O (2 mL) was stirred at 100° C. for 1 h. The mixture was concentrated, and purified by prep-HPLC to provide the product. Chemical Formula: calculated for (M+H⁺) C₂₂H₂₂FN₄O₃, Molecular Weight: 408.43, Found: 408.8. ¹H NMR (400 MHz, CDCl₃) δ 8.59 (dd, J=9.9, 5.1 Hz, 1H), 7.40 (dd, J=7.9, 5.8 Hz, 2H), 7.15 (s, 1H), 7.06 (dd, J=12.0, 5.4 Hz, 3H), 5.22 (d, J=3.5 Hz, 2H), 4.91 (s, 1H), 4.78 (s, 1H), 4.38 (t, J=5.3 Hz, 1H), 4.31 (d, J=5.6 Hz, 1H), 4.20 (d, J=5.5 Hz, 1H), 4.07-4.00 (m, 1H), 2.24 (d, J=32.1 Hz, 3H), 2.10 (d, J=3.5 Hz, 3H).

### Example 20. Preparation of Compound 1-24

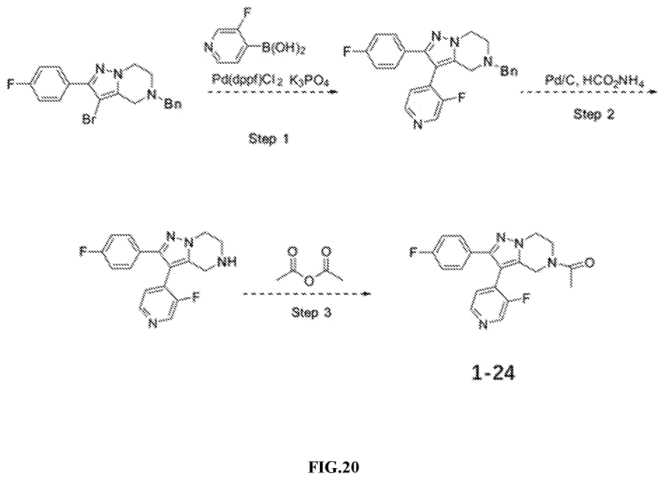

FIG. 20 illustrates the synthetic scheme of compound 1-24. As shown in FIG. 20, the specific synthesis steps are as follows:

Step 1: 5-benzyl-2-(4-fluorophenyl)-3-(3-fluoropyridin-4-yl)-4,5,6,7-tetrahydropyrazolo[1,5-a]pyrazine

SM2(110 mg, 0.78 mmol), Cs₂CO₃(508 mg, 1.56 mmol) and Pd(dppf)Cl₂(38 mg, 0.052 mmol) were added to a solution of 5-benzyl-3-bromo-2-(4-fluorophenyl)-4,5,6,7-tetrahydropyrazolo[1,5-a]pyrazine (200 mg, 0.52 mmol) in dioxane (5 mL) and water (0.5 mL). The reaction mixture was stirred at 100° C. for 3 h. The reaction mixture was concentrated under pressure. The crude material was added to a silica gel column and was eluted with PE/EtOAc (1:1). Chemical Formula: calculated for (M+H⁺) Chemical Formula: C₂₄H₂₁F₂N₄, Molecular Weight: 402.45, Found: 402.8.

Step 2: 2-(4-fluorophenyl)-3-(3-fluoropyridin-4-yl)-4,5,6,7-tetrahydropyrazolo[1,5-a]pyrazine

HCOONH₄(156 mg, 2.48 mmol) and Pd/C(10 mg) were added to a solution of 5-benzyl-2-(4-fluorophenyl)-3-(3-fluoropyridin-4-yl)-4,5, 6,7-tetrahydropyrazolo[1,5-a]pyrazine (100 mg, 0.248 mmol) in (5 mL). The reaction mixture was stirred at 50° C. for 5 h. The solution was filtered, and the filtrate was collected. The reaction mixture was concentrated under pressure. The product was confirmed by (consistent with) LCMS. Chemical Formula: calculated for (M+H⁺) Chemical Formula: C₁₇H₁₅F₂N₄, Molecular Weight: 312.32, Found: 312.8.

Step 3: 1-(2-(4-fluorophenyl)-3-(3-fluoropyridin-4-yl)-6,7-dihydropyrazolo[1,5-a]pyrazin-5(4H)-yl)ethan-1-one

Ac₂O(45.8 mg, 0.448 mmol) was added to a solution of 2-(4-fluorophenyl)-3-(3-fluoropyridin-4-yl)-4,5,6,7-tetrahydropyrazolo[1,5-a]pyrazine (70 mg, 0.224 mmol) in THF(5 mL). The reaction mixture was stirred at 25° C. for 15 h. The reaction mixture was concentrated under pressure. The crude material was added to a silica gel column and was eluted with CH₂Cl₂/MeOH(10:1). The residue was purified via Prep-TLC (CH₂Cl₂/MeOH)=(6:1) to provide the product. The residue was purified via Chiral-Prep-HPLC to provide the product. The residue was purified via Genal-Prep-HPLC to provide the product. ¹H NMR (400 MHz, MeOD) δ 8.54 (d, J=68.1 Hz, 2H), 7.49-7.26 (m, 3H), 7.08 (t, J=8.8 Hz, 2H), 4.81 (d, J=14.3 Hz, 2H), 4.34 (dt, J=36.5, 5.5 Hz, 2H), 4.15 (dt, J=11.0, 5.5 Hz, 2H), 2.22 (d, J=35.9 Hz, 3H). Chemical Formula: calculated for (M+H⁺) Chemical Formula: C₁₉H₁₇F₂N₄O, Molecular Weight: 354.36, Found: 354.9.

### Example 21. Preparation of Compound 7-1

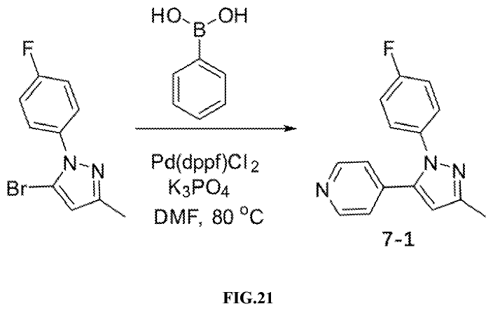

FIG. 21 illustrates the synthetic scheme of compound 7-1. As shown in FIG. 21, the specific synthesis steps are as follows:

Step 1: 4-(1-(4-fluorophenyl)-3-methyl-1H-pyrazol-5-yl) pyridine

Pyridin-4-ylboranediol (71.907 mg, 0.585 mmol), K3PO4 (165.36 mg, 0.78 mmol), and Pd(dppf)Cl2 (28.509 mg, 0.039 mmol) were added to a solution of 5-bromo-1-(4-fluorophenyl)-3-methylpyrazole (100 mg, 0.39 mmol) in DMF (11 mL). The reaction mixture was stirred at 100° C. for 5 h. The reaction was completed and examined with LCMS. The reaction was quenched with H₂O (30 mL), the mixture was then concentrated. The mixture was extracted with EA (50 mL*3). The combined organic layers were washed with brine (50 mL), dried over sodium sulfate, filtered and concentrated to provide the crude product. The residue was purified by combi-flash with PE/EA (1:1) to 4-(1-(4-fluorophenyl)-3-methyl-1H-pyrazol-5-yl) pyridine (60 mg, 509.5% yield) as a white solid product, which was determined by LCMS, MS (ESI): mass calculated for C₁₅H₁₂FN₃ 253.1, m/z found 254.1 [M+H]⁺. ¹H NMR (400 MHz, DMSO) δ 8.54 (dd, J=4.6, 1.4 Hz, 2H), 7.31 (t, J=7.1 Hz, 4H), 7.18 (dd, J=4.6, 1.4 Hz, 2H), 6.69 (s, 1H), 2.29 (s, 3H).

### Example 22. Preparation of Compound 1-44

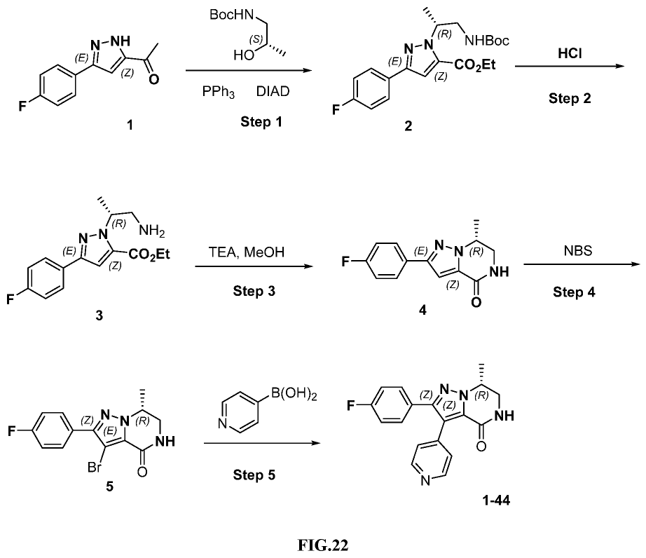

As shown in FIG. 22, the specific synthesis steps are as follows:

Step 1: ethyl (R)-1-(1-((tert-butoxycarbonyl)amino)propan-2-yl)-3-(4-fluorophenyl)-1H-pyrazole-5-carboxylate (2)

PPh₃ (3360 mg, 12.81 mmol) and DIAD (5180 mg, 25.62 mmol) in THF (50 mL), (R)-1-(Boc-amino)-2-propanol (1795 mg, 10.25 mmol) was added to a solution of 1-(3-(4-fluorophenyl)-1H-pyrazol-5-yl)ethan-1-one (2000 mg, 8.54 mmol). The reaction mixture was stirred at 25° C. for 15 h. Saturated NH₄Cl aqueous (50 mL) was added. The residue was extracted with EA (30mL*2). The reaction mixture was concentrated under pressure. The crude material was added to a silica gel column and was eluted with PE/EtOAc (4:1) to give product.

Chemical Formula: calculated for (M+H⁺) C₂₀H₂₆FN₃O₄, Molecular Weight: 391.44, Found: 391.8

Step 2: ethyl (R)-1-(1-((tert-butoxycarbonyl)amino)propan-2-yl)-3-(4-fluorophenyl)-1H-pyrazole-5-carboxylate (3)

TFA (1747 mg, 15.32 mmol) was added to a solution of ethyl (R)-1-(1-((tert-butoxycarbonyl)amino)propan-2-yl)-3-(4-fluorophenyl)-1H-pyrazole-5-carboxylate (3000 mg, 7.66 mmol) in DCM (30 mL). The reaction mixture was stirred at 25° C. for 15 h. The reaction mixture was concentrated under pressure to give product.

Chemical Formula: calculated for (M+H⁺): C₁₅H₁₈FN₃O₂, Molecular Weight: 291.33, Found: 291.8

Step 3: (R)-2-(4-fluorophenyl)-7-methyl-6,7-dihydropyrazolo[1,5-a]pyrazin-4(5H)-one (4)

TEA (1390 mg, 13.74 mmol) was added to a solution of ethyl (R)-1-(1-((tert-butoxycarbonyl)amino)propan-2-yl)-3-(4-fluorophenyl)-1H-pyrazole-5-carboxylate (2000 mg, 6.87 mmol) in MeOH (5 mL). The reaction mixture was stirred at 50° C. for 5 h. The reaction mixture was concentrated under pressure to give product.

Chemical Formula: calculated for (M+H⁺): C₁₃H₁₂FN₃O, Molecular Weight: 245.26, Found: 245.7

Step 4: (R)-3-bromo-2-(4-fluorophenyl)-7-methyl-6,7-dihydropyrazolo[1,5-a]pyrazin-4(5H)-one (5)

NBS (878 mg, 4.935 mmol) was added to a solution of (R)-2-(4-fluorophenyl)-7-methyl-6,7-dihydropyrazolo[1,5-a]pyrazin-4(5H)-one (1000 mg, 3.29 mmol) in THF (20 mL). The reaction mixture was stirred at 50° C. for 20 h. The reaction mixture was concentrated under pressure to give product.

Chemical Formula: calculated for (M+H⁺): C₁₃H₁₁BrFN₃O, Molecular Weight: 324.15, Found: 324.7

Step 5: (R)-2-(4-fluorophenyl)-7-methyl-3-(pyridin-4-yl)-6,7-dihydropyrazolo[1,5-a]pyrazin-4(5H)-one

To a solution of (R)-3-bromo-2-(4-fluorophenyl)-7-methyl-6,7-dihydropyrazolo[1,5-a]pyrazin-4(5H)-one (1000 mg, 3.09 mmol) in Dimethoxyethane (20 mL) and water (3 mL), was added pyridin-4-ylboronic acid (570 mg, 4.63 mmol), Pd(PPh₃)₄ (357 mg, 0.309 mmol) and Na₂CO₃ (667 mg, 6.18 mmol). The reaction mixture was stirred at 100° C. for 5 h. The reaction mixture was concentrated under pressure. The crude material was added to a silica gel column and was eluted with CH₂Cl₂/MeOH (10:1) to give product.

Chemical Formula: calculated for (M+H⁺): C₁₈H₁₅FN₄O, Molecular Weight: 322.34, Found: 322.8

### Example 23. Preparation of Compounds 1-45 and 1-46

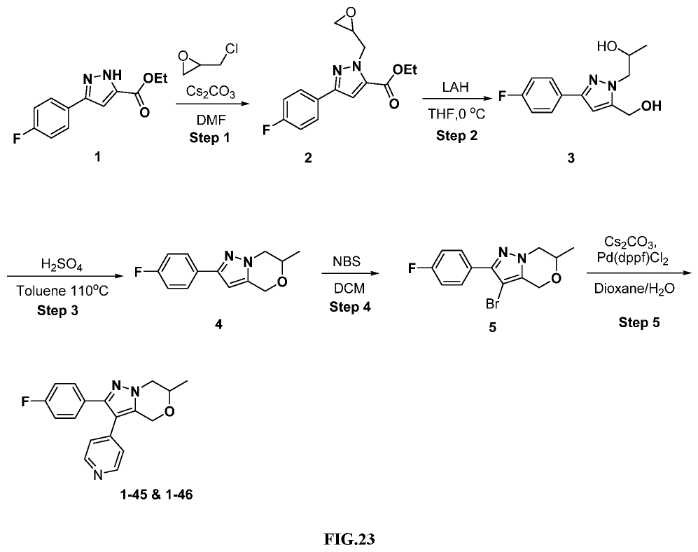

As shown in FIG. 23, the specific synthesis steps are as follows:

Step 1: ethyl 3-(4-fluorophenyl)-1-(oxiran-2-ylmethyl)-1H-pyrazole-5-carboxylate

To a mixture of ethyl 5-(4-fluorophenyl)-2H-pyrazole-3-carboxylate (2.34 g, 10 mmol) in DMF (30 mL) was added 2-(chloromethyl) oxirane (9.26 g, 100mmol), Cs₂CO₃(4.89 g, 15 mmol). The reaction was stirred at 25° C. for 16 h, LCMS showed desired MS was detected as main peak. The mixture was extracted with EA (50 mL*2) and H₂O (50 mL). The organic layer was washed with brine (50 mL), dried over anhydrous Na₂SO₄, filtered and concentrated, then purified by column chromatography (PE/EA=2/1) to give a white solid (1.96 g, yield: 67.5%). Chemical Formula: calculated for (M+H⁺) C₁₅H₁₅FN₂O₃: 290.11, Found: 291.1. ¹H NMR (400 MHz, DMSO) δ 7.94-7.90 (m, 2H), 7.41 (s, 1H), 7.26 (t, J=8.9 Hz, 2H), 4.87 (dd, J=14.5, 3.6 Hz, 1H), 4.61 (dd, J=14.5, 5.5 Hz, 1H), 4.34 (q, J=7.1 Hz, 2H), 3.42-3.38 (m, 1H), 2.80-2.77 (m, 1H), 2.52-2.51 (m, 1H), 1.34 (t, J=7.1 Hz, 3H).

Step 2: 1-(3-(4-fluorophenyl)-5-(hydroxymethyl)-1H-pyrazol-1-yl)propan-2-ol

To a mixture of ethyl 5-(4-fluorophenyl)-2-(oxiran-2-ylmethyl) pyrazole-3-carboxylate (1.45 g, 5.0 mmol) in THF (20 mL) was added Lithium aluminum hydride (380 mg, 10.0 mmol) at 0° C. The reaction was stirred at 0° C. for 4 h, LCMS showed desired MS was detected as main peak. The reaction was quenched by ice, the mixture was extracted with EA (50 mL*2) and H₂O (50 mL). The organic layer was washed with brine (50 mL), dried over anhydrous Na₂SO₄, filtered and concentrated to give a white solid (1.29 g, purity: 90%, yield: 92%). Chemical Formula: calculated for (M+H⁺) C₁₃H₁₅FN₂O₂: 250.11, Found: 251.0. ¹H NMR (400 MHz, DMSO) δ 7.82-7.77 (m, 2H), 7.21 (t, J=8.9 Hz, 2H), 6.59 (s, 1H), 5.29 (t, J=5.6 Hz, 1H), 5.00 (d, J=4.7 Hz, 1H), 4.56 (dd, J=11.1, 6.3 Hz, 2H), 4.07-4.01 (m, 3H), 1.10 (d, J=5.9 Hz, 3H).

Step 3: 2-(4-fluorophenyl)-6-methyl-6,7-dihydro-4H-pyrazolo[5,1-c][1,4]oxazine

To a mixture of 1-[3-(4-fluorophenyl)-5-(hydroxymethyl)pyrazol-1-yl]propan-2-ol(1.0 g, 4.0 mmol) in Toluene (20 mL) was added sulfuric acid (1.96 g, 20 mmol).The reaction was stirred at 110° C. for 4 h. The mixture was concentrated to remove organic, diluted with H₂O (20 mL), treated by 1M NaOH to pH˜7, then extracted with EA (50 mL*2) and H₂O (50 mL). The organic layer was washed with brine (50 mL), dried over anhydrous Na₂SO₄, filtered and concentrated, purified by column chromatography (DCM/MeOH=30/1) to give a white solid (305 mg, yield: 32%). Chemical Formula: calculated for (M+H⁺) C₁₃H₁₃FN₂O: 232.10, Found: 233.1. ¹H NMR (400 MHz, DMSO) δ 7.82-7.78 (m, 2H), 7.22 (t, J=8.9 Hz, 2H), 6.50 (s, 1H), 4.94 (d, J=15.0 Hz, 1H), 4.75 (d, J=15.1 Hz, 1H), 4.22 (dd, J=12.4, 3.1 Hz, 1H), 4.08-4.01 (m, 1H), 3.76 (dd, J=12.1, 10.7 Hz, 1H), 1.31 (d, J=6.2 Hz, 3H).

Step 4: 3-bromo-2-(4-fluorophenyl)-6-methyl-6,7-dihydro-4H-pyrazolo[5,1-c][1,4]oxazine

To a mixture of 2-(4-fluorophenyl)-6-methyl-4H,6H,7H-pyrazolo[3,2-c] [1,4] oxazine (296 mg, 1.27 mmol) in DCM (5 mL) was added NBS (340 mg, 1.905 mmol) stirred at RT for 16 h. LCMS showed desired MS was detected as main peak. The mixture was extracted with DCM (50 mL*2) and H₂O (50 mL). The organic layer was washed with brine (50 mL), dried over anhydrous Na₂SO₄, filtered and concentrated, then purified by column chromatography (PE/EA=2/1) to give a white solid (340 mg, yield: 86%). Chemical Formula: calculated for (M+H⁺) C₁₃H₁₂BrFN₂O: 310.01, Found: 310.9. ¹H NMR (400 MHz, DMSO) δ 7.87-7.81 (m, 2H), 7.34-7.28 (m, 2H), 4.85 (d, J=15.2 Hz, 1H), 4.72 (d, J=15.2 Hz, 1H), 4.23 (dd, J=12.4, 3.0 Hz, 1H), 4.12-4.04 (m, 1H), 3.80 (dd, J=12.2, 10.6 Hz, 1H), 1.32 (d, J=6.2 Hz, 3H).

Step 5: 2-(4-fluorophenyl)-6-methyl-3-(pyridin-4-yl)-6,7-dihydro-4H-pyrazolo[5,1-c][1,4]oxazine

To a solution of 3-bromo-2-(4-fluorophenyl)-6-methyl-4H,6H,7H-pyrazolo[3,2-c][1,4]oxazine(155 mg, 0.55 mmol) in dioxane/H2O (20 mL/4 mL) was added pyridin-4-ylboranediol(185 mg, 1.5 mmol), Cs₂CO₃ (245 mg, 0.75 mmol) and Pd(dppd)Cl₂ (82 mg, 0.1 mmol), and the mixture was stirred at 100° C. under dry N₂ for 4 h. The mixture was concentrated to remove organic, diluted with H₂O (20 mL), then extracted with EA (50 mL*2) and H₂O (50 mL). The organic layer was washed with brine (50 mL), dried over anhydrous Na₂SO₄, filtered and concentrated, purified by column chromatography (DCM/MeOH=30/1) to give a crude product, then purified by SFC (chiralpak-OJ, CO2-MEOH(DEA) to give Compound 1-45 (crude) and Compound 1-46 (27.2 mg, HPLC: 99.47%). Compound 1-45 was purified by HPLC(Gemini-C18 150×21.2 mm, Sum, ACN—H₂O 0.1% FA, gradient 10%˜40%) to give Compound 1-45 (44.8 mg, HPLC: 100%). Chemical Formula: calculated for (M+H⁺) C₁₈H₁₆FN₃O: 309.13, Found: 310.1.

Compound 1-45: ¹H NMR (400 MHz, DMSO) δ 8.50 (d, J=5.3 Hz, 2H), 7.41-7.36 (m, 2H), 7.23-7.18 (m, 2H), 7.10 (d, J=6.0 Hz, 2H), 4.93 (t, J=19.5 Hz, 2H), 4.30 (dd, J=12.6, 3.1 Hz, 1H), 4.16-4.08 (m, 1H), 3.88-3.81 (m, 1H), 1.34 (d, J=6.2 Hz, 3H).

Compound 1-46: ¹H NMR (400 MHz, DMSO) δ 8.50 (dd, J=4.5, 1.6 Hz, 2H), 7.41-7.36 (m, 2H), 7.21 (t, J=8.9 Hz, 2H), 7.10 (dd, J=4.5, 1.6 Hz, 2H), 4.95 (d, J=23.8 Hz, 2H), 4.30 (dd, J=12.5, 3.1 Hz, 1H), 4.14-4.09 (m, 1H), 3.87-3.82 (m, 1H), 1.35 (d, J=6.2 Hz, 3H).

### Example 24. Preparation of Compound 1-47

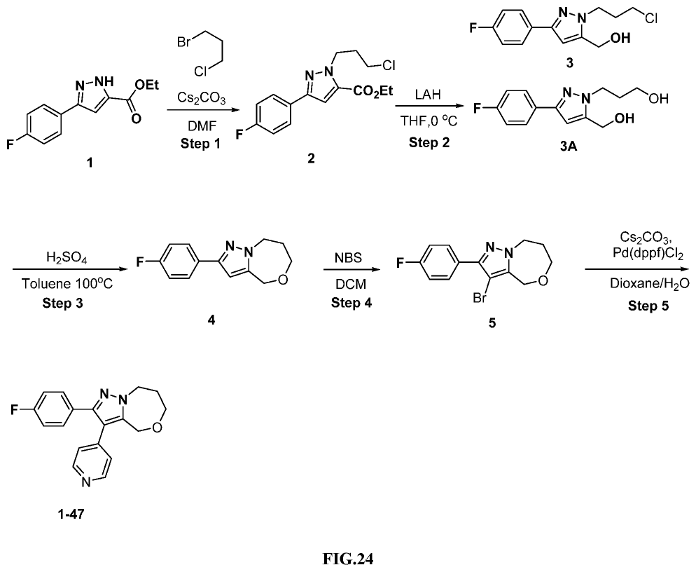

As shown in FIG. 24, the specific synthesis steps are as follows:

Step 1: ethyl 1-(3-chloropropyl)-3-(4-fluorophenyl)-1H-pyrazole-5-carboxylate

To a mixture of ethyl 5-(4-fluorophenyl)-2H-pyrazole-3-carboxylate (2.34 g, 10 mmol) in DMF (20 mL) was added 1-bromo-3-chloropropane (2.37 g, 15 mmol), Cs₂CO₃ (4.89 g, 15 mmol). The reaction was stirred at 25° C. for 16 h, LCMS showed desired MS was detected as main peak. The mixture was extracted with EA (50 mL*2) and H₂O (50 mL). The organic layer was washed with brine (50 mL), dried over anhydrous Na₂SO₄, filtered and concentrated, then purified by column chromatography (PE/EA=2/1) to give a white solid (1.9 g, yield: 58%). Chemical Formula: calculated for (M+H⁺) C₁₅H₁₆ClFN₂O₂: 310.09, Found: 311.1. ¹H NMR (400 MHz, DMSO) δ 7.94-7.89 (m, 2H), 7.38 (s, 1H), 7.29-7.23 (m, 2H), 4.67 (t, J=6.9 Hz, 2H), 4.34 (q, J=7.1 Hz, 2H), 3.67 (t, J=6.3 Hz, 2H), 2.32-2.24 (m, 2H), 1.34 (t, J=7.1 Hz, 3H).

Step 2: (1-(3-chloropropyl)-3-(4-fluorophenyl)-1H-pyrazol-5-yl)methanol and 3-(3-(4-fluorophenyl)-5-(hydroxymethyl)-1H-pyrazol-1-yl)propan-1-ol

To a mixture of ethyl 1-(3-chloropropyl)-3-(4-fluorophenyl)-1H-pyrazole-5-carboxylate (1.24 g, 4.0 mmol) in THF (20 mL) was added Lithium aluminum hydride (304 mg, 8.0 mmol) at 0° C. The reaction was stirred at 0° C. for 4 h, LCMS showed desired MS was detected as main peak. The reaction was quenched by ice, the mixture was extracted with EA (50 mL*2) and H₂O (50 mL). The organic layer was washed with brine (50 mL), dried over anhydrous Na₂SO₄, filtered and concentrated to give two white solids (compound 3,730 mg) and (compound 3A, 80 mg). Compound 3: Chemical Formula: calculated for (M+H⁺) C₁₃H₁₄ClFN₂O: 268.08, Found: 269.1. ¹H NMR (400 MHz, DMSO) δ 7.82-7.78 (m, 2H), 7.24-7.19 (m, 2H), 6.62 (s, 1H), 5.50-5.15 (m, 1H), 4.54 (s, 2H), 4.25 (t, J=6.9 Hz, 2H), 3.69 (t, J=6.5 Hz, 2H), 2.27 (p, J=6.6 Hz, 2H). Compound 3A: Chemical Formula: calculated for (M+H⁺) C₁₃H₁₅FN₂O₂: 250.11, Found: 251.1. ¹H NMR (400 MHz, DMSO) δ 7.79 (dd, J=8.9, 5.6 Hz, 2H), 7.21 (t, J=8.9 Hz, 2H), 6.58 (s, 1H), 5.35 (t, J=5.5 Hz, 1H), 4.61 (t, J=5.1 Hz, 1H), 4.53 (d, J=5.5 Hz, 2H), 4.16 (t, J=7.1 Hz, 2H), 3.45-3.39 (m, 2H), 1.98-1.91 (m, 2H).

Step 3: 2-(4-fluorophenyl)-7,8-dihydro-4H,6H-pyrazolo[5,1-c][1,4]oxazepine

To a mixture of 3-[3-(4-fluorophenyl)-5-(hydroxymethyl)pyrazol-1-yl]propan-1-ol (80 mg, 0.31 mmol) in Toluene (10 mL) was added sulfuric acid (157 mg, 1.59 mmol). The reaction was stirred at 110° C. for 4 h. The mixture was concentrated to remove organic, diluted with H₂O (20 mL), treated by 1M NaOH to pH˜7, then extracted with EA (30 mL*2) and H₂O (30 mL). The organic layer was washed with brine (50 mL), dried over anhydrous Na₂SO₄, filtered and concentrated, purified by column chromatography (DCM/MeOH=30/1) to give a white solid (20 mg, yield: 27%). Chemical Formula: calculated for (M+H⁺) C₁₃H₁₃FN₂O: 232.10, Found: 233.1. ¹H NMR (400 MHz, DMSO) δ 7.79-7.75 (m, 2H), 7.21 (t, J=8.9 Hz, 2H), 6.63 (s, 1H), 4.63 (s, 2H), 4.46-4.42 (m, 2H), 3.98-3.95 (m, 2H), 1.89-1.84 (m, 2H).

Step 4: 3-bromo-2-(4-fluorophenyl)-7,8-dihydro-4H,6H-pyrazolo[5,1-c][1,4]oxazepine

To a mixture of 2-(4-fluorophenyl)-4H,6H,7H,8H-pyrazolo[3,2-c] [1,4] oxazepane (20 mg, 0.09 mmol) in DCM (5 mL) was added NBS (25 mg, 0.135 mmol) stirred at room temperature for 16 h. LCMS showed desired MS was detected as main peak. The mixture was extracted with DCM (30 mL*2) and H₂O (30 mL). The organic layer was washed with brine (50 mL), dried over anhydrous Na₂SO₄, filtered and concentrated, then purified by column chromatography (PE/EA=2/1) to give a white solid (16 mg, yield: 56%). Chemical Formula: calculated for (M+H⁺) C₁₃H₁₂BrFN₂O: 310.01, Found: 311.0. ¹H NMR (400 MHz, DMSO) δ 7.84-7.79 (m, 2H), 7.33-7.27 (m, 2H), 4.68 (s, 2H), 4.53-4.49 (m, 2H), 4.02-3.98 (m, 2H), 1.94-1.87 (m, 2H).

Step 5: 2-(4-fluorophenyl)-3-(pyridin-4-yl)-7,8-dihydro-4H,6H-pyrazolo[5,1-c][1,4]oxazepine

To a solution of 3-bromo-2-(4-fluorophenyl)-7,8-dihydro-4H,6H-pyrazolo[5,1-c][1,4]oxazepine (16 mg, 0.05 mmol) in dioxane/H₂O (5 mL/1 mL) was added pyridin-4-ylboranediol(19 mg, 0.15 mmol), Cs₂CO₃ (25 mg, 0.075 mmol) and Pd(dppd)Cl₂ (9 mg, 0.01 mmol), and the mixture was stirred at 100° C. under dry N₂ for 4 h. The mixture was concentrated to remove organic solvent, diluted with H₂O (20 mL), then extracted with EA (30 mL×2) and H₂O (30 mL). The organic layer was washed with brine (50 mL), dried over anhydrous Na₂SO₄, filtered and concentrated, purified by column chromatography (DCM/MeOH=30/1) to give a crude product, then purified by HPLC(Gemini-C18 150×21.2 mm, Sum, ACN—H₂O 0.1% FA, gradient 10%˜40%) to give a white solid (3.2 mg, yield: 20%, HPLC: 99.72%). Chemical Formula: calculated for (M+H⁺) C₁₈H₁₆FN₃O: 309.13, Found: 310.1. ¹H NMR (400 MHz, DMSO) δ 8.52 (s, 2H), 7.39-7.34 (m, 2H), 7.22 (d, J=4.7 Hz, 2H), 7.08 (t, J=8.8 Hz, 2H), 4.71 (s, 2H), 4.61-4.58 (m, 2H), 4.13-4.10 (m, 2H), 2.08-2.02 (m, 2H).

### Example 25. Preparation of Compound 5-2

As shown in FIG. 25, the specific synthesis steps are as follows:

Step 1: 3-(4-fluorophenyl)-1-methyl-4-(1H-pyrazolo[3,4-b]pyridin-4-yl)-1H-pyrrole-2-carbonitrile

4-bromo-1H-pyrazolo[3,4-b]pyridine (46 mg, 0.23 mmol), 2 M Na₂CO₃ aqueous solutions (0.5 mL) and Pd(dppf)Cl₂ (22 mg, 0.03 mmol) were added to a solution of 3-(4-fluorophenyl)-1-methyl-4-(4,4,5,5-tetramethyl-1,3,2-dioxaborolan-2-yl)-1H-pyrrole-2-carbonitrile (50 mg, 0.15 mmol) in EtOH/toluene=8:2 (5 mL). The mixture was stirred at 100° C. for 1 h under nitrogen atmosphere on microwave. The reaction mixture was concentrated under pressure. The residue was purified via Prep-TLC PE/EA (1:10) and give the product. Chemical Formula: calculated for (M+H⁺) C₁₈H₂FN₅: 317.33, Found: 317.8. ¹H NMR (400 MHz, CDCl₃) δ 12.43 (s, 1H), 8.49 (d, J=4.9 Hz, 1H), 7.76 (s, 1H), 7.31 (dd, J=6.0, 2.8 Hz, 2H), 7.25 (s, 1H), 7.04 (t, J=8.7 Hz, 2H), 6.87 (d, J=4.9 Hz, 1H), 3.97 (s, 3H).

### Example 26. Preparation of Compound 1-48

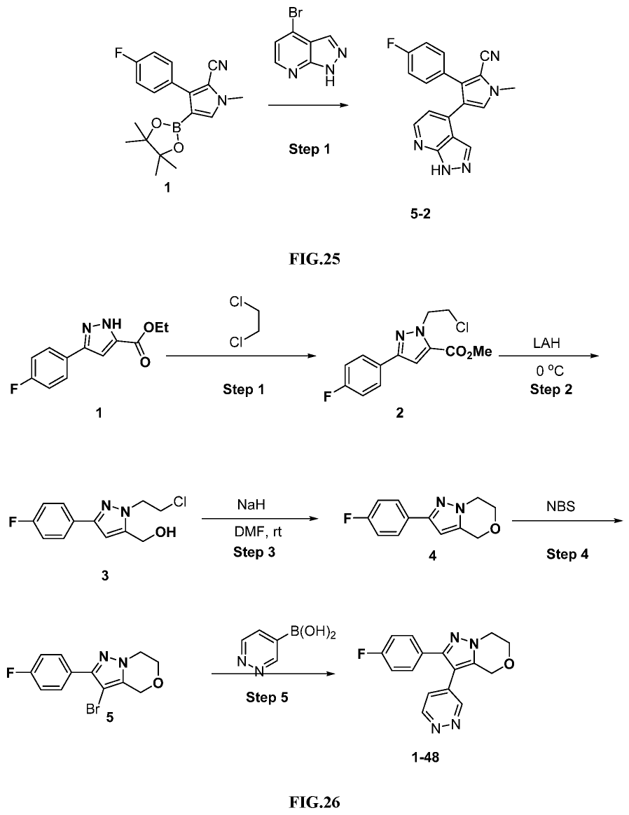

As shown in FIG. 26, the specific synthesis steps are as follows:

Step 1: ethyl 1-(2-chloroethyl)-3-(4-fluorophenyl)-1H-pyrazole-5-carboxylate (2)

To a solution of ethyl 5-(4-fluorophenyl)-2H-pyrazole-3-carboxylate (1.8 g, 0.0077 mol) in acetone (20 mL) was added 1-bromo-2-chloroethane (5.5213 g, 0.0385 mol) and K₂CO₃ (3.1878 g, 0.0231 mol). The reaction mixture was stirred at 70° C. for 4 h. The mixture was complete was detected by LCMS. The mixture was concentrated, then was added H₂O, filtered and filter cake was concentrated. Chemical Formula: calculated for (M+H^{'}) C₁₃H₁₂ClFN₂O₂: 282.1, Found: 283.0

Step 2: (1-(2-chloroethyl)-3-(4-fluorophenyl)-1H-pyrazol-5-yl)methanol (3)

To a solution of ethyl 2-(2-chloroethyl)-5-(4-fluorophenyl) pyrazole-3-carboxylate (1.6 g, 0.0054 mol) in tetrahydrofuran (30 mL) was added LAH (0.2160 g, 0.0054 mol) at 0° C. The reaction mixture was stirred at 0-25° C. for 2 h. The reaction was complete was detected by LCMS. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The crude material was added to a silica gel column and was eluted with PE/EtOAc (1:1). Chemical Formula: calculated for (M+H⁺) C₁₂H₁₂ClFN₂O: 254.1, Found: 255.0

Step 3: 2-(4-fluorophenyl)-6,7-dihydro-4H-pyrazolo [5,1-c][1,4]oxazine (4)

To a solution of [2-(2-chloroethyl)-5-(4-fluorophenyl)pyrazol-3-yl]methanol (900 mg, 3.53 mmol) in DMF (10 mL) was added NaH (84.72 mg, 3.53 mmol) at 0° C. and the reaction mixture was stirred at 0-25° C. for 2 h. The reaction was complete was detected by 1 cms. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The crude material was added to a silica gel column and was eluted with PE/EtOAc (1:1). Chemical Formula: calculated for (M+H⁺) C₁₂H₁₁FN₂O: 218.1, Found: 219.0

Step 4: 3-bromo-2-(4-fluorophenyl)-6,7-dihydro-4H-pyrazolo[5,1-c][1,4]oxazine (5)

To a solution of 2-(4-fluorophenyl)-4H,6H,7H-pyrazolo[3,2-c][1,4]oxazine (350 mg, 1.6 mmol) in DCM (10 mL) was added NBS (284.8 mg, 1.6 mmol). The reaction mixture was stirred at 25° C. for 16 h. The reaction was complete detected by 1 cms. The reaction was quenched by H₂O (50 mL). The mixture was concentrated. The mixture was extracted with EA (50 mL*3). The combined organic layers were washed with brine (50 mL), dried over sodium sulfate, filtered and concentrated to give the crude product. The crude material was added to a silica gel column and was eluted with PE/EtOAc (1:1). Chemical Formula: calculated for (M+H⁺) C₁₂H₁₀BrFN₂O: 290.0, Found: 296.9

Step 5: 2-(4-fluorophenyl)-3-(pyridin-4-yl)-6,7-dihydro-4H-pyrazolo[5,1-c][1,4]oxazine

To a solution of 2-(4-fluorophenyl)-3-(4,4,5,5-tetramethyl-1,3,2-dioxaborolan-2-yl)-4H,6H,7H-pyrazolo[3,2-c][1,4]oxazine (50 mg, 0.15 mmol) in 1,2-Dimethoxyethane (5 mL) was added 4-bromopyridazine (47.70 mg, 0.3 mmol), Na₂CO₃ (47.70 mg, 0.4499 mmol) and Pd(dtbpf)Cl₂ (9.76 mg, 0.015 mmol). The reaction mixture was stirred at 90° C. for 16 h. The reaction was complete detected by LCMS. The mixture was filtered and concentrated to give the product. The crude material was added to a silica gel column and was eluted with PE/EtOAc (1:1). The residue was purified via Genal-Prep-HPLC and give the product.

¹H NMR (400 MHz, DMSO) δ 9.24 (dd, J=5.3, 1.2 Hz, 1H), 9.05 (dd, J=2.3, 1.2 Hz, 1H), 7.56 (dd, J=5.3, 2.4 Hz, 1H), 7.46 (dd, J=15.2, 8.7 Hz, 1H), 7.39 (dd, J=8.8, 5.5 Hz, 2H), 7.23 (t, J=8.9 Hz, 2H), 5.82 (d, J=15.2 Hz, 1H), 5.70 (s, 1H), 5.07 (d, J=8.7 Hz, 1H), 4.53 (d, J=5.5 Hz, 2H).

### Example 27. Preparation of Compound 5-3

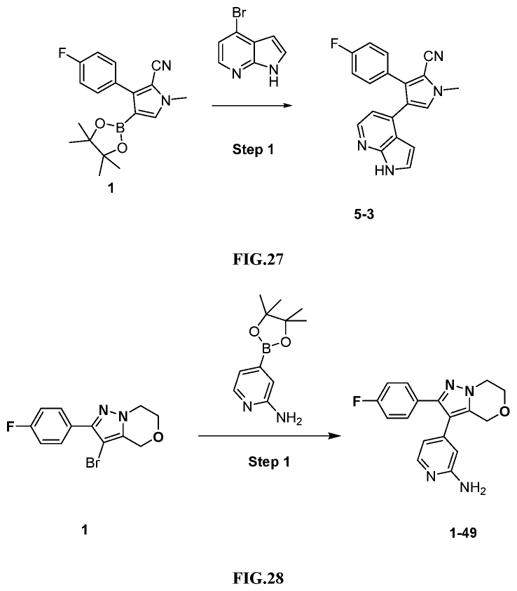

As shown in FIG. 27, the specific synthesis steps are as follows:

Step 1: 3-(4-fluorophenyl)-1-methyl-4-(1H-pyrrolo[2,3-b]pyridin-4-yl)-1H-pyrrole-2-carbonitrile

4-bromo-1H-pyrrolo[2,3-b]pyridine (46 mg, 0.23 mmol), 2 MNa₂CO₃ aqueous solutions (0.5 mL) and Pd(dppf)Cl₂ (22 mg, 0.03 mmol) were added to a solution of 3-(4-fluorophenyl)-1-methyl-4-(4,4,5,5-tetramethyl-1,3,2-dioxaborolan-2-yl)-1H-pyrrole-2-carbonitrile (50 mg, 0.15 mmol) in EtOH/toluene=8:2 (5 mL). The mixture was stirred at 100° C. for 1 h under nitrogen atmosphere on microwave. The reaction mixture was concentrated under pressure. The residue was purified via Prep-TLC PE/EA (1:10) and give the product. Chemical Formula: calculated for (M+H⁺) C₁₉H₁₃FN₄: 316.34, Found: 316.8. ¹H NMR (400 MHz, CDCl₃) δ 9.66 (s, 1H), 7.32-7.29 (m, 2H), 7.27 (d, J=2.0 Hz, 2H), 7.21 (s, 1H), 7.01 (t, J=8.7 Hz, 2H), 6.81 (s, 1H), 6.31 (s, 1H), 3.95 (s, 3H).

### Example 28. Preparation of Compound 1-49

As shown in FIG. 28, the specific synthesis steps are as follows:

Step 1: 4-(2-(4-fluorophenyl)-6,7-dihydro-4H-pyrazolo[5,1-c][1,4]oxazin-3-yl)pyridin-2-amine

To a solution of 3-bromo-2-(4-fluorophenyl)-4H,6H,7H-pyrazolo[3,2-c][1,4]oxazine (500 mg, 1.68 mmol) in EtOH/PhMe/H₂O=8:4:1 (13 mL) was added 4-(4,4,5,5-tetramethyl-1,3,2-dioxaborolan-2-yl) pyridin-2-amine (739.46 mg, 3.36 mmol), Na₂CO₃ (534.24 mg, 5.04 mmol) and Pd(dppf)Cl₂ (245.62 mg, 0.336 mmol). The reaction mixture was stirred at 100° C. for 1 h. The reaction was complete detected by LCMS. The mixture was filtered and concentrated to give the product. The crude material was added to a silica gel column and was eluted with EtOAc. ¹H NMR (301 MHz, DMSO) δ 7.81 (dd, J=4.8, 1.2 Hz, 1H), 7.45-7.38 (m, 2H), 7.22-7.15 (m, 2H), 6.21-6.17 (m, 2H), 5.87 (s, 2H), 4.81 (s, 2H), 4.17 (d, J=4.8 Hz, 2H), 4.13 (d, J=4.8 Hz, 2H).

### Example 29. Preparation of Compound 1-50

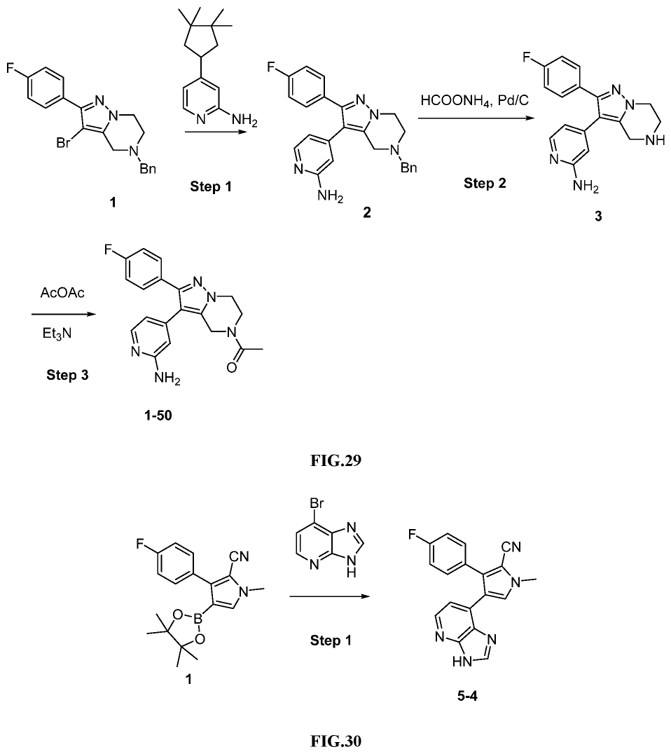

As shown in FIG. 29, the specific synthesis steps are as follows:

Step 1: 4-(5-benzyl-2-(4-fluorophenyl)-4,5,6,7-tetrahydropyrazolo[1,5-a]pyrazin-3-yl)pyridin-2-amine

4-(3,3,4,4-tetramethylcyclopentyl)pyridin-2-amine (102 mg, 0.46 mmol), 2 M Na₂CO₃ aqueous solutions (0.5 mL) and Pd(dppf)Cl₂ (45 mg, 0.06 mmol) were added to a solution of 5-benzyl-3-bromo-2-(4-fluorophenyl)-4,5,6,7-tetrahydropyrazolo[1,5-a]pyrazine (120 mg, 0.31 mmol) in EtOH/toluene=8:2 (5 mL). The mixture was stirred at 100° C. for 1 h under nitrogen atmosphere on microwave. The reaction mixture was concentrated under pressure. The residue was purified via Prep-TLC PE/EA (1:4) and give the product. Chemical Formula: calculated for (M+H⁺) C₂₄H₂₂FN₅: 399.47, Found: 399.8.

Step 2: 4-(2-(4-fluorophenyl)-4,5,6,7-tetrahydropyrazolo[1,5-a]pyrazin-3-yl)pyridin-2-amine

HCOONH₄ (157 mg, 2.5 mmol) and Pd/C (200 mg) were added to a solution of 4-(5-benzyl-2-(4-fluorophenyl)-4,5, 6,7-tetrahydropyrazolo[1,5-a]pyrazin-3-yl)pyridin-2-amine (100 mg, 0.25 mmol) in MeOH (10 mL). The mixture was stirred at 60° C. for 2 h. The solution was filtered, filtrate was collected and the solution was concentrated under pressure. Chemical Formula: calculated for (M+H⁺) C₁₇H₁₆FN₅: 309.35, Found: 309.8.

Step 3: 1-(3-(2-aminopyridin-4-yl)-2-(4-fluorophenyl)-6,7-dihydropyrazolo[1,5-a]pyrazin-5(4H)-yl)ethan-1-one

AcOAc (29 mg, 0.29 mmol) and Et₃N (88 mg, 0.87 mmol) were added to a solution of 4-(2-(4-fluorophenyl)-4,5,6,7-tetrahydropyrazolo[1,5-a]pyrazin-3-yl)pyridin-2-amine (90 mg, 0.29 mmol) in DCM (10 mL). The mixture was stirred at room temperature for 2 h. The reaction mixture was concentrated under pressure. The residue was purified via Prep-TLC DCM/MeOH (20:1) and give the product. Chemical Formula: calculated for (M+H⁺) C₁₉H₁₈FN₅O: 351.39, Found: 351.8. ¹H NMR (400 MHz, CDCl₃) δ 7.71 (d, J=16.3 Hz, 1H), 7.39 (dd, J=8.4, 5.0 Hz, 2H), 7.04 (dd, J=20.6, 12.0 Hz, 2H), 6.59 (d, J=10.3 Hz, 1H), 6.39 (d, J=5.6 Hz, 1H), 4.85 (d, J=39.9 Hz, 2H), 4.34 (dd, J=20.7, 15.4 Hz, 2H), 4.23-3.95 (m, 2H), 2.37-2.14 (m, 3H).

### Example 30. Preparation of Compound 5-4

As shown in FIG. 30, the specific synthesis steps are as follows:

Step 1: 3-(4-fluorophenyl)-4-(3H-imidazo[4,5-b]pyridin-7-yl)-1-methyl-1H-pyrrole-2-carbonitrile

7-bromo-3H-imidazo[4,5-b]pyridine (137 mg, 0.69 mmol), 2 M Na₂CO₃ aqueous solutions (0.5 mL) and Pd(dppf)C12 (67 mg, 0.092 mmol) were added to a solution of 3-(4-fluorophenyl)-1-methyl-4-(4,4,5,5-tetramethyl-1,3,2-dioxaborolan-2-yl)-1H-pyrrole-2-carbonitrile (150 mg, 0.46 mmol) in EtOH/toluene=8:2 (5 mL). The mixture was stirred at 100° C. for 1 h under nitrogen atmosphere on microwave. The reaction mixture was concentrated under pressure. The residue was purified via Prep-TLC PE/EA (1:1) and give the product. Chemical Formula: calculated for (M+H⁺) C₁₈H₁₂FN₅: 317.33, Found: 317.8. ¹H NMR (400 MHz, CDCl₃) δ 8.19 (s, 1H), 7.93 (d, J=8.4 Hz, 1H), 7.32 (td, J=5.6, 2.1 Hz, 3H), 7.04 (dd, J=12.0, 5.3 Hz, 2H), 6.98 (d, J=8.4 Hz, 1H), 3.90 (s, 3H).

### Example 31. Preparation of Compound 1-51

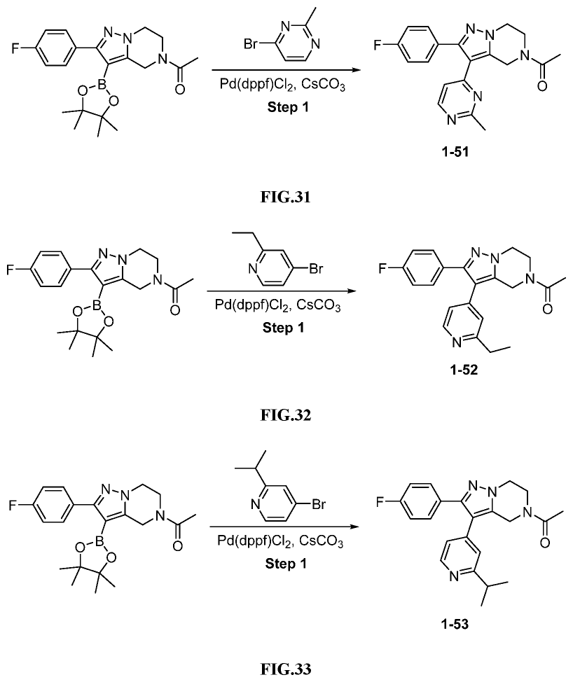

As shown in FIG. 31, the specific synthesis steps are as follows:

Step1: 1-(2-(4-fluorophenyl)-3-(2-methylpyrimidin-4-yl)-6, 7-dihydropyrazolo[1,5-a]pyrazin-5 (4H)-yl)ethan-1-one

Pd(dppf)Cl₂ (28.5 mg, 0.039 mmol), 4-bromo-2-methylpyrimidine (101.2 mg, 0.585 mmol), Cs₂CO₃ (381 mg, 1.17 mmol) was added to a solution of 1-(2-(4-fluorophenyl)-3-(4,4,5,5-tetramethyl-1,3,2-dioxaborolan-2-yl)-6,7-dihydropyrazolo [1,5-a]pyrazin-5(4H)-yl)ethan-1-one (150 mg, 0.39 mmol) in dioxane/H₂O (20:4 mL). The mixture was allowed to react at 90° C. under N₂ for 16 h. The reaction mixture was cooled to room temperture and concentrated under pressure. The crude material was added to a silica gel column and was eluted with MeOH/DCM (25:1) to give product as yellow solid (36 mg, yield: 25.7%). Chemical Formula: calculated for (M+H⁺) Chemical Formula: C₁₉H₁₈FN₅O: 351.15, Found:352.¹H NMR (400 MHz, CDCl₃) δ 8.37 (d, J=5.4 Hz, 1H), 7.52-7.40 (m, 2H), 7.12 (q, J=8.7 Hz, 2H), 6.77 (dd, J=14.4, 5.3 Hz, 1H), 5.17 (d, J=8.5 Hz, 2H), 4.42-4.23 (m, 2H), 4.16 (t, J=5.4 Hz, 1H), 4.01 (t, J=5.4 Hz, 1H), 2.74 (d, J=4.6 Hz, 3H), 2.28 (d, J=11.0 Hz, 3H).

### Example 32. Preparation of Compound 1-52

As shown in FIG. 32, the specific synthesis steps are as follows:

Step1: 1-(3-(2-ethylpyridin-4-yl)-2-(4-fluorophenyl)-6,7-dihydropyrazolo[1,5-a]pyrazin-5(4H)-yl)ethan-1-one

Pd(dppf)Cl₂ (41.7 mg, 0.057 mmol), 4-bromo-2-ethylpyridine (159 mg, 0.855 mmol), Cs₂CO₃ (557 mg, 1.71 mmol) was added to a solution of 1-[2-(4-fluorophenyl)-3-(4,4,5,5-tetramethyl-1,3,2-dioxaborolan-2-yl)-4H,6H,7H-pyrazolo[1,5-a]pyrazin-5-yl]ethanone (220 mg, 0.57 mmol) in dioxane/H₂O (20:4 mL). The mixture was allowed to react at 90° C. under N₂ for 16 h. The reaction mixture was cooled to room temperture and concentrated under pressure. The crude material was added to a silica gel column and was eluted with MeOH/DCM(25:1) to give product as yellow solid (15.5 mg, yield: 7.4%). Chemical Formula: calculated for (M+H⁺) Chemical Formula: C₂₁H₂₁FN₄O: 364.17, Found:365. ¹H NMR (400 MHz, CDCl₃) δ 8.50 (dd, J=17.6, 4.7 Hz, 1H), 7.39 (dd, J=8.6, 5.5 Hz, 2H), 7.01 (t, J=8.7 Hz, 2H), 6.93 (s, 2H), 4.81 (d, J=51.7 Hz, 2H), 4.32 (dt, J=27.2, 5.5 Hz, 2H), 4.09 (dt, J=61.0, 5.5 Hz, 2H), 2.79 (q, J=7.5 Hz, 2H), 2.21 (d, J=36.5 Hz, 3H), 1.25 (t, J=7.6 Hz, 3H).

### Example 33. Preparation of Compound 1-53

As shown in FIG. 33, the specific synthesis steps are as follows:

Step 1: 1-(3-(6-aminopyrimidin-4-yl)-2-(4-fluorophenyl)-6,7-dihydropyrazolo[1,5-a]pyrazin-5(4H)-yl)ethan-1-one

Pd(dppf)Cl₂ (28.5 mg, 0.039 mmol), 4-bromo-2-isopropylpyridine (117 mg, 0.585 mmol), Cs₂CO₃ (381 mg, 1.17 mmol) was added to a solution of 1-[2-(4-fluorophenyl)-3-(4,4,5,5-tetramethyl-1,3,2-dioxaborolan-2-yl)-4H,6H,7H-pyrazolo[1,5-a]pyrazin-5-yl]ethanone (150 mg, 0.39 mmol) in dioxane/H₂O (20:4 mL), the mixture was allowed to react at 90° C. under N₂ for 16 h. The reaction mixture was cooled to room temperture and concentrated under pressure. The crude material was added to a silica gel column and was eluted with MeOH/DCM (25:1) to give product as yellow solid (52 mg, yield: 35%). Chemical Formula: calculated for (M+H⁺) Chemical Formula: C₂₂H₂₃FN₄O: 378.19, Found:379. ¹H NMR (400 MHz, CDCl₃) δ 8.53 (dd, J=15.1, 5.0 Hz, 1H), 7.39 (dd, J=8.2, 5.7 Hz, 2H), 7.06-6.91 (m, 4H), 4.82 (d, J=51.0 Hz, 2H), 4.32 (dt, J=27.0, 5.5 Hz, 2H), 4.09 (dt, J=60.3, 5.5 Hz, 2H), 3.03 (dt, J=13.8, 6.9 Hz, 1H), 2.21 (d, J=35.0 Hz, 3H), 1.23 (d, J=6.9 Hz, 6H).

### Example 34. Preparation of Compound 1-54

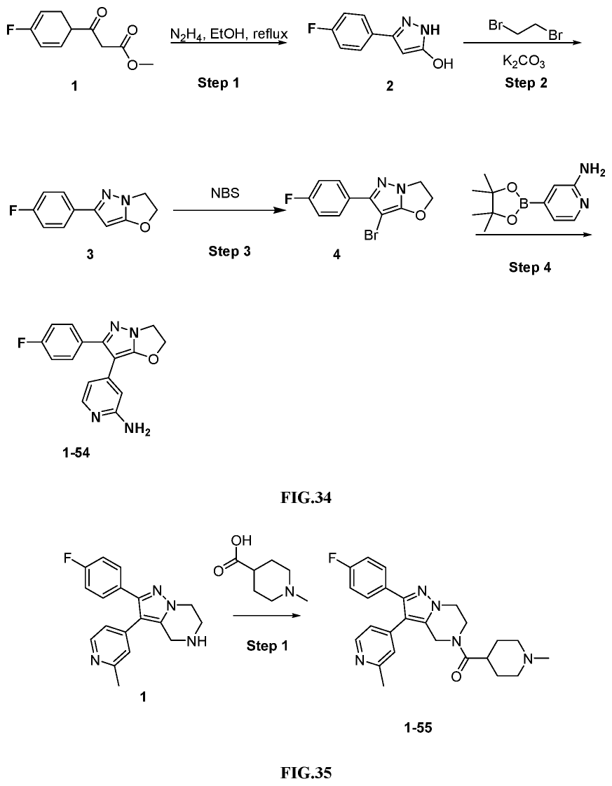

As shown in FIG. 34, the specific synthesis steps are as follows:

Step 1: 3-(4-fluorophenyl)-1H-pyrazol-5-ol

To a mixture of methyl 3-(4-fluorophenyl)-3-oxopropanoate (20 g, 101.9 mmol) in EtOH (250 mL) was added hydrogen diazene (6.3 g, 203.8 mmol) at 25° C. The reaction was stirred at 60° C. for 3 h. LCMS showed desired MS was detected as main peak. The mixture was concentrated and purified by column chromatography (PE/EA=6/1) to give a purple solid (17.2 g, yield: 98%). Chemical Formula: calculated for (M+H⁺) C₉H₇FN₂O: 178.05, Found: 179

Step 2: 6-(4-fluorophenyl)-2,3-dihydropyrazolo[5,1-b]oxazole

To a mixture of 3-(4-fluorophenyl)-1H-pyrazol-5-ol (1200 mg, 6.74 mmol) in ACN (50 mL) was added 1,2-dibromoethane (2500 mg, 13.48 mmol) and K₂CO₃ (3728 mg, 27 mmol) at 25° C., The reaction was stirred at 80° C. for 3 h. LCMS showed desired MS was detected as main peak. The reaction mixture was cooled to room temperature and concentrated under pressure. diluted with water (50 mL). Extration with DCM (50 mL*3), filtured and concentrated under pressure. The crude material was added to a silica gel column and was eluted with PE/EtOAc (10:1) to give product as a pink solid (3700 mg, yield: 80.6%). Chemical Formula: calculated for (M+H⁺) C₁₁H₉FN₂O: 204.07, Found: 205

Step 3: 7-bromo-6-(4-fluorophenyl)-2,3-dihydropyrazolo[5,1-b]oxazole

To a stirred solution of 6-(4-fluorophenyl)-2,3-dihydropyrazolo[5,1-b] oxazole (3600 mg, 17.63 mmol) in acetonitrile (100 mL) was added NBS (1883 mg, 10.57 mmol) with ice-cooling. The mixture was stirred at 25° C. for 1 h. LCMS showed desired MS was detected as main peak. The reaction mixture was concentrated under pressure and diluted with water (50 mL). Extration with EA(50 mL*3), filtured and concentrated under pressure. The crude material was added to a silica gel column and was eluted with PE/EtOAc (4:1) to give product as a yellow solid (1080 mg, yield: 19.48%). Chemical Formula: calculated for (M+H⁺) C₁₁H₈BrFN₂O: 283.18, Found: 284

Step 4: 4-(6-(4-fluorophenyl)-2,3-dihydropyrazolo[5,1-b]oxazol-7-yl)pyridin-2-amine

Pd(dppf)Cl₂ (38.78 mg, 0.053 mmol), 4-(4,4,5,5-tetramethyl-1,3,2-dioxaborolan-2-yl)pyridin-2-amine (134.2 mg, 0.61 mmol), Cs₂CO₃ (518 mg, 1.59 mmol) was added to a solution of 7-bromo-6-(4-fluorophenyl)-2,3-dihydropyrazolo[5,1-b]oxazole (150 mg, 0.53 mmol) in dioxane/H₂O (15:3 mL), the mixture was allowed to react at 90° C. under N₂ for 16 h. The reaction mixture was cooled to room temperature and concentrated under pressure. The crude material was added to a silica gel column and was eluted with MeOH/DCM (25:1) to give product as yellow solid (14.9 mg, yield: 9.3%). Chemical Formula: calculated for (M+H⁺) Chemical Formula: C₁₆H₁₃FN₄O: 296.11, Found:297. ¹H NMR (400 MHz, CDCl₃) δ 7.70 (d, J=6.0 Hz, 1H), 7.51-7.43 (m, 2H), 7.11 (t, J=8.7 Hz, 2H), 6.58 (d, J=6.0 Hz, 1H), 6.46 (s, 1H), 5.71 (s, 2H), 5.26-5.19 (m, 2H), 4.46-4.40 (m, 2H).

### Example 35. Preparation of Compound 1-55

As shown in FIG. 35, the specific synthesis steps are as follows:

Step 1: (2-(4-fluorophenyl)-3-(2-methylpyridin-4-yl)-6,7-dihydropyrazolo[1,5-a]pyrazin-5(4H)-yl)(1-methylpiperidin-4-yl)methanone

methylpiperidine-4-carboxylic acid (41.8 mg, 0.29 mmol), T3P (155 mg, 0.49 mmol) and DIPEA (150.9 mg, 1.17 mmol) were added to a solution of 2-(4-fluorophenyl)-3-(2-methylpyridin-4-yl)-4,5,6,7-tetrahydropyrazolo[1,5-a]pyrazine (60 mg, 0.19 mmol) in DCM (5 mL). The mixture was stirred at 25° C. for 3 h. The reaction mixture was concentrated under pressure. The residue was purified via Prep-TLC DCM/MeOH (5:1) and give the product. Chemical Formula: calculated for (M+H⁺) C₂₅H₂₈FN₅O: 433.53, Found: 433.8. ¹H NMR (400 MHz, CDCl₃) δ 8.48 (d, J=25.6 Hz, 1H), 8.34 (s, 1H), 7.37 (s, 2H), 7.12-6.84 (m, 3H), 4.79 (d, J=43.8 Hz, 2H), 4.33 (d, J=28.8 Hz, 2H), 4.11 (d, J=48.7 Hz, 2H), 3.30 (s, 2H), 3.03 (s, 2H), 2.66 (d, J=20.2 Hz, 4H), 2.54 (s, 3H), 2.40-1.92 (m, 4H).

### Example 36. Preparation of Compound 1-56

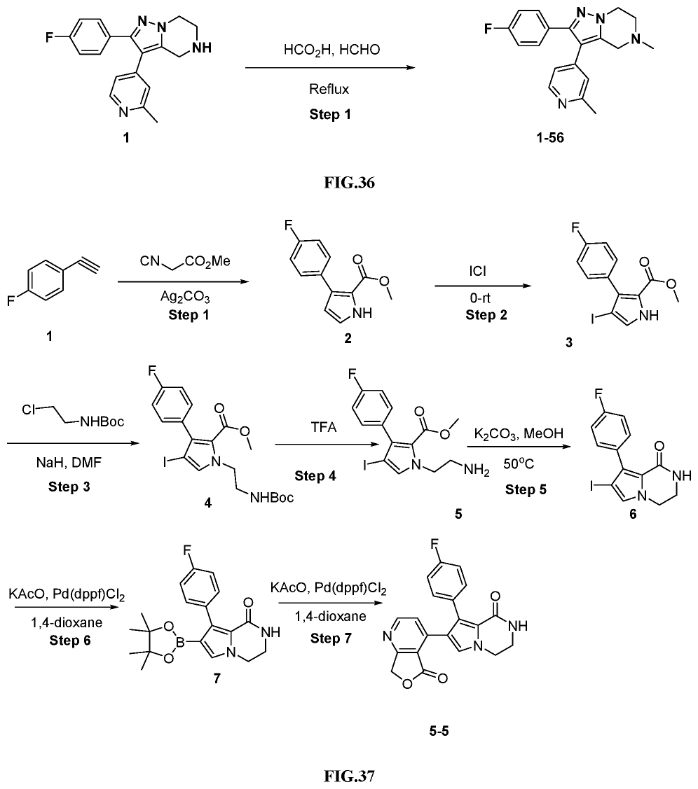

As shown in FIG. 36, the specific synthesis steps are as follows:

Step 1: 2-(4-fluorophenyl)-5-methyl-3-(2-methylpyridin-4-yl)-4,5,6,7-tetrahydropyrazolo[1,5-a]pyrazine

To a solution of 4-[2-(4-fluorophenyl)-4H,5H,6H,7H-pyrazolo[1,5-a]pyrazin-3-yl]-2-methylpyridine (50 mg, 0.16 mmol) in HCOOH (3 mL) was added HCHO (24 mg, 0.8 mmol). The reaction mixture was stirred at 70° C. for 2 h. The reaction was not complete detected by LCMS. The reaction mixture was concentrated under pressure. The mixture was adjusted to pH=8 with Na₂CO₃. The organic phase was washed with water (5 mL). The residue was extracted with EA (5mL*3). Solvent was dried over sodium sulphate and dried under a stream of nitrogen in the Radleys blowdown apparatus to give the crude product. The solution was filtered, filtrate was collected. The reaction mixture was concentrated under pressure. The residue was purified via Genal-Prep-HPLC and give the product.

¹H NMR (400 MHz, CDCl₃) δ 8.42 (d, J=5.2 Hz, 1H), 7.42-7.36 (m, 2H), 7.05-6.97 (m, 2H), 6.94 (s, 1H), 6.90 (d, J=4.8 Hz, 1H), 4.30 (t, J=5.6 Hz, 2H), 3.67 (s, 2H), 3.01-2.94 (m, 2H), 2.54 (d, J=4.0 Hz, 6H).

### Example 37. Preparation of Compound 5-5

As shown in FIG. 37, the specific synthesis steps are as follows:

Step 1: methyl 3-(4-fluorophenyl)-1H-pyrrole-2-carboxylate

To a solution of 1-ethynyl-4-fluorobenzene (3000 mg, 24.97 mmol) in NMP (50 mL), was added methyl 2-isocyanoacetate (3711.3410 mg, 37.455 mmol), Ag₂CO₃ (2756.6880 mg, 9.988 mmol), the reaction mixture was stirred at 80° C. for 1 h. The reaction was complete detected by 1 cms. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the crude product. The crude material was added to a silica gel column and was eluted with PE/EtOAc(1:1). Chemical Formula: calculated for (M+H⁺) C₁₂H₁₀FNO₂: 219.1, Found: 220.0

Step 2: methyl 3-(4-fluorophenyl)-4-iodo-1H-pyrrole-2-carboxylate

To a solution of methyl 3-(4-fluorophenyl)-1H-pyrrole-2-carboxylate [800 mg, 3.65 mmol] in CCl₄ [10 mL], then was added acetyl chloride [592.6030 mg, 3.65 mmol] at 0° C., The reaction mixture was stirred at 0° C. for 15 mins. The reaction was complete detected by 1 cms. The reaction mixture was concentrated under pressure. The crude material was added to a silica gel column and was eluted with PE/EtOAc(1:1). Chemical Formula: calculated for (M+H⁺) C₁₂H₉FINO₂: 345.0, Found: 345.9

Step 3: methyl 1-(2-((tert-butoxycarbonyl)amino)ethyl)-3-(4-fluorophenyl)-4-iodo-1H-pyrrole-2-carboxylate

To a solution of methyl 3-(4-fluorophenyl)-4-iodo-1H-pyrrole-2-carboxylate [400 mg, 1.16 mmol] in DMF [10 mL], then was added tert-butyl N-(2-chloroethyl)carbamate [416.776 mg, 2.32 mmol], K₂CO₃ [480.24 mg, 3.48 mmol], The reaction mixture was stirred at 100° C. for 16 h. The reaction was complete detected by 1 cms. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the product. Chemical Formula: calculated for (M+Na⁺) C₁₉H₂₂FIN₂O₄: 488.1, Found: 510.9

Step 4: methyl 1-(2-aminoethyl)-3-(4-fluorophenyl)-4-iodo-1H-pyrrole-2-carboxylate

To a solution of methyl 1-(2-{[(tert-butoxy)carbonyl]amino}ethyl)-3-(4-fluorophenyl)-4-iodopyrrole-2-carboxylate [500 mg, 1.0240 mmol] in DCM [3 mL], then was added HCl-dioxane [3 mL], The reaction mixture was stirred at 25° C. for 2h. The reaction was complete detected by 1 cms. The reaction mixture was concentrated under pressure. The mixture was adjusted to PH=8 with Na₂CO₃. The residue was extracted with EA(×5mL). Solvent was dried over sodium sulphate and dried under a stream of nitrogen in the Radleys blowdown apparatus to give the crude product. The solution was filtered ,filtrate was collected. The reaction mixture was concentrated under pressure . Chemical Formula: calculated for (M+H⁺) C₁₄H₁₄FIN₂O₂: 388.0, Found: 388.9

Step 5: 8-(4-fluorophenyl)-7-iodo-3,4-dihydropyrrolo[1,2-a]pyrazin-1(2H)-one

To a solution of methyl 1-(2-aminoethyl)-3-(4-fluorophenyl)-4-iodopyrrole-2-carboxylate [300 mg, 0.77 mmol] in MeOH [5 mL], then was added K₂CO₃ [159.39 mg], The reaction mixture was stirred at 50° C. for 2h. The reaction was complete detected by 1 cms. The reaction was quenched by H₂O (50 mL), The mixture was concentrated. The mixture was extracted with EA (50 mL*3). The combined organic layers were washed with brine (50 mL), dried over sodium sulfate, filtered and concentrated to give the product. calculated for (M+H⁺) C₁₃H₁₀FIN₂O: 356.0, Found: 356.8

Step 6: 8-(4-fluorophenyl)-7-(4,4,5,5-tetramethyl-1,3,2-dioxaborolan-2-yl)-3,4-dihydropyrrolo[1,2-a] pyrazin-1(2H)-one

To a solution of 8-(4-fluorophenyl)-7-iodo-2H,3H,4H-pyrrolo[1,2-a]pyrazin-1-one [120 mg, 0.34 mmol] in 1,4-Dioxane [5 mL], then was added 4,4,5,5-tetramethyl-2-(4,4,5,5-tetramethyl-1,3,2-dioxaborolan-2-yl)-1,3,2-dioxaborolane [129.5089 mg, 0.51 mmol], KAcO [99.9600 mg, 1.02 mmol], Pd(dppf)Cl₂ [49.7080 mg], The reaction mixture was stirred at 100° C. for 16 h. The reaction was complete detected by 1 cms. The mixture was extracted with EA (50 mL*3). The combined organic layers were washed with brine (50 mL), dried over sodium sulfate, filtered and concentrated to give the product. calculated for (M+H⁺) C₁₉H₂₂BFN₂O₃: 356.2, Found: 357.0

Step 7: 4-(8-(4-fluorophenyl)-1-oxo-1,2,3,4-tetrahydropyrrolo[1,2-a] pyrazin-7-yl) furo[3,4-b] pyridin-5(7H)-one

To a solution of 8-(4-fluorophenyl)-7-(4,4,5-trimethyl-1,3,2-dioxaborolan-2-yl)-2H,3H,4H-pyrrolo[1,2-a]pyrazin-1-one [50 mg, 0.14 mmol] in 1,4-Dioxane [5 mL], then was added 4-bromo-7H-furo[3,4-b]pyridin-5-one [44.94 mg, 0.2100 mmol], Na₂CO₃ [44.52 mg, 0.42 mmol], Pd(dppf)Cl₂ [20.47 mg, 0.0280 mmol], The reaction mixture was stirred at 100° C. for 16 h. The reaction was complete detected by 1 cms. The solution was filtered, filtrate was collected. The reaction mixture was concentrated under pressure. The residue was purified via Genal-Prep-HPLC and give the product.

¹H NMR (400 MHz, CDCl₃) δ 8.39 (d, J=5.2 Hz, 1H), 8.00 (s, 1H), 7.69 (s, 1H), 7.52 (s, 1H), 7.01 (dd, J=10.0, 6.8 Hz, 2H), 6.72 (d, J=5.2 Hz, 1H), 5.82 (s, 1H), 5.28 (s, 1H), 4.37-4.23 (m, 2H), 3.76 (s, 2H).

### Example 38. Preparation of Compound 1-57

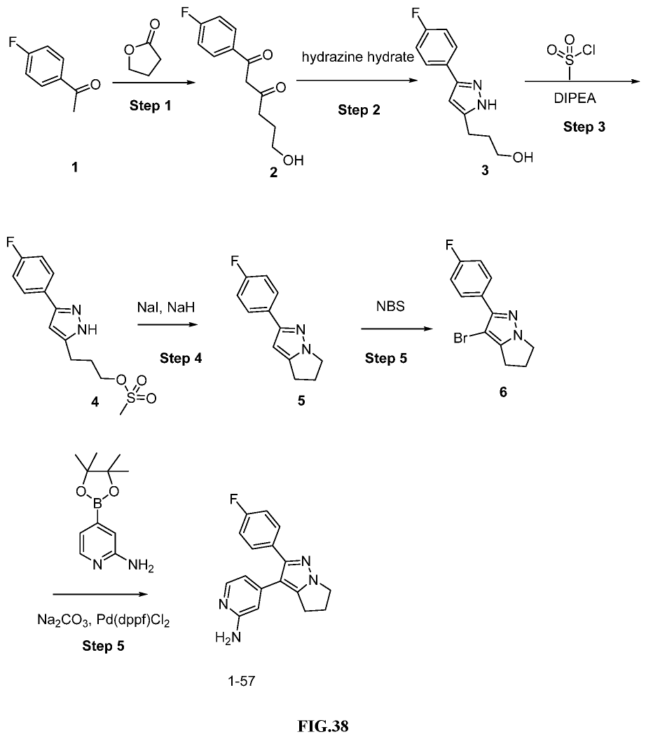

As shown in FIG. 38, the specific synthesis steps are as follows:

Step 1:1-(4-fluorophenyl)-6-hydroxyhexane-1,3-dione

To a mixture of NaH (60%, 0.56 g, 14.4 mmol) in dry diethyl ether (15 mL) at 0° C. under argon, ethanol (2 mL) was added followed by dihydrofuran-2(3H)-one (0.62 g, 7.2 mmol). Then a solution of 1-(4-fluorophenyl)ethan-1-one (1 g, 7.2 mmol) in diethyl ether (5 mL) was added slowly at 0° C. And the resulting suspension was allowed to warm slowly to room temperature. The reaction mixture was stirred 72 h at 25° C. The reaction mixture was concentrated under pressure. Chemical Formula: calculated for (M+H⁺) C₁₂H₁₃FO₃: 224.23, Found: 224.8.

Step 2:3-(3-(4-fluorophenyl)-1H-pyrazol-5-yl)propan-1-ol

To a solution of 1-(4-fluorophenyl)-6-hydroxyhexane-1,3-dione (500 mg, 2.23 mmol) in EtOH (20 mL), hydrazine hydrate (wt 80%, 178 mg, 4.46 mmol) were added. The mixture was stirred at 25° C. for 1 h. The reaction mixture was concentrated under pressure. Chemical Formula: calculated for (M+H⁺) C₁₂H₁₃FN₂O: 220.25, Found: 220.8.

Step 3: 3-(3-(4-fluorophenyl)-1H-pyrazol-5-yl)propyl methanesulfonate

To a solution of 3-(3-(4-fluorophenyl)-1H-pyrazol-5-yl)propan-1-ol (500 mg, 2.27 mmol) in DCM (20 mL) at 0° C., methanesulfonyl chloride (0.264 mL, 3.4 mmol) and DIPEA (880 mg, 6.8 mmol) were added. The mixture was stirred at 25° C. for 1 h. The reaction mixture was concentrated under pressure. Chemical Formula: calculated for (M+H⁺) C₁₃H₁₅FN₂O₃S: 298.33, Found: 298.8.

Step 4: 2-(4-fluorophenyl)-5,6-dihydro-4H-pyrrolo[1,2-b]pyrazole

To a solution of 3-(3-(4-fluorophenyl)-1H-pyrazol-5-yl)propyl methanesulfonate (300 mg, 1.0 mmol) in DMF (15 mL) at 0° C., NaI (15.08 mg, 0.1 mmol) and NaH (29 mg, 1.2 mmol) were added. The mixture was added at 0° C. for 0.5 h and then stirred at 25° C. for 1 h. The reaction mixture was concentrated under pressure. Chemical Formula: calculated for (M+H⁺) C₁₂H₁₁FN₂: 202.23, Found: 202.8.

Step 5: 3-bromo-2-(4-fluorophenyl)-5,6-dihydro-4H-pyrrolo[1,2-b]pyrazole

To a solution of 2-(4-fluorophenyl)-5,6-dihydro-4H-pyrrolo[1,2-b]pyrazole (200 mg, 0.99 mmol) in CH₃CN (15 mL), NBS (264 mg, 1.48 mmol) was added. The mixture was stirred at 25° C. for 2 h. The reaction mixture was concentrated under pressure. Chemical Formula: calculated for (M+H⁺) C₁₂H₁₀BrFN₂: 281.13, Found: 280.8.

Step 6: 4-(2-(4-fluorophenyl)-5,6-dihydro-4H-pyrrolo[1,2-b]pyrazol-3-yl)pyridin-2-amine

To a solution of 3-bromo-2-(4-fluorophenyl)-5,6-dihydro-4H-pyrrolo[1,2-b]pyrazole (250 mg, 0.89 mmol) in EtOH/toluene=8:2 (15 mL), 4-(4,4,5,5-tetramethyl-1,3,2-dioxaborolan-2-yl)pyridin-2-amine (294 mg, 1.3 mmol), 2M Na₂CO₃ aqueous solutions (1.5 mL) and Pd(dppf)Cl₂ (130 mg, 0.18 mmol) were added. The mixture was stirred at 100° C. for 1 h under nitrogen atmosphere on microwave. The reaction mixture was concentrated under pressure. Chemical Formula: calculated for (M+H⁺) C₁₇H₁₅FN₄: 294.33, Found: 294.8. ¹H NMR (400 MHz, CDCl₃) δ 8.45 (s, 2H), 7.65-7.50 (m, 1H), 7.48-7.35 (m, 2H), 7.07 (ddd, J=8.7, 5.3, 1.9 Hz, 2H), 6.61-6.36 (m, 2H), 4.25 (t, J=7.1 Hz, 2H), 3.09 (t, J=7.1 Hz, 2H), 2.80-2.64 (m, 2H).

### Example 39. Preparation of Compound 1-58

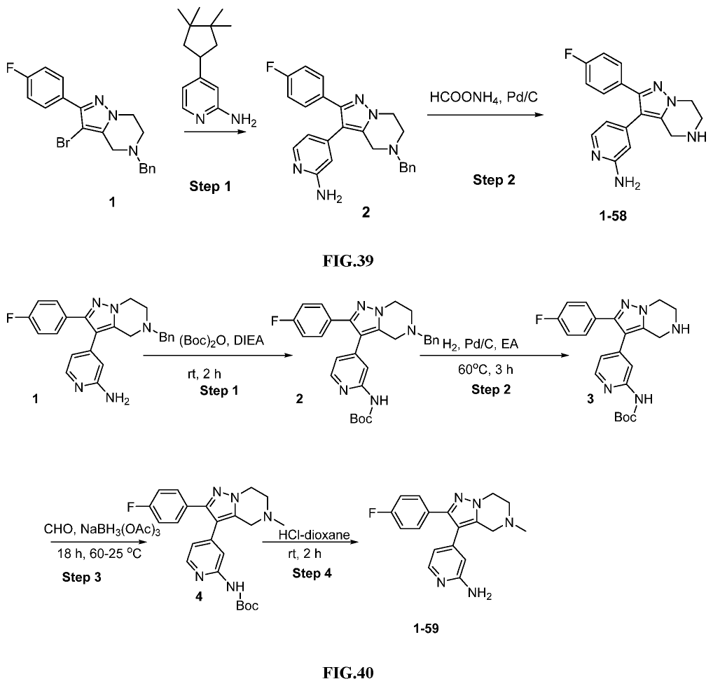

As shown in FIG. 39, the specific synthesis steps are as follows:

Step 1: 4-(5-benzyl-2-(4-fluorophenyl)-4,5,6,7-tetrahydropyrazolo[1,5-a]pyrazin-3-yl)pyridin-2-amine

4-(3,3,4,4-tetramethylcyclopentyl)pyridin-2-amine (102 mg, 0.46 mmol), 2 M Na₂CO₃ aqueous solutions (0.5 mL) and Pd(dppf)Cl₂ (45 mg, 0.06 mmol) were added to a solution of 5-benzyl-3-bromo-2-(4-fluorophenyl)-4,5,6,7-tetrahydropyrazolo[1,5-a]pyrazine (120 mg, 0.31 mmol) in EtOH/toluene=8:2 (5 mL). The mixture was stirred at 100° C. for 1 h under nitrogen atmosphere on microwave. The reaction mixture was concentrated under pressure. The residue was purified via Prep-TLC PE/EA (1:4) and give the product. Chemical Formula: calculated for (M+H^{'}) C₂₄H₂₂FN₅: 399.47, Found: 399.8.

Step 2: 4-(2-(4-fluorophenyl)-4,5,6,7-tetrahydropyrazolo[1,5-a]pyrazin-3-yl)pyridin-2-amine

HCOONH₄ (157 mg, 2.5 mmol) and Pd/C (200 mg) were added to a solution of 4-(5-benzyl-2-(4-fluorophenyl)-4,5,6,7-tetrahydropyrazolo[1,5-a]pyrazin-3-yl)pyridin-2-amine (100 mg, 0.25 mmol) in MeOH (10 mL). The mixture was stirred at 60° C. for 2 h. The solution was filtered, filtrate was collected and the solution was concentrated under pressure. Chemical Formula: calculated for (M+H⁺) C₁₇H₁₆FN₅: 309.35, Found: 309.8.

### Example 40. Preparation of Compound 1-59

As shown in FIG. 40, the specific synthesis steps are as follows:

Step 1: tert-butyl (4-(5-benzyl-2-(4-fluorophenyl)-4,5,6,7-tetrahydropyrazolo[1,5-a]pyrazin-3-yl)pyridin-2-yl)carbamate

To a solution of 4-[2-(4-fluorophenyl)-5-(1-methylphenyl)-4H,6H,7H-pyrazolo[1,5-a]pyrazin-3-yl]pyridin-2-amine (2000 mg, 4.99 mmol) in DCM (20 mL), (Boc)20 (1631.7300 mg, 7.485 mmol), DIPEA (1287.4200 mg, 9.98 mmol), DMAP (121.7560 mg, 0.9980 mmol) was added. The reaction mixture was stirred at 25° C. for 2 h. The reaction was complete detected by 1 cms. The reaction mixture was concentrated under pressure. The crude material was added to a silica gel column and was eluted with PE/EtOAc (1:1). Chemical Formula: calculated for (M+H⁺) C₂₉H₃₀FN₅O₂: 499.2, Found: 500.1

Step 2: tert-butyl (4-(2-(4-fluorophenyl)-4,5,6,7-tetrahydropyrazolo[1,5-a]pyrazin-3-yl)pyridin-2-yl)carbamate

To a solution of tert-butyl (4-(5-benzyl-2-(4-fluorophenyl)-4,5,6,7-tetrahydropyrazolo [1,5-a]pyrazin-3-yl) pyridin-2-yl) carbamate (1200 mg, 2.39 mmol) in EA (10 mL), Pd/C (800 mg) and AcOH (0.5 mL) was added. The reaction mixture was stirred at 25° C. under H₂ for 2 h. The reaction was complete detected by 1 cms. The solution was filtered, filtrate was collected. The reaction mixture was concentrated under pressure. The organic phase was washed with 2M sodium carbonate solution (5 mL). The residue was extracted with EA (10 mL*3). The combined organic layers were washed with brine (10 mL), dried over sodium sulfate, filtered and concentrated to give the product. Chemical Formula: calculated for (M+H⁺) C₂₂H₂₄FN₅O₂: 409.2, Found: 410.1

Step 3: tert-butyl (4-(2-(4-fluorophenyl)-5-methyl-4,5,6,7-tetrahydropyrazolo[1,5-a]pyrazin-3-yl)pyridin-2-yl)carbamate

To a solution of tert-butyl (4-(2-(4-fluorophenyl)-4,5,6,7-tetrahydropyrazolo[1,5-a] pyrazin-3-yl) pyridin-2-yl) carbamate (230 mg, 0.5603 mmol) in THF (5 mL), formaldehyde (84.05 mg, 2.8015 mmol) was added. The reaction mixture was stirred at 70° C. for 16 h. Sodium triacetoxyborohydride (142.54 mg, 0.6723 mmol) was added. The reaction mixture was stirred at 25° C. for 2 h. The reaction was complete detected by 1 cms. The reaction mixture was concentrated under pressure. The organic phase was washed with water (5 mL). The residue was extracted with EA (5 mL*3). The combined organic layers were washed with brine (10 mL), dried over sodium sulfate, filtered and concentrated to give the product. The crude material was added to a silica gel column and was eluted with PE/EtOAc (1:1). Chemical Formula: calculated for (M+H⁺) C₂₃H₂₆FN₅O₂: 423.2, Found: 424.0

Step 4: 4-(2-(4-fluorophenyl)-5-methyl-4,5,6,7-tetrahydropyrazolo[1,5-a]pyrazin-3-yl)pyridin-2-amine

To a solution of tert-butyl (4-(2-(4-fluorophenyl)-5-methyl-4,5,6,7-tetrahydropyrazolo [1,5-a]pyrazin-3-yl) pyridin-2-yl) carbamate (200 mg, 0.47 mmol) in DCM (4 mL), then HCl-dioxane (4 mL) was added. The reaction mixture was stirred at 25° C. for 2 h. The reaction was complete detected by 1 cms. The reaction mixture was concentrated under pressure. The organic phase was washed with 2M sodium carbonate solution (5 mL). The residue was extracted with EA (5 mL*3). The combined organic layers were washed with brine (10 mL), dried over sodium sulfate, filtered and concentrated to give the product. The residue was purified via Genal-Prep-HPLC and give the product.

¹H NMR (400 MHz, DMSO) δ 7.83 (d, J=5.2 Hz, 1H), 7.46-7.37 (m, 2H), 7.21-7.14 (m, 2H), 6.21 (dd, J=7.4, 2.0 Hz, 2H), 5.90 (s, 2H), 4.16 (t, J=5.4 Hz, 2H), 3.57 (s, 2H), 2.90 (t, J=5.5 Hz, 2H), 2.41 (s, 3H).

### Example 41. Preparation of Compound 1-60

As shown in FIG. 41, the specific synthesis steps are as follows:

Step 1: 1-(2-(4-fluorophenyl)-3-(1H-pyrrolo[2,3-b] pyridin-4-yl)-6,7-dihydropyrazolo [1,5-a]pyrazin-5(4H)-yl) ethan-1-one

To a solution of 1-[2-(4-fluorophenyl)-3-(4,4,5,5-tetramethyl-1,3,2-dioxaborolan-2-yl)-4H,6H,7H-pyrazolo[1,5-a]pyrazin-5-yl]ethanone (1000 mg, 2.5958 mmol) in 1,4-Dioxane/H₂O=10: 1 (30 mL), 4-bromo-1H-pyrrolo[2,3-b]pyridine (767.18 mg, 3.8937 mmol), Na₂CO₃ (825.46 mg, 7.7874 mmol), Pd(dppf)Cl₂ (379.51 mg, 0.5191 mmol) was added. The reaction mixture was stirred at 100° C. for 16 h. The reaction was complete detected by 1 cms. The solution was filtered, filtrate was collected. The reaction mixture was concentrated under pressure. The crude material was added to a silica gel column and was eluted with DCM/MeOH (10:1). Chemical Formula: calculated for (M+H⁺) C₂₁H₁₈FN₅O: 375.1, Found: 376.0

Step 2: 4-(5-acetyl-2-(4-fluorophenyl)-4,5,6,7-tetrahydropyrazolo [1,5-a] pyrazin-3-yl)-3,3-dibromo-1,3-dihydro-2H-pyrrolo [2,3-b] pyridin-2-one

To a solution of 1-(2-(4-fluorophenyl)-3-(1H-pyrrolo[2,3-b] pyridin-4-yl)-6,7-dihydropyrazolo [1,5-a] pyrazin-5(4H)-yl) ethan-1-one (500 mg, 1.3319 mmol) in t-BuOH (10 mL), Py-HBr₃ (1704.83 mg, 5.3276 mmol) was added in small portions over 6 h at room temperature. The reaction mixture was concentrated under pressure. The organic phase was washed with water (5 mL). The residue was extracted with EA (5 mL*3). The combined organic layers were washed with brine (50 mL), dried over sodium sulfate, filtered and concentrated to give the product. Chemical Formula: calculated for (M+H⁺) C₂₁H₁₆Br₂FN₅O₂: 547.0, Found: 547.5

Step 3: 4-(5-acetyl-2-(4-fluorophenyl)-4,5,6,7-tetrahydropyrazolo[1,5-a]pyrazin-3-yl)-1,3-dihydro-2H-pyrrolo[2,3-b]pyridin-2-one

To a solution of 4-(5-acetyl-2-(4-fluorophenyl)-4,5,6,7-tetrahydropyrazolo [1,5-a] pyrazin-3-yl)-3,3-dibromo-1,3-dihydro-2H-pyrrolo [2,3-b] pyridin-2-one (220 mg, 0.4006 mmol) in EA (5 mL), Pd/C (100 mg) was added. The reaction mixture was stirred at 25° C. under H₂ for 5 h. The reaction was complete detected by 1 cms. The solution was filtered, filtrate was collected. The reaction mixture was concentrated under pressure. The residue was purified via Genal-Prep-HPLC and give the product.

¹H NMR (400 MHz, MeOD) δ 8.09 (dd, J=50.2, 26.5 Hz, 2H), 7.45-7.34 (m, 2H), 7.09 (t, J=8.8 Hz, 2H), 7.02 (dd, J=14.8, 5.2 Hz, 1H), 4.78 (d, J=12.4 Hz, 2H), 4.31 (dt, J=36.0, 5.6 Hz, 2H), 4.12 (dt, J=11.0, 5.6 Hz, 2H), 2.86 (s, 2H), 2.19 (d, J=39.6 Hz, 3H).

### Example 42. Preparation of Compound 1-61

As shown in FIG. 42, the specific synthesis steps are as follows:

Step 1: (4-bromopyridin-2-yl)acetic acid

To a solution of 4-bromo-2-methylpyridine (4.0 g, 0.0233 mol) in THF (30 mL) was added dimethyl carbonate (2.52 g, 0.0279 mol) and LDA (13.8 mL) at −78° C. The mixture was stirred for 1 h at −78° C. and stirred for 5 h at rt. LCMS showed the reaction was completed. The reaction was quenched with saturated aqueous solution of NH4C1 and washed with water, extracted with EA. Dried over Na₂SO₄. The crude product was purified by silica gel column (PE: EtOAc=4:1) to give the product. Chemical Formula: C₈H₈BrNO₂. calculated for (M+H⁺): 230.06, Found: 231.1

Step 2: methyl 2-{4-[2-(4-fluorophenyl)-5-(1-methylphenyl)-4H,6H,7H-pyrazolo[1,5-a]pyrazin-3-yl]pyridin-2-yl} acetate

To a solution of methyl 2-(4-bromopyridin-2-yl)acetate (150 mg, 0.652 mmol) in DMF(5 mL) was added 2-(4-fluorophenyl)-5-(1-methylphenyl)-3-(4,4,5,5-tetramethyl-1,3 ,2-dioxaborolan-2-yl)-4H,6H,7H-pyrazolo[1,5-a]pyrazine (566.37 mg, 1.304 mmol), Pd(dppf)Cl₂ (53.2 mg, 0.0652 mmol) and K₂CO₃ (180.23 mg, 1.304 mmol) at rt. The mixture was stirred for 3 h at 90° C. under N₂. LCMS showed the reaction was completed. The solution was washed with brine and extracted with EA. Dried over Na₂SO₄. The crude product was purified by silica gel column (PE: EtOAc=10:1) to give the product. Chemical Formula: C₂₇H₂₅FN₄O₂. calculated for (M+H⁺) :456.52, Found: 457.1.

Step 3: methyl 2-(4-(2-(4-fluorophenyl)-5-methyl-4,5,6,7-tetrahydropyrazolo[1,5-a]pyrazin-3-yl)pyridin-2-yl)acetate

To a solution of methyl 2-{4-[2-(4-fluorophenyl)-5-(1-methylphenyl)-4H,6H,7H-pyrazolo[1,5-a]pyrazin-3-yl]pyridin-2-yl}acetate (300 mg, 0.6557 mmol) in MeOH (10 mL) was added Pd/C (150mg). The mixture was stirred for 6 h at rt. The reaction was filtered, and the filtrate was concentrated in vacuo. The residue was purified via Genal-Prep-HPLC and give the product. Chemical Formula: C₂₁H₂₁FN₄O₂. calculated for (M+H⁺): 380.42, Found: 381.1.

¹H NMR (400 MHz, DMSO) δ 8.41 (d, J=4.0 Hz, 1H), 7.41-7.34 (m, 2H), 7.18 (t, J=8.9 Hz, 2H), 7.12-7.09 (m, 1H), 7.02 (dd, J=5.1, 1.5 Hz, 1H), 4.19 (t, J=4.0 Hz, 2H), 3.80 (s, 2H), 3.65 (s, 2H), 3.60 (s, 3H), 2.92 (t, J=5.4 Hz, 2H), 2.41 (s, 3H).

### Example 43. Preparation of Compound 1-62

As shown in FIG. 43, the specific synthesis steps are as follows:

Step 1: 1-(4-bromopyridin-2-yl)propan-2-one

To a solution of 4-bromo-2-methylpyridine (3 g, 17.4 mmol) in THF (30 mL), LDA (13 mL, 26 mmol, 2N) was added. The reaction mixture was stirred at −78° C. for 1 h. N-methoxy-N-methylacetamide (2.69 g, 26 mmol) was added. The reaction mixture was stirred at 25° C. for 15 h. The reaction mixture was concentrated under pressure. The crude material was added to a silica gel column and was eluted with PE/EtOAc (10:1) to give product. Chemical Formula: calculated for (M+H⁺) C₈H₈BrNO: 214.06, Found: 214.0.

Step 2: 1-(4-(5-benzyl-2-(4-fluorophenyl)-4,5,6,7-tetrahydropyrazolo[1,5-a]pyrazin-3-yl)pyridin-2-yl)propan-2-one

To a solution of 2-(4-fluorophenyl)-5-(1-methylphenyl)-3-(4,4,5,5-tetramethyl-1,3,2-dioxaborolan-2-yl)-4H,6H,7H-pyrazolo[1,5-a]pyrazine (6 g, 13.9 mmol) in dioxane (30 mL) and water (5 mL), 1-(4-bromopyridin-2-yl)propan-2-one (2 g, 9.3 mmol), potassium carbonate(2.57 g, 18.6 mmol) and tetrakis(triphenylphosphine)palladium (1 g, 0.9 mmol) was added. The reaction mixture was stirred at 100° C. for 18 h. The reaction mixture was concentrated under pressure. The crude material was added to a silica gel column and was eluted with PE/EtOAc (1:1) to give product. Chemical Formula: calculated for (M+H⁺) C₂₇H₂₅FN₄O: 440.52, Found: 441.1.

Step 3: 1-(4-(2-(4-fluorophenyl)-4,5,6,7-tetrahydropyrazolo[1,5-a]pyrazin-3-yl)pyridin-2-yl)propan-2-ol

To a solution of 1-{4-[2-(4-fluorophenyl)-5-(1-methylphenyl)-4H,6H,7H-pyrazolo[1,5-a]pyrazin-3-yl]pyridin-2-yl}propan-2-one (1200 mg) in MeOH (30 mL), Pd/C (100 mg) was added. The reaction mixture was stirred at 25° C. for 15 h. The solution was filtered, filtrate was collected. The reaction mixture was concentrated under pressure to give product. Chemical Formula: calculated for (M+H⁺) C₂₀H₂₁FN₄O: 352.41, Found: 353.8.

Step 4: 1-(2-(4-fluorophenyl)-3-(2-(2-hydroxypropyl)pyridin-4-yl)-6,7-dihydropyrazolo[1,5-a]pyrazin-5(4H)-yl)ethan-1-one

To a solution of 1-{4-[2-(4-fluorophenyl)-4H,5H,6H,7H-pyrazolo[1,5-a]pyrazin-3-yl]pyridin-2-yl}propan-2-ol (300 mg, 0.85 mmol) and NaHCO₃ (143 mg, 1.7 mmol) in THF (10 mL) and water (10 mL), acetyl acetate (174 mg, 1.7 mmol) was added at 0° C. The reaction mixture was stirred at 25° C. for 5 h. The residue was extracted with THF (10 mL*2). The reaction mixture was concentrated under pressure. The residue was purified via Genal-Prep-HPLC and give the product.

¹H NMR (400 MHz, MeOD) δ 8.40 (d, J=5.3 Hz, 1H), 8.32 (s, 1H), 7.41 (dd, J=8.7, 5.4 Hz, 2H), 7.18-7.00 (m, 4H), 4.30 (dt, J=34.8, 5.2 Hz, 2H), 4.10 (ddd, J=20.0, 12.4, 5.7 Hz, 3H), 2.82 (qd, J=13.3, 6.5 Hz, 2H), 2.21 (d, J=35.0 Hz, 3H), 1.21-1.11 (m, 3H).

### Example 44. Preparation of Compound 1-63

As shown in FIG. 44, the specific synthesis steps are as follows:

Step 1: 7-(2-(4-fluorophenyl)-4,5, 6,7-tetrahydropyrazolo[1,5-a]pyrazin-3-yl)-2,3-dihydrofuro[3,2-b]pyridine

To a solution of 3-bromo-2-(4-fluorophenyl)-4,5,6,7-tetrahydropyrazolo[1,5-a]pyrazine (100 mg, 0.34 mmol) in Dioxane (5 mL) and Water (0.5 ml) , 7-(3,3,4,4-tetramethylborolan-1-yl)-2,3-dihydrofuro[3,2-b]pyridine (85.11 mg, 0.35 mmol), Pd dppf) Cl2(10mg,10%w/w)) and K₃PO₄ (144.34mg,0.68 mmol) were added in . The mixture was stirred at 90° C. under N2 atmosphere for 4 h. The reaction mixture was diluted with water, and the water phase was extracted with EA twice time, the organic phase was combined and concentrated under pressure. The residue was purified via Prep-TLC and give the product as off-white solid (77.92 mg,68.6%)

Chemical Formula: calculated for (M+H⁺): C₁₉H₁₇FN₄O: 336.37, Found:337.

Step 2: 1-(3-(2,3-dihydrofuro[3,2-b]pyridin-7-yl)-2-(4-fluorophenyl)-6,7-dihydropyrazolo[1,5-a]pyrazin-5(4H)-yl)ethan-1-one

To a solution of 7-(2-(4-fluorophenyl)-4,5,6,7-tetrahydropyrazolo[1,5-a]pyrazin-3-yl)-2,3-dihydrofuro[3,2-b]pyridine (35 mg,0.10 mmol) in DCM (65 mL), TEA (20.24 mg, 0.20 mmol) was added in and then acetyl chloride (8.64 mg, 0.11 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Genal-Prep-HPLC and give the product as white solid (30.95 mg,78.6%)

Chemical Formula: calculated for (M+H⁺): C₂₁H₁₉FN₄O₂,378.41, Found:339.

### Example 45. Preparation of Compound 1-64

As shown in FIG. 45, the specific synthesis steps are as follows:

Step1: 1-(3-(2,3-dihydrofuro[3,2-b]pyridin-7-yl)-2-(4-fluorophenyl)-6,7-dihydropyrazolo[1,5-a]pyrazin-5(4H)-yl)ethan-1-one

To a stirred mixture of 7-bromo-2,3-dihydrofuro[3,2-b]pyridine (68 mg, 0.34 mmol, 1.0 equiv) and 2-(4-fluorophenyl)-5-methyl-3-(4,4,5, 5-tetramethyl-1,3,2-dioxaborolan-2-yl)-4,5,6,7-tetrahydropyrazolo[1,5-a]pyrazine (121 mg, 0.34 mmol, 1.0 equiv) in toluene (15 mL) were added Cs₂CO₃ (230 mg, 0.69 mmol, 2.0 equiv) and Pd(dppf)Cl₂ CH₂Cl₂ (28 mg, 0.034 mmol, 0.1 equiv) at room temperature under nitrogen atmosphere. The resulting mixture was stirred for 2 h at 100° C. under nitrogen atmosphere. The resulting mixture was concentrated under reduced pressure. The residue was purified by silica gel column chromatography, eluted with PE/EA (1: 2) to afford 1-(3-(2,3-dihydrofuro[3,2-b]pyridin-7-yl)-2-(4-fluorophenyl)-6,7-dihydropyrazolo[1,5-a]pyrazin-5(4H)-yl)ethan-1-one (36 mg, 28%) as a light brown solid.

### Example 46. Preparation of Compound 1-65

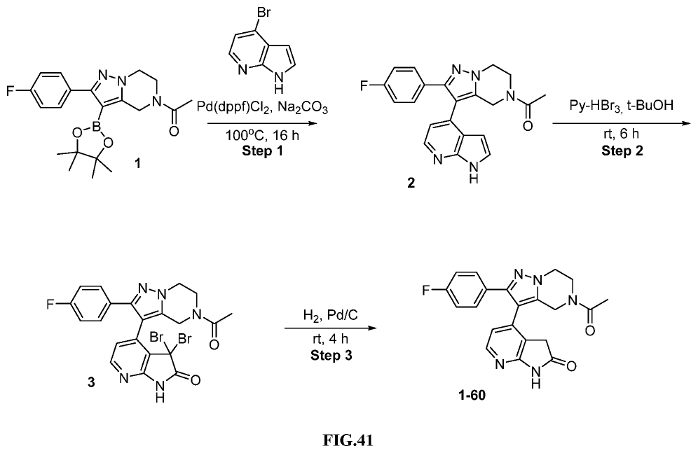

As shown in FIG. 46, the specific synthesis steps are as follows:

Step 1:1-(3-bromo-2-(4-fluorophenyl)-6,7-dihydropyrazolo[1,5-a]pyrazin-5(4H)-yl)ethan-1-one

To a solution of 7-(2-(4-fluorophenyl)-4,5,6,7-tetrahydropyrazolo[1,5-a]pyrazin-3-yl)-2-methyl-2,3-dihydroisoxazolo[4,5-b]pyridine(150 mg, 0.51 mmol) in DCM (10 mL) , TEA (103.21 mg, 1.02 mmol) was added in and then acetyl chloride (40.04 mg, 0.51 mmol) was dropped in at 0° C. The mixture was stirred at 25° C. for 2 h. The reaction mixture was diluted with water, and the water phase was extracted with DCM twice time, the organic phase was combined and concentrated under pressure. The residue was purified via Prep-TLC and give the product as off-white solid (147.14 mg,85.9%)

Chemical Formula: calculated for (M+H⁺) C₁₄H₁₃BrFN₃O: 338.18, Found:339.

Step 2: 7-(2-(4-fluorophenyl)-4,5,6,7-tetrahydropyrazolo[1,5-a]pyrazin-3-yl)-2-methyl-2,3-dihydroisoxazolo[4,5-b]pyridine

To a solution of 1-(3-bromo-2-(4-fluorophenyl)-6,7-dihydropyrazolo[1,5-a]pyrazin-5(4H)-yl)ethan-1-one (35 mg,0.10 mmol) in Dioxane (3 mL) and Water (0.5 ml), 2-methyl-7-(3,3,4,4-tetramethylborolan-1-yl)-2,3-dihydroisoxazolo[4,5-b]pyridine (28.40 mg, 0.11 mmol), Pd (dppf) Cl2 (3.5mg,10%w/w)) and K₃PO₄ (42.45 mg,0.20 mmol) were added in . The mixture was stirred at 90° C. under N2 atmosphere for 4 h. The reaction mixture was diluted with water, and the water phase was extracted with EA twice time, the organic phase was combined and concentrated under pressure. The residue was purified via Genal-Prep-HPLC and give the product as white solid(15.27 mg,37.5%)

Chemical Formula: calculated for (M+H⁺) C₂₁H₂₀FN₅O₂: 393.42, Found:394.

### Example 47. Preparation of Compound 1-66

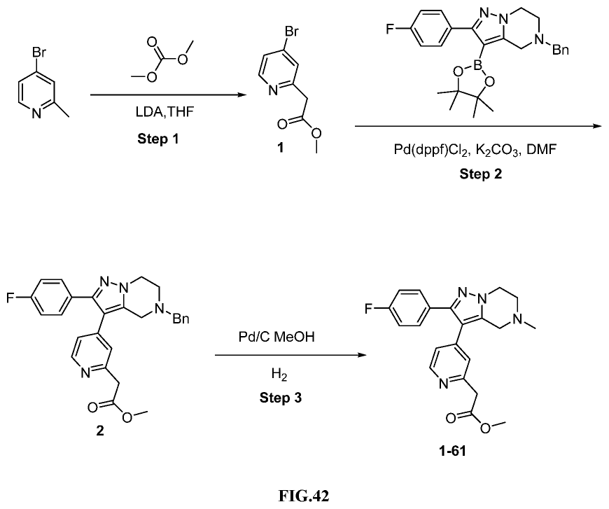

As shown in FIG. 47, the specific synthesis steps are as follows:

Step 1:7-(2-(4-fluorophenyl)-4,5,6,7-tetrahydropyrazolo[1,5-a]pyrazin-3-yl)-2-methyl-2,3-dihydroisoxazolo[4,5-b]pyridine

To a solution of 3-bromo-2-(4-fluorophenyl)-4,5,6,7-tetrahydropyrazolo[1,5-a]pyrazine (100 mg, 0.34 mmol) in Dioxane (5 mL) and Water (0.5 ml), 2-methyl-7-(3,3,4,4-tetramethylborolan-1-yl)-2,3-dihydroisoxazolo[4,5-b]pyridine (87.78 mg, 0.34 mmol), Pd (dppf) Cl₂ (10 mg,10%w/w)) and K₃PO₄ (144.34 mg,0.68 mmol) were added in. The mixture was stirred at 85° C. under N2 atmosphere for 6 h. The reaction mixture was diluted with water, and the water phase was extracted with EA twice time, the organic phase was combined and concentrated under pressure. The residue was purified via Prep-TLC and give the product as gray solid(85.31 mg,71.9%)

Chemical Formula: calculated for (M+H⁺) C₁₉H₁₈FN₅O: 351.39, Found:352.

Step 2: 7-(2-(4-fluorophenyl)-5-methyl-4,5,6,7-tetrahydropyrazolo[1,5-a]pyrazin-3-yl)-2-methyl-2,3-dihydroisoxazolo[4,5-b]pyridine

To a solution of 7-(2-(4-fluorophenyl)-4,5,6,7-tetrahydropyrazolo[1,5-a]pyrazin-3-yl)-2-methyl-2,3-dihydroisoxazolo[4,5-b]pyridine (50 mg, 0.14 mmol) in THF (65 mL) , paraformaldehyde (4.50 mg, 0.15 mmol) was added in and the mixture was stirred at 25° C. for 30 min. Then sodium cyanoborohydride (17.60 mg, 0.28 mmol) was added in at 0° C. The mixture was stirred at 25° C. for 2 h. The reaction was quenched with water, and was extracted with EA twice time, the organic phase was combined and concentrated under pressure. The residue was purified via Genal-Prep-HPLC and give the product as white solid(24.91 mg,47.9%)

Chemical Formula: calculated for (M+H⁺) C₂₀H₂₀FN₅O: 365.41, Found:366.

### Example 48. Preparation of Compound 1-67

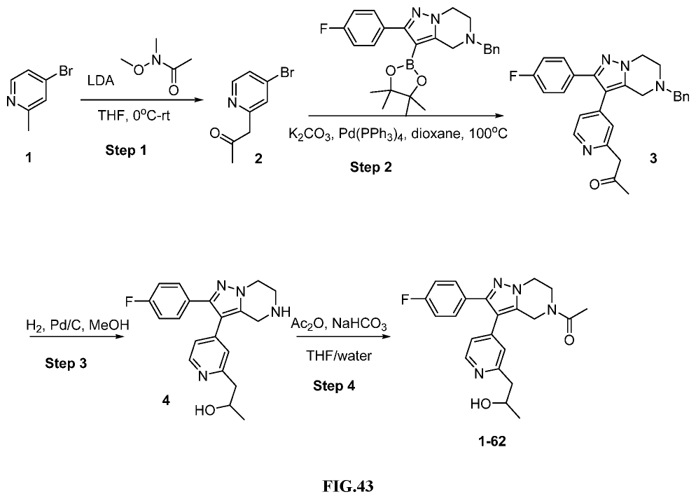

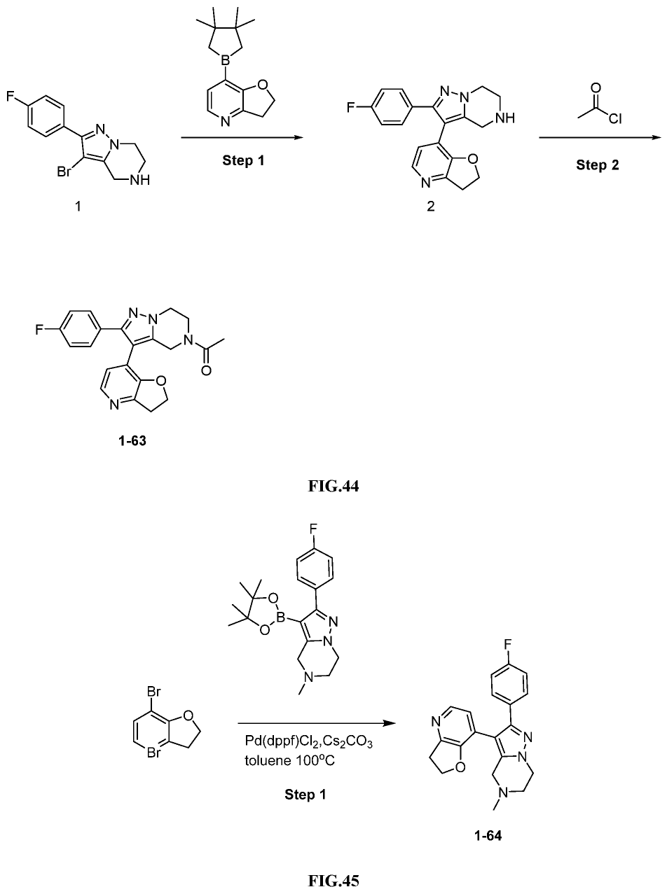

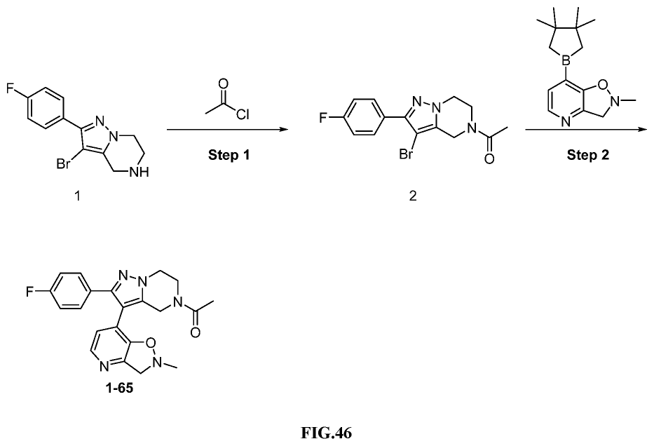

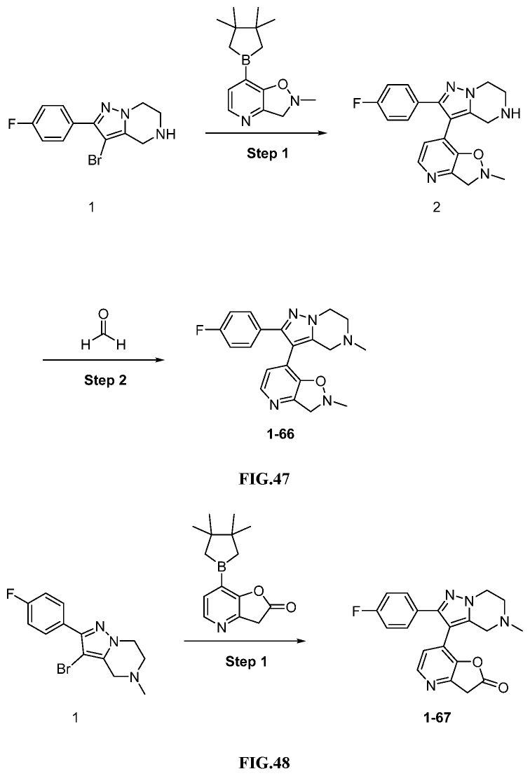

As shown in FIG. 48, the specific synthesis steps are as follows:

Step 1:7-(2-(4-fluorophenyl)-5-methyl-4,5,6,7-tetrahydropyrazolo[1,5-a]pyrazin-3-yl)furo[3,2-b]pyridin-2(3H)-one

To a solution of 3-bromo-2-(4-fluorophenyl)-5-methyl-4,5,6,7-tetrahydropyrazolo[1,5-a]pyrazine (30 mg,0.10 mmol) in Dioxane (3 mL) and Water (0.3 ml), 7-(3,3,4,4-tetramethylborolan-1-yl)furo[3,2-b]pyridin-2(3H)-one (28.29 mg,0.11 mmol), Pd dppf) Cl₂(3 mg,10%w/w) and K₃PO₄(42.45 mg, 0.20 mmol) were added in . The mixture was stirred at 90° C. under N₂ atmosphere for 4 h. The reaction mixture was diluted with water, and the water phase was extracted with EA twice time, the organic phase was combined and concentrated under pressure. The residue was purified via Prep-TLC and give the product as yellow solid(16.14 mg,45.8%)

Chemical Formula: calculated for (M+H+)C₂₀H₁₇FN₄O₂: 364.38, Found:365.

### Example 49. Preparation of Compound 1-68

As shown in FIG. 49, the specific synthesis steps are as follows:

Step 1: 7-(5-acetyl-2-(4-fluorophenyl)-4,5,6,7-tetrahydropyrazolo[1,5-a]pyrazin-3-yl)furo[3,2-b]pyridin-2(3H)-one

To a solution of 1-[2-(4-fluorophenyl)-3-(4,4,5,5-tetramethyl-1,3,2-dioxaborolan-2-yl)-4H,6H,7H-pyrazolo[1,5-a]pyrazin-5-yl]ethanone (100 mg, 0.259 mmol) in 1,4-Dioxane/H₂O=10:1 (30 mL), 7-bromofuro[3,2-b]pyridin-2(3H)-one (83.23 mg, 0.389 mmol), Na₂CO₃ (82.546 mg, 0.778 mmol), Pd(dppf)Cl₂ (37.951 mg, 0.0519 mmol) was added. The reaction mixture was stirred at 100° C. for 16 h. The reaction was complete detected by 1 cms. The solution was filtered, filtrate was collected. The reaction mixture was concentrated under pressure. The crude material was added to a silica gel column and was eluted with DCM/MeOH (10:1). Chemical Formula: calculated for (M+H⁺) C₂₁H₁₇FN₄O₃: 392.3, Found: 393.0

### Example 50. Preparation of Compound 3-14

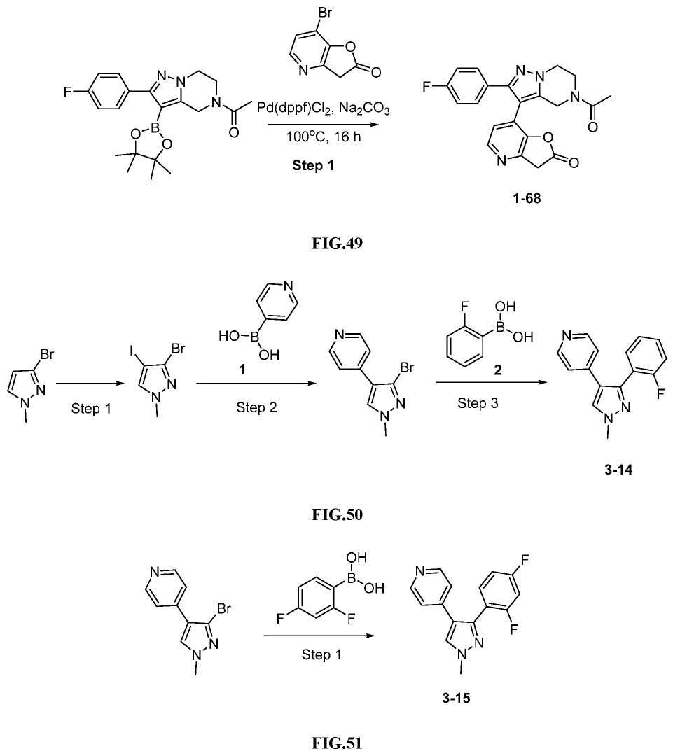

As shown in FIG. 50, the specific synthesis steps are as follows:

Step 1: 3-bromo-4-iodo-1-methylpyrazole

To a stirred solution of 3-bromo-1-methylpyrazole(10.00 g, 62.111 mmol, 1.00 equiv) in ACN(200.00 mL) was added NIS(27.95 g, 124.222 mmol, 2.00 equiv) in portions at room temperature under nitrogen atmosphere. The mixture was stirred for 3 h at 40° C. The reaction monitored by LCMS. The mixture allowed cool down to room temperature. The mixture was concentrated under reduced pressure and the residue was diluted with water(150 mL), the resulting mixture was extracted with EA (2×150 mL). The combined organic layers were dried over anhydrous Na₂SO₄. After filtration, the filtrate was concentrated under reduced pressure. The residue was purified by silica gel column chromatography, eluted with PE/EA (5:1) to afford 3-bromo-4-iodo-1-methylpyrazole(11.59 g, 65.04%) as a light yellow solid.

¹H NMR (400 MHz, Chloroform-d) δ 7.34 (s, 1H), 3.90 (s, 3H).

LC/MS (ESI, m/z): [(M+1)]⁺=286.9, 288.9.

Step 2: 4-(3-bromo-1-methylpyrazol-4-yl)pyridine

To a stirred solution of 3-bromo-4-iodo-1-methylpyrazole(1.00 g, 3.486 mmol, 1.00 equiv),pyridin-4-ylboronic acid(0.43 g, 3.486 mmol, 1 equiv) and K₂CO₃(0.96 g, 6.946 mmol, 1.99 equiv) in H₂O (5.00 mL) /dioxane(15.00 mL) was added Pd(dppf)Cl₂CH₂Cl₂(0.26 g, 0.355 mmol, 0.10 equiv) in portions at room temperature under nitrogen atmosphere. The resulting mixture was stirred for 2 h at 85° C. under nitrogen atmosphere. The resulting mixture was concentrated under reduced pressure. The residue was purified by silica gel column chromatography, eluted with PE/EA(1:1) to afford 4-(3-bromo-1-methylpyrazol-4-yl)pyridine(400 mg, 48.20%) as a green solid.

LC/MS (ESI, m/z): [(M+1)]⁺=238.0, 240.0.

Step 3: 4-[3-(2-fluorophenyl)-1-methylpyrazol-4-yl]pyridine

Into a 8 mL vial were added 4-(3-bromo-1-methylpyrazol-4-yl)pyridine(100.00 mg, 0.420 mmol, 1.00 equiv), 2-fluorophenylboronic acid(88.15 mg, 0.630 mmol, 1.50 equiv), Pd(dppf)Cl₂CH₂Cl₂ (34.22 mg, 0.042 mmol, 0.1 equiv), K₂CO₃ (174.14 mg, 1.260 mmol, 3.0 equiv) and dioxane (3.00 mL)/H₂O (1.00 mL) at room temperature. The resulting mixture stirred for 3 h at 90° C. The resulting mixture was concentrated under reduced pressure. The residue purified by silica gel column chromatography, eluted with DCM/MeOH (10:1). This resulted in 4-[3-(2-fluorophenyl)-1-methylpyrazol-4-yl]pyridine (71.7 mg, 67.40%) as an off-white solid.

¹H NMR (300 MHz, Chloroform-d) δ 8.51-8.43 (m, 2H), 7.73 (s, 1H), 7.56-7.35 (m, 2H), 7.23 (td, J=7.5, 1.2 Hz, 1H), 7.17-7.05 (m, 3H), 4.05 (s, 3H).

LC/MS (ESI, m/z): [(M+1)]⁺=254.1.

### Example 51. Preparation of Compound 3-15

As shown in FIG. 51, the specific synthesis steps are as follows:

Step 1: 4-[3-(2,4-difluorophenyl)-1-methylpyrazol-4-yl]pyridine

Into a 8 mL vial were added 4-(3-bromo-1-methylpyrazol-4-yl)pyridine(100.00 mg, 0.420 mmol, 1.00 equiv), 2,4-difluorophenylboronic acid(99.49 mg, 0.630 mmol, 1.50 equiv), Pd(dppf)Cl₂. CH₂Cl₂(34.22 mg, 0.042 mmol, 0.1 equiv), K₂CO₃(174.14 mg, 1.260 mmol, 3.0 equiv) and dioxane(3.00 mL)/H₂O(1.00 mL) under nitrogen atmosphere at room temperature. The resulting mixture stirred for 3 h at 90° C. The resulting mixture was concentrated under reduced pressure. The residue purified by reverse flash chromatography with the following conditions: column, C18 silica gel; mobile phase, ACN in 0.1% FA aq., 10% to 50% gradient in 10 min; detector, UV 254 nm. This resulted in 4-[3-(2,4-difluorophenyl)-1-methylpyrazol-4-yl]pyridine (87.4 mg, 76.71%) as an off-white solid.

¹H NMR (400 MHz, Chloroform-d) δ 8.50-8.44 (m, 2H), 7.71 (s, 1H), 7.47 (td, J=8.4, 6.4 Hz, 1H), 7.14-7.08 (m, 2H), 6.96 (dddd, J=8.8, 8.0, 2.5, 1.0 Hz, 1H), 6.86 (ddd, J=9.9, 8.8, 2.5 Hz, 1H), 4.02 (s, 3H).

LC/MS (ESI, m/z): [(M+1)]⁺=272.1.

### Example 52. Preparation of Compound 3-16

As shown in FIG. 52, the specific synthesis steps are as follows:

Into a 8 mL vial were added 4-(3-bromo-1-methylpyrazol-4-yl)pyridine (100.00 mg, 0.420 mmol, 1.00 equiv), 3,4-difluorophenylboronic acid(99.49 mg, 0.630 mmol, 1.50 equiv), Pd(dppf)Cl₂.CH₂Cl₂ (34.22 mg, 0.042 mmol, 0.1 equiv), K₂CO₃(174.14 mg, 1.260 mmol, 3.0 equiv) and dioxane (3.00 mL)/H₂O (1.00 mL) at room temperature under nitrogen atmosphere. The resulting mixture stirred for 3 h at 90° C. The resulting mixture was concentrated under reduced pressure. The residue was purified by silica gel column chromatography, eluted with DCM/MeOH(10:1). This resulted in 4-[3-(3,4-difluorophenyl)-1-methylpyrazol-4-yl]pyridine (96.6 mg, 84.78%) as an off-white solid.

¹H NMR (300 MHz, Chloroform-d) δ 8.55 (d, J=5.1 Hz, 2H), 7.63 (s, 1H), 7.42-7.29 (m, 1H), 7.25-7.08 (m, 4H), 4.02 (s, 3H).

LC/MS (ESI, m/z): [(M+1)]⁺=272.1.

### Example 53. Preparation of Compound 3-17

As shown in FIG. 53, the specific synthesis steps are as follows:

Into a 8 mL vial were added 4-(3-bromo-1-methylpyrazol-4-yl)pyridine (100.00 mg, 0.420 mmol, 1.00 equiv), 3,5-difluorophenylboronic acid(99.49 mg, 0.630 mmol, 1.50 equiv), Pd(dppf)Cl₂.CH₂Cl₂ (34.22 mg, 0.042 mmol, 0.1 equiv), K₂CO₃ (174.14 mg, 1.260 mmol, 3.0 equiv) and dioxane (3.00 mL)/H₂O(1.00 mL) at room temperature. The resulting mixture was stirred for 3 h at 90° C. The resulting mixture was concentrated under reduced pressure. The residue was purified by silica gel column chromatography, eluted with DCM/MeOH(10:1). This resulted in 4-[3-(3,5-difluorophenyl)-1-methylpyrazol-4-yl]pyridine (87.1 mg, 76.45%) as an off-white solid.

¹H NMR (300 MHz, Chloroform-d) δ 8.57 (d, J=5.5 Hz, 2H), 7.62 (s, 1H), 7.25-7.17 (m, 2H), 7.03 (dddd, J=6.9, 5.8, 4.6, 2.3 Hz, 2H), 6.81 (tt, J=8.9, 2.4 Hz, 1H), 4.03 (s, 3H).

LC/MS (ESI, m/z): [(M+1)]⁺=272.1.

### Example 54. Preparation of Compound 3-18

As shown in FIG. 54, the specific synthesis steps are as follows:

Into a 8 mL vial were added 4-(3-bromo-1-methylpyrazol-4-yl)pyridine (100.00 mg, 0.420 mmol, 1.00 equiv), 2-chloro-4-fluorophenylboronic acid(109.85 mg, 0.630 mmol, 1.50 equiv), Pd(dppf)Cl₂.CH₂Cl₂ (34.22 mg, 0.042 mmol, 0.1 equiv), K₂CO₃ (174.14 mg, 1.260 mmol, 3.0 equiv) and dioxane (3.00 mL)/H₂O (1.00 mL) in at room temperature. The resulting mixture stirred for 3 h at 90° C. The resulting mixture was concentrated under reduced pressure. The residue product was purified by silica gel column chromatography, eluted with DCM/MeOH(10:1). This resulted in 4-[3-(2-chloro-4-fluorophenyl)-1-methylpyrazol-4-yl]pyridine (57.9 mg, 47.91%) as an off-white solid.

¹H NMR (300 MHz, Chloroform-d) δ 8.46 (d, J=5.8 Hz, 2H), 7.78 (s, 1H), 7.45 (dd, J=8.5, 6.1 Hz, 1H), 7.31-7.18 (m, 1H), 7.16-7.01 (m, 3H), 4.05 (s, 3H).

LC/MS (ESI, m/z): [(M+1)]⁺=288.1.

### Example 55. Preparation of Compound 3-19

As shown in FIG. 55, the specific synthesis steps are as follows:

Into a 8 mL vial were added 4-(3-bromo-1-methylpyrazol-4-yl)pyridine (100.00 mg, 0.420 mmol, 1.00 equiv), 3-fluorophenylboronic acid(88.15 mg, 0.630 mmol, 1.50 equiv), Pd(dppf)Cl₂.CH₂Cl₂ (34.22 mg, 0.042 mmol, 0.1 equiv), K₂CO₃ (174.14 mg, 1.260 mmol, 3.0 equiv) add dioxane (3.00 mL)/H₂O (1.00 mL) at room temperature. The resulting mixture stirred for 3 h at 90° C. The resulting mixture was concentrated under reduced pressure. The residue was purified by silica gel column chromatography, eluted with DCM/MeOH (10:1) to afford 4-[3-(3-fluorophenyl)-1-methylpyrazol-4-yl]pyridine(68.6 mg, 64.49%) as an off-white solid.

¹H NMR (400 MHz, Chloroform-d) δ 8.54-8.48 (m, 2H), 7.60 (s, 1H), 7.35-7.26 (m, 2H), 7.26-7.14 (m, 3H), 7.09-6.99 (m, 1H), 4.00 (s, 3H).

LC/MS (ESI, m/z): [(M+1)]⁺=254.1.

### Example 56. Preparation of Compound 3-20

As shown in FIG. 56, the specific synthesis steps are as follows:

Step 1: P-fluorobenzaldoxime

To a stirred solution of benzaldehyde, 4-fluoro-(5.00 g, 40.286 mmol, 1.00 equiv) and hydroxylamine (1.46 g, 44.314 mmol, 1.1 equiv) in EtOH (9.00 mL)/H₂O (27.00 mL) was added 50% NaOH aq. (25.8 g, 322.284 mmol, 8.00 equiv) dropwise at 0° C. under air atmosphere. The resulting mixture was stirred for 1 h at room temperature under air atmosphere. The reaction was monitored by LCMS. The mixture was acidified to pH 4 with HCl (3M). The resulting mixture was extracted with DCM (3×100 mL). The combined organic layers were washed with brine (1×200 mL), dried over anhydrous Na₂SO₄. After filtration, the filtrate was concentrated under reduced pressure. The residue was purified by silica gel column chromatography, eluted with PE/EA (5:1) to afford P-fluorobenzaldoxime (4.5 g, 80.29%) as a white solid.

¹H NMR (300 MHz, Chloroform-d) δ 8.15 (s, 1H), 7.66-7.53 (m, 2H), 7.18-7.04 (m, 2H).

LC/MS (ESI, m/z): [(M+1)]⁺=140.1

Step 2: 4-fluoro-N-hydroxybenzenecarbonimidoyl chloride

To a stirred solution of P-fluorobenzaldoxime(2.40 g, 17.250 mmol, 1.00 equiv) in DCM(24. 00 mL) was added NCS(2.42 g, 18.113 mmol, 1.05 equiv) at 0° C. under air atmosphere. The resulting mixture was stirred for 1 h at room temperature under air atmosphere. The reaction was monitored by LCMS. The residue was purified by silica gel column chromatography, eluted with PE/EA (10:1) to afford 4-fluoro-N-hydroxybenzenecarbonimidoyl chloride (2 g, 66.80%) as a white solid.

¹H NMR (300 MHz, Chloroform-d) δ 8.20-8.07 (m, 1H), 7.96-7.80 (m, 2H), 7.21-7.06 (m, 2H).

Step 3: 4-[3-(4-fluorophenyl)-1,2-oxazol-4-yl]pyridine

To a stirred solution of 4-fluoro-N-hydroxybenzenecarbonimidoyl chloride(300.00 mg, 1.728 mmol, 1.00 equiv) and 4-ethynylpyridine(267.36 mg, 2.593 mmol, 1.50 equiv) in dioxane(5.00 mL) was added K₂CO₃(716.63 mg, 5.185 mmol, 3 equiv) at room temperature under nitrogen atmosphere. The resulting mixture was stirred for 2 h at 80° C. under nitrogen atmosphere. The reaction was monitored by LCMS. The residue was purified by reverse flash chromatography with the following conditions: column, C18 silica gel; mobile phase, ACN in 0.1% FA aq., 30% to 60% gradient in 30 min; detector, UV 254 nm to afford 4-[3-(4-fluorophenyl)-1,2-oxazol-4-yl]pyridine (11.4 mg, 2.75%) as an off-white solid.

¹H NMR (300 MHz, Chloroform-d) δ 8.85-8.77 (m, 2H), 7.95-7.82 (m, 2H), 7.77-7.69 (m, 2H), 7.26-7.18 (m, 2H), 7.01 (s, 1H).

LC/MS (ESI, m/z): [(M+1)]⁺=241.1

### Example 57. Examination of biological activities

The CK1δ kinase assay was performed with a buffer (40 μL, pH 7.5) containing 50 mM Tris, 10 mM MgCl₂, 1 mM dithiothreitol, 100 μg/mL BSA with 10 μM ATP, 2 nM wild type CK1δ, and 42 μM peptide substrate PLSRTLpSVASLPGL (Flotow et al., 1990) in the presence of 1 μL of a CK1δ inhibitor (e.g., a compound of the present application) or 4% DMSO (e.g., as control). The reaction mixture was incubated for 85 min at 25° C.; detection was carried out as described for the Kinase-Glo Assay (Promega). Luminescent output was measured on the Perkin Elmer Envision plate reader (PerkinElmer, Waltham, Mass.).

Bmal1-dLuc or Per2-dLuc U2OS cells were suspended in the culture medium (DMEM supplemented with 10% fetal bovine serum, 0.29 mg/mL L-glutamine, 100 units/mL penicillin, and 100 mg/mL streptomycin) and plated onto 96-well white solid-bottom plates at 200 μL (10,000 cells) per well. After 2 days, 100 μL of the explant medium (DMEM supplemented with 2% B27, 10 mM HEPES, 0.38 mg/mL sodium bicarbonate, 0.29 mg/mL L-glutamine, 100 units/mL penicillin, 100 mg/mL streptomycin, 0.1 mg/mL gentamicin, and 1 mM luciferin, pH 7.2) was dispensed to each well, followed by the application of 1 μL of a compound of the present application (dissolved in DMSO; final concentration was 0.7% in DMSO). The plate was covered with an optically clear film and set to microplate reader (Infinite M200, Tecan). The luminescence was recorded every 1 h for 3-4 days. The period parameter was obtained from the luminescence rhythm by curve fitting program CellulaRhythm or MultiCycle (Actimetrics), both of which generated similar results.

The CK1δ inhibition results (IC50) are summarized in table 8.

TABLE 8

| Number | Compound | CK1δ inhibition (IC50, nM) |
|---|---|---|
| 1. | 1-8 | * |
| 2. | 1-5 | 499.1 |
| 3. | 1-6 | 253.8 |
| 4. | 1-7 | 613.9 |
| 5. | 1-13 | 67.32 |
| 6. | 1-14 | 61.47 |
| 7. | 2-2 | 212 |
| 8. | 1-12 | 74.93 |
| 9. | 3-4 | 480 |
| 10. | 3-5 | 7712 |
| 11. | 3-1 | 180 |
| 12. | 3-2 | 11098 |
| 13. | 2-5 | 408 |
| 14. | 3-7 | 7136 |
| 15. | 3-8 | 97422 |
| 16. | 3-9 | 21357 |
| 17. | 3-6 | 178 |
| 18. | 6-1 | 93 |
| 19. | 1-16 | 9778 |
| 20. | 4-1 | 214.8 |
| 21. | 5-1 | 132 |
| 22. | 1-31 | 81.29 |
| 23. | 1-32 | 367.9 |
| 24. | 1-33 | 176.9 |
| 25. | 1-27 | 163.3 |
| 26. | 1-42 | 160.7 |
| 27. | 1-36 | 278.9 |
| 28. | 1-34 | * |
| 29. | 1-44 | 1841 |
| 30. | 1-45 | 369.8 |
| 31. | 1-46 | 273.4 |
| 32. | 1-47 | 685.8 |
| 33. | 5-2 | 168.1 |
| 34. | 1-48 | 10000 |
| 35. | 5-3 | 10000 |
| 36. | 1-49 | 319.7 |
| 37. | 1-50 | 355.2 |
| 38. | 5-4 | 2199 |
| 39. | 1-51 | 962.8 |
| 40. | 1-52 | 3107 |
| 41. | 1-53 | 16961 |
| 42. | 1-54 | 800.1 |
| 43. | 1-55 | 495.8 |
| 44. | 1-56 | 106 |
| 45. | 5-5 | 319.5 |
| 46. | 1-57 | 131.1 |
| 47. | 1-58 | 722.8 |
| 48. | 1-59 | 39.7 |
| 49. | 1-60 | 10000 |
| 50. | 1-61 | 10000 |
| 51. | 1-62 | 10000 |
| 52. | 1-63 | 10000 |
| 53. | 1-64 | 10000 |
| 54. | 1-65 | 10000 |
| 55. | 1-66 | 10000 |
| 56. | 1-67 | 10000 |
| 57. | 1-68 | 10000 |
| 58. | 3-14 | 1080 |
| 59. | 3-15 | 530.8 |
| 60. | 3-16 | 497.2 |
| 61. | 3-17 | 1024 |
| 62. | 3-18 | 4394 |
| 63. | 3-19 | 281.5 |
| 64. | 3-20 | 3658 |

The “*” in table 8 indicates that the value exceeds the measurement range, for example, the value is >100000.

The CK1δ inhibition results (EC50) are summarized in table 9.

TABLE 9

| Number | Compound | CK1δ (EC50, μM) |
|---|---|---|
| 1. | 1-1 | 7.9 |
| 2. | 6-1 | 19.3 |
| 3. | 1-12 | * |
| 4. | 1-16 | 15.4 |
| 5. | 1-5 | 20.2 |
| 6. | 1-6 | 14.8 |
| 7. | 7-1 | 11.8 |
| 8. | 2-2 | 11.2 |
| 9. | 2-5 | 17.4 |
| 10. | 1-7 | * |
| 11. | 1-13 | 10.8 |
| 12. | 1-14 | 9.4 |
| 13. | 1-8 | * |
| 14. | 3-8 | * |
| 15. | 3-7 | * |
| 16. | 3-9 | * |
| 17. | 3-6 | 15.4 |
| 18. | 3-4 | * |
| 19. | 3-5 | * |
| 20. | 3-1 | * |
| 21. | 3-2 | * |
| 22. | 4-1 | * |
| 23. | 5-1 | 7.5 |
| 24. | 1-31 | 12 |
| 25. | 1-32 | 9.7 |
| 26. | 1-33 | 5.3 |
| 27. | 1-27 | 4.5 |
| 28. | 1-42 | 9.2 |
| 29. | 1-36 | 5.5 |
| 30. | 1-34 | * |
| 31. | 1-37 | 17.9 |
| 32. | 1-35 | 7.4 |
| 33. | 1-45 | 5.6 |
| 34. | 1-46 | 3.14 |
| 35. | 1-47 | 6.8 |
| 36. | 5-2 | 2.2 |
| 37. | 1-49 | 1.9 |
| 38. | 1-50 | 2.5 |
| 39. | 1-51 | 5.2 |
| 40. | 1-54 | 3.3 |
| 41. | 1-55 | 8.1 |
| 42. | 1-56 | 9 |
| 43. | 5-5 | 5 |
| 44. | 1-57 | 0.7 |
| 45. | 1-58 | 2.5 |
| 46. | 1-59 | 4.5 |
| 47. | 3-14 | 12.9 |
| 48. | 3-15 | 7.4 |
| 49. | 3-16 | 5 |
| 50. | 3-17 | 15.2 |
| 51. | 3-18 | 42.3 |
| 52. | 3-19 | 13.4 |

The “*” in table 9 indicates that the value exceeds the measurement range, for example, the value is >30.

### Example 58. Antitumor activity

Cell proliferation assay

MDA-MB-231 cells treated with a compound of the present application (e.g., compound 1-1, 2-5, 1-14 and 3-8, respectively) were seeded at a density of 5×10⁴cells/well in 12-well plate and incubated at 37° C. with 5% CO₂. After 72 hours of culturing, the cells were washed with PBS, fixed in 4% formaldehyde for 15 minutes and stained with 0.1% crystal violet for 20 minutes. Cells were washed with water and the staining was extracted with 10% acetic acid for 20 minutes and absorbance was measured at 590 nm. The experiments were performed in triplicates.

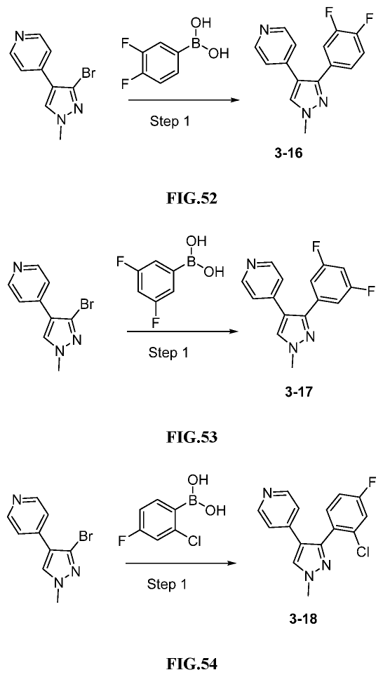

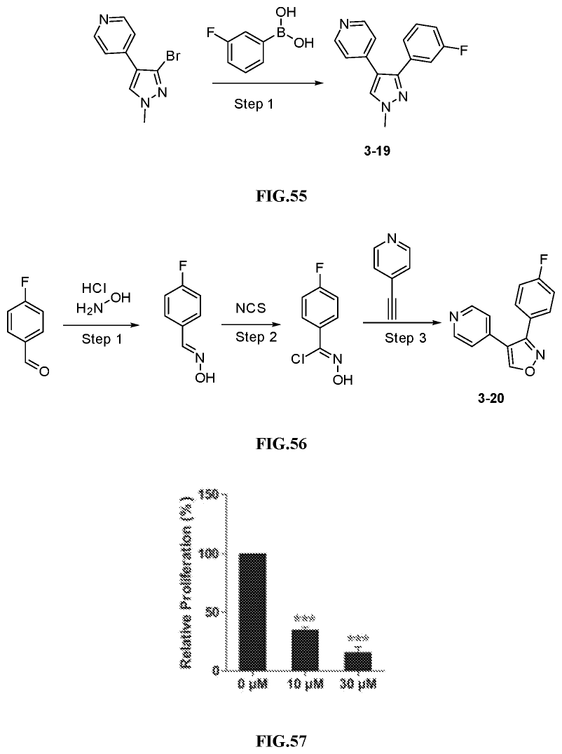

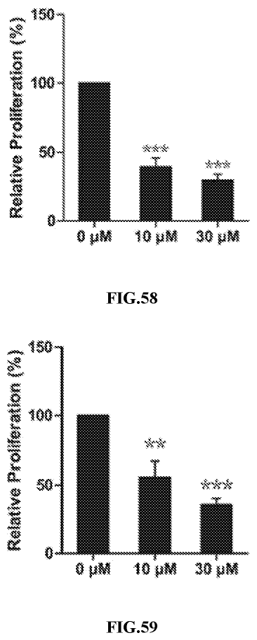

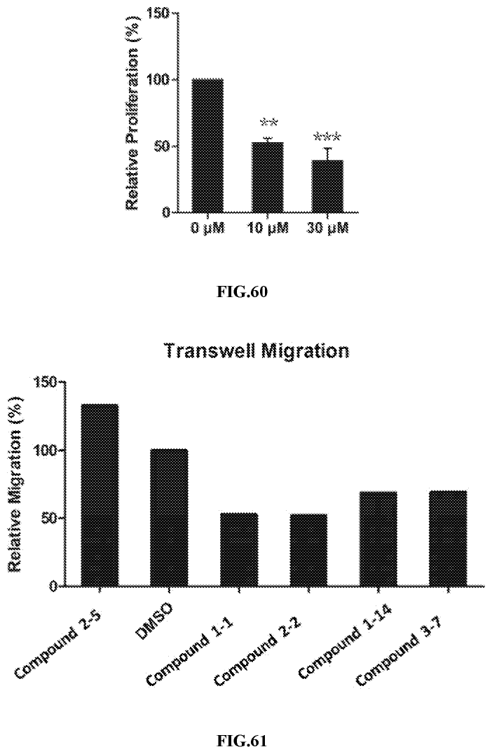

FIGS. 57-60 demonstrate the relative proliferation (%) of cells after treating with the compounds 1-1, 2-5, 1-14 and 3-8 of the present application, respectively. As shown in FIG. 57 (for the compound 1-1), FIG. 58 (for the compound 2-5), FIG. 59 (for the compound 1-14), and FIG. 60 (for the compound 3-8), the MDA-MB-231 cells were treated with various concentrations of compounds 1-1, 2-5, 1-14 and 3-8, and the proliferation of the MDA-MB-231 cells was affected in a concentration dependent manner.

Transwell cell migration assay

MDA-MB-231 cells (breast cancer cells) were starved in 0.5% serum-DMEM for 8 hours. For the migration assay, a total of 5×10⁴cells were suspended in 500 μL of serum-free DMEM medium and seeded into the upper chamber of a 8 μM pore size insert. Then, 750 μL of DMEM (containing 10% FBS) with or without the compound of the present application was added to the lower compartment. After incubation at 37° C. for 24 h, migrated cells were washed with PBS, fixed in 4% PFA and stained for 30 min in crystal violet solution (0.1% crystal violet). Cells that did not migrate to the lower compartment were removed with a cotton swab. Each insert was photographed in 3 random fields at a magnification of 40×.

FIG. 61 shows the relative migration (%) of cells after treating with compound 2-5, DMSO, compound 1-1, compound 2-2, compound 1-14 and compound 3-7. As shown in FIG. 61, the migration of MDA-MB-231 was reduced in the presence of compounds 2-5, 1-1, 2-2, 1-14 and 3-7.

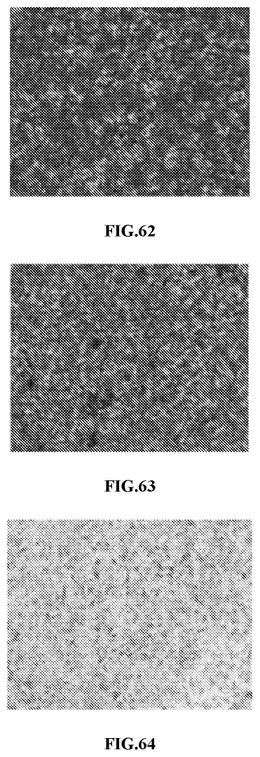

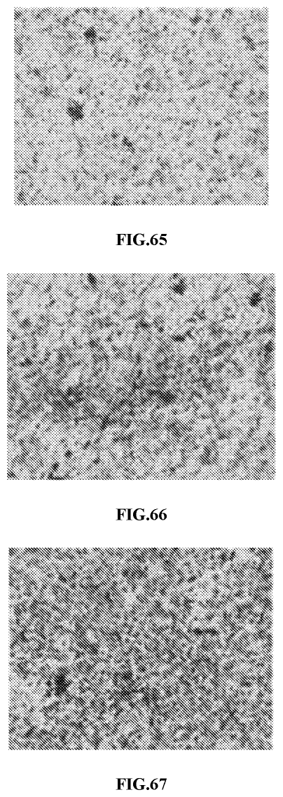

FIG. 62 (for compound 2-5), FIG. 63 (for DMSO, as a control), FIG. 64 (for compound 1-1), FIG. 65 (for compound 2-2), FIG. 66 (for compound 1-14) and FIG. 67 (for compound 3-7) demonstrate the migrated cells after treating with compound 2-5, DMSO, and compounds 1-1, 2-2, 1-14 and 3-7, respectively. As shown in FIGS. 62-67, treatment with the compounds 2-5, 1-1, 2-2, 1-14 and 3-7 suppressed the migration of MDA-MB-231 cells.

These results suggest that the compounds of the present application could suppress formation of cancer cell colonies, as well as the migration and invasion of the cancer cells.

### Example 59. Assay of Drug Transport

1. Preparation of Caco-2 Cells

1) 50 μL and 25 mL of cell culture medium were added to each well of the Transwell insert and reservoir, respectively. And then the HTS transwell plates were incubated at 37° C., 5% CO₂ for 1 hour before cell seeding.

2) Caco-2 cells were diluted to 6. 86×10⁵ cells/mL with culture medium and 50 μL of cell suspension were dispensed into the filter well of the 96-well HTS Transwell plate. Cells were cultivated for 14-18 days in a cell culture incubator at 37° C., 5% CO₂, 95% relative humidity. Cell culture medium was replaced every other day, beginning no later than 24 hours after initial plating.

2. Preparation of Stock Solutions

10 mM stock solutions of test compounds were prepared in DMSO. The stock solutions of positive controls were prepared in DMSO at the concentration of 10 mM. Digoxin and propranolol were used as control compounds in this assay.

3. Assessment of Cell Monolayer Integrity

1) Medium was removed from the reservoir and each Transwell insert and replaced with prewarmed fresh culture medium.

2) Transepithelial electrical resistance (TEER) across the monolayer was measured using Millicell Epithelial Volt-Ohm measuring system (Millipore, USA).

3) The Plate was returned to the incubator once the measurement was done.

The TEER value was calculated according to the following equation: TEER measurement (ohms) * Area of membrane (cm²)=TEER value (ohm·cm²) TEER value should be greater than 230 ohm·cm², which indicates the well-qualified Caco-2 monolayer.

4. Assay Procedures

1) The Caco-2 plate was removed from the incubator and washed twice with pre-warmed HMS (10 mM HEPES, pH 7.4), and then incubated at 37° C. for 30 minutes.

2) The stock solutions of control compounds and test compounds were diluted in DMSO to get 1 mM solutions and then diluted with HMS (10 mM HEPES, pH 7.4) get 5 μM working solutions. The final concentration of DMSO in the incubation system was 0.5%.

3) To determine the rate of drug transport in the apical to basolateral direction. 125 μL of 5 μM working solution of control compound and test compounds were added to the Transwell insert (apical compartment), and transfer 50 μL sample (D0 sample) immediately from the apical compartment to a new 96-well plate. Fill the wells in the receiver plate (basolateral compartment) with 235 μL of MSS (10 mM HEPES, pH 7.4).

4) To determine the rate of drug transport in the basolateral to apical direction. 285 μL of 5 μM working solution of control compound and test compounds were to the receiver plate wells (basolateral compartment), and transfer 50 μL sample (D0 sample) immediately from the basolateral compartment to a new 96-well plate. Fill the wells in the Transwell insert (apical compartment) with 75 μL of MSS (10 mM HEPES, pH 7.4). The assay was performed in duplicate.

5) The plates were incubated at 37° C. for 2 hours.

6) At the end of the incubation, 50 μL samples from donor sides (apical compartment for Ap→Bl flux, and basolateral compartment for Bl→Ap) and receiver sides (basolateral compartment for Ap→Bl flux, and apical compartment for Bl→Ap) were transferred to wells of a new 96-well plate, followed by the addition of 4 volume of cold methanol containing appropriate internal standards (IS). Samples were Vortexed for 5 minutes and then centrifuged at 3,220 g for 40 minutes. An aliquot of 100 μL of the supernatant was mixed with an appropriate volume of ultra-pure water before LC-MS/MS analysis.

7) To determine the Lucifer Yellow leakage after 2-hour transport period, stock solution of Lucifer yellow was prepared in water and diluted with HMS (10 mM HEPES, pH 7.4) to reach the final concentration of 100 μM. 100 μL of the Lucifer yellow solution was added to each Transwell insert (apical compartment), followed by filling the wells in the receiver plate (basolateral compartment) with 300 μL of HBSS (10 mM HEPES, pH 7.4). The plates were Incubated at 37° C. for 30 mins. 80 μL samples were removed directly from the apical and basolateral wells (using the basolateral access holes) and transferred to wells of new 96 wells plates. The Lucifer Yellow fluorescence (to monitor monolayer integrity) signal was measured in a fluorescence plate reader at 485 nM excitation and 530 nM emission.

5. Data Analysis

The apparent permeability coefficient (Pₐₚₚ), in units of centimeter per second, can be calculated for Caco-2 drug transport assays using the following equation:

Pₐₚₚ=(V_{A}×[drug]_{acceptor})/(Area×Time×[drug]_{initial,donor})

Where V_{A} is the volume (in mL) in the acceptor well, Area is the surface area of the membrane (0.143 cm² for Transwell-96 Well Permeable Supports), and time is the total transport time in seconds.

The efflux ratio will be determined using the following equation:

Efflux Ratio=P_{app(B-A)}/P_{app(A-B)}

Where P_{app (B-A)} indicates the apparent permeability coefficient in basolateral to apical direction, and P_{app (A-B)} indicates the apparent permeability coefficient in apical to basolateral direction.

The recovery can be determined using the following equation:

Recovery %=(V_{A}×[drug]_{acceptor}+V_{D}×[drug]_{donor})/(V_{D}×[drug]_{initial, donor})

Where V_{A} is the volume (in mL) in the acceptor well (0.235 mL for Ap→Bl flux, and 0.075 mL for Bl→Ap), V_{D} is the volume (in mL) in the donor well (0.075 mL for Ap→Bl flux, and 0.235 mL for Bl→Ap)

The leakage of Lucifer Yellow, in unit of percentage (%), can be calculated using the following equation:

%LY leakage=100×[LY]_{acceptor}/([LY]_{donor}+[LY]_{acceptor})

LY leakage of <1% is acceptable to indicate the well-qualified Caco-2 monolayer.

The P_{app (B-A)}, P_{app (A-B)} and Efflux ratio are summarized in Table 10.

TABLE 10

| | P_{app(A-B)} | P_{app(B-A)} | Efflux |
|---|---|---|---|
| Compound | (10⁻⁶, cm/s) | (10⁻⁶, cm/s) | Ratio |
| Digoxin | 0.30 | 16.98 | 56.52 |
| Propranolol | 26.55 | 13.63 | 0.51 |
| 1-50 | 8.5 | 30.8 | 3.6 |
| 1-57 | 19.25 | 17.32 | 0.9 |
| 1-59 | 22.76 | 23.87 | 1.05 |

### Example 60. Assay of Intrinsic Clearance

1. The master solution was prepared according to Table 11.

TABLE 11

| Reagent | Stock Concentration | Volume | Final Concentration |
|---|---|---|---|
| Phosphate buffer | 200 mM | 200 μL | 100 mM |
| Ultra-pure H₂O | — | 106 μL | — |
| MgCl₂ solution | 50 mM | 40 μL | 5 mM |

2. Three separated experiments were performed as follows. a) With NADPH: 10 μL of 20 mg/mL liver microsomes and 40 μL of 10 mM NADPH were added to the incubations. The final concentrations of microsomes and NADPH were 0.5 mg/mL and 1 mM, respectively. b) Without NADPH: 10 μL of 20 mg/mL liver microsomes and 40 μL of ultra-pure H₂O were added to the incubations. The final concentration of microsomes was 0.5 mg/mL. c) Heat-inactivated microsomes without NADPH: 10 μL of 20 mg/mL heat-inactivated liver microsomes and 40 μL of ultra-pure H₂O were added to the incubations. The final concentration of microsomes was 0.5 mg/mL.

3. The reaction was started with the addition of 4 μL of 200 μM test compound solution or control compound solution at the final concentration of 2 μM and carried out at 37° C.

4. Aliquots of 50 μL were taken from the reaction solution at 0, 15, 30, 45 and 60 min. The reaction was stopped by the addition of 4 volumes of cold acetonitrile with IS (100 nM alprazolam, 200 nM labetalol, 200 nM caffeine and 2 μM ketoprofen). Samples were centrifuged at 3, 220 g for 40 minutes. Aliquot of 100 μL of the supernatant was mixed with 100 μL of ultra-pure H₂O and then used for LC-MS/MS analysis.

5. Data Analysis

All calculations were carried out using Microsoft Excel.

Peak areas were determined from extracted ion chromatograms. The slope value, k, was determined by linear regression of the natural logarithm of the remaining percentage of the parent drug vs. incubation time curve.

The in vitro half-life (in vitro t_{1/2}) was determined from the slope value:

in vitro t_{1/2}=−(0.693/k)

Conversion of the in vitro t_{1/2} (min) into the in vitro intrinsic clearance (in vitro CLᵢₙₜ, in μL/min/mg protein) was done using the following equation (mean of duplicate determinations):

in vitro CLᵢₙₜ=(0.693 * volume of incubation(μl))/(in vitro t_{1/2} * amount of proteins(mg))

Conversion of the in vitro t_{1/2} (min) into the scale-up unbound intrinsic clearance (Scale-up CLᵢₙₜ, in mL/min/kg) was done using the following equation (mean of duplicate determinations):

The Scaling Factors for Intrinsic Clearance Prediction in Liver Microsomes are summarized in Table 12.

TABLE 12

| | Liver Weight (g | Microsomal | Liver blood | |
|---|---|---|---|---|
| | liver/kg body | Concentration | flow (Q, mL/ | Scaling |
| Species | weight)^{a} | (mg/g liver)^{b} | min/kg)^{a} | Factor |
| Human | 25.7 | 48.8 | 20.7 | 1254.2 |
| Monkey | 30.0 | 50.0 | 43.6 | 1500.0 |
| Dog | 32.0 | 77.9 | 30.9 | 2492.8 |
| Rat | 40.0 | 44.8 | 55.2 | 1792.0 |
| Mouse | 88.0 | 50.0 | 90.0 | 4400.0 |

^{a}Iwatsubo et al, Davies and Morris, 1993, 10 (7) pp 1093-1095.
^{b}Barter et al, 2007, Curr Drug Metab, 8(1), pp 33-45; Iwatsubo et al, 1997, JPET, 283 pp 462-469.

The Papp (B-A), Papp (A-B) and Efflux ratio are summarized in Table 13.

TABLE 13

| | | in vitro t_{1/2} | in vitro CLᵢₙₜ |
|---|---|---|---|
| Compound | Species | (min) | (μL/min/mg protein) |
| 1-46 | Human | 23.05 | 60.14 |
| | Mouse | 30.88 | 44.88 |
| 5-2 | Human | 174.69 | 7.93 |
| | Mouse | 55.01 | 25.2 |
| 1-49 | Human | 27.98 | 49.53 |
| | Mouse | 13.35 | 103.83 |
| 1-57 | Human | 13.38 | 103.61 |
| | Mouse | 4.86 | 285.08 |
| 1-59 | Human | 142.96 | 9.7 |
| | Mouse | 62.29 | 22.25 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A compound of Formula I, Formula IL Formula III, Formula IV, Formula V, or Formula VI, or a pharmaceutically acceptable salt thereof:
wherein a structure of the Formula I is: 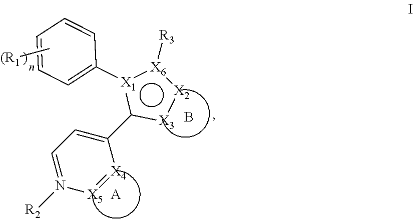
wherein R₁ is a halogen;
n is 0, 1, or 2;
X₁, X₂, X₃, X₄, X₅ and X₆ are each independently C or N;
R₂ is absent or O;
R₃ is absent or —CN; and 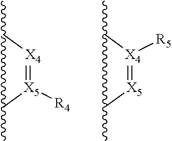
A is absent, A is represented by the preceding formulae, or ring A;
wherein R₄ is at least one selected from the group consisting of: —NH₂, C₁-C₆ alkyl, alkyl-COO-alkyl, alkyl-NH-alkyl and alkyl-OH;
R₅ is a halogen; and
ring A is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—; and
the ring A is optionally substituted with a R₈ substituent; and
R₈ is ═O;
wherein B is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—; and
B is optionally substituted with one or more R₆ substituents;
the R₆ is further optionally substituted with a R₇ substituent;
wherein each R₆ is independently selected from the group consisting of: heterocycloalkyl, C₁-C₆ alkyl, CO-alkyl, CO-heterocycloalkyl, acyl-alkyl, benzyl, p-methoxybenzyl, O and CO-alkylcyano; and
R₇ is C₁-C₆ alkyl;
wherein a structure of the Formula II is: 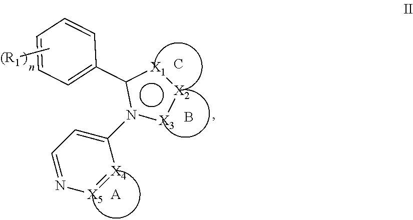
wherein R₁ is a halogen;
n is 0, 1, or 2;
X₁, X₂, and X₃ are each independently C or N; and 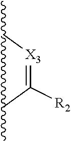
A is absent, A is represented by the preceding formula, or ring A;
wherein ring A is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—; and
ring A is optionally substituted with a R₃ substituent; and
R₃ is ═O;
R₂ is —NH₂ or C₁-C₆ alkyl; 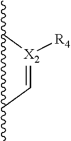
B is absent, B is represented by the preceding formula, or ring B;
R₄ is C₁-C₆ alkyl;
wherein ring B is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—; 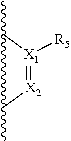
C is absent, C is represented by the preceding formula, or ring C;
wherein R₅ is absent, a cyano or an amide group;
ring C is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—; and
the ring C is optionally substituted with a R₆ substituent; and
R₆ is ═O;
wherein a structure of the Formula III is: 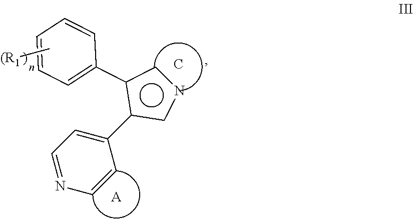
wherein R₁ is a halogen;
n is 0, 1, or 2;
A is absent or ring A;
the ring A is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—; and
the ring A is optionally substituted with a R₄ substituent, R₄ is ═O; 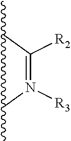
C is represented by the preceding formula or ring C;
wherein R, is —CN, —CONH₂, or —COO-alkyl;
R₃ is absent or C₁-C₆ alkyl;
the ring C is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—, and
the ring C is optionally substituted with a R₄ substituent;
R₄ is ═O;
wherein a structure of the Formula IV is: 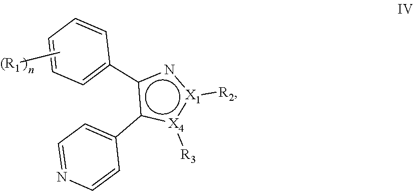
wherein R₁ is a halogen;
n is 0, 1, or 2;
X₁ is C, O, or N;
X₄ is C or N;
R₂ is absent or C₁-C₆ alkyl; and 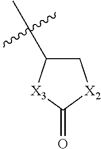
R₃ is absent, PMB, C₁-C₆ alkyl, or represented by the preceding formula; and
X₂, X₃ are each independently C or O;
wherein a structure of the Formula V is: 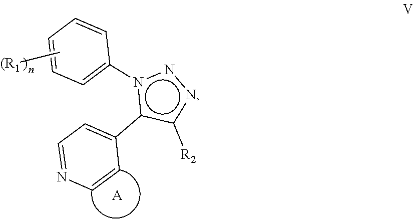
wherein R₁ is a halogen;
n is 0, 1, or 2;
R₂ is absent, —COO-alkyl, or —CO—R₃;
R₃ is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—; and
R₃ is optionally substituted with a R₄ substituent;
R₄ is C₁-C₆ alkyl; and
A is absent or ring A; and
the ring A is a 4- to 7-membered cycloalkyl or heterocycloalkyl or a 5- to 6-membered heteroaryl, wherein up to 2 carbon atoms are replaced with at least one heteroatom selected from the group consisting of ═N— and —O—;
wherein a structure of the Formula VI is: 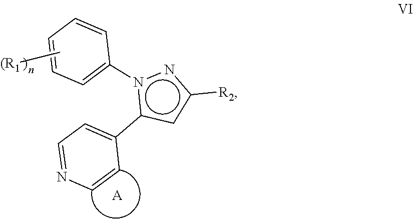
wherein R₁ is a halogen;
n is 0, 1, or 2;
R₂ is C₁-C₆ alkyl; and
A is a 4- to 7-membered cycloalkyl or heterocycloalkyl or a 5- to 6-membered heteroaryl, wherein up to 2 carbon atoms are replaced with at least one heteroatom selected from the group consisting ═N— and —O—; and
A is optionally substituted with a R₃ substituent, and R₃ is ═O.

2-6. (canceled)

7. The compound according to claim 1 any one of claims wherein A in the Formula I is at least one selected from the group consisting of:

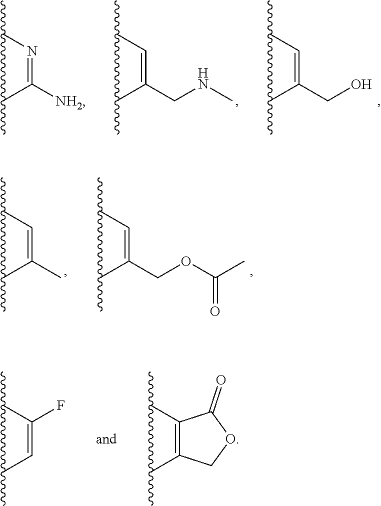

8-11. (canceled)

12. The compound according to claim 1, wherein B in the Formula I is at least one selected from the group consisting of:

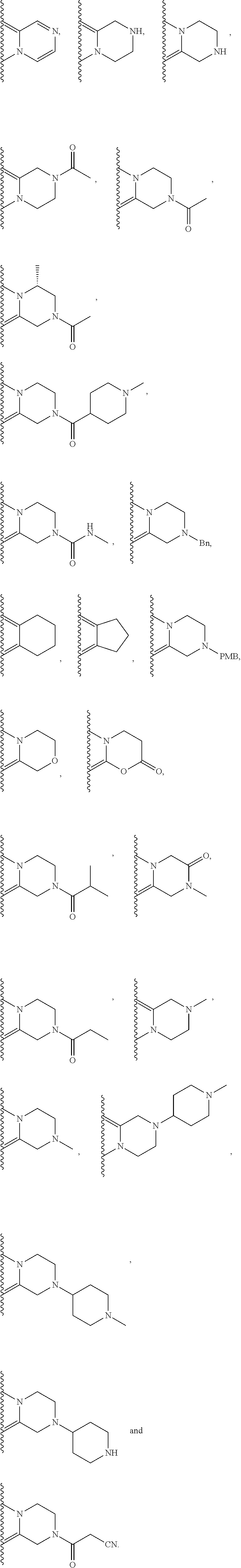

13. The compound according to claim 1, wherein said A in Formula I is

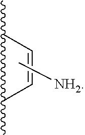

14-16. (canceled)

17. The compound according to claim 1, wherein the compound is at least one selected from the group consisting of:

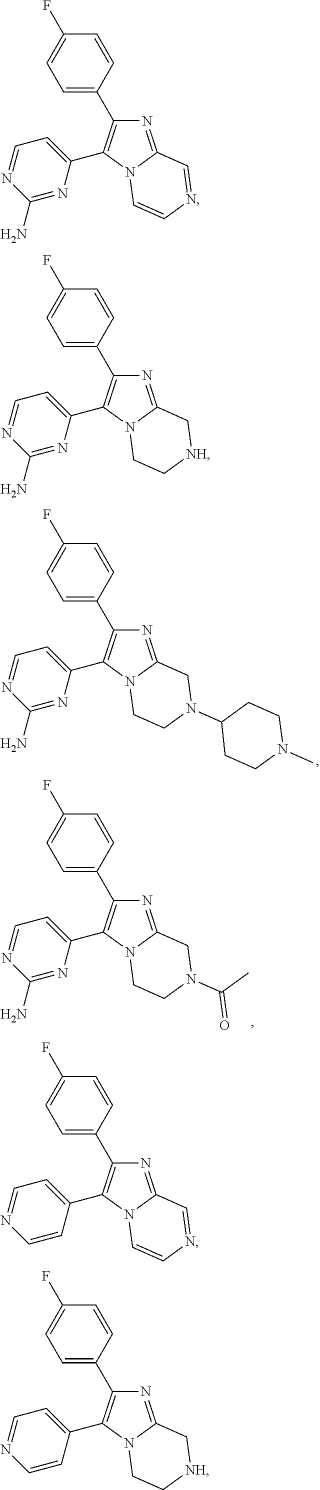

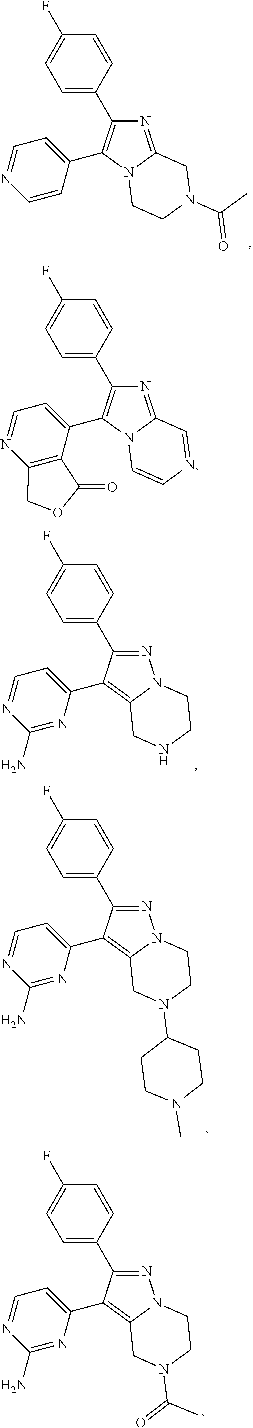

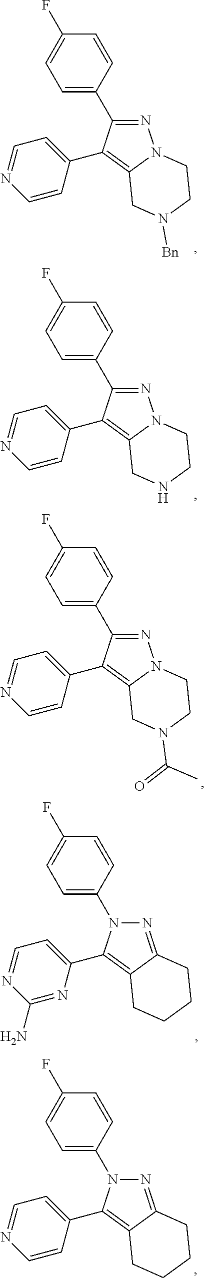

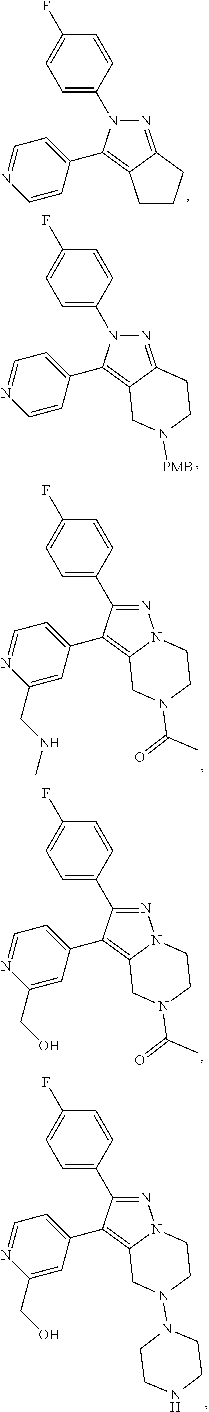

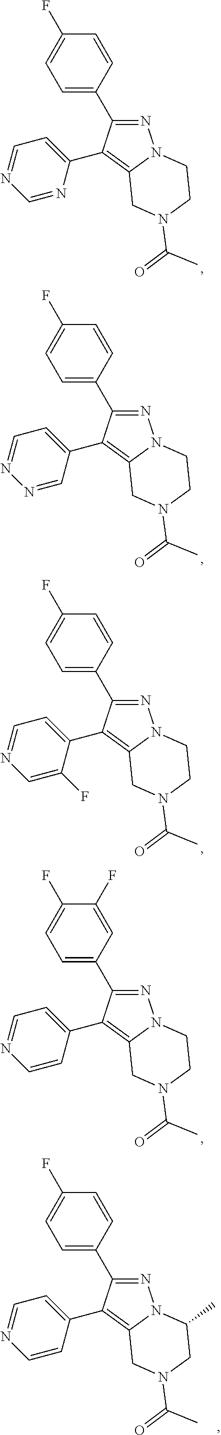

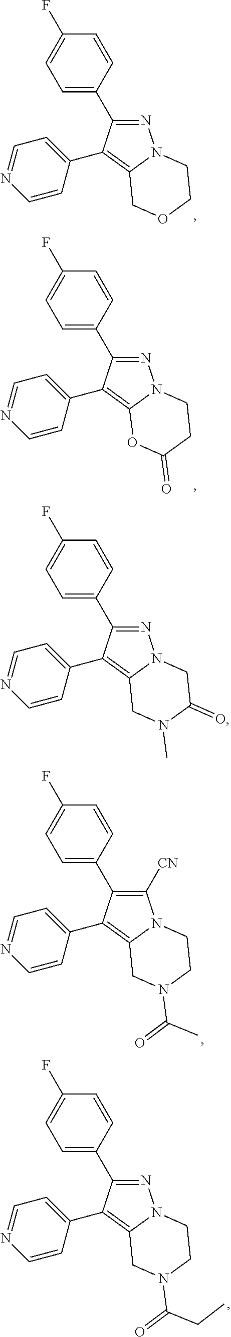

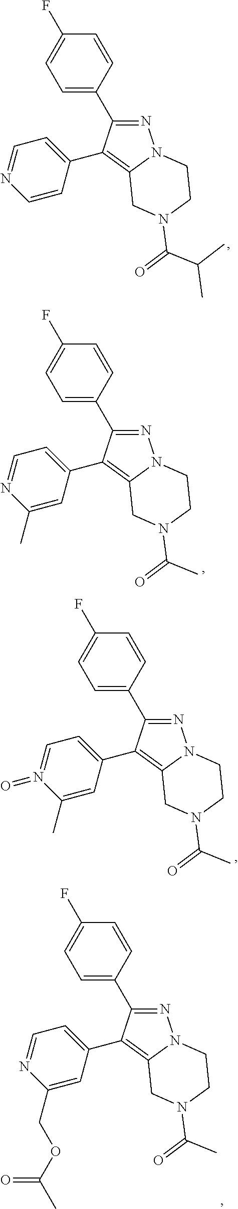 [SMILES: CC(C)C(=O)N1CCn2nc(-c3ccc(F)cc3)c(-c3ccncc3)c2C1 ; CC(=O)N1CCn2nc(-c3ccc(F)cc3)c(-c3ccnc(C)c3)c2C1 ; CC(=O)N1CCn2nc(-c3ccc(F)cc3)c(-c3cc[n+]([O-])c(C)c3)c2C1 ; CC(=O)OCc1cc(-c2c(-c3ccc(F)cc3)nn3c2CN(C(C)=O)CC3)ccn1]

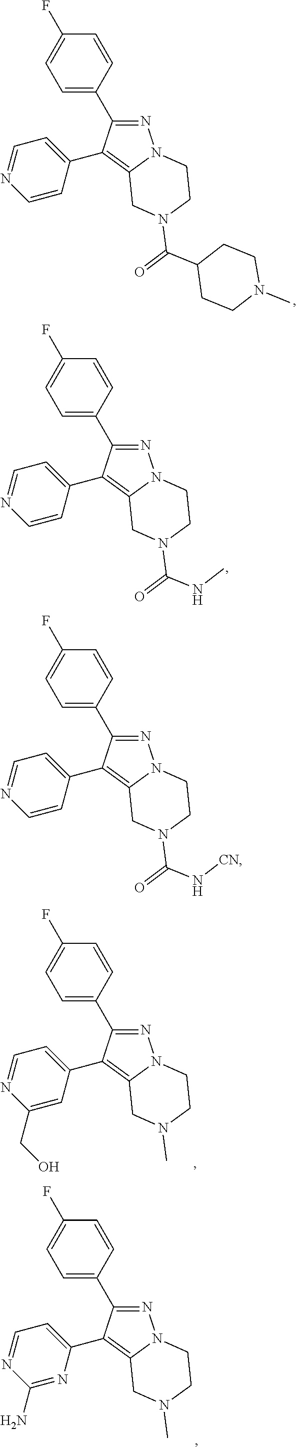

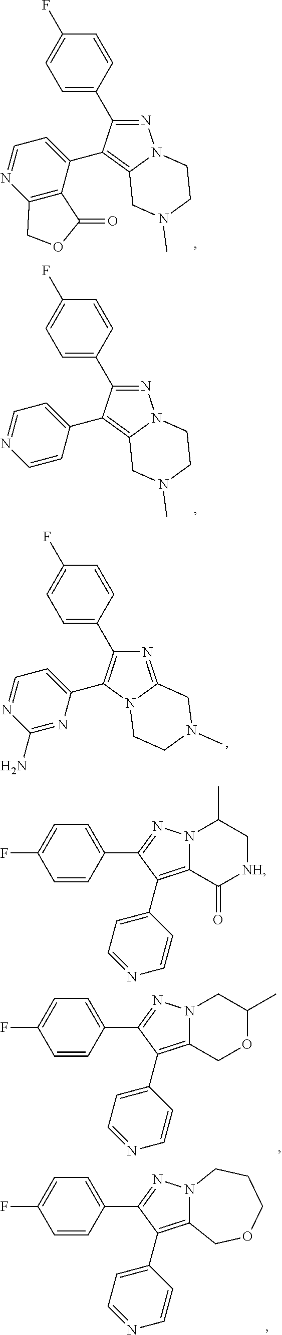

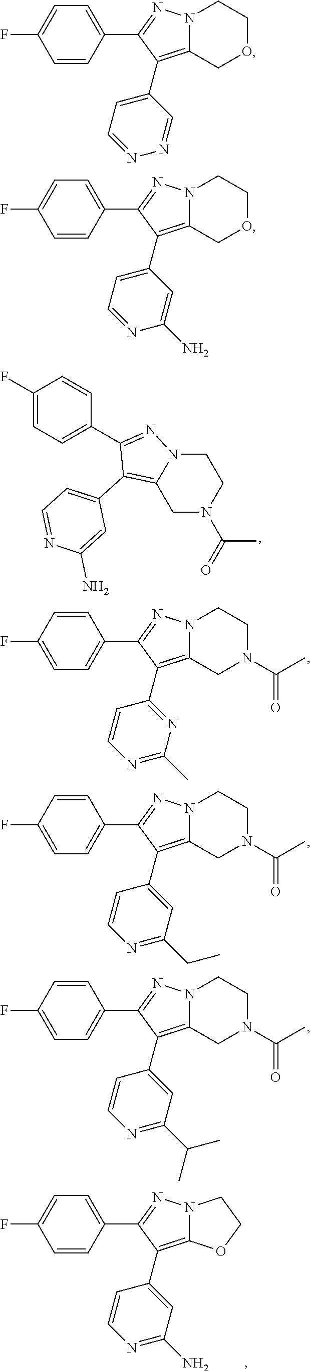

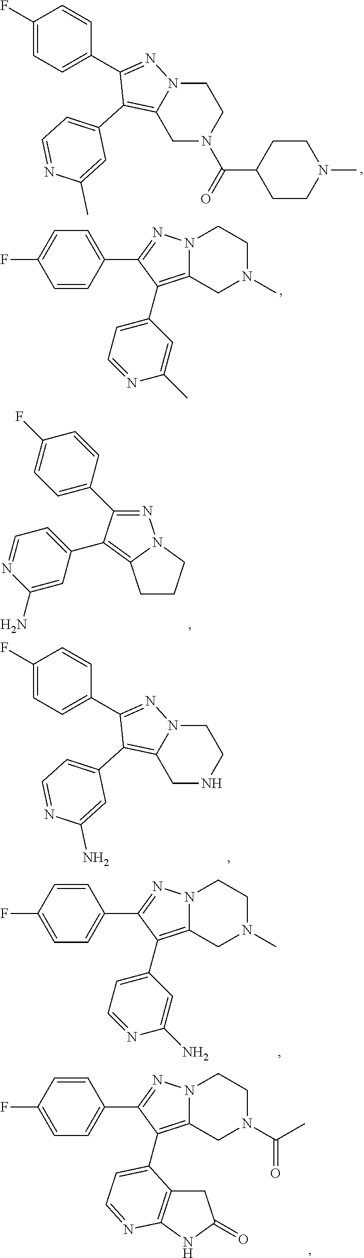

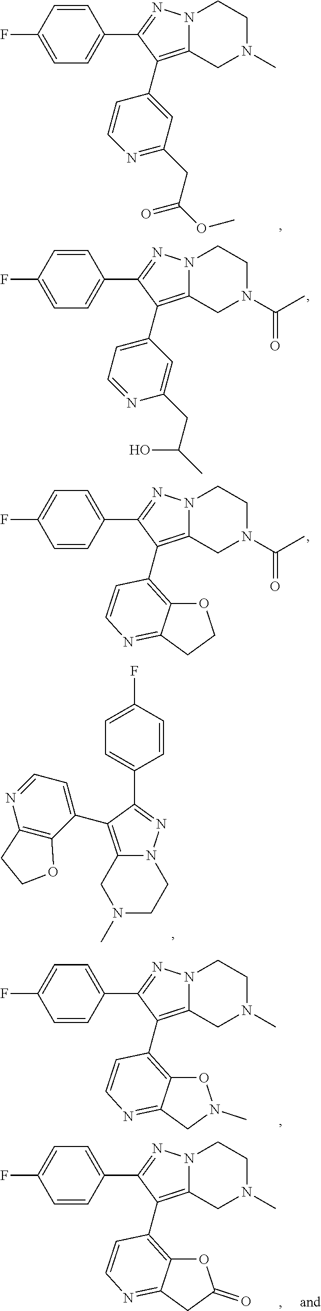

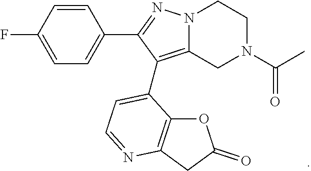 [SMILES: CC(=O)N1CCn2nc(-c3ccc(F)cc3)c(-c3ccnc4c3OC(=O)C4)c2C1]

18-26. (canceled)

27. The compound according to claim 1, wherein the compound is at least one selected from the group consisting of:

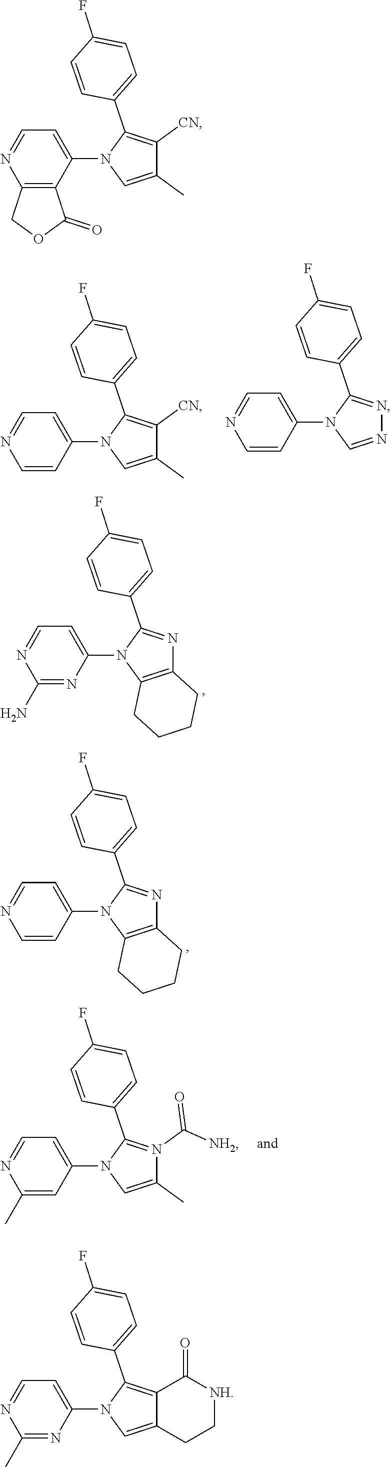

28-37. (canceled)

38. The compound according to claim 1, wherein the compound is at least one selected from the group consisting of:

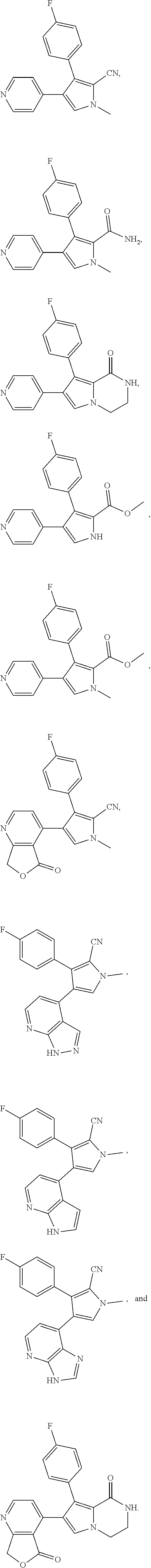

39-47. (canceled)

48. The compound according to claim 1, wherein the compound is at least one selected from the group consisting of: 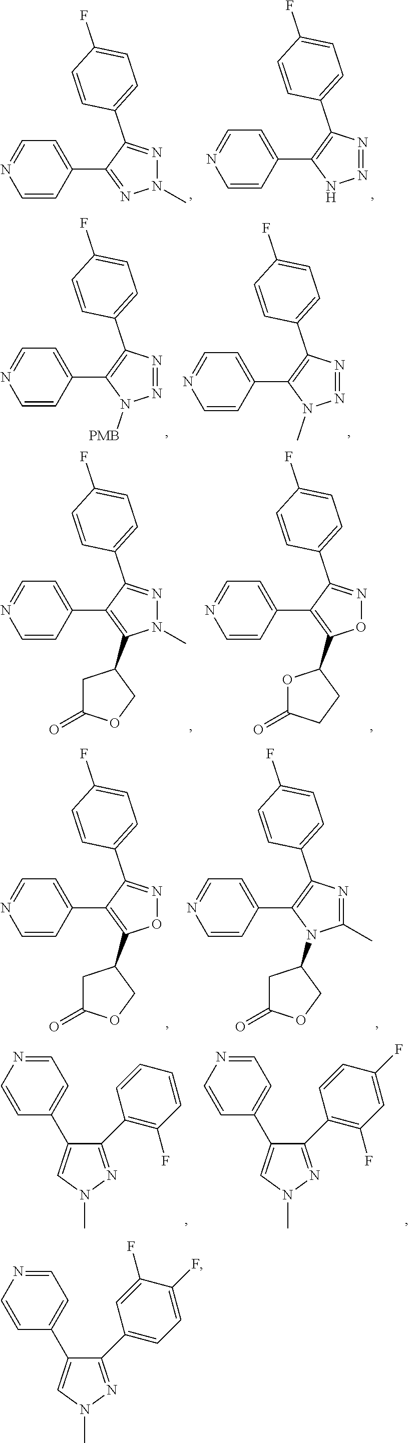 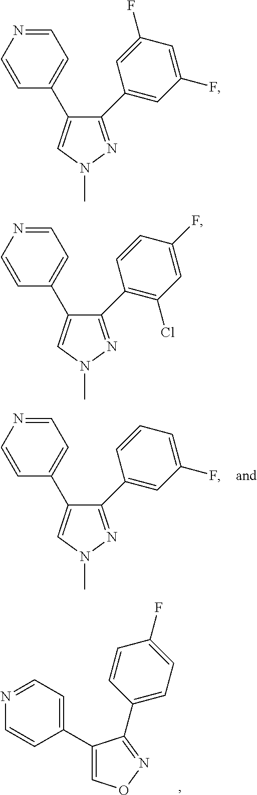
wherein PMB represents group

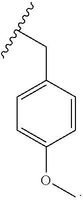 [SMILES: *Cc1ccc(OC)cc1]

49-55. (canceled)

56. The compound according to claim 1, wherein the compound is at least one selected from the group consisting of:

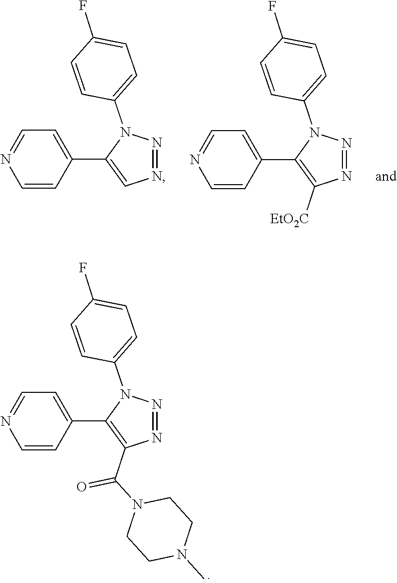

57-61. (canceled)

62. The compound according to claim 1, wherein the compound is

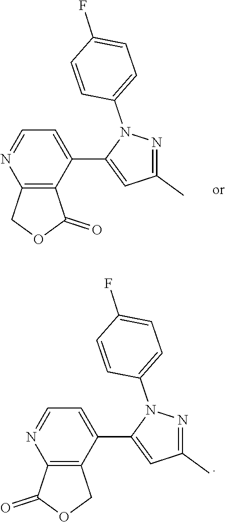 [SMILES: Cc1cc(-c2ccnc3c2C(=O)OC3)n(-c2ccc(F)cc2)n1 ; Cc1cc(-c2ccnc3c2COC3=O)n(-c2ccc(F)cc2)n1]

63. A compound of 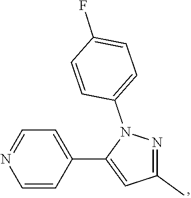 [SMILES: Cc1cc(-c2ccncc2)n(-c2ccc(F)cc2)n1]
or a pharmaceutically acceptable salt thereof.

64. A pharmaceutical composition, comprising the compound of claim 1 or 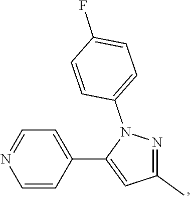 [SMILES: Cc1cc(-c2ccncc2)n(-c2ccc(F)cc2)n1]
or the pharmaceutically acceptable salt thereof, and optionally a pharmaceutically acceptable carrier.

65. A method for inhibiting CK1 delta or CK1 epsilon activity, comprising administering an effective amount of the compound according to claim 1 or 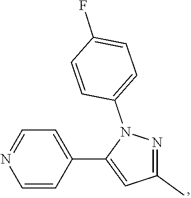 [SMILES: Cc1cc(-c2ccncc2)n(-c2ccc(F)cc2)n1]
or a pharmaceutically acceptable salt thereof, or the pharmaceutical composition comprising the compound or the pharmaceutically acceptable salt thereof.

66. The method according to claim 65, wherein the method is an in vitro method, an ex vivo method, or an in vivo method.

67. A method for treating a neurological and/or psychiatric disease or disorder in a mammal, comprising:
administering to the mammal a therapeutically effective amount of a compound of claim 1 or 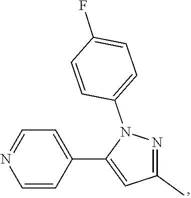 [SMILES: Cc1cc(-c2ccncc2)n(-c2ccc(F)cc2)n1]
or a pharmaceutically acceptable salt thereof, or the pharmaceutical composition comprising the compound or the pharmaceutically acceptable salt thereof.

68. The method according to claim 67, wherein the disease or disorder is a mood disorder, a sleep disorder, or a circadian disorder.

69. The method according to claim 68, wherein the mood disorder is at least one selected from the group consisting of: a depressive disorder and a bipolar disorder.

70. A method for treating cancer in a mammal, comprising:
administering to the mammal a therapeutically effective amount of a compound of claim 1 or 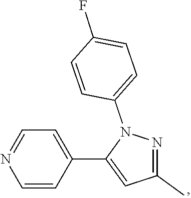 [SMILES: Cc1cc(-c2ccncc2)n(-c2ccc(F)cc2)n1]
a pharmaceutically acceptable salt thereof, or the pharmaceutical composition comprising the compound or the pharmaceutically acceptable salt thereof.

71. The method according to claim 70, wherein the cancer is a solid tumor, a blood cancer, or a lymphoma.

72. The method according to claim 70, wherein the cancer is at least one selected from the group consisting of breast cancer, melanoma, leukemia, liver cancer, and brain cancer.
